(19) United States
(12) Patent Application Publication
(10) Pub. No.: US 2007/0259906 A1
(43) Pub. Date: Nov. 8, 2007

(54) Method of treatment of diarrhea-predominant irritable bowel syndrome in a subject

(76) Inventor: Steven David Caras, Marietta, GA (US)

Correspondence Address: MAYER, BROWN, ROWE & MAW LLP, 1909 K STREET, N.W., WASHINGTON, DC, 20006, US

(21) Appl. No.: 11/362,534

(22) Filed: Feb. 27, 2006

Related U.S. Application Data
(60) Provisional application No. 60/656,103, filed on Feb. 25, 2005.
(60) Provisional application No. 60/656,093, filed on Feb. 25, 2005.
(60) Provisional application No. 60/656,094, filed on Feb. 25, 2005.
(60) Provisional application No. 60/656,095, filed on Feb. 25, 2005.
(60) Provisional application No. 60/656,098, filed on Feb. 25, 2005.
(60) Provisional application No. 60/656,099, filed on Feb. 25, 2005.
(60) Provisional application No. 60/656,100, filed on Feb. 25, 2005.
(60) Provisional application No. 60/656,101, filed on Feb. 25, 2005.
(60) Provisional application No. 60/656,102, filed on Feb. 25, 2005.
(60) Provisional application No. 60/656,106, filed on Feb. 25, 2005.
(60) Provisional application No. 60/656,423, filed on Feb. 25, 2005.
(60) Provisional application No. 60/656,431, filed on Feb. 25, 2005.
(60) Provisional application No. 60/656,434, filed on Feb. 25, 2005.
(60) Provisional application No. 60/656,437, filed on Feb. 25, 2005.
(60) Provisional application No. 60/656,438, filed on Feb. 25, 2005.
(60) Provisional application No. 60/656,439, filed on Feb. 25, 2005.
(60) Provisional application No. 60/656,440, filed on Feb. 25, 2005.
(60) Provisional application No. 60/656,441, filed on Feb. 25, 2005.
(60) Provisional application No. 60/656,444, filed on Feb. 25, 2005.
(60) Provisional application No. 60/679,674, filed on May 11, 2005.
(60) Provisional application No. 60/679,677, filed on May 11, 2005.
(60) Provisional application No. 60/708,768, filed on Aug. 17, 2005.
(60) Provisional application No. 60/708,800, filed on Aug. 17, 2005.
(60) Provisional application No. 60/708,799, filed on Aug. 17, 2005.
(60) Provisional application No. 60/721,080, filed on Sep. 28, 2005.

(51) Int. Cl. A61K 31/437; A61P 1/00

(52) U.S. Cl. 514/284

(57) ABSTRACT

A method of treatment of diarrhea-predominant IBS in a subject, comprising administering a composition comprising cilansetron or a pharmaceutically acceptable derivative thereof to achieve a plasma cilansetron concentration between about 0.1 ng/mL and about 25 ng/mL.

### CROSS REFERENCE TO RELATED APPLICATIONS

This application claims the benefit of U.S. Provisional Application Ser. Nos. 60/656,103; 60/656,093; 60/656,094; 60/656,095; 60/656,098; 60/656,099; 60/656,100; 60/656,101; 60/656,102; 60/656,106; 60/656,423; 60/656,431; 60/656,434; 60/656,437; 60/656,438; 60/656,439; 60/656,440; 60/656,441, and 60/656,444, all of which were filed Feb. 25, 2005. These applications, in their entirety, are incorporated herein by reference.

### FIELD OF THE INVENTION

The invention relates to the use of cilansetron and, more particularly, to a method for treating diarrhea-predominant irritable bowel syndrome in a subject.

### BACKGROUND OF THE INVENTION

Irritable bowel syndrome (IBS) affects approximately 10-20% of the general population. It is the most common disease diagnosed by gastroenterologists and one of the most common disorders seen by primary care physicians.

Despite the high incidence of IBS among men and women, however, treatments for IBS and for diarrhea-predominant and nonconstipated forms of IBS are only partially effective in providing adequate symptom relief to patients.

Accordingly, there remains a need for improved methods of treatment of IBS in patients in need thereof.

### SUMMARY OF THE INVENTION

This invention provides a method of treatment of diarrhea-predominant IBS in a subject, comprising administering a composition comprising cilansetron or a pharmaceutically acceptable derivative thereof to achieve a plasma cilansetron concentration between about 0.1 ng/mL and about 25 ng/mL.

In another aspect, the invention provides a method of treatment of diarrhea-predominant IBS in a subject, comprising administering a sufficient amount of a composition comprising cilansetron or a pharmaceutically acceptable derivative thereof to reach a mean plasma cilansetron concentration across a statistically significant population of subjects of between about 0.1 ng/mL and about 25 ng/mL.

In another aspect, the invention provides a method of treatment of diarrhea-predominant IBS in a subject, comprising administering a daily dose of a composition comprising cilansetron or a pharmaceutically acceptable derivative thereof in an amount sufficient to substantially maintain a plasma cilansetron concentration of between about 0.1 ng/mL and about 25 ng/mL.

In another aspect, the invention provides a method for treatment of nonconstipated IBS in subject, comprising administering a composition comprising cilansetron or a pharmaceutically acceptable derivative thereof to achieve a plasma cilansetron concentration between about 0.1 ng/mL and about 25 ng/mL.

In another aspect, the invention provides a method for treatment of nonconstipated IBS in subject, comprising administering a sufficient amount of a composition comprising cilansetron or a pharmaceutically acceptable derivative thereof to reach a mean plasma cilansetron concentration across a statistically significant population of subjects of between about 0.1 ng/mL and about 25 ng/mL.

In another aspect, the invention provides a method for treatment of nonconstipated IBS in subject, comprising administering a daily dose of a composition comprising cilansetron or a pharmaceutically acceptable derivative thereof in an amount sufficient to substantially maintain a plasma cilansetron concentration of between about 0.1 ng/mL and about 25 ng/mL.

In another aspect, the invention provides a method of improving quality of life in a subject having diarrhea-predominant IBS, comprising administering a composition comprising cilansetron or a pharmaceutically acceptable derivative thereof to achieve a plasma cilansetron concentration between about 0.1 ng/mL and about 25 ng/mL.

In another aspect, the invention provides a method of improving quality of life in a subject having diarrhea-predominant IBS, comprising administering a sufficient amount of a composition comprising cilansetron or a pharmaceutically acceptable derivative thereof to reach a mean plasma cilansetron concentration across a statistically significant population of subjects of between about 0.1 ng/mL and about 25 ng/mL.

In another aspect, the invention provides a method of improving quality of life in a subject having diarrhea-predominant IBS, comprising administering a daily dose of a composition comprising cilansetron or a pharmaceutically acceptable derivative thereof in an amount sufficient to substantially maintain a plasma cilansetron concentration of between about 0.1 ng/mL and about 25 ng/mL.

In another aspect, the invention provides a method of treatment of diarrhea-predominant IBS in a subject receiving selective serotonin reuptake inhibitor (SSRI) therapy, comprising administering a composition comprising cilansetron or a pharmaceutically acceptable derivative thereof to achieve a plasma cilansetron concentration between about 0.1 ng/mL and about 25 ng/mL.

These and other aspects of the present invention are describe more fully herein below.

### BRIEF DESCRIPTION OF THE DRAWINGS

FIGS. 1(a)-(d) show dissolution profile plots for cilansetron 4 mg capsules.

FIG. 2(a)-(d) show dissolution profile plots for cilansetron 8 mg capsules.

FIG. 3 is a comparative dissolution profile plot of cilansetron 8 mg tablet and 8 mg capsule formulations

FIG. 4 is a Linear and Semi-Logarithmic Geometric Mean plot of cilansetron mean plasma concentrations for tablet and oral solutions.

FIG. 5 is a Linear and Semi-Logarithmic Geometric Mean plot of (4R)-hydroxy cilansetron mean plasma concentrations for tablet and oral solutions.

FIG. 6 is a Linear and Semi-Logarithmic Geometric Mean plot of (4S)-hydroxy cilansetron mean plasma concentrations for tablet and oral solutions.

FIG. 7 is a Linear and Semi-Logarithmic Geometric Mean plot of cilansetron mean plasma concentrations under fasted and high-fat conditions.

FIG. 8 is a Linear and Semi-Logarithmic Geometric Mean plot of (4R)-hydroxy cilansetron mean plasma concentrations under fasted and high-fat conditions.

FIG. 9 is a Linear and Semi-Logarithmic Geometric Mean plot of (4S)-hydroxy cilansetron mean plasma concentrations under fasted and high-fat conditions.

FIG. 10 is a Linear and Semi-Logarithmic Geometric Mean plot of cilansetron mean plasma concentrations following a single dose or at steady state.

FIG. 11 is a Linear and Semi-Logarithmic Geometric Mean plot of (4R)-hydroxy cilansetron mean plasma concentrations following a single dose or at steady state.

FIG. 12 is a Linear and Semi-Logarithmic Geometric Mean plot of (4S)-hydroxy cilansetron mean plasma concentrations following a single dose or at steady state.

FIG. 13(a)-(d) shows plots of plasma trough concentrations of cilansetron and its 4-hydroxy metabolites following oral treatment.

FIG. 14 is a box plot of cilansetron AUC(0-inf) following 4 mg to 16 mg administration.

FIG. 15 is a box plot of cilansetron dose-normalized AUC(0-inf) following 4 mg to 16 mg administration.

FIG. 16 is a box plot of cilansetron AUC(0-inf) following 2 mg to 64 mg administration.

FIG. 17 is a box plot of cilansetron Cmax following 2 mg to 64 mg administration.

FIG. 18 is a box plot of cilansetron dose-normalized AUC (0-inf) following 2 mg to 64 mg administration.

FIG. 19 is a box plot of cilansetron dose-normalized Cmax following 2 mg to 64 mg administration.

FIG. 20 is a box plot of cilansetron AUC (0-T) following 2 mg to 32 mg TID administration.

FIG. 21 is box plot of cilansetron Cmax following 2 mg to 32 mg TID administration.

FIG. 22 is a box plot of cilansetron dose-normalized AUC (0-i) following 2 mg to 32 mg TID administration.

FIG. 23 is a box plot of cilansetron dose-normalized Cmax following 2 mg to 32 mg TID administration.

FIG. 24 is a plot of mean ¹⁴C-radioactivity and unchanged cilansetron plasma concentration following a single IV dose of ¹⁴C-labeled cilansetron.

FIG. 25 is a plot of mean ¹⁴C-radioactivity and unchanged cilansetron plasma concentration following a single oral dose of ¹⁴C-labeled cilansetron.

FIG. 26 shows a metabolic scheme for cilansetron in humans and other animal species.

FIG. 27 is a Linear and Semi-Logarithmic Geometric Mean plot of cilansetron mean plasma concentrations in young and elderly male and female subjects following 8 mg administration.

FIG. 28 is a Linear and Semi-Logarithmic Geometric Mean plot of cilansetron mean plasma concentrations in young and elderly male and female subjects following 8 mg TID administration.

FIG. 29(a)-(b) show pharmacokinetic values for cilansetron in healthy and renally-impaired subjects following 2 mg TID administration.

FIG. 30(a)-(b) show pharmacokinetic values for (4R)-hydroxy cilansetron in healthy and renally-impaired subjects following 2 mg TID administration.

FIG. 31(a)-(b) show pharmacokinetic values for (4S)-hydroxy cilansetron in healthy and renally-impaired subjects following 2 mg TID administration.

FIG. 32 is regression analysis plot for individual cilansetron AUC(0-T) values versus creatinine clearance.

FIG. 33 is regression analysis plot for individual (4R)-hydroxy cilansetron AUC(0-T) values versus creatinine clearance.

FIG. 34 is regression analysis plot for individual (4S)-hydroxy cilansetron AUC(0-,) values versus creatinine clearance.

FIG. 35 is a Linear and Semi-Logarithmic Geometric Mean plot of cilansetron mean plasma concentrations in hepatically impaired and healthy subjects.

FIG. 36 is a Linear and Semi-Logarithmic Geometric Mean plot of (4R)-hydroxy cilansetron and (4S)-hydroxy cilansetron mean plasma concentrations in hepatically impaired and healthy subjects.

FIG. 37 is a plot of mean plasma cilansetron concentrations following administration of cilansetron alone or in combination with Maalox liquid.

FIG. 38 is a plot of mean plasma ethinyl estradiol concentrations following administration of Ortho Tri-Cyclen alone or in combination with cilansetron.

FIG. 39 is a plot of mean plasma norgestrel concentrations following administration of Ortho Tri-Cyclen alone or in combination with cilansetron.

FIG. 40 is a plot of mean plasma 17-deacetyl-norgestimate concentrations following administration of Ortho Tri-Cyclen alone or in combination with cilansetron.

FIG. 41(a)-(c) shows plots of mean serum concentrations or follicle stimulating hormone, luteinizing hormone and progesterone following administration of Ortho Tri-Cyclen alone or in combination with cilansetron.

FIG. 42 is a plot of mean plasma cilansetron concentrations following administration of cilansetron alone or in combination with fluvoxamine.

FIG. 43 is a plot of mean plasma (4R)-hydroxy cilansetron concentrations following administration of cilansetron alone or in combination with fluvoxamine.

FIG. 44 is a plot of mean plasma (4S)-hydroxy cilansetron concentrations following administration of cilansetron alone or in combination with fluvoxamine.

FIG. 45 is a box plot of AUC(0-τ) values for cilansetron and its 4-hydroxy metabolites following administration of cilansetron alone or in combination with fluvoxamine.

FIG. 46 is a plot of mean cilansetron concentration following administration of cilansetron alone or in combination with ketoconazole.

FIG. 47 is a plot of mean (4R)-hydroxy cilansetron concentration following administration of cilansetron alone or in combination with ketoconazole.

FIG. 48 is a plot of mean (4S)-hydroxy cilansetron concentration following administration of cilansetron alone or in combination with ketoconazole.

FIG. 49(a)-(c) show box plots of AUC(0-τ) values for cilansetron and its 4-hydroxy metabolites following administration of cilansetron alone or in combination with ketoconazole.

FIG. 50 is a plot of mean cilansetron concentration following administration of cilansetron alone or in combination with paroxetine.

FIG. 51 is a plot of mean (4R)-hydroxy cilansetron concentration following administration of cilansetron alone or in combination with paroxetine.

FIG. 52 is a plot of mean (4S)-hydroxy cilansetron concentration following administration of cilansetron alone or in combination with paroxetine.

FIG. 53(a)-(c) show box plots of AUC(0-τ) values for cilansetron and its 4-hydroxy metabolites following administration of cilansetron alone or in combination with paroxetine.

FIG. 54 is a scatter plot of observed plasma cilansetron concentration versus post-dose time for 1, 2, 8, and 16 mg cilansetron treatments.

FIG. 55 is a scatter plot of observed plasma (4R)-hydroxy cilansetron concentration versus post-dose time for 1, 2, 8, and 16 mg cilansetron treatments.

FIG. 56 is a scatter plot of observed plasma (4S)-hydroxy cilansetron concentration versus post-dose time for 1, 2, 8, and 16 mg cilansetron treatments.

FIG. 57 is a scatter plot of individual placebo-corrected QTc intervals versus cilansetron concentrations following 16 mg cilansetron administration.

FIG. 58 is a plot summary of adequate relief of IBS symptoms by week for the intent-to-treat (ITT) population of subjects.

FIG. 59 is a plot summary of adequate relief of abdominal pain/and/or discomfort by week for the ITT population of subjects.

FIG. 60 is a plot summary of adequate relief of abnormal bowel habits by week for the ITT population of subjects.

FIG. 61 is a plot of the percentage of subjects with adequate relief of IBS symptoms by week during a four-month treatment period.

FIG. 62 is a plot of the percentage of subjects with adequate relief of abdominal pain and/or discomfort by week during a four-month treatment period.

FIG. 63 is a plot of the percentage of subjects with adequate relief of abnormal bowel habits by week during a four-month treatment period.

FIG. 64 is a plot of change in mean adequate relief of IBS symptoms with cilansetron or placebo treatment.

FIG. 65 is a plot of change in mean adequate relief of abdominal pain and/or discomfort with cilansetron or placebo treatment.

FIG. 66 is a plot of change in mean adequate relief of abnormal bowel habits with cilansetron or placebo treatment.

FIG. 67 is a plot of change in mean daily abdominal pain and/or discomfort with cilansetron or placebo treatment.

FIG. 68 is a plot of change in mean daily abdominal pain and/or discomfort with cilansetron or placebo treatment.

FIG. 69 is a plot of change in mean daily abdominal pain and/or discomfort with cilansetron or placebo treatment.

FIG. 70 is a plot of change in mean daily stool consistency with cilansetron or placebo treatment.

FIG. 71 is a plot of change in mean daily stool consistency with cilansetron or placebo treatment.

FIG. 72 is a plot of change in mean daily stool consistency with cilansetron or placebo treatment.

FIG. 73 is a plot of change in mean daily stool frequency with cilansetron or placebo treatment.

FIG. 74 is a plot of change in mean daily stool frequency with cilansetron or placebo treatment.

FIG. 75 is a plot of change in mean daily stool frequency with cilansetron or placebo treatment.

FIG. 76 is a plot of change in daily urgency with cilansetron or placebo treatment.

FIG. 77 is a plot of change in mean urgency with cilansetron or placebo treatment.

FIG. 78 is a plot of change in mean urgency with cilansetron or placebo treatment.

FIG. 79 is a plot of change in mean bloating or abdominal distention with cilansetron or placebo treatment.

FIG. 80 is a plot of change in mean bloating or abdominal distention with cilansetron or placebo treatment.

FIG. 81 is a plot of change in mean bloating or abdominal distention with cilansetron or placebo treatment.

FIG. 82 is a plot of mean change in overall quality of life from baseline to endpoint of treatment with cilansetron or placebo.

FIG. 83 is a plot of mean change in overall quality of life from baseline to endpoint of treatment with cilansetron or placebo.

FIG. 84 is a plot of mean change in overall quality of life from baseline to endpoint of treatment with cilansetron or placebo.

FIG. 85 is a plot of mean interruption of activities from baseline to endpoint of treatment with cilansetron or placebo.

FIG. 86 is a plot of mean interruption of activities from baseline to endpoint of treatment with cilansetron or placebo.

FIG. 87 is a plot of mean interruption of activities from baseline to endpoint of treatment with cilansetron or placebo.

### DESCRIPTION OF THE INVENTION

While the present invention is capable of being embodied in various forms, the description below of several embodiments is made with the understanding that the present disclosure is to be considered as an exemplification of the invention, and is not intended to limit the invention to the specific embodiments illustrated. Headings are provided for convenience only and are not to be construed to limit the invention in any way. Embodiments illustrated under any heading may be combined with embodiments illustrated under any other heading.

The use of numerical values in the various ranges specified in this application, unless expressly indicated otherwise, are stated as approximations as though the minimum and maximum values within the stated ranges were both preceded by the word “about.” In this manner, slight variations above and below the stated ranges can be used to achieve substantially the same results as values within the ranges. As used herein, the terms “about” and “approximately” when referring to a numerical value shall have their plain and ordinary meanings to one skilled in the art of pharmaceutical sciences or the art relevant to the range or element at issue. The amount of broadening from the strict numerical boundary depends upon many factors. For example, some of the factors to be considered may include the criticality of the element and/or the effect a given amount of variation will have on the performance of the claimed subject matter, as well as other considerations known to those of skill in the art. Thus, as a general matter, “about” or “approximately” broaden the numerical value. For example, in some cases, “about” or “approximately” may mean ±5%, or ±10%, or ±20%, or ±30% depending on the relevant technology. Also, the disclosure of ranges is intended as a continuous range including every value between the minimum and maximum values recited.

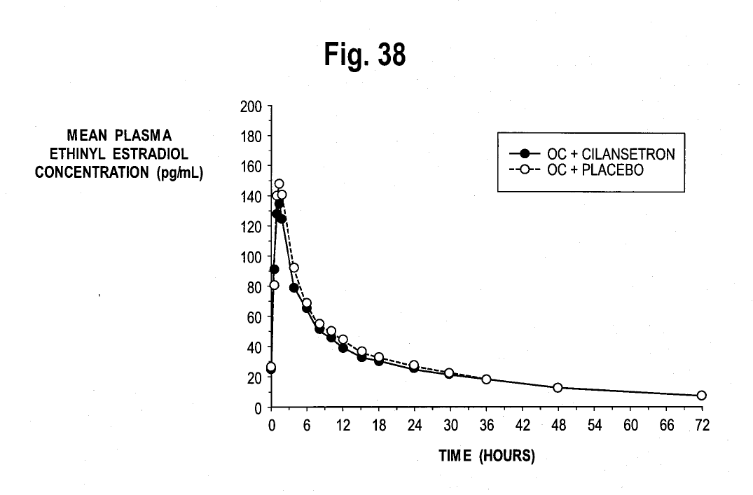

It is to be understood that any ranges, ratios and ranges of ratios that can be formed by any of the numbers or data present herein represent further embodiments of the present invention. This includes ranges that can be formed that do or do not include a finite upper and/or lower boundary. For example, by way of illustration and not limitation, referring to FIG. 38, wherein it is illustrated that the difference in mean plasma concentration of EE between OC+cilansetron and OC administration alone is less than about 10 pg/mL or even less than about 1 pg/mL. Accordingly, the skilled person will appreciate that such ratios, ranges and values are unambiguously derivable from the data presented herein.

The following acronyms are used herein: AAG (Alpha-1 acid glycoprotein); ANOVA (Analysis of variance); AUC (Area under curve); AUC(0-inf) (Area under curve from time zero to infinity); AUC(0-t) (Area under curve from time zero to the concentration time point t); AUC (O-last) (Area under curve over the dosing interval); AUC (0-τ) (Area under curve over the dosing interval); AUC(0-8) (Area under curve over the dosing interval for a three times a daily dosing regimen); BA (Biovailability); BID (Twice daily); Cl (Confidential interval); CL (Systemic clearance); CL/F (Apparent oral clearance); Cm (Centimeter); Cmax (Maximum concentration observed); Cmin (Minimum concentration observed); CLcr (Creatinine concentration observed); CLr (Renal clearance); CO₂ (Carbon dioxide); % CV(Coefficient of variation (in percent)); CYP (Cytochrome P450 enzyme); DN (Dose normalized); % Dose (urine) (Percent of administered dose excreted in urine); ECG (Electrocardiogram); EE (Ethinyl estradio); F (Females); FDA (Food and Drug Administration); FSH (Follicle-stimulating hormone); GI (Gastrointestinal); 5-HT3 (5-hydroxy tryptamine 3); IBS (Irritable Bowel Syndrome); H (Hour); HPLC (High performance liquid chromatography); IC50 (Concentration producing 50% inhibition); ITT (Intent-to-treat population); IV (Intravenous); ka (Oral absorption rate constant); Ka (Affinity constant for protein binding); KC9945 ((+)-(10S) isomer of cilansetron); L (Liter); LH (Luteinizing hormone); m² (Square meter); M (Males); μCi (Microcurie); mg (Milligram); μg (Microgram); Min (Minute); mL (Milliliter); μM (Micromolar); mmHg (Millimeters mercury); mol (Mole); ms (Millisecond); NA (Not applicable); NE (Not estimable); NC (Not calculated); NCCP (Non-cardiac chest pain); ng (Nanogram); ng ([equiv]) (Nanogram equivalents); NGL (Norgestrel); NGM (Norgestimate); 17-d-NGM (17-d-norgestimate); ORC (Ortho Tri-Cyclen®); P450 (Cytochrome P45 Progesterone); PG (Progesterone); PK (Pharmacokinetic); PR (PR interval of an electrocardiogram); QD (Once a day); QRS (QRS interval of an electrocardiogram); QT (QT interval of an electrocardiogram); QTc (QT interval, corrected for heart rate using Bazett's correction); (4R)-OH ((4R)-hydroxy cilansetron); RR (RR interval of an electrocardiogram); SD (Standard deviation); sec (Seconds); Soln (Solution); (4S)-OH ((4S)-hydroxy cilansetron); T1/2 (Half-life); TID (Three times daily); Tlag (Lag time); Tmax (Maximum concentration time); Trt (Treatment); Vss (Steady-state volume of distribution); Vz (Apparent volume of distribution, dependent on terminal rate constant (intravenous administration)); VZ/f (Apparent volume of distribution, dependent on terminal rate constant (oral administration)); V/F (Apparent volume of distribution (model based, oral administration)); and Years (y).

“Cilansetron”, as used herein, is understood to refer to (R)-(−)-4,5,6,8,9,10-hexahydro-10-[(2-methyl-1H-imidazol-1-yl)methyl]-11H-pyrido-[3,2,1-jk]-carbazol-11-one (alternative name: (10R)-5,6,9,10-tetrahydro-10-[(2-methyl-1H-imidazol-1-yl)methyl]-4H-pyrido [3,2,1-jk]carbazol-11(8H)-one), which is disclosed, for example, in U.S. Pat. No. 6,566,369, the contents of which are incorporated herein by reference.

Cilansetron can be administered in any suitable dose, and using any suitable dosing schedule. The dosage of cilansetron administered according to the methods of the present invention may be, for example, from about 0.1 mg to about 40 mg daily, such as from about 1 mg to about 38 mg daily, from about 2 mg to about 36 mg daily, from about 2 mg to about 34 mg daily, from about 2 mg to about 32 mg daily, from about 2 mg to about 30 mg daily, from about 2 mg to about 28 mg daily, from about 2 mg to about 26 mg daily, from about 2 mg to about 24 mg daily, from about 2 mg to about 22 mg daily, from about 2 mg to about 20 mg daily, from about 2 mg to about 18 mg daily, from about 2 mg to about 16 mg daily, from about 2 mg to about 14 mg daily, from about 2 mg to about 12 mg daily, from about 2 mg to about 10 mg daily, from about 2 mg to about 8 mg daily (such as or from about 4 mg to about 8 mg daily, from about 2 mg to about 6 mg daily, or from about 2 mg to about 4 mg daily. Moreover, cilansetron may be administered one or more times a day, such as two or more, three or more, four or more, five or more, or even six or more times daily. In a preferred embodiment, 2 mg cilansetron is administered three times daily (TI)). Moreover, cilansetron can be administered on alternate days or on consecutive days. Cilansetron can also be administered in any suitable formulation such as, for example, a tablet, capsule, gelcap, or solution (e.g., injectable or inhalable solution). In one embodiment, cilansetron is in the form of a 2 mg, 4 mg or 8 mg capsule. In another embodiment, cilansetron is in the form of a 4 mg/2 mL or 8 mg/4 mL solution. In another embodiment, cilansetron is in the form of a 1 mg, 2 mg, 4 mg, 8 mg, 16 mg or 32 mg tablet.

Cilansetron may be administered in the form of a any pharmacologically acceptable acid addition salts, as described for example in U.S. Pat. No. 6,566,369 (the entire contents of which are incorporated herein by reference. In one embodiment, cilansetron is administered in the form of cilansetron hydrochloride or cilansetron hydrochloride monohydrate. Additionally, cilansetron can be administered in any form and in combination with any known diluent, filler, salt, buffer, stabilizer, solubilizer, lipid, or other material, as disclosed, for example in the '369 patent. In one embodiment, cilansetron is administered in a composition comprising: 4 parts cilansetron hydrochloride monohydrate, 30 parts corn starch, 70 parts lactose, 5 parts Kollidon 25®, 2 parts magnesium stearate, and 3 parts talcum, as described, for example in U.S. Pat. No. 6,566,369 (the entire contents of which are incorporated herein by reference). In an embodiment, cilansetron is administered in a 2 mg film-coated tablet for oral use in humans, comprising
(i) about 1.5 mg to about 3 mg cilansetron.HCl.H₂0,
(ii) about 40 mg to about 60 mg corn starch,
(iii) about 70 mg to about 100 mg mannitol,
(iv) about 3 mg to about 7 mg povidone,
(v) about 0.05 mg to about 1 mg citric acid monohydrate,
(vi) about 1 mg to about 5 mg crospovidone,
(vii) about 0.05 mg to about 2 mg colloidal silica, and
(viii) about 1 mg to about 3 mg stearic acid.
   In another embodiment, cilansetron is administered in a 2 mg film-coated tablet for oral use in humans, comprising: 2.34 mg cilansetron.HCl.H₂0, 51.78 mg maize starch, 84.48 mg mannitol; 4.90 mg povidone; 0.50 mg citric acid monohydrate; 3.0 5 mg crospovidone; 1.0 mg colloidal anhydrous silica; 2.0 mg stearic acid; and a coating containing Opadry® 03B28686 (white) or Opadry® Y-1-7000 (white).

This invention provides a method of treatment of diarrhea-predominant IBS in a subject, comprising providing a composition comprising cilansetron or a pharmaceutically acceptable derivative thereof, and administering a dose of the composition to the subject in an amount sufficient for the cilansetron to reach the bloodstream of the subject wherein a plasma cilansetron concentration between about 0.1 ng/mL and about 25 ng/mL is achieved between about 0.01 hours and about 24 hours following the administering of the composition to the subject. In one aspect, the plasma cilansetron concentration achieved by the method is achieved between (i) a first value that is greater than about 0.1 hours, greater than about 0.2 hours, greater than about 0.3 hours, greater than about 0.4 hours, greater than about 0.5 hours, greater than about 0.6 hours, greater than about 0.7 hours, greater than about 0.8 hours, greater than about 0.9 hours, greater than about 1 hour, greater than about 1.1 hours, greater than about 1.2 hours, greater than about 1.3 hours, greater than about 1.4 hours, or greater than about 1.5 hours, and (ii) a second value that is less than about 23 hours, less than about 22 hours, less than about 21 hours, less than about 20 hours, less than about 19 hours, less than about 18 hours, less than about 17 hours, less than about 16 hours, less than about 15 hours, less than about 14 hours, less than about 13 hours, less than about 12 hours, less than about 11 hours, less than about 10 hours, less than about 9 hours, less than about 8 hours, less than about 7 hours, less than about 6 hours, less than about 5 hours, less than about 4 hours, less than about 3 hours, or less than about 2 hours, following administration. In an aspect, the plasma cilansetron concentration achieved by the method is achieved between about 0.01 hours and about 20 hours, between about 0.1 hours and about 18 hours, between about 0.1 hours and about 15 hours, between about 0.1 hours and about 12 hours, between about 0.1 hours and about 8 hours, or between about 0.1 hours and about 4 hours, following administration.

In another aspect, cilansetron is administered in an amount sufficient to achieve a plasma cilansetron concentration between (i) a first value that is greater than about 0.2 ng/mL, greater than about 0.4 ng/mL, greater than about 0.6 ng/mL, greater than about 0.8 ng/mL, greater than about 1.0 ng/mL, greater than about 1.2 ng/mL, greater than about 1.5 ng/mL, greater than about 2.0 ng/mL, greater than about 2.5 ng/mL, greater than about 3.0 ng/mL, greater than about 3.5 ng/mL, greater than about 4.0 ng/mL, greater than about 4.5 ng/mL, or greater than about 5.0 ng/mL and (ii) a second value that is less than about 25 ng/mL, less than about 24 ng/mL, less than about 23 ng/mL, less than about 22 ng/mL, less than about 21 ng/mL, less than about 20 ng/mL, less than about 19 ng/mL, less than about 18 ng/mL, less than about 17 ng/mL, less than about 16 ng/mL, less than about 15 ng/mL, less than about 14 ng/mL, less than about 13 ng/mL, less than about 12 ng/mL, less than about 11 ng/mL, less than about 10 ng/mL, less than about 9 ng/mL, less than about 8 ng/mL, less than about 7 ng/mL, or less than about 6 ng/mL. In an aspect, for example, cilansetron is administered in an amount sufficient to achieve a plasma cilansetron concentration between about 0.5 ng/mL and about 20 ng/mL, between about 1 ng/mL and about 15 ng/mL, between about 3 ng/mL and about 12 ng/mL, or between about 4 ng/mL and about 10 ng/mL, following administration.

In another aspect, the invention provides a method of treatment of diarrhea-predominant IBS in a subject, comprising providing a composition comprising cilansetron or a pharmaceutically acceptable derivative thereof, and administering a dose of the composition to the subject in an amount sufficient for the cilansetron to reach the bloodstream of the subject wherein a plasma cilansetron concentration between about 3 ng/mL and about 12 ng/mL is achieved between about 0.1 hours and about 12 hours following the administering of the composition to the subject.

In another embodiment, the invention provides a method of treatment of diarrhea-predominant IBS in a subject, comprising providing a composition comprising cilansetron or a pharmaceutically acceptable derivative thereof, and administering a dose of the composition to the subject in an amount sufficient for the cilansetron to reach the bloodstream of the subject wherein the mean plasma cilansetron concentration across a statistically significant population of subjects is between about 0.1 ng/mL and about 25 ng/mL. As used herein, “a statistically significant population” is understood to refer to two or more, three or more, four or more, five or more, ten or more, twenty or more, forty or more, sixty or more, eighty or more, or even one hundred or more persons. For example, a mean plasma cilansetron concentration between 4 ng/mL and about 9 ng/mL across a statistically significant population of subjects would be understood by skill to refer to a measured mean plasma cilansetron concentration of between 4 ng/mL and about 9 ng/mL as measured across a statistically significant number of subjects.

In one aspect, the mean plasma cilansetron concentration achieved in a mean time between (i) a first value that is greater than about 0.1 hours, greater than about 0.2 hours, greater than about 0.3 hours, greater than about 0.4 hours, greater than about 0.5 hours, greater than about 0.6 hours, greater than about 0.7 hours, greater than about 0.8 hours, greater than about 0.9 hours, greater than about 1 hour, greater than about 1.1 hours, greater than about 1.2 hours, greater than about 1.3 hours, greater than about 1.4 hours, or greater than about 1.5 hours, and (ii) a second value that is less than about 24 hours, less than about 23 hours, less than about 22 hours, less than about 21 hours, less than about 20 hours, less than about 19 hours, less than about 18 hours, less than about 17 hours, less than about 16 hours, less than about 15 hours, less than about 14 hours, less than about 13 hours, less than about 12 hours, less than about 11 hours, less than about 10 hours, less than about 9 hours, less than about 8 hours, less than about 7 hours, less than about 6 hours, less than about 5 hours, less than about 4 hours, less than about 3 hours, or less than about 2 hours, following administration. In aspect, the mean plasma cilansetron concentration achieved in a mean time between about 0.01 hours and about 24 hours, between about 0.01 hours and about 20 hours, between about 0.1 hours and about 18 hours, between about 0.1 hours and about 15 hours, between about 0.1 hours and about 12 hours, between about 0.1 hours and about 8 hours, or between about 0.1 hours and about 4 hours, following administration.

In another aspect, cilansetron is administered in an amount sufficient to achieve a mean plasma cilansetron concentration between (i) a first value that is greater than about 0.2 ng/mL, greater than about 0.4 ng/mL, greater than about 0.6 ng/mL, greater than about 0.8 ng/mL, greater than about 1.0 ng/mL, greater than about 1.2 ng/mL, greater than about 1.5 ng/mL, greater than about 2.0 ng/mL, greater than about 2.5 ng/mL, greater than about 3.0 ng/mL, greater than about 3.5 ng/mL, greater than about 4.0 ng/mL, greater than about 4.5 ng/mL, or greater than about 5.0 ng/mL and (ii) a second value that is less than about 25 ng/mL, less than about 24 ng/mL, less than about 23 ng/mL, less than about 22 ng/mL, less than about 21 ng/mL, less than about 20 ng/mL, less than about 19 ng/mL, less than about 18 ng/mL, less than about 17 ng/mL, less than about 16 ng/mL, less than about 15 ng/mL, less than about 14 ng/mL, less than about 13 ng/mL, less than about 12 ng/mL, less than about 11 ng/mL, less than about 10 ng/mL, less than about 9 ng/mL, less than about 8 ng/mL, less than about 7 ng/mL, or less than about 6 ng/mL. In an aspect, for example, cilansetron is administered in an amount sufficient to achieve a mean plasma cilansetron concentration between about 0.5 ng/mL and about 20 ng/mL, between about 1 ng/mL and about 15 ng/mL, between about 3 ng/mL and about 12 ng/mL, or between about 4 ng/mL and about 10 ng/mL, following administration.

In another aspect, the invention provides a method of treatment of diarrhea-predominant IBS in a subject, comprising providing a composition comprising cilansetron or a pharmaceutically acceptable derivative thereof, and administering a daily dose of the composition to the subject in an amount sufficient for the cilansetron to reach the bloodstream of the subject wherein a plasma cilansetron concentration is substantially maintained between about 0.1 ng/mL and about 25 ng/mL for at least 12 hours after the subject has been administered the daily dose for at least 7 consecutive days.

In another aspect, the plasma cilansetron concentration is substantially maintained between (i) a first value that is greater than about 0.2 ng/mL, greater than about 0.4 ng/mL, greater than about 0.6 ng/mL, greater than about 0.8 ng/mL, greater than about 1.0 ng/mL, greater than about 1.2 ng/mL, greater than about 1.5 ng/mL, greater than about 2.0 ng/mL, greater than about 2.5 ng/mL, greater than about 3.0 ng/mL, greater than about 3.5 ng/mL, greater than about 4.0 ng/mL, greater than about 4.5 ng/mL, or greater than about 5.0 ng/mL and (ii) a second value that is less than about 25 ng/mL, less than about 24 ng/mL, less than about 23 ng/mL, less than about 22 ng/mL, less than about 21. ng/mL, less than about 20 ng/mL, less than about 19 ng/mL, less than about 18 ng/mL, less than about 17 ng/mL, less than about 16 ng/mL, less than about 15 ng/mL, less than about 14 ng/mL, less than about 13 ng/mL, less than about 12 ng/mL, less than about 11 ng/mL, less than about 10 ng/mL, less than about 9 ng/mL, less than about 8 ng/mL, less than about 7 ng/mL, or less than about 6 ng/mL. In another aspect, for example, cilansetron is administered in an amount sufficient wherein a plasma cilansetron concentration between about 0.5 ng/mL and about 20 ng/mL, between about 1 ng/mL and about 15 ng/mL, between about 3 ng/mL and about 12 ng/mL, or between about 4 ng/mL and about 10 ng/mL, following administration, is substantially maintained for at least 12 hours after the subject has been administered the daily dose for at least 7 consecutive days.

In another embodiment, the plasma cilansetron concentration is substantially maintained between about 0.1 ng/mL and about 25 ng/mL for at least 6 hours, for at least 7 hours, for at least 8 hours, for at least 10 hours, for at least 12 hours, for at least 24 hours, for at least 36 hours, or for at least 48 hours, after the subject has been administered the daily dose for at least 2 consecutive days, for at least 3 consecutive days, for at least 4 consecutive days,.for at least 5 consecutive days, for at least 7 consecutive days, for at least 10 consecutive days, for at least 14 consecutive days, or for at least 21 consecutive days.

In another aspect, the invention provides a method for treatment of nonconstipated IBS (such as alternating diarrhea-predominant/constipative EBS or diarrhea-predominant IBS) in subject, comprising providing a composition comprising cilansetron or a pharmaceutically acceptable derivative thereof, and administering a dose of the composition to the subject in an amount sufficient for the cilansetron to reach the bloodstream of the subject wherein a plasma cilansetron concentration between about 0.1 ng/mL and about 25 ng/mL (or any suitable serum concentration described above) is achieved between about 0.01 hours and about 18 hours (or any suitable period of time described above) following the administering of the composition to the subject.

In another aspect, the invention provides a method for treatment of nonconstipated IBS in subject, comprising providing a composition comprising cilansetron or a pharmaceutically acceptable derivative thereof, and administering a dose of the composition to the subject in an amount sufficient for the cilansetron to reach the bloodstream of the subject wherein a plasma cilansetron concentration between about 3 ng/mL and about 12 ng/mL (or any suitable serum concentration described above) is achieved between about 0.1 hours and about 12 hours (or any suitable period of time described above) following the administering of the composition to the subject.

In another aspect, the invention provides a method for treatment of nonconstipated EBS in subject, comprising providing a composition comprising cilansetron or a pharmaceutically acceptable derivative thereof, and administering a dose of the composition to the subject in an amount sufficient for the cilansetron to reach the bloodstream of the subject wherein the mean plasma cilansetron concentration across a statistically significant population of subjects is between about 0.1 ng/mL and about 25 ng/mL (or any suitable mean serum concentration described above). In another embodiment, the mean serum concentration is achieved in any suitable mean time or period as discussed above.

In another aspect, the invention provides a method for treatment of nonconstipated IBS in subject, comprising providing a composition comprising cilansetron or a pharmaceutically acceptable derivative thereof, and administering a daily dose of the composition to the subject in an amount sufficient for the cilansetron to reach the bloodstream of the subject wherein a plasma cilansetron concentration is substantially maintained between about 0.1 ng/mL and about 25 ng/mL for at least 12 hours (or any suitable duration as discussed above) after the subject has been administered the daily dose for at least 7 consecutive days (or any suitable number of days as discussed above).

In another aspect, the invention provides a method of improving quality of life in a subject having diarrhea-predominant IBS, comprising providing a composition comprising cilansetron or a pharmaceutically acceptable derivative thereof, and administering a dose of the composition to the subject in an amount sufficient for the cilansetron to reach the bloodstream of the subject wherein a plasma cilansetron concentration between about 0.1 ng/mL and about 25 ng/mL (or any suitable serum concentration described above) is achieved between about 0.01 hours and about 18 hours (or any suitable period of time as described above) following the administering of the composition to the subject. Non-limiting examples of improvements in quality of life include decreasing interruption in daily activities, enhancing body image, decreasing food avoidance, enhancing interpersonal relationships, enhancing sexual performance capacity, improving social functioning, improving physical functioning, improving general health, improving vitality, enhancing social functioning, and improving mental health.

In another aspect, the invention provides a method of improving quality of life in a subject having diarrhea-predominant IBS, comprising providing a composition comprising cilansetron or a pharmaceutically acceptable derivative thereof, and administering a dose of the composition to the subject in an amount sufficient for the cilansetron to reach the bloodstream of the subject wherein a plasma cilansetron concentration between about 3 ng/mL and about 12 ng/mL (or any suitable serum concentration described above) is achieved between about 0.1 hours and about 12 hours (or any suitable period of time as described above) following the administering of the composition to the subject.

In another aspect, the invention provides a method of improving quality of life in a subject having diarrhea-predominant IBS, comprising providing a composition comprising cilansetron or a pharmaceutically acceptable derivative thereof, and administering a dose of the composition to the subject in an amount sufficient for the cilansetron to reach the bloodstream of the subject wherein the mean plasma cilansetron concentration across a statistically significant population of subjects is between about 0.1 ng/mL and about 25 ng/mL (or any suitable mean serum concentration described above). In another embodiment, the mean serum concentration is achieved in any suitable mean time or period as discussed above.

In another aspect, the invention provides a method of improving quality of life in a subject having diarrhea-predominant IBS, comprising providing a composition comprising cilansetron or a pharmaceutically acceptable derivative thereof, and administering a daily dose of the composition to the subject in an amount sufficient for the cilansetron to reach the bloodstream of the subject wherein a plasma cilansetron concentration is substantially maintained between about 0.1 ng/mL and about 25 ng/mL for at least 12 hours (or any suitable duration as discussed above) after the subject has been administered the daily dose for at least 7 consecutive days (or any suitable number of days as discussed above).

In another aspect, the invention provides a method for treatment of abnormal bowel habits in a subject afflicted with diarrhea-predominant IBS, comprising providing a composition comprising cilansetron or a pharmaceutically acceptable derivative thereof, and administering a dose of the composition to the subject in an amount sufficient for the cilansetron to reach the bloodstream of the subject wherein a plasma cilansetron concentration between about 0.1 ng/mL and about 25 ng/mL (or any suitable serum concentration as described above) is achieved between about 0.01 hours and about 18 hours (or any suitable period of time described above) following the administering of the composition to the subject. Non-limiting examples of improvements in bowel habits include improving stool consistency, improving bowel movement frequency, decreasing urgency, and decreasing urgency.

In another aspect, the invention provides a method for treatment of abnormal bowel habits in a subject afflicted with diarrhea-predominant IBS, comprising providing a composition comprising cilansetron or a pharmaceutically acceptable derivative thereof, and administering a dose of the composition to the subject in an amount sufficient for the cilansetron to reach the bloodstream of the subject wherein a plasma cilansetron concentration between about 3 ng/mL and about 12 ng/mL is achieved between about 0.1 hours and about 12 hours following the administering of the composition to the subject.

In another aspect, the invention provides a method for treatment of abnormal bowel habits in a subject afflicted with diarrhea-predominant IBS, comprising providing a composition comprising cilansetron or a pharmaceutically acceptable derivative thereof, and administering a dose of the composition to the subject in an amount sufficient for the cilansetron to reach the bloodstream of the subject wherein the mean plasma cilansetron concentration across a statistically significant population of subjects is between about 0.1 ng/mL and about 25 ng/mL (or any suitable mean serum concentration described above). In another embodiment, the mean serum concentration is achieved in any suitable mean time or period as discussed above.

In another aspect, the invention provides a method for treatment of abnormal bowel habits in a subject afflicted with diarrhea-predominant IBS, comprising providing a composition comprising cilansetron or a pharmaceutically acceptable derivative thereof, and administering a daily dose of the composition to the subject in an amount sufficient for the cilansetron to reach the bloodstream of the subject wherein a plasma cilansetron concentration is substantially maintained between about 0.1 ng/mL and about 25 ng/mL for at least 12 hours (or any suitable duration as discussed above) after the subject has been administered the daily dose for at least 7 consecutive days (or any suitable number of days as discussed above).

In another aspect, the invention provides a method for treatment of diarrhea-predominant IBS in a female subject, comprising providing a composition comprising cilansetron or a pharmaceutically acceptable derivative thereof, and administering a dose of the composition to the subject in an amount sufficient for the cilansetron to reach the bloodstream of the subject wherein a plasma cilansetron concentration between about 0.1 ng/mL and about 25 ng/mL (or any suitable serum concentration as described above) is achieved between about 0.01 hours and about 18 hours (or any suitable period of time described above) following the administering of the composition to the subject

In another aspect, the invention provides a method for treatment of diarrhea-predominant IBS in a female subject, comprising providing a composition comprising cilansetron or a pharmaceutically acceptable derivative thereof, and administering a dose of the composition to the subject in an amount sufficient for the cilansetron to reach the bloodstream of the subject wherein a plasma cilansetron concentration between about 3 ng/mL and about 12 ng/mL is achieved between about 0.1 hours and about 12 hours following the administering of the composition to the subject.

In another aspect, the invention provides a method for treatment of diarrhea-predominant IBS in a female subject, comprising providing a composition comprising cilansetron or a pharmaceutically acceptable derivative thereof, and administering a dose of the composition to the subject in an amount sufficient for the cilansetron to reach the bloodstream of the subject wherein the mean plasma cilansetron concentration across a statistically significant population of subjects is between about 0.1 ng/mL and about 25 ng/mL (or any suitable mean serum concentration described above). In another embodiment, the mean serum concentration is achieved in any suitable mean time or period as discussed above.

In another aspect, the invention provides a method for treatment of diarrhea-predominant IBS in a female subject, comprising providing a composition comprising cilansetron or a pharmaceutically acceptable derivative thereof, and administering a daily dose of the composition to the subject in an amount sufficient for the cilansetron to reach the bloodstream of the subject wherein a plasma cilansetron concentration is substantially maintained between about 0.1 ng/mL and about 25 ng/mL for at least 12 hours (or any suitable duration as discussed above) after the subject has been administered the daily dose for at least 7 consecutive days (or any suitable number of days as discussed above).

In another aspect, the invention provides a method for treatment of diarrhea-predominant IBS in a male subject, comprising providing a composition comprising cilansetron or a pharmaceutically acceptable derivative thereof, and administering a dose of the composition to the subject in an amount sufficient for the cilansetron to reach the bloodstream of the subject wherein a plasma cilansetron concentration between about 0.1 ng/mL and about 25 ng/mL (or any suitable serum concentration as described above) is achieved between about 0.01 hours and about 18 hours (or any suitable period of time described above) following the administering of the composition to the subject

In another aspect, the invention provides a method for treatment of diarrhea-predominant IBS in a male subject, comprising providing a composition comprising cilansetron or a pharmaceutically acceptable derivative thereof, and administering a dose of the composition to the subject in an amount sufficient for the cilansetron to reach the bloodstream of the subject wherein a plasma cilansetron concentration between about 3 ng/mL and about 12 ng/mL is achieved between about 0.1 hours and about 12 hours following the administering of the composition to the subject.

In another aspect, the invention provides a method for treatment of diarrhea-predominant IBS in a male subject, comprising providing a composition comprising cilansetron or a pharmaceutically acceptable derivative thereof, and administering a dose of the composition to the subject in an amount sufficient for the cilansetron to reach the bloodstream of the subject wherein the mean plasma cilansetron concentration across a statistically significant population of subjects is between about 0.1 ng/mL and about 25 ng/mL (or any suitable mean serum concentration described above). In another embodiment, the mean serum concentration is achieved in any suitable mean time or period as discussed above.

In another aspect, the invention provides a method for treatment of diarrhea-predominant IBS in a male subject, comprising providing a composition comprising cilansetron or a pharmaceutically acceptable derivative thereof, and administering a daily dose of the composition to the subject in an amount sufficient for the cilansetron to reach the bloodstream of the subject wherein a plasma cilansetron concentration is substantially maintained between about 0.1 ng/mL and about 25 ng/mL for at least 12 hours (or any suitable duration as discussed above) after the subject has been administered the daily dose for at least 7 consecutive days (or any suitable number of days as discussed above).

In another aspect, the invention provides a method for treatment of extended-duration diarrhea-predominant IBS in a subject in need thereof comprising providing a composition comprising cilansetron or a pharmaceutically acceptable derivative thereof, and administering a dose of the composition to the subject in an amount sufficient for the cilansetron to reach the bloodstream of the subject wherein a plasma cilansetron concentration between about 0.1 ng/mL and about 25 ng/mL (or any suitable serum concentration as described above) is achieved between about 0.01 hours and about 18 hours (or any suitable period of time described above) following the administering of the composition to the subject. As used here, “extended duration diarrhea-predominant IBS” is understood to refer to a manifesting by a subject of at least one symptom of IBS-D for a period of time exceeding 12 months, 18 months, 24 months, 30 months, 36 months, 42 months, 48 months, 54 months, 60 months, 66 months, 72 months, 78 months, 84 months, 90 months, 96 months, 102 months, 108 months, 114 months, or 120 months prior to administering of cilansetron or a pharmaceutically acceptable derivative thereof.

In another aspect, the invention provides a method for treatment of extended-duration diarrhea-predominant IBS in a subject in need thereof, comprising providing a composition comprising cilansetron or a pharmaceutically acceptable derivative thereof, and administering a dose of the composition to the subject in an amount sufficient for the cilansetron to reach the bloodstream of the subject wherein a plasma cilansetron concentration between about 3 ng/mL and about 12 ng/mL is achieved between about 0.1 hours and about 12 hours following the administering of the composition to the subject.

In another aspect, the invention provides a method for treatment of extended-duration diarrhea-predominant 1IBS in a subject in need thereof, comprising providing a composition comprising cilansetron or a pharmaceutically acceptable derivative thereof, and administering a dose of the composition to the subject in an amount sufficient for the cilansetron to reach the bloodstream of the subject wherein the mean plasma cilansetron concentration across a statistically significant population of subjects is between about 0.1 ng/mL and about 25 ng/mL (or any suitable mean serum concentration described above). In another embodiment, the mean serum concentration is achieved in any suitable mean time or period as discussed above.

In another aspect, the invention provides a method for treatment of extended-duration diarrhea-predominant IBS in a subject in need thereof, comprising providing a composition comprising cilansetron or a pharmaceutically acceptable derivative thereof, and administering a daily dose of the composition to the subject in an amount sufficient for the cilansetron to reach the bloodstream of the subject wherein a plasma cilansetron concentration is substantially maintained between about 0.1 ng/mL and about 25 ng/mL for at least 12 hours (or any suitable duration as discussed above) after the subject has been administered the daily dose for at least 7 consecutive days (or any suitable number of days as discussed above).

In another aspect, the invention provides a method for treatment of diarrhea-predominant EBS in a subject having a body mass index (BMI) of less than about 40 kg/m², less than about 38 kg/m², less than about 36 kg/m², less than about 34 kg/m², less than about 32 kg/m², less than about 30 kg/m², less than about 28 kg/m², less than about 26 kg/m², less than about 24 kg/m², less than about 22 kg/m², or less than about 20 kg/m², comprising providing a composition comprising cilansetron or a pharmaceutically acceptable derivative thereof, and administering a dose of the composition to the subject in an amount sufficient for the cilansetron to reach the bloodstream of the subject wherein a plasma cilansetron concentration between about 0.1 ng/mL and about 25 ng/mL (or any suitable serum concentration as described above) is achieved between about 0.01 hours and about 18 hours (or any suitable period of time described above) following the administering of the composition to the subject.

In another aspect, the invention provides a method for treatment of diarrhea-predominant IBS in a subject having a body mass index (BMI) of less than about 30 kg/m², comprising providing a composition comprising cilansetron or a pharmaceutically acceptable derivative thereof, and administering a dose of the composition to the subject in an amount sufficient for the cilansetron to reach the bloodstream of the subject wherein a plasma cilansetron concentration between about 3 ng/mL and about 12 ng/mL is achieved between about 0.1 hours and about 12 hours following the administering of the composition to the subject.

In another aspect, the invention provides a method for treatment of diarrhea-predominant IBS in a subject having a body mass index (BMI) of less than about 30 kg/m², comprising providing a composition comprising cilansetron or a pharmaceutically acceptable derivative thereof, and administering a dose of the composition to the subject in an amount sufficient for the cilansetron to reach the bloodstream of the subject wherein the mean plasma cilansetron concentration across a statistically significant population of subjects is between about 0.1 ng/mL and about 25 ng/mL (or any suitable mean serum concentration described above). In another embodiment, the mean serum concentration is achieved in any suitable mean time or period as discussed above.

In another aspect, the invention provides a method for treatment of diarrhea-predominant IBS in a subject having a body mass index (BMI) of less than about 30 kg/m², comprising providing a composition comprising cilansetron or a pharmaceutically acceptable derivative thereof, and administering a daily dose of the composition to the subject in an amount sufficient for the cilansetron to reach the bloodstream of the subject wherein a plasma cilansetron concentration is substantially maintained between about 0.1 ng/mL and about 25 ng/mL for at least 12 hours (or any suitable duration as discussed above) after the subject has been administered the daily dose for at least 7 consecutive days (or any suitable number of days as discussed above).

In another aspect, the invention provides a method of treatment of diarrhea-predominant IBS in a subject receiving selective serotonin reuptake inhibitor (SSRI) therapy, comprising providing a composition comprising cilansetron or a pharmaceutically acceptable derivative thereof, and administering a dose of the composition to the subject in an amount sufficient for the cilansetron to reach the bloodstream of the subject wherein a plasma cilansetron concentration between about 0.1 ng/mL and about 25 (or any suitable serum concentration as described above) is achieved between about 0.01 hours and about 18 hours (or any suitable period of time described above) following the administering of the composition to the subject. Any suitable SSRI can be used in the context of the present invention. Non-limiting examples of SSRI's include paroxetine, fluvoxamine (e.g., fluvoxamine maleate), citaloprarn, escitalopram oxalate, fluoxetine, and sertraline. In an embodiment, the SSRI is selected from paroxetine and fluvoxamine.

In another aspect, the invention provides a method of treatment of diarrhea-predominant IBS in a subject receiving selective serotonin reuptake inhibitor (SSRI) therapy, comprising providing a composition comprising cilansetron or a pharmaceutically acceptable derivative thereof, and administering a dose of the composition to the subject in an amount sufficient for the cilansetron to reach the bloodstream of the subject wherein a plasma cilansetron concentration between about 3 ng/mL and about 12 ng/mL is achieved between about 0.1 hours and about 12 hours following the administering of the composition to the subject.

In another aspect, the invention provides a method of treatment of diarrhea-predominant IBS in a subject receiving selective serotonin reuptake inhibitor (SSRI) therapy, comprising providing a composition comprising cilansetron or a pharmaceutically acceptable derivative thereof, and administering a dose of the composition to the subject in an amount sufficient for the cilansetron to reach the bloodstream of the subject wherein the mean plasma cilansetron concentration across a statistically significant population of subjects is between about 0.1 ng/mL and about 25 ng/mL (or any suitable mean serum concentration described above). In another embodiment, the mean serum concentration is achieved in any suitable mean time or period as discussed above.

In another aspect, the invention provides a method of treatment of diarrhea-predominant IBS in a subject receiving selective serotonin reuptake inhibitor (SSRI) therapy, comprising providing a composition comprising cilansetron or a pharmaceutically acceptable derivative thereof, and administering a daily dose of the composition to the subject in an amount sufficient for the cilansetron to reach the bloodstream of the subject wherein a plasma cilansetron concentration is substantially maintained between about 0.1 ng/mL and about 25 ng/mL for at least 12 hours (or any suitable duration as discussed above) after the subject has been administered the daily dose for at least 7 consecutive days (or any suitable number of days as discussed above).

In another aspect, the invention provides a method for treatment of diarrhea-predominant IBS in a subject having renal impairment, comprising providing a composition comprising cilansetron or a pharmaceutically acceptable derivative thereof, and administering a dose of the composition to the subject in an amount sufficient for the cilansetron to reach the bloodstream of the subject wherein a plasma cilansetron concentration between about 0.1 ng/mL and about 25 ng/mL (or any suitable serum concentration as described above) is achieved between about 0.01 hours and about 18 hours (or any suitable period of time described above) following the administering of the composition to the subject.

In another aspect, the invention provides a method for treatment of diarrhea-predominant IBS in a subject having renal impairment, comprising providing a composition comprising cilansetron or a pharmaceutically acceptable derivative thereof, and administering a dose of the composition to the subject in an amount sufficient for the cilansetron to reach the bloodstream of the subject wherein a plasma cilansetron concentration between about 3 ng/mL and about 12 ng/mL is achieved between about 0.1 hours and about 12 hours following the administering of the composition to the subject.

In another aspect, the invention provides a method for treatment of diarrhea-predominant IBS in a subject having renal impairment, comprising providing a composition comprising cilansetron or a pharmaceutically acceptable derivative thereof, and administering a dose of the composition to the subject in an amount sufficient for the cilansetron to reach the bloodstream of the subject wherein the mean plasma cilansetron concentration across a statistically significant population of subjects is between about 0.1 ng/mL and about 25 ng/mL (or any suitable mean serum concentration described above). In another embodiment, the mean serum concentration is achieved in any suitable mean time or period as discussed above.

In another aspect, the invention provides a method for treatment of diarrhea-predominant IBS in a subject having renal impairment (or severe renal impairment), comprising providing a composition comprising cilansetron or a pharmaceutically acceptable derivative thereof, and administering a daily dose of the composition to the subject in an amount sufficient for the cilansetron to reach the bloodstream of the subject wherein a plasma cilansetron concentration is substantially maintained between about 0.1 ng/mL and about 25 ng/mL for at least 12 hours (or any suitable duration as discussed above) after the subject has been administered the daily dose for at least 7 consecutive days (or any suitable number of days as discussed above).

Clinical test data (set forth in the examples set forth below) prove the surprising suitability of cilansetron for the treatment of IBS-D in a subject.

### EXAMPLES

Intravenous injection formulations and oral solution formulations of cilansetron at strengths of 2 mg/40 mL, 4 mg/2 mL and 8 mg/4 mL can also be used. Table 2.7.1.1.1 presents a formulation for the 4 mg and 8 mg cilansetron capsules. Compositions of the injectable and oral formulations are presented in Table 2.7.1.1.2.

The capsule formulation proved to be stable for at least 12 months, if stored in polyvinyl chloride/polyvinyl dichloride/Al (PVC/PVDC/A1) blisters or in high density polyethylene (HDPE) bottles. There was only a small increase in the S-enantiomer content, and no organic impurities were found.

TABLE 2.7.1.1.1
Composition of Cilansetron 4 and 8 mg Capsule Formulations
Used in Clinical Trials

| | 4 mg/Capsule | 8 mg/Capsule |
|---|---|---|
| Cilansetron.HCl•H₂O | 4.7 (4.0) | 9.4 (8.0) |
| (=cilansetron base) | | |
| Mannitol | 225.3 | 220.6 |
| Maize starch | 130 | 130 |
| Povidone | 13 | 13 |
| Crospovidone | 13.5 | 13.5 |
| Colloidal silicon dioxide | 2.5 | 2.5 |
| Stearic acid | 11.0 | 11.0 |
| Total | 400.0 | 400.0 |

^{a}See Table 2.7.1.3.1 in Appendix 2.7.1.3
Supporting Data: Section 3.2.P.2.2, (Left. Vol. 9 Righ)

TABLE 2.7.1.1.2
Composition of Cilansetron Injectable and Oral Solutions

| | Injection | Oral Solution |
|---|---|---|
| | (mg/mL) | (mg/mL) |
| Cilansetron.HCl•H₂O (=cilansetron base) | 2.34 (2.0) | 2.34 (2.0) |
| Sodium chloride | 9.0 | — |
| Sodium hydroxide | 0.05 | — |
| Citric acid monohydrate | 0.60 | — |
| Water for injection | ad 1.00 mL | ad 40 mL |

Supporting Data: Section 3.2.P.2.2, (Left. Vol. 9 Righ)

1 mg, 2 mg, 4 mg, 8 mg, 16 mg, and 32 mg tablet formulations can also be used. Table 2.7.1.1.3 shows a comparison of the composition of the 4 mg capsule and tablet formulations. As is evident, the total weight for the tablets was lowered but the amounts of the excipients remained essentially proportional for both formulations.

TABLE 2.7.1.1.3
Comparison of Cilansetron 4 mg Capsule and Tablet
Formulations

| | Capsule | Tablet | Capsule | Tablet |
|---|---|---|---|---|
| | (mg) | (mg) | (%) | (%) |
| Cilansetron.HCl•H₂0 | 4.7 | 4.7 | 1 | 3 |
| Maize starch | 130 | 50.9 | 33 | 34 |
| Mannitol | 225.3 | 83.0 | 56 | 55 |
| Povidone | 13 | 4.9 | 3 | 3 |
| Citric acid monohydrate | 0 | 0.5 | 0 | 0.3 |
| Crospovidone | 13.5 | 3.0 | 3 | 2 |
| Colloidal anhydrous silica | 2.5 | 1.0 | 1 | 1 |
| Stearic acid | 11 | 2.0 | 3 | 1 |
| Total Weight | 400 | 150 | 100 | 100 |

Supporting Data: Section 3.2.P.2.2, (Left. Vol. 9 Righ)

Table 2.7.1.1.4 presents a nonlimiting overview of cilansetron tablet formulations that can be used.

TABLE 2.7.1.1.4
Composition of Cilansetron Tablets Used in Clinical and Stability Studies

| | Formulation codes | | | | | | | |
|---|---|---|---|---|---|---|---|---|
| | | T2a^{a}, | | | | | | |
| | T1^{a} | T2b^{b} | T2c^{c} | T4 | T8 | T16a | T16b^{d} | T32 |
| Tablet Strength (mg) | 1 | 2 | 2 | 4 | 8 | 16 | 16 | 32 |

Tablet Core

| | | | | | | | | |
|---|---|---|---|---|---|---|---|---|
| Cilansetron.HCl•H₂O | 1.17 | 2.34 | 2.34 | 4.68 | 9.37 | 18.74 | 18.74 | 37.47 |
| Maize starch | 52.23 | 51.78 | 51.78 | 50.89 | 49.11 | 45.55 | 45.17 | 38.44 |
| Mannitol | 85.20 | 84.48 | 84.48 | 83.03 | 80.12 | 74.31 | 73.69 | 62.69 |
| Povidone | 4.90 | 4.90 | 4.90 | 4.90 | 4.90 | 4.90 | 4.90 | 4.90 |
| Citric acid monohydrate | 0.50 | 0.50 | 0.50 | 0.50 | 0.50 | 0.50 | 0.50 | 0.50 |
| Crospovidone | 3.00 | 3.00 | 3.00 | 3.00 | 3.00 | 3.00 | 3.00 | 3.00 |
| Colloidal anhydrous | 1.00 | 1.00 | 1.00 | 1.00 | 1.00 | 1.00 | 1.00 | 1.00 |
| silica | | | | | | | | |
| Stearic acid | 2.00 | 2.00 | 2.00 | 2.00 | 2.00 | 2.00 | 3.00 | 2.00 |

Coating

| | | | | | | | | |
|---|---|---|---|---|---|---|---|---|
| Opadry ® Y-1-7000, white | 4 | 4 | | 4 | 4 | 4 | 4 | 4 |
| Opadry ® 03B28686, white | | | 4 | | | | | |
| Total weight | 154 | 154 | 154 | 154 | 154 | 154 | 154 | 154 |

^{a}For early 1 and 2 mg tablet batches (codes T1 and T2a), a different granulation procedure was followed than for higher strength tablets. For 1 and 2 mg tablet batches, granulates for 4 mg tablets and a placebo tablet were made. Subsequently, for the 2 mg tablet both granulates were mixed in equal quantities, and for the 1 mg tablet both granulates were mixed in the placebo to 4 mg ratio of 3 to 1.
^{b}2-mg batches made in May 2001 and later were made by directly producing a 2 mg granulate. The composition of the formulation did not change; moreover, the Frewitt oscillating bar sieve was replaced by the Quadro Comil U-10 rotating impeller mill for the breaking step of the dried granular material.
^{c}The film-coating changed from ‘Opadry ® Y-1-7000 white’ to ‘Opadry ® 03B28686 white’. The quantitative composition and properties of the coating remained the same. The only difference was that ‘Opadry ® 03B28686 white’ contained hypromellose (6 cP) from Shin-Etsu, whereas ‘Opadry ® Y-1-7000’ contained hypromellose (5 cP) from Dow. Both hypromellose qualities were of the 2910 type. This change was introduced in May 2002.
^{d}The amount of stearic acid was changed from 2.00 to 3.00 mg per tablet for the 16 mg tablet resulting in formulation T16b. The maize starch and mannitol amounts were adapted to keep the tablet weight unchanged. This change was introduced in April 2000.
Supporting Data: Section 3.2.P.2.2, (Left. Vol. 9 Righ)

Comparative Dissolution Studies
Dissolution Profiles of Cilansetron 4 and 8 mg Capsules

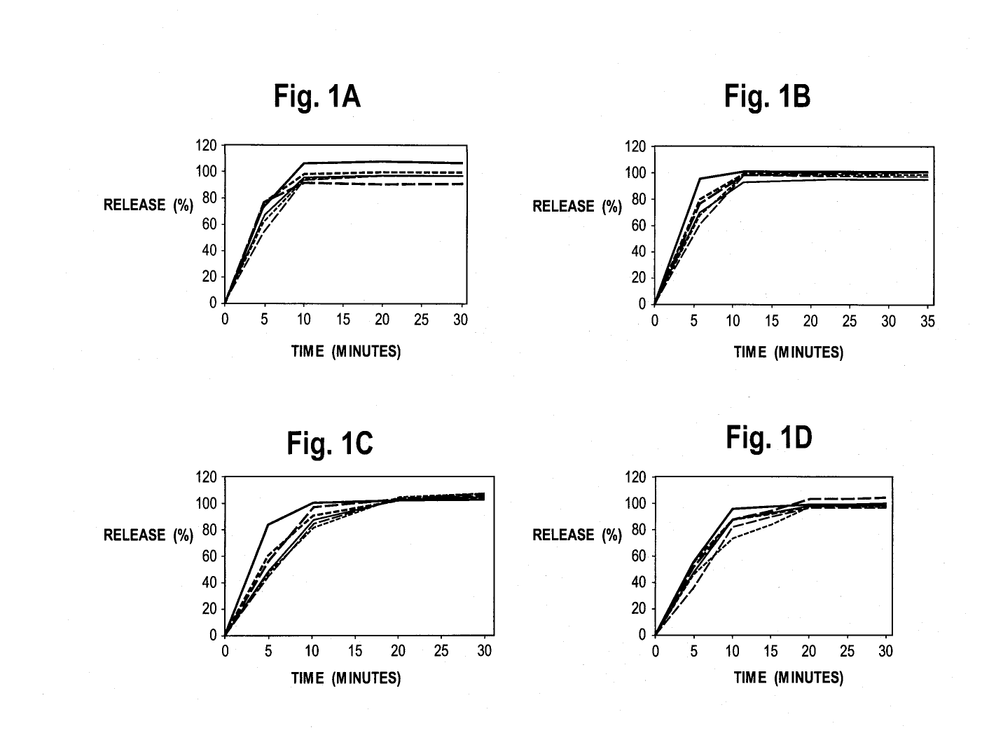

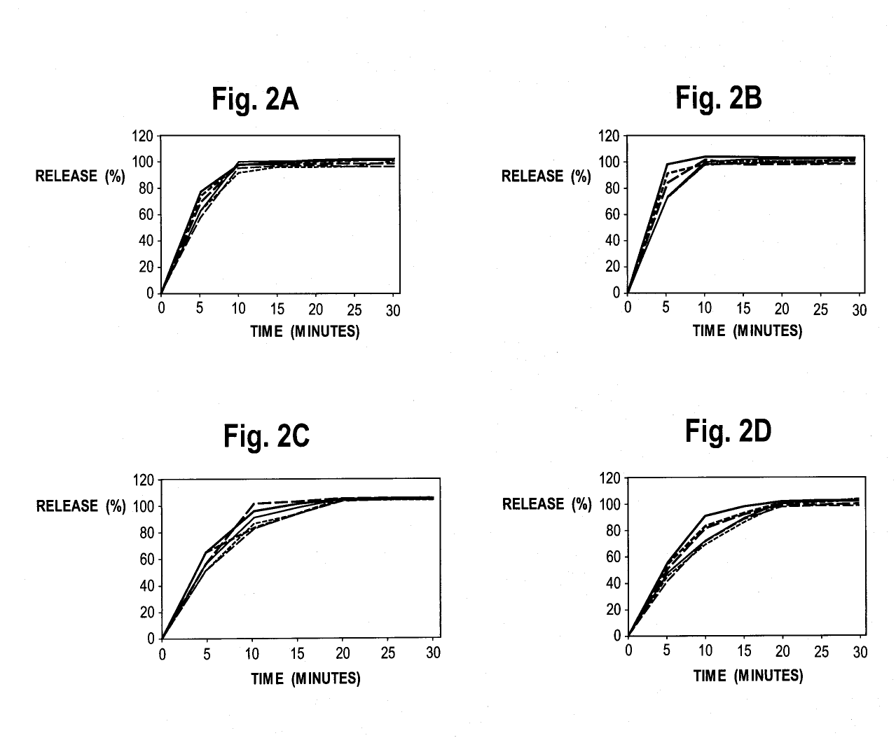

FIGS. 1a-d and FIGS. 2a-d provide dissolution profiles of cilansetron 4 and 8 mg capsules, respectively. In vitro dissolution of cilansetron from these capsules was rapid. Using the United States Pharmacopeia (USP) basket method at 100 revolutions per minute (rpm), complete dissolution in degassed simulated gastric fluid was typically observed within 10 to 20 minutes.

Dissolution Profiles of Cilansetron 8 mg Tablet and 8 mg Capsule

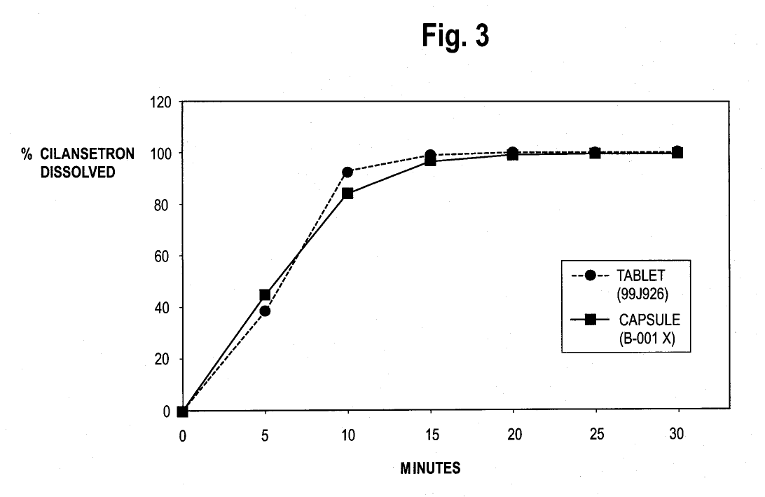

A comparison was made of the in vitro dissolution of 8 mg capsules and 8 mg tablets. The composition of the 8 mg tablets is presented in Table 2.7.1.1.4. The results are presented in FIG. 3, which show that dissolution of cilansetron from both capsules and tablets is rapid and complete in approximately 15 minutes. No significant differences in dissolution between the two formulations were observed.

Solubility

It has also been demonstrated that the dissolution profile of cilansetron 2 mg film-coated tablets is independent of the pH value of the dissolution medium. Moreover, cilansetron 2 mg film-coated tablets can be classified as rapidly dissolving because more than 85% of cilansetron is dissolved within 15 minutes. In in vivo studies, it was observed that absorption of orally administered cilansetron is rapid and higher than 90%.

Bioanalytical Methods

This section describes the bioanalytical methods that were instituted to support the clinical development of cilansetron.

All bioanalytical methods were adopted and validated to support the human pharmacokinetic studies for cilansetron and to assure accurate and precise determination of the analytes in the declared ranges. These methods are summarized below. In addition, long-term storage stability was found for cilansetron in frozen human plasma for 20 months and urine for 12 months; for the 4-hydroxy metabolites of cilansetron in plasma, stability was shown for a storage period of 24 months. In serum, cilansetron and its 4-hydroxy metabolites were shown to be stable for up to 14 months.

Cilansetron in Plasma

HPLC/UV methods were used to perform serum analyses for cilansetron. A lower limit of quantitation (LLOQ) was established at 0.89 ng/mL for both concentration ranges. At the LLOQ QC level, within-run precision [percent coefficient of variation (%CV)] ranged from 8.49 to 15.81% for the 3-run validation and was 19.86% for the 1-run validation to extend the range of the assay.Between-run precision at the LLOQ QC level for the 3-run validation was 6.86%. Within- and between-run precisions of the remaining QC levels ranged from 2.60 to 5.81% and 1.59 to 2.29%, respectively. Within-run accuracy ranged from 86.70 to 114.23% for all QC levels in all 4 validation runs. Between-run accuracy ranged from 90.37 to 105.89% for the 1 to 100 ng/mL range.

Cilansetron in Urine

Analysis of cilansetron in human urine was performed by HPLC/UV. A validation procedure incorporated a 4-run validation on different days to obtain within- and between-run accuracy and precision data in the concentration range of 10 to 1000 ng/mL. Two additional 1-run validations were conducted at a later date to improve the parameters for analyzing authentic samples. For these 6 validation runs, calibration curves were constructed from 7 calibration standards and 6 replicates at each of four QC levels were prepared (near-LLOQ, LOW, MID, and HIGH). In order to further validate the LLOQ, additional validation runs were subsequently conducted using the original method parameters to evaluate accuracy and precision of an LLOQ QC prepared at 8.67 ng/mL. The HPLC[UV method was accurate, precise, and sensitive. Precision and accuracy data were within laboratory-specified limits. The LLOQ was established at 8.67 ng/mL. Within- and between-run precisions (% CV) of the near-LLOQ, LOW, MID, and HIGH QC's ranged from 1.20 to 14.52% and 2.38 to 10.32%, respectively, in all 4 validation runs using the original parameters. Within-run accuracy varied between 93.48 and 111.19% for the LOW, MID, and HIGH QCs; for the near-LLOQ QC, within-run accuracy varied between 100.06 and 123.71%. Between-run accuracy varied between 103.53 and 112.72% for all QC levels. During validation of the LLOQ QC level, the initial run displayed high results for precision and accuracy which could not be explained. Four subsequent validation runs produced the following results for the LLOQ QC: within-run precision ranged from 2.08 to 4.45% and between-run precision was 11.23%; within-run accuracy varied between 85.79 and 110.40% and between-run accuracy was 102.97%. For the two 1-run validations of modified parameters, within-run precision ranged from 0.77 to 6.85% and within-run accuracy ranged from 89.41 to 98.57%.

LC/MS/MS Method

Separate LC/MS/MS methods for the determination of cilansetron and its 4-hydroxymetabolites in human plasma, serum and ultra filtrate (from serum) were performed.

Cilansetron in Plasma

Validation of a method for the analysis of cilansetron in human plasma was performed by LC/MS/MS. A validation procedure incorporated a 3-run validation on different days to obtain within- and between-run accuracy and precision data in the concentration range of 0.1 to 52.5 ng/mL. For each validation run, calibration curves were constructed from 9 calibration standards and 6 replicates at each of 4 QC levels were prepared (LLOQ, LOW, MID, and HIGH). The LC/MS/MS method was found to be accurate, precise, and sensitive. Precision and accuracy data were within laboratory-specified limits. Within- and between-run precisions (% CV) at all QC levels ranged from 1.67 to 11.5% and 3.35 to 7.63%, respectively. Within-run accuracy ranged from 96.7 to 113.7% for all QC levels, and between-run accuracy ranged from 103.4 to 110.8%. Moreover, an LC/MS/MS method with a modification using Turbolonspray was used. Precision and accuracy data obtained on the method modification were within laboratory-specified limits. Within-run precision (% CV) for all QC levels ranged from 2.29 to 10.8%, and within-run accuracy ranged from 86.2 to 109.5%. The LLOQ was confirmed at 0.1 ng/mL.

4-Hydroxy Metabolites in Plasma

An analysis for (4S)-hydroxy cilansetron and (4R)-hydroxycilansetron in human plasma was performed by LC/MS/MS. A validation was performed by FOCUS Clinical Drug Development Gmbh. The validation incorporated a 3-run validation to determine within- and between-run accuracy and precision over a concentration range of 0.1 to 20 ng/mL. For each validation run, calibration curves were constructed from 8 calibration standards and 6 replicates at each of 4 QC levels were prepared (LLOQ, LOW, MID, and HIGH). The LC/MS/MS method was found to be accurate, precise, and sensitive. Precision and accuracy data were within laboratory-specified limits. The LLOQ was established at 0.1 ng/mL for both metabolites. The within-run precisions (% CV) for all QC levels ranged from 1.28 to 11.13% for (4S)-hydroxy cilansetron and 1.44 to 10.69% for (4R)-hydroxycilansetron; the corresponding between-run precisions (% CV) ranged from 4.04 to 8.94%and 2.05 to 6.37%, respectively. The within- and between-run accuracies for (4S)-hydroxycilansetron in this concentration range varied between 84.5 to 109.7% and 89.0 to 104.3%, respectively. The corresponding ranges for (4R)-hydroxy cilansetron were 89.5 to 113.7% and 92.3 to 107.5%.

Cilansetron and 4-Hydroxy Metabolites in Plasma

An analysis for cilansetron and its 4-hydroxy metabolites in human potassium edetic acid (EDTA) plasma was performed by LC/MS/MS. The validation method employed a single sample preparation and two separate LC/MS/MS methods. The validation consisted of a total of 9 analytical runs. Five of these runs were used to assess accuracy and precision, and included calibration curves constructed from 8 calibration standards and 5 replicates at each of the included QC levels (LLOQ, LOW, MID, and HIGH). The method was suitable for the measurement of cilansetron and its main metabolites. The LC/MS/MS method was found to be precise and accurate over a concentration range of approximately 0.1 to 50 ng/mL for each analyte, with an LLOQ of 0.1 ng/mL for each analyte. The within-run precisions ranged from 1.2 to 7.5%, 0.5 to 6.5%,and 1.6 to 6.6% for cilansetron, (4S)-hydroxy cilansetron, and (4R)-hydroxy cilansetron, respectively, while the between-run precisions ranged from 3.0 to 7.1%, 2.3 to 5.9%, and 3.9 to 5.7%, respectively. The within-run accuracies ranged from −1.4 to 16.0%, −4.6 to 10.0%, and −4.4 to 7.0% for cilansetron, (4S)-hydroxy cilansetron, and (4R)-hydroxycilansetron, respectively, while the between-run accuracies ranged from 0.4 to 7.0%, −3.6 to 4.0%, and −2.2 to 4.0%, respectively.

Cilansetron and 4-Hydroxy Metabolites in Serum

An analysis for cilansetron and its 4-hydroxy metabolites in human serum was performed by LC/MS/MS. A validation method employed a single sample preparation and two separate LC/MS/MS methods. The validation consisted of a total of 7 analytical runs. Five of these runs were used to assess accuracy and precision, and included calibration curves constructed from 8 calibration standards and 5 replicates at each of the included QC levels (LLOQ, LOW, MID, and HIGH). The method was suitable for the measurement of cilansetron and its main metabolites. The LC/MS/MS method was found to be precise and accurate over a concentration range of approximately 0.1 to 50 ng/mL for each analyte, with an LLOQ of 0.1 ng/mL for each analyte. The within-run precisions ranged from 1.1 to 6.6%, 1.8 to 12.1%, and 1.4 to 12.2% for cilansetron, (4S)-hydroxy cilansetron, and (4R)-hydroxycilansetron, respectively, while the between-run precisions ranged from 2.6 to 5.7%, 3.7 to 11.2%, and 4.0 to 9.8%, respectively. The within-run accuracies ranged from 0.3 to 12.2%, −10.9 to 7.0%, and −8.5 to 8.0% for cilansetron, (4S)-hydroxy cilansetron, and (4R)-hydroxycilansetron, respectively, while the between-run accuracies ranged from 4.1 to 7.6%, −6.6 to 2.0%, and −3.0 to 0.0%, respectively.

Cilansetron and 4-Hydroxy Metabolites in Protein-Free Filtrate Derived from Serum

An analysis for cilansetron and its 4-hydroxy metabolites in protein-free filtrate (derived from human serum) was performed. A validation method employed a single sample preparation and two separate LC/MS/MS methods. The validation consisted of a total of 7 analytical runs. Four of the serums were used to assess accuracy and precision, and included calibration curves constructed from 8 calibration standards and 5 replicates at each of the included QC levels (LLOQ, LOW, MID, and HIGH). The method was suitable for the measurement of cilansetron and its main metabolites. The LC/MS/MS method was found to be precise and accurate over a concentration range of approximately 0.04 to 20 ng/mL for each analyte, with an LLOQ of 0.04 ng/mL for each analyte. The within-run precisions ranged from 2.1 to 9.5%, 3.4 to 8.7%, and 3.8 to 9.6% for cilansetron, (4S)-hydroxy cilansetron, and (4R)-hydroxycilansetron, respectively, while the between-run precisions ranged from 5.4 to 13.2%, 5.2 to 8.0%, and 4.6 to 10.3%, respectively. The within-run accuracies ranged from −9.0 to 17.8%, −4.6 to 7.4%, and −8.5 to 13.8% for cilansetron, (4S)-hydroxy cilansetron, and (4R)-hydroxycilansetron, respectively, while the between-run accuracies ranged from 1.3 to 7.5%, −2.5 to 0.7%, and −1.7 to 6.0%, respectively.

Validation Methods Used in ¹⁴C-Mass Balance Study (S241.1.103)

Methods for the quantitative determination of ¹⁴C-radioactivity levels in human plasma, whole blood, urine, and feces were also performed. Samples from the mass balance study were also analyzed for determination of unchanged cilansetron in plasma and urine by a HPLCfUV method. This method was based on the HPLC/UV method developed by FOCUS Clinical Drug Development GmbH, and was validated at XenoBiosis prior to use for sample analysis. The validation procedure incorporated 3-run validations for each matrix to obtain within- and between-run accuracy and precision data in the concentration range of 2 to 500 ng/mL. For each validation run, calibration curves were constructed from 8 calibration standards. The plasma validation runs contained duplicates at each of three QC levels (LOW, MID, and HIGH), while the urine validation runs contained triplicate QCs at the LOW level and duplicate QCs at the MID and HIGH levels. The HPLC methods for plasma and urine were accurate, precise, and sensitive. Within-run precisions (% CV) of the LOW, MID, and HIGH QC levels ranged from 2.20 to 4.51% in plasma and 2.76 to 6.96% in urine; between-run precisions ranged from 3.47 to 5.88% in plasma and 4.15 to 14.0% in urine. Between-run accuracy ranged from −9.6 to 0.74% in plasma and −9.3 to 3.6% in urine.

Additional Validation Method Used

Concentrations of cilansetron and its 4-hydroxy metabolites were also assayed from human female plasma using a LC/MS/MS method. In addition, concentrations of ethinyl estradiol (EE) were assayed from human female plasma using a validated gas chromatography/mass spectroscopy (GC/MS) method. Norgestrel (NGL), norgestimate (NGM), and 17-d-norgestimate (17-d-NGM) were assayed from human female plasma using a validated LC/MSIMS method.

EE Results. The GC/MS method was found to be accurate, precise, and sensitive. Precision and accuracy data were within laboratory-specified limits. The LLOQ was established at 2.05 pg/mL. The within- and between-run precision (% CV) for all QC levels ranged from 1.9 to 8.3% and 2.0 to 9.0%. The within- and between-run accuracy (% nominal) for all QC levels ranged from 86.3 to 101.5% and 91.9 to 111.8%.

NGL, NGM, and 17-d-NGM Results. The LC/MS/MS method was found to be accurate, precise, and sensitive. Precision and accuracy data were within laboratory-specified limits. LLOQs were established at 79.8, 20.0, and 19.9 pg/mL for NGL, NGM,and 17-d-NGM respectively. The within- and between-run precision (% CV) for all QC levels ranged from 4.6 to 7.5% and 3.9 to 13.6% for NGL; 2.4 to 8.6% and 2.8 to 16.8% for NGM; and 0.7 to 10.8% and 2.9 to 8.4% for 17-d-NGM. The within- and between-run accuracy (% nominal) for all QC levels ranged from 87.3 to 101.6% and 95.5 to 101.8% for NGL; 95.9 to 106.1% and 94.0 to 117.7% for NGM; and 105.2 to 112.5% and 105.8 to 113.4% for 17-d-NGM.

Relative Bioavailabilitv (BA)

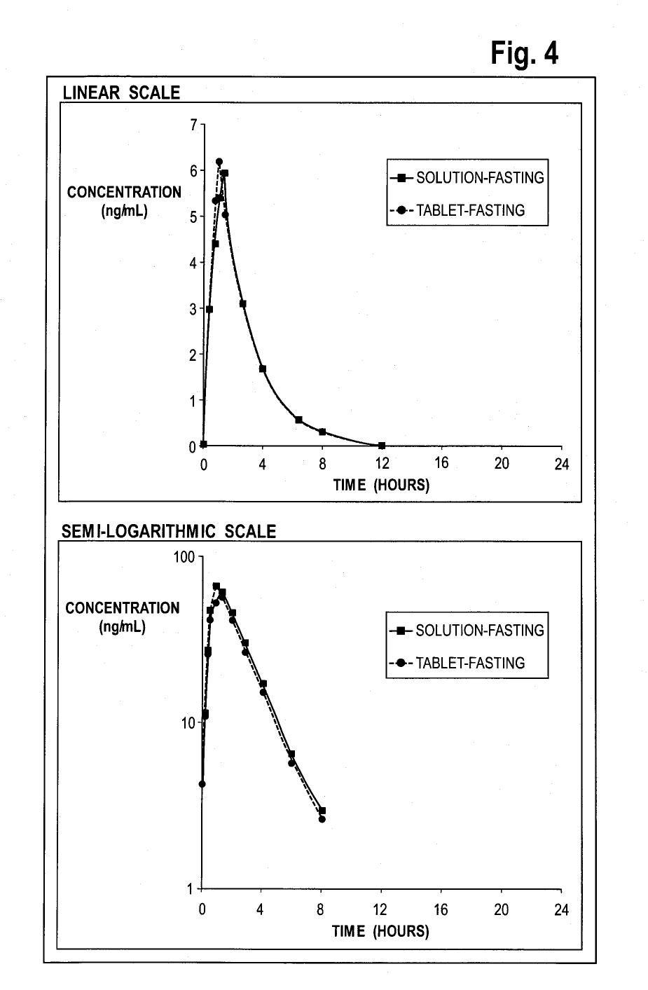

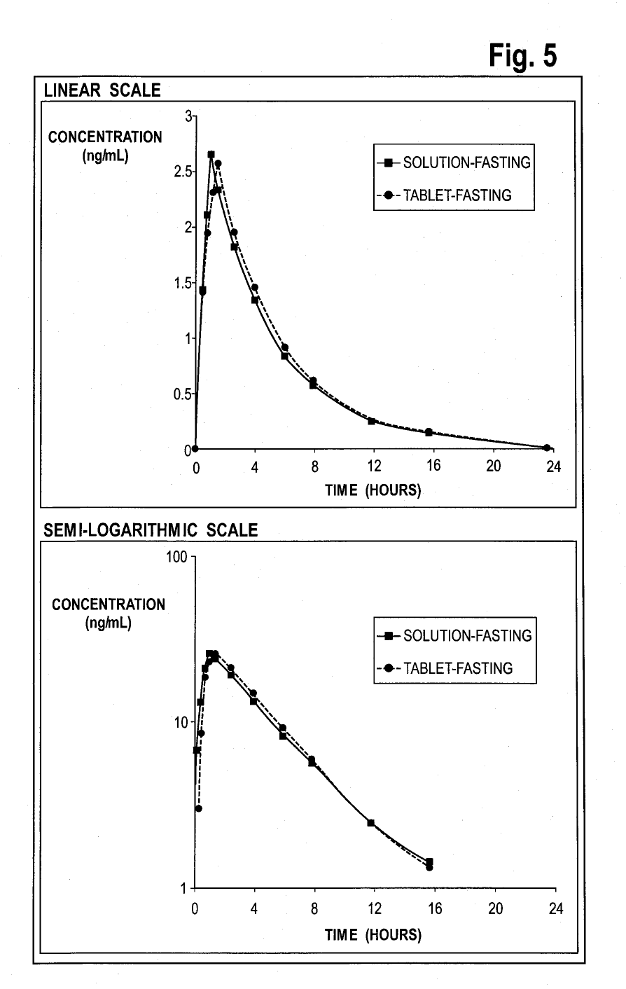

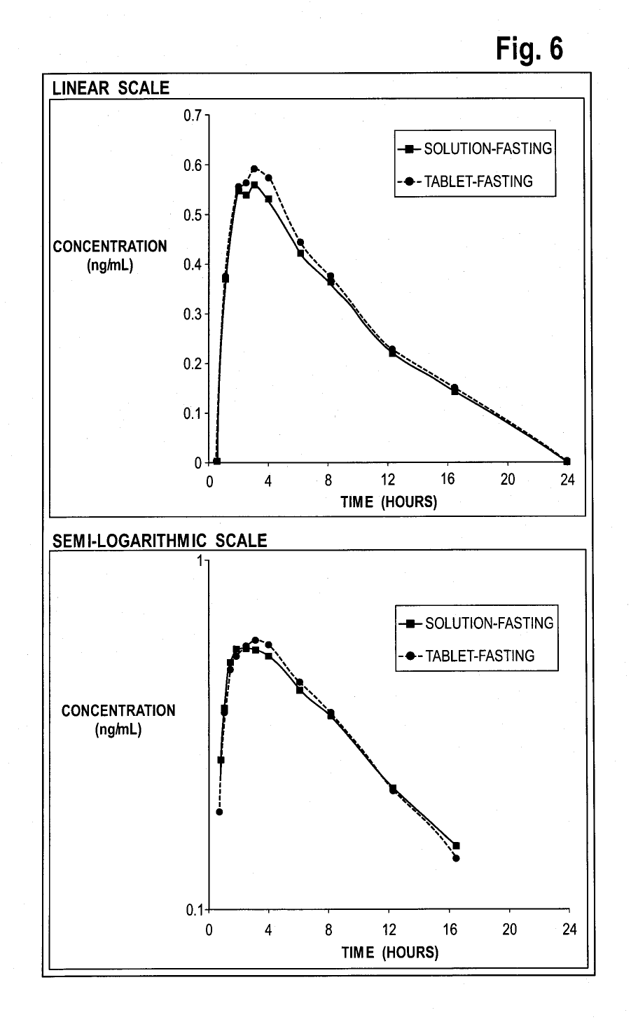

The relative bioavailability of the cilansetron 2 mg tablet, has been evaluated in comparison to a 2 mg oral solution in a randomized, open-label, single dose, three-period, crossover study in 12 male and 6 female volunteers, aged 21 to 41 years. Subjects were randomized to receive a cilansetron 2 mg tablet after an overnight fast, a cilansetron 2 mg tablet after a high-fat breakfast, and a cilansetron 2 mg oral solution after an overnight fast during the three treatment periods, with a 7-day washout between periods. Serial blood sampling was performed up to 24 hours following each treatment, and plasma pharmacokinetics parameters for cilansetron and its metabolites, (4R)-hydroxy cilansetron and (4S)-hydroxy cilansetron, were estimated. FIG. 4, FIG. 5, and FIG. 6 present mean plasma concentration-time profiles for cilansetron and its metabolites following treatment with the cilansetron tablet and oral solution. Table 2.7.1.2.2 presents descriptive statistics for the pharmacokinetic parameters of cilansetron and its metabolites for the tablet and oral solution, and the results of statistical comparisons between treatments (tablet versus solution). The results showed that the 2 mg tablet formulation is bioequivalent to the oral solution. The mean concentration-time profiles for cilansetron and its 4-hydroxy metabolites were generally superimposable for the two treatments, and the geometric least squares mean ratios and 90% confidence intervals for both cilansetron Cmax and AUC(0-inf) were within the regulatory 80-125% bioequivalence limits. Descriptive statistics indicated that the pharmacokinetic parameters for the 4-hydroxy metabolites were also very similar between treatments.

TABLE 2.7.1.2.2
Arithmetic Mean (% CV) of Pharmacokinetic Parameters and
Statistical Comparisons for Cilansetron and Its Metabolites
Following Administration of Single Doses of the “To-Be-
Marketed” Formulation, Cilansetron 2 mg Tablet, and a 2 mg
Oral Solution (Study S241.1.111)

| | | | | | | Statistical | |
|---|---|---|---|---|---|---|---|
| | | | | | | Comparisons | |
| | | | | | | (Treatment | |
| | | Arithmetic Mean (% CV) | | | | B/A)^{b} | |
| | | 2 mg Tablet | | 2 mg Oral | | Ratio | |
| Analyte | Parameter | (N = 18) | | Solution (N = 18) | | (%) | 90% CI |
| Cilansetron | AUC(0-last) | 22.4 | (89) | 20.2 | (70) | NC | NC |
| | (ng · h/mL) | | | | | | |
| | AUC(0-inf) | 22.8 | (86) | 20.7 | (68) | 99 | 89, 110 |
| | (ng · h/mL) | | | | | | |
| | Cmax (ng/mL) | 8.24 | (67) | 7.55 | (72) | 108 | 95, 124 |
| | Tmax (h)^{a} | 1.00 | (0.75, 1.75) | 1.00 | (0.75, 1.50) | NC | NC |
| | T½ (h) | 1.42 | (20) | 1.48 | (19) | NC | NC |
| (4R)—OH | AUC(0-last) | 13.7 | (26) | 13.5 | (27) | NC | NC |
| | (ng · h/mL) | | | | | | |
| | AUC(0-inf) | 14.4 | (25) | 14.3 | (26) | NC | NC |
| | (ng · h/mL) | | | | | | |
| | Cmax (ng/mL) | 2.87 | (20) | 2.82 | (26) | NC | NC |
| | Tmax (h)^{a} | 1.25 | (0.75, 2.00) | 1.00 | (0.75, 1.75) | NC | NC |
| | T½ (h) | 3.90 | (22) | 3.90 | (19) | NC | NC |
| (4S)—OH | AUC(0-last) | 5.98 | (22) | 5.82 | (24) | NC | NC |
| | (ng · h/mL) | | | | | | |
| | AUC(0-inf) | 7.31 | (22) | 7.20 | (23) | NC | NC |
| | (ng · h/mL) | | | | | | |
| | Cmax (ng/mL) | 0.637 | (17) | 0.607 | (19) | NC | NC |
| | Tmax (h)^{a} | 3.00 | (1.50, 4.00) | 3.00 | (1.75, 4.00) | NC | NC |
| | T½ (h) | 7.26 | (32) | 7.13 | (25) | NC | NC |

Treatment A = cilansetron 2 mg oral solution (batch # 63174 for powder);
Treatment B = cilansetron 2 mg tablet (batch # 61874); both treatments were administered under fasted conditions.
(4R)—OH = (4R)-hydroxy cilansetron;
(4S)—OH = (4S)-hydroxy cilansetron
NC = not calculated;
% CV = coefficient of variation on arithmetic mean in percent.
^{a}Results presented are median (min, max).
^{b}Geometric least squares mean ratio and 90% confidence interval (CI) from ANOVA comparisons of log-transformed data.
Supporting Data: S241.1.111 clinical study report, (Left. Vol. 9 Righ)

The relative bioavailability of a cilansetron 8 mg capsule has been evaluated in comparison to an 8 mg oral solution in a randomized, open-label, single-dose, three-period, crossover study in 18 male volunteers, aged 21 to 38 years. Subjects were randomized to receive cilansetron 8 mg IV solution (30-minute infusion), cilansetron 8 mg oral solution, and a cilansetron 8 mg capsule during the three treatment periods, with a 23-day washout between periods. All doses were administered under fasted conditions. Serial blood sampling was performed up to 9 hours following each dose, and cilansetron pharmacokinetic parameters were evaluated. Table 2.7.1.2.3 presents descriptive statistics for the pharmacokinetic parameters of cilansetron capsule and oral solution, and the results of statistical comparisons between treatments (capsule versus solution). Although the confidence intervals for AUC and Cmax were outside the 80-125% limits for statistical bioequivalence, the geometric least squares mean ratios demonstrated that the bioavailability of the 8 mg capsule was similar to the oral solution. The mean extent of exposure [AUC(0-inf) and AUC(0-last)] of the 8 mg capsule was comparable to the oral solution, and the mean Cmax was only slightly lower (12%) than that of the solution. Median Tmax was also similar between the capsule and oral solution.

TABLE 2.7.1.2.3
Arithmetic Mean (% CV) Single-Dose Pharmacokinetic Parameters for
Cilansetron Following Administration of Cilansetron 8 mg as an Oral
Capsule and Oral Solution (Study K.241.5015)

| | | | Statistical | |
|---|---|---|---|---|
| | | | Comparisons^{c} | |
| | | | Oral Capsule: | |
| | | | Oral Solution | |
| | Oral Capsule^{b} | Oral Solution^{b} | Ratio | 90% CI |
| Parameter | (N = 18) | (N = 17) | (%) | (%) |
| AUC (0-inf) | 66.0 (47) | 73.8 (69) | 93 | 72, 120 |
| (ng · h/mL) | | | | |
| AUC (0-last) | 62.1 (48) | 69.3 (73) | 96 | 73, 128 |
| (ng · h/mL) | | | | |
| Cmax (ng/mL) | 31.4 (49) | 39.2 (67) | 88 | 64, 120 |
| Tmax (h)^{a} | 1.00 (0.67, 2.50) | 0.83 (0.33, 1.25) | NC | NC |
| T½ (h) | 1.28 (22) | 1.47 (21) | NC | NC |
| CL/F (mL/min) | 2824 (80) | 2648 (60) | NC | NC |

NC = not calculated;
% CV = coefficient of variation on arithmetic mean in percent.
^{a}Results presented are median (min, max).
^{b}Cilansetron 8 mg oral capsule, Batch No. 044P; Cilansetron 8 mg oral solution, Batch No. 014N.
^{c}Results of ANOVA comparisons of log-transformed data (N = 17).
Note:
Data for treatment with 8 mg IV solution are not presented in this table. See Section 2.7.2.3.1 Single and Multiple Dose Pharmacokinetics, (Left. Vol. 9 Righ) for data on this treatment.
Supporting Data: K.241.5015 clinical study report, (Left. Vol. 9 Righ)

Bioavailability/Food Effect

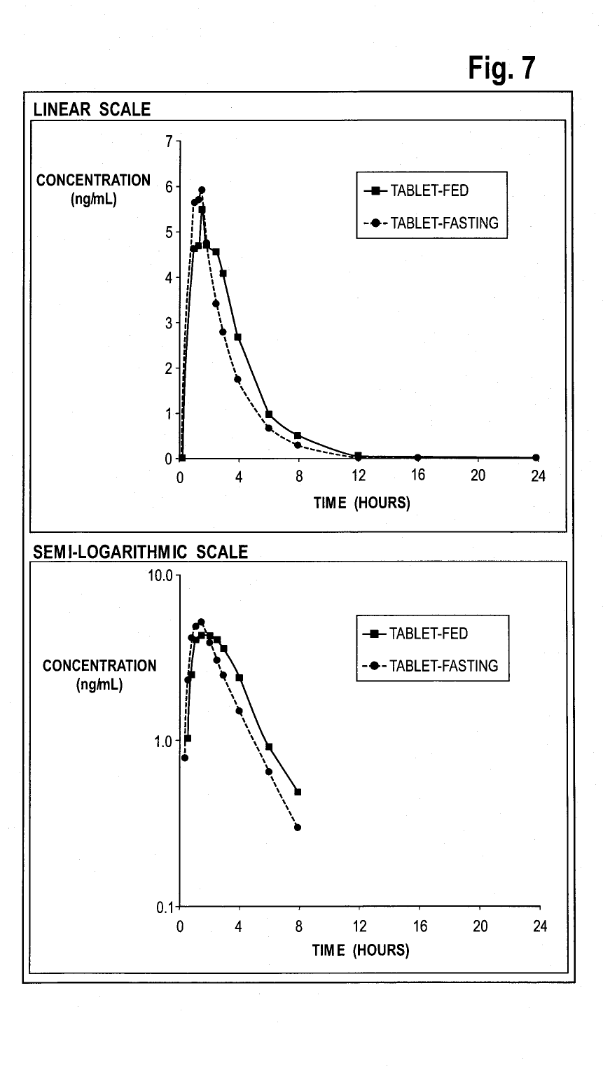

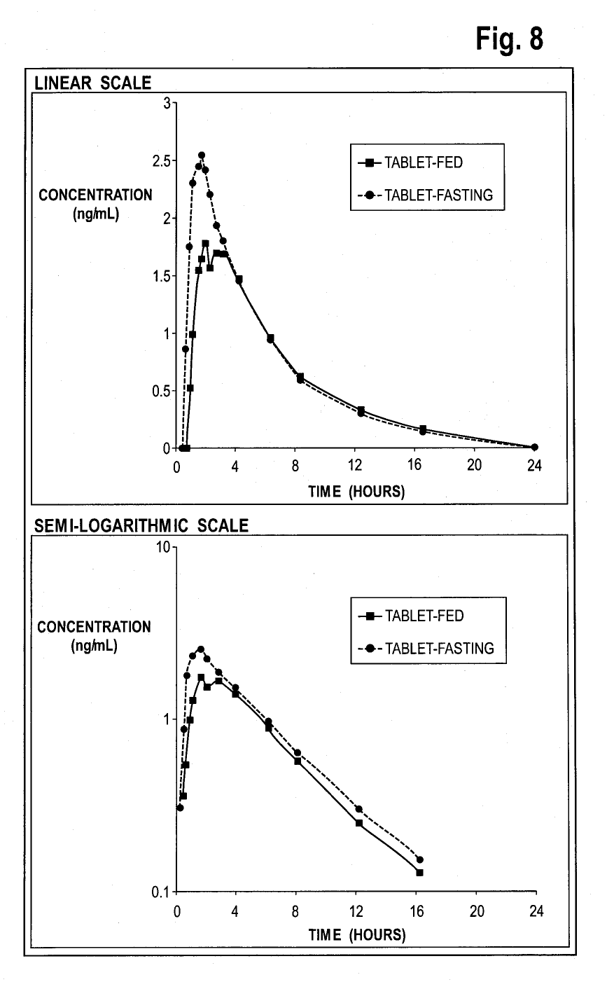

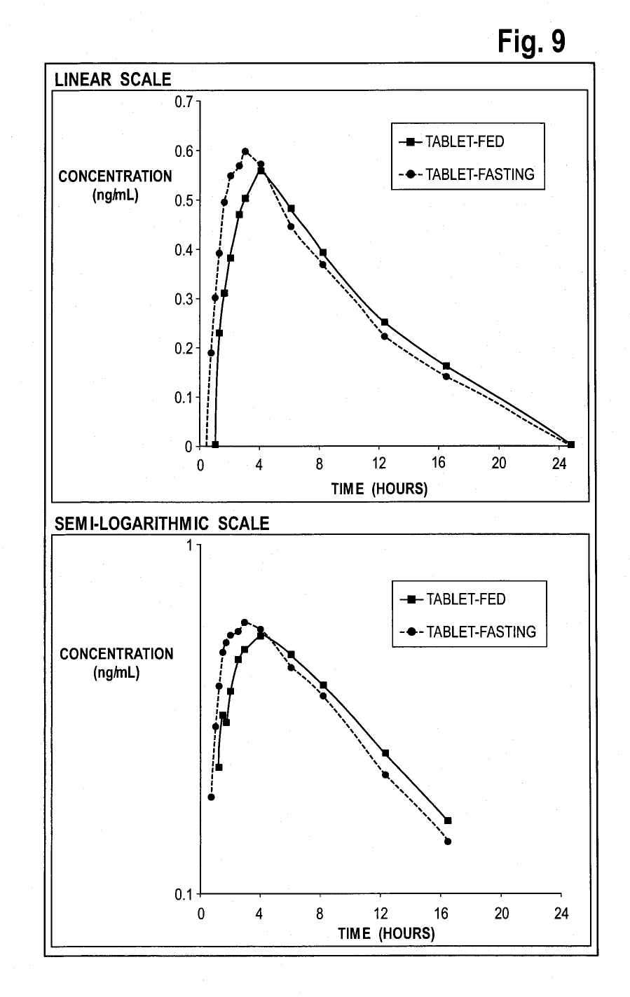

The effect of a high-fat breakfast on the bioavailability of the cilansetron 2 mg tablet, has been evaluated in a randomized, open-label, single-dose, three period, crossover study in 12 male and 6 female volunteers, aged 21 to 41 years. Subjects were randomized to receive a cilansetron 2 mg tablet after an overnight fast, a cilansetron 2 mg tablet after a high-fat breakfast, and a cilansetron 2 mg oral solution after an overnight fast during the three treatment periods, with a 7-day washout between periods. Serial blood sampling was performed up to 24 hours following each treatment and plasma pharmacokinetics parameters for cilansetron and its metabolites, (4R)-hydroxy cilansetron and (4S)-hydroxy cilansetron, were estimated. FIG. 7, FIG. 8, and FIG. 9 present mean plasma concentration-time profiles for cilansetron and its metabolites following treatment with the cilansetron tablet under fasted conditions and following a high-fat meal. Table 2.7.1.2.4 presents descriptive statistics for the pharmacokinetic parameters of cilansetron and its metabolites for the tablet under fed and fasted conditions, and the results of statistical comparisons between treatments (fed versus fasted). The results showed that administration with a high-fat meal caused a small but clinically insignificant increase in the bioavailability of cilansetron. No change was observed in cilansetron Cmax, but AUC(0-inf) was increased by 20% on average and there was a small increase in Tmax under fed conditions. Although not statistically compared, descriptive statistics indicated that AUC values for the two 4-hydroxy metabolites were essentially unchanged under fed and fasting conditions. A slight reduction in Cmax and a lengthening in Tmax were observed for the metabolites in the fed state.

TABLE 2.7.1.2.4
Arithmetic Mean (% CV) Pharmacokinetic Parameters and
Statistical Comparisons for Cilansetron and Its Metabolites
Following Administration of Single Doses of the “To-Be-
Marketed” Formulation, Cilansetron 2 mg Tablet, Under Fasted
Conditions and Following a High-Fat Breakfast (Study S241.1111)

| | | | | | | Statistical | |
|---|---|---|---|---|---|---|---|
| | | | | | | Comparisons | |
| | | Arithmetic Mean (% CV) | | | | (Treatment B/A)^{b} | |
| | | Tablet Fed | | Tablet Fasted | | Ratio | |
| Analyte | Parameter | (N = 18) | | (N = 18) | | (%) | 90% CI |
| Cilansetron | AUC(0-last) | 24.2 | (72) | 22.4 | (89) | NC | NC |
| | (ng · h/mL) | | | | | | |
| | AUC(0-inf) | 24.8 | (70) | 22.8 | (86) | 121 | 108, 134 |
| | (ng · h/mL) | | | | | | |
| | Cmax (ng/mL) | 7.83 | (59) | 8.24 | (67) | 101 | 88, 116 |
| | Tmax (h)^{a} | 1.38 | (0.75, 4.00) | 1.00 | (0.75, 1.75) | NC | NC |
| | T½ (h) | 1.51 | (19) | 1.42 | (20) | NC | NC |
| (4R)—OH | AUC(0-last) | 12.8 | (27) | 13.7 | (26) | NC | NC |
| | (ng · h/mL) | | | | | | |
| | AUC(0-inf) | 13.6 | (26) | 14.4 | (25) | NC | NC |
| | (ng · h/mL) | | | | | | |
| | Cmax (ng/mL) | 2.23 | (26) | 2.87 | (20) | NC | NC |
| | Tmax (h)^{a} | 1.75 | (0.75, 4.00) | 1.25 | (0.75, 2.00) | NC | NC |
| | T½ (h) | 4.46 | (31) | 3.90 | (22) | NC | NC |
| (4S)—OH | AUC(0-last) | 6.10 | (19) | 5.98 | (22) | NC | NC |
| | (ng · h/mL) | | | | | | |
| | AUC(0-inf) | 7.54 | (19) | 7.31 | (22) | NC | NC |
| | (ng · h/mL) | | | | | | |
| | Cmax (ng/mL) | 0.602 | (17) | 0.637 | (17) | NC | NC |
| | Tmax (h)^{a} | 4.00 | (2.00, 8.00) | 3.00 | (1.50, 4.00) | NC | NC |
| | T½ (h) | 7.94 | (34) | 7.26 | (32) | NC | NC |

Treatment A = cilansetron 2 mg tablet (batch # 61874) after an overnight fast;
Treatment B = cilansetron 2 mg tablet (batch # 61874), administered 5 minutes after completion of a high-calorie, high-fat breakfast.
(4R)—OH = (4R)-hydroxy cilansetron;
(4S)—OH = (4S)-hydroxy cilansetron
NC = not calculated;
% CV = coefficient of variation on arithmetic mean in percent.
^{a}Results presented are median (min, max).
^{b}Geometric least squares mean ratio and 90% confidence interval (CI) from ANOVA comparisons of log-transformed data.
Supporting Data: S241.1.111 clinical study report, (Left. Vol. 9 Righ)

The effect of food on the bioavailability of cilansetron (8 mg capsule) has also been evaluated in a randomized, open-label, single-dose, two-period, crossover study in 20 male volunteers, aged 23 to 35 years. Subjects were randomized to receive a cilansetron 8 mg capsule after an overnight fast and a cilansetron 8 mg capsule after a high-fat breakfast during the two treatment periods, with a 3-day washout between periods. Serial blood sampling and urine collection was performed up to 24 hours following each dose, and cilansetron pharmacokinetic parameters were evaluated. The results of these studies are summarized in Table 2.7.1.2.5. The results showed that administration of the capsule with a high-fat meal had no effect on the AUC of cilansetron, but produced a small decrease (21% on average) in Cmax. Tmax was prolonged by approximately 1.5 hours under fed conditions.

TABLE 2.7.1.2.5
Arithmetic Mean (% CV) Pharmacokinetic Parameters and
Statistical Comparisons for Cilansetron Following Administration
of Single Doses of Cilansetron 8 Mg Capsule Under Fasted
Conditions and Following a High-Fat Breakfast
(Study K.241.5006)

| | | | Statistical | |
|---|---|---|---|---|
| | | | Comparisons | |
| | Arithmetic Mean (% CV) | | (Treatment B/A)^{b} | |
| | Capsule Fed | Capsule Fasted | Ratio | |
| Parameter | (N = 20) | (N = 20) | (%) | 95% CI |
| AUC (0-inf) | 80.9 (65) | 77.6 (66) | 104 | 84, 130 |
| (ng · h/mL) | | | | |
| AUC (0-last) | 77.7 (68) | 72.8 (72) | 108 | 86, 135 |
| (ng · h/mL) | | | | |
| Cmax (ng/mL) | 24.5 (55) | 31.7 (58) | 79 | 61, 102 |
| Tmax (h)^{a} | 2.50 (0.75, 4.00) | 1.00 (0.50, 2.00) | NC | NC |
| T½ (h) | 1.50 (34) | 1.43 (40) | NC | NC |
| CL/F (mL/min) | 2556 (70) | 2698 (77) | NC | NC |
| Vz/F (L) | 299 (72) | 309 (76) | NC | NC |
| % Dose (urine) | 0.13 (69) | 0.21 (91) | NC | NC |
| CLr (mL/min) | 2.33 (63) | 3.86 (90) | NC | NC |

Treatment A = cilansetron 8 mg tablet (batch # 021N) after an overnight fast;
Treatment B = cilansetron 8 mg capsule (batch # 021N), administered after completion of a high-fat breakfast.
NC = not calculated;
% CV = coefficient of variation on arithmetic mean in percent.
^{a}Results presented are median (min, max).
^{b}Geometric least squares mean ratio and 95% confidence interval (CI) from ANOVA comparisons of log-transformed data.
Supporting Data: K.241.5006 clinical study report, (Left. Vol. 9 Righ)

Overview of Pharmacokinetic Clinical Studies

The pharmacokinetics of cilansetron have been evaluated in 20 clinical studies in approximately 382 healthy subjects and 224 non-constipated IBS patients. The results of these studies are detailed and summarized below. In brief, these studies (as set forth more fully below) have shown that:
Cilansetron is rapidly absorbed following oral administration, with a median Tmax of 1.0 to 1.75 hours following single and multiple dosing. Peak concentrations of its main metabolites, (4R)-hydroxy and (4S)-hydroxy cilansetron, are achieved at approximately 2 hours and 3-4 hours following dosing, respectively.
Cilansetron is completely absorbed from the gastrointestinal tract, based on a study utilizing orally administered ¹⁴C-cilansetron. It is a high extraction drug, with a low absolute bioavailability of 25%, indicating that it undergoes significant first pass metabolism on oral administration. It has a systemic plasma clearance of approximately 550-800 mL/min.
Steady-state conditions for cilansetron and its 4-hydroxy metabolites are achieved by the third day of three times per day (TID) dosing.
Cilansetron and its 4-hydroxy metabolites exhibit linear pharmacokinetics between single and multiple dosing. Consistent with their respective half-lives of approximately 1-2 hours, 4 hours, and 7.5 hours, cilansetron does not accumulate but (4R)-hydroxy cilansetron shows 35-47% accumulation and (4S)-hydroxy cilansetron shows greater than 2-fold accumulation following TID dosing.
The pharmacokinetics of cilansetron are highly variable after oral administration with intersubject coefficients of variation (% CVs) of 44-56% at the proposed dose of 2 mg TID. Intrasubject % CVs are about 19-25% following a 2 mg oral dose.
The steady-state volume of distribution (Vss) of cilansetron is about 56 L, which is slightly higher than total body water, suggesting limited distribution into tissues.
Cilansetron is approximately 96% plasma protein bound in humans over the therapeutic concentration range. (4R)-hydroxy cilansetron is 72-75% protein bound and (4S)-hydroxy cilansetron is 42-48% bound in plasma. The protein binding of cilansetron and its 4-hydroxy metabolites is not affected by mild to severe renal impairment. Protein binding of cilansetron is not affected by hepatic impairment.
Cilansetron is rapidly cleared from plasma with a mean elimination half-life of approximately 1-2 hours. Half-lives of (4R)-hydroxy and (4S)-hydroxy cilansetron are approximately 4 hours and 7.5 hours, respectively.
On oral dosing, 28% of the radioactivity from a dose of ¹⁴C-cilansetron is recovered in feces and 63% is recovered in urine over a period of 144 hours (<1% as unchanged drug and >30% as 4-hydroxy metabolites).
Cilansetron is highly metabolized, and in vitro studies have shown that the enzymes CYP3A4, CYP2D6, CYP1A1, CYP1A2, and CYP2C19 may be involved in its metabolism.
In a clinical study, fluvoxamine (inhibitor of CYP1A2, but also of CYP2C9, CYP2C19, and CYP3A) caused a 3.7- to 6.6-fold increase in cilansetron exposure. A 40-51% increase in exposure was observed with the CYP3A4 inhibitor, ketoconazole, and no significant effect was observed with the CYP2D6 inhibitor, paroxetine. These data suggest that CYP3A4, CYP1A2, and CYP2C19 are involved in the metabolism of cilansetron in humans.
Drug interaction studies have shown that the antacid, liquid Maalox®, does not affect the pharmacokinetics of cilansetron, and that cilansetron does not affect the pharmacokinetics or pharmacodynamics of the commonly used oral contraceptive, Ortho Tri-Cyclen® (containing ethinyl estradiol and norgestimate).
Cilansetron pharmacokinetics are dose proportional over a single IV dose range of 4-16 mg, and a single and multiple oral dose range of 2-32 mg.
The cilansetron 2 mg tablet is bioequivalent to an oral solution with respect to both cilansetron Cmax and AUC.
Administration of a high fat meal does not have a clinically significant effect on the bioavailability of the 2 mg tablet. Cmax of cilansetron is unchanged and AUC(0-inf) is only modestly increased (20%) under fed compared to fasted conditions.
Chiral inversion of cilansetron to its S-enantiomer does not occur in human plasma.
Age does not significantly affect the pharmacokinetics of cilansetron. No clear gender effects have been observed.
The pharmacokinetics of cilansetron are not affected by renal impairment, but the pharmacokinetics of its 4-hydroxy metabolites are significantly affected, particularly in the case of severe impairment. (4R)-hydroxy cilansetron exposure is increased by approximately 2-3 fold and (4S)-hydroxy cilansetron exposure is increased by approximately 5-fold with severe renal impairment.
Hepatic impairment causes an increase in cilansetron exposure (88% increase in cilansetron AUC and a 22% increase in Cmax). Only small decreases (up to 30-36%) are seen in (4R)-hydroxy and (4S)-hydroxy cilansetron exposure.
The pharmacokinetics of cilansetron and its 4-hydroxy metabolites in IBS patients are similar to that in healthy subjects.
Mean apparent oral clearance of cilansetron is increased by approximately 32% in IBS patients who are smokers.
IBS patients with higher exposure (AUC and Cmax) to cilansetron and its 4-hydroxy metabolites may be more likely to experience straining and/or harder stools.
Cilansetron has no effect on the mean or maximum QTc interval (Bazett's correction) following single or TID dosing in healthy subjects.

In addition, 16 studies were performed in healthy subjects and in selected patient populations to examine the pharmacodynamics of Cilansetron, these studies being described below.

Comparison and Analyses of Clinical Study Results

Single and Multiple Dose Pharmacokinetics

The pharmacokinetics of cilansetron have been evaluated in a single and multiple dose study at the proposed dose of 2 mg TID. In an open-label, randomized, parallel-group study in 36 healthy, male and female subjects (19-45 years), subjects received a 2 mg single dose of cilansetron on Day 1 and 2 mg TID on Days 2-7. The morning doses on Days 1 and 7 were administered immediately after a standard breakfast. Serial blood samples for the measurement of single-dose and steady-state pharmacokinetics of cilansetron and its main metabolites, (4R)-hydroxy cilansetron and (4S)-hydroxy cilansetron, were collected on Day 1 (single dose) up to 24 hours postdose and on Day 7 (multiple dose) up to 8 hours postdose.

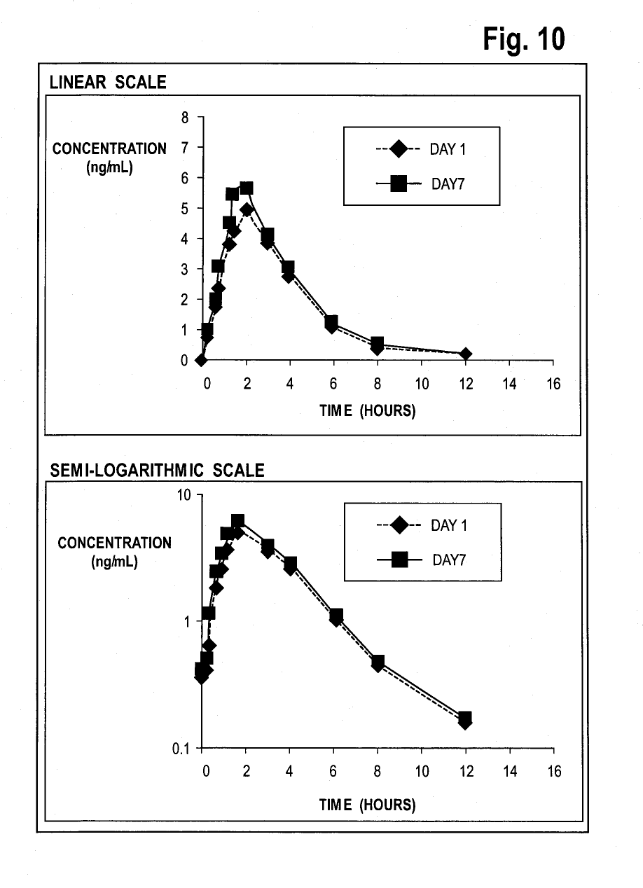

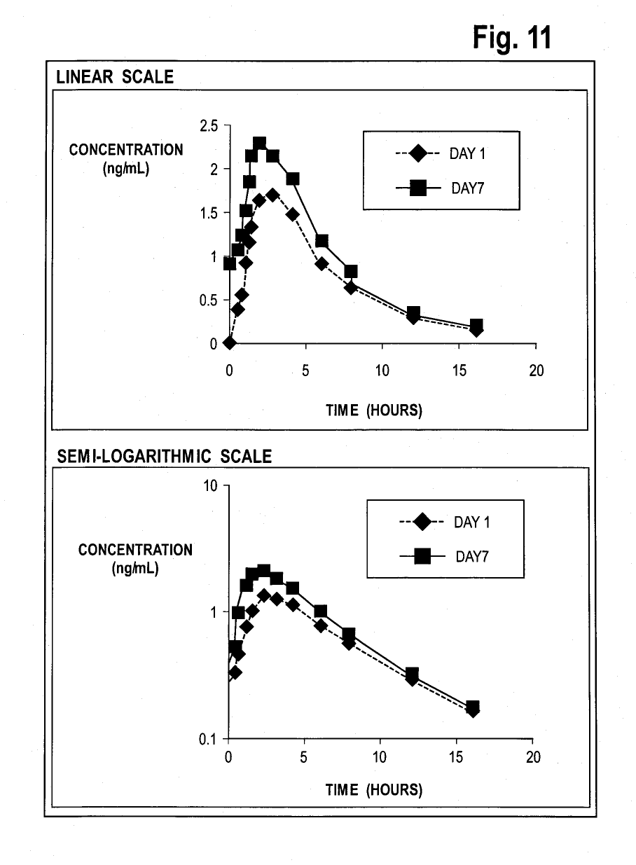

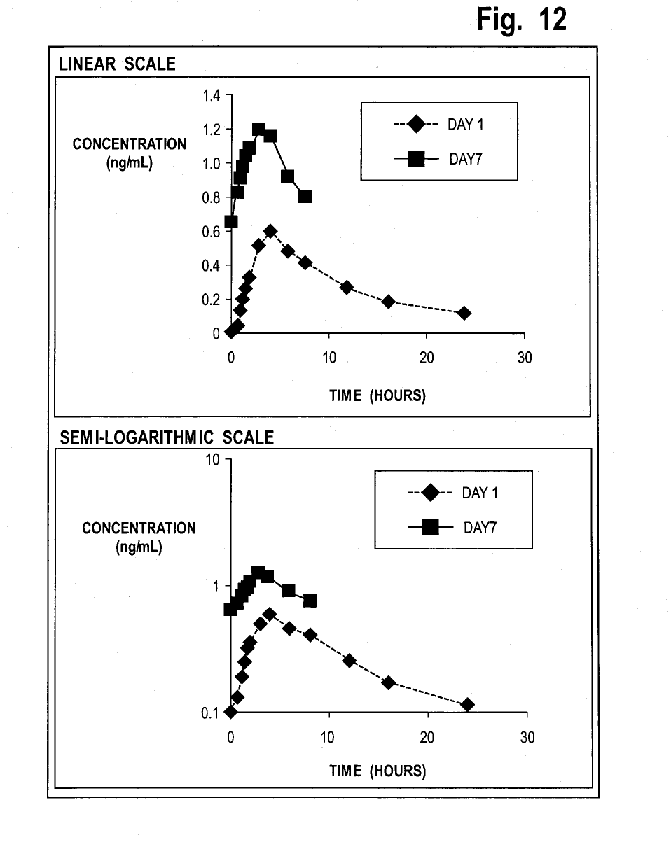

FIG. 10, FIG. 11, and FIG. 12 present geometric mean plasma concentration-time profiles for cilansetron and its 4-hydroxy metabolites following single dosing and at steady state. Table 2.7.2.3.1 presents descriptive statistics for the pharmacokinetic parameters of cilansetron and its 4-hydroxy metabolites, and the results of statistical comparisons of parameters on Day 7 and Day 1 (steady state versus single dose).

The results of this study showed that cilansetron is rapidly absorbed following oral administration, with a median Tmax of 1.5 to 1.75 hours following single and multiple dosing. After achieving maximum concentrations, cilansetron is rapidly cleared from plasma with a mean elimination half-life of 1.6 hours. Cilansetron concentrations undergo primarily monoexponential decline in plasma. Cilansetron exhibits linear pharmacokinetics between single and multiple dosing, as evidenced by the AUC(0-τ) (Day 7) to AUC(0-inf) (Day 1) ratio, which was close to unity. In keeping with its short half-life, no significant accumulation of cilansetron is seen following TID dosing, as demonstrated by the comparison of AUC(0-τ) and Cmax values on Day 7 to Day 1, which are only 5-10% higher on average on Day 7 compared to Day 1. The pharmacokinetics of cilansetron are highly variable, with intersubject % CVs for AUC and Cmax ranging from 44-56% in this study.

Unlike cilansetron, its two 4-hydroxy metabolites show some accumulation following multiple dosing, which is generally consistent with the half-lives of these metabolites. Peak concentrations of (4R)-hydroxy and (4S)-hydroxy cilansetron are achieved at 2 hours and 3-4 hours, respectively (median Tmax values), followed by a generally monoexponential decline with mean half-lives of approximately 4 hours and 7.5 hours, respectively. The AUC(0-τ) (Day 7) to AUC(0-inf) (Day 1) ratios are only slightly above unity (mean ratios of 1.07 and 1.08), demonstrating that both metabolites exhibit linear pharmacokinetics between single and multiple dosing. Based on AUC(0-τ) and Cmax values, accumulation (Day 7: Day 1) of the (4R)-hydroxy cilansetron is limited (mean increase of 35-47%). Accumulation of (4S)-hydroxy cilansetron is greater than 2-fold (mean increase of 105-139%) following TID dosing. The pharmacokinetics of both metabolites are not as variable as cilansetron, with intersubject % CVs for AUC and Cmax of approximately 18-27% in this study.

TABLE 2.7.2.3.1
Arithmetic mean (% CV) single dose and steady state pharmacokinetic
parameters for cilansetron and its 4-hydroxy metabolites and results of
statistical comparisons between steady-state (2 mg TID; Day 7) and single
dose (2 mg; Day 1) parameters following oral dosing (Study S.241.1.109)

| | | Arith. Mean (% CV) | | Statistical | |
|---|---|---|---|---|---|
| | | Cilansetron | Cilansetron | Comparisons | |
| | | 2 mg Single Dose | 2 mg TID | (Day 7/Day 1)^{c} | |
| Analyte | Parameter | Day 1 (n = 35) | Day 7 (n = 35) | Ratio (%) | 90% CI |
| Cilansetron | AUC(0-τ) (ng · h/mL) | 21.0 (50) | 23.5 (44) | 105^{d} | 94, 117^{d} |
| | AUC(0-inf) (ng · h/mL) | 22.3 (50) | NA | NA | NA |
| | Cmax (ng/mL) | 6.63 (56) | 7.55 (48) | 110^{e} | 98, 124^{e} |
| | Tmax (h) | 1.75 (0.50, 4.00)^{b} | 1.50 (0.50, 4.00)^{b} | NC | NC |
| | T½ (h) | 1.64 (18) | NC | NC | NC |
| | Linearity Ratio^{a} | NA | NA | 99 | 89, 110 |
| (4R)—OH | AUC(0-τ) (ng · h/mL) | 8.43 (21) | 12.9 (18) | 147^{d} | 138, 156^{d} |
| | AUC(0-inf) (ng · h/mL) | 11.7 (18) | NA | NA | NA |
| | Cmax (ng/mL) | 1.92 (27) | 2.66 (22) | 135^{e} | 124, 147^{e} |
| | Tmax (h) | 2.00 (0.50, 4.00)^{b} | 2.00 (0.75, 4.00)^{b} | NC | NC |
| | T½ (h) | 3.89 (15) | NC | NC | NC |
| | Linearity Ratio^{a} | NA | NA | 107 | 102, 111 |
| (4S)—OH | AUC(0-τ) (ng · h/mL) | 3.26 (19) | 7.91 (19) | 239^{d} | 223, 256^{d} |
| | AUC(0-inf) (ng · h/mL) | 7.42 (20) | NA | NA | NA |
| | Cmax (ng/mL) | 0.605 (22) | 1.25 (18) | 205^{e} | 192, 219^{e} |
| | Tmax (h) | 4.00 (1.50, 6.00)^{b} | 3.00 (1.75, 4.00)^{b} | NC | NC |
| | T½ (h) | 7.48 (26) | NC | NC | NC |
| | Linearity Ratio^{b} | NA | NA | 108 | 103, 113 |

NC—not calculated;
NA—not applicable;
% CV - coefficient of variation on arithmetic mean in percent;
AUC(0-τ) - AUC(0-8)
^{a}Statistical comparison of AUC(0-τ) on Day 7 to AUC(0-inf) on Day 1.
^{b}Results presented are median (min, max).
^{c}Results of ANOVA comparisons of log-transformed data (multiple dose/single dose) (n = 35).
^{d}AUC accumulation ratio (statistical comparison of AUC(0-τ) on Day 7:Day 1).
^{e}Cmax accumulation ratio (statistical comparison of Cmax on Day 7:Day 1).

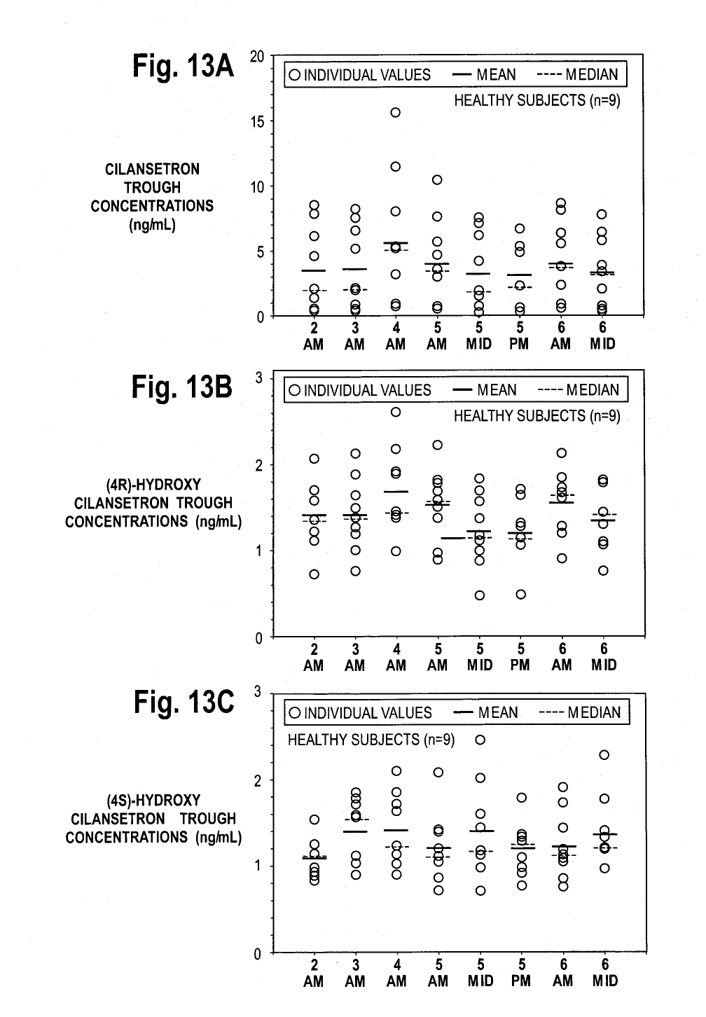

Time to reach steady state following dosing with cilansetron 2 mg TID has been evaluated in a study in 9 healthy male and female subjects. Plots of trough concentrations of cilansetron and its 4-hydroxy metabolites measured prior to morning dosing on Days 2, 3, and 4, prior to all doses on Day 5, and prior to the morning and scheduled mid-day dose on Day 6 are shown in FIG. 13. Based on statistical and graphical evaluations of trough concentrations, steady state was achieved by approximately the third day of dosing for cilansetron and its 4-hydroxy metabolites, which is consistent with the estimated half-lives of these analytes.

Two randomized, double-blind, placebo controlled, rising dose, crossover studies evaluated the single-dose pharmacokinetics of oral cilansetron over a 4 mg to 64 mg dose range. In one study, 24 male subjects (aged 21-36 years) received, at intervals of 21 week, single oral doses of 4, 8, and 16 mg cilansetron in ascending order and a randomized placebo. In the other study, 24 male subjects received, at intervals of ≧1 week, single oral doses of 16, 32, and 64 mg cilansetron in ascending order and a randomized placebo. All doses were administered under fasted conditions. Serial blood sampling and urine collection was performed up to 24 hours following each dose and cilansetron pharmacokinetic parameters were evaluated. The results of these studies are summarized in Table 2.7.2.3.2 below. Again, the data showed that cilansetron is rapidly absorbed following oral administration, with a median Tmax of 1 to 1.25 hours over the entire dose range. After achieving maximum concentrations, cilansetron is rapidly cleared from plasma with a mean half-life of 1.2 to 1.6 hours, regardless of dose administered. The pharmacokinetics of cilansetron were highly variable, with intersubject % CVs for AUC and Cmax ranging from approximately 62-103%. Renal excretion of cilansetron was negligible across all doses, with undetectable or very low amounts (<1% of the dose) excreted unchanged in urine.

TABLE 2.7.2.3.2
Arithmetic mean (% CV) pharmacokinetic parameters for cilansetron
following administration of single oral rising doses of 4 mg to 64 mg
(Study K.241.5001 and Study K.241.5002)

| | Study K.241.5001 (N = 24) | | | | | | Study K.241.5002 (N = 24) | | | | | |
|---|---|---|---|---|---|---|---|---|---|---|---|---|
| Parameter | 4 mg | | 8 mg | | 16 mg | | 16 mg | | 32 mg | | 64 mg | |
| AUC(0-inf) | 26.7 | (70) | 59.4 | (77) | 138 | (97) | 141 | (79) | 330 | (72) | 813 | (80) |
| (ng · h/mL) | | | | | | | | | | | | |
| DN-AUC(0-inf) | 6.67 | (70) | 7.43 | (77) | 8.63 | (97) | 8.83 | (79) | 10.3 | (72) | 12.7 | (80) |
| (ng · h/mL) | | | | | | | | | | | | |
| AUC(0-last) | 23.2 | (78) | 55.5 | (81) | 129 | (103) | 136 | (83) | 324 | (74) | 807 | (81) |
| (ng · h/mL) | | | | | | | | | | | | |
| DN-AUC(0-last) | 5.80 | (78) | 6.94 | (81) | 8.06 | (103) | 8.50 | (83) | 10.1 | (74) | 12.6 | (81) |
| (ng · h/mL) | | | | | | | | | | | | |
| Cmax (ng/mL) | 9.88 | (68) | 22.1 | (65) | 49.9 | (85) | 61.0 | (77) | 143 | (62) | 349 | (64) |
| DN-Cmax (ng/mL) | 2.47 | (68) | 2.76 | (65) | 3.12 | (85) | 3.80 | (77) | 4.46 | (62) | 5.45 | (64) |
| Tmax (h)^{a} | 1.25 | (0.50, 2.00) | 1.00 | (0.50, 2.00) | 1.00 | (0.50, 2.50) | 1.00 | (0.67, 2.00) | 1.00 | (0.67, 2.00) | 1.00 | (0.67, 2.00) |
| T½ (h) | 1.56 | (24) | 1.47 | (30) | 1.34 | (28) | 1.23 | (26) | 1.28 | (22) | 1.30 | (25) |
| CL/F (mL/min) | 3864 | (69) | 3851 | (103) | 3589 | (71) | 3242 | (74) | 2487 | (79) | 1910 | (60) |
| Vz/F (L) | 522 | (86) | 472 | (106) | 397 | (78) | 323 | (68) | 269 | (75) | 204 | (58) |

| | | | | | | | | | |
|---|---|---|---|---|---|---|---|---|---|
| % Dose (urine) | NE | NE | NE | 0.16 | (88) | 0.29 | (105) | 0.28 | (92) |
| CLr (mL/min) | NE | NE | NE | 3.39 | (89) | 5.00 | (90) | 4.50 | (102) |

DN - dose normalized to a 1 mg dose;
NE - not estimable in many subjects - many urine concentrations were below assay detection limits;
% CV - coefficient of variation on arithmetic mean in percent.
^{a}Results presented are median (min, max).

The single- and multiple-dose pharmacokinetics of cilansetron at oral doses of 4 mg and 8 mg were evaluated in a randomized, double-blind, placebo controlled, parallel group study in 32 male subjects (14 subjects received 4 mg cilansetron, 14 received 8 mg cilansetron, and 4 received placebo). Subjects received a single dose of cilansetron at their assigned dose on the first day of the study (Day 1), followed by a 7-day washout, after which they received cilansetron TID at their assigned dose for 6 days and a single dose on the morning of the seventh day (Day 14). Morning doses on all days were administered under fasted conditions. Serial blood sampling and urine collection was performed up to 12 hours following dosing on Day 1 and up to 24-30 hours following dosing on Day 14 and cilansetron pharmacokinetic parameters were evaluated. The results of this study are summarized in Table 2.7.2.3.3 below. Cilansetron was rapidly absorbed following oral administration, with a median Tmax of 1 to 1.4 hours following single and multiple dosing and was rapidly cleared from plasma with a mean half-life of 1.6 to 1.9 hours. No differences were observed in half-life between single and multiple dosing. Comparison of cilansetron AUC between multiple versus single dosing showed a slightly greater than expected exposure for multiple dosing [mean AUC(0-τ) (Day 14) to AUC(0-inf) (Day 1) ratios were 111% and 123% for 4 mg and 8 mg doses, respectively]. Intersubject % CVs for AUC and Cmax were large, ranging from approximately 36-82%, and intrasubject % CVs on AUC were 33-55%. Renal excretion of cilansetron was negligible following single and multiple dosing at both doses, with less than 1% of the dose excreted unchanged in urine.

TABLE 2.7.2.3.3
Arithmetic mean (% CV) single- and multiple-dose pharmacokinetic
parameters for cilansetron following administration of oral cilansetron
4 mg and 8 mg as single doses and TID for 7 days (Study K.241.5003)

| | Cilansetron 4 mg (n = 14) | | Cilansetron 8 mg (n = 14) | |
|---|---|---|---|---|
| Parameter | Single Dose | Multiple Dose | Single Dose | Multiple Dose |
| AUC (ng · h/mL)^{a} | 24.3 (36) | 30.3 (39) | 52.4 (69) | 58.1 (61) |
| DN-AUC (ng · h/mL)^{a} | 6.08 (36) | 7.57 (39) | 6.55 (69) | 7.27 (61) |
| Cmax (ng/mL) | 8.83 (37) | 11.5 (49) | 23.1 (61) | 28.8 (82) |
| DN-Cmax (ng/mL) | 2.21 (37) | 2.87 (49) | 2.89 (61) | 3.61 (82) |
| Tmax (h)^{b} | 1.38 (0.66, 2.00) | 1.00 (0.66, 3.00) | 1.00 (0.66, 2.00) | 1.00 (0.66, 2.00) |
| Cmin (ng/mL) | NA | 1.31 (114) | NA | 1.65 (147) |
| T½ (h) | 1.55 (35) | 1.88 (40) | 1.92 (108) | 1.70 (95) |
| CL/F (mL/min) | 3226 (51) | NC | 3559 (62) | NC |
| Vz/F (L) | 406 (49) | NC | 483 (88) | NC |
| % Dose (urine)^{c} | 0.18 (91) | 0.13 (111) | 0.14 (79) | 0.15 (87) |
| CLr (mL/min) | 7.47 (143) | 3.36 (106) | 4.68 (92) | 4.24 (95) |
| Linearity Ratio (%)^{d} | NA | 111 (86, 142) | NA | 123 (86, 176) |

NC—not calculated;
NA—not applicable;
% CV - coefficient of variation on arithmetic mean in percent;
DN—dose normalized to a 1 mg dose.
^{a}AUC is AUC (0-inf) for single dose and AUC (0-τ) (i.e. AUC (0-8)) for multiple dose.
^{b}Results presented are median (min, max).
^{c}Percent of dose excreted in urine over a 12-hour interval postdose for single dosing, and over an 8-hour interval postdose for multiple dosing.
^{d}Statistical comparison of AUC (0-τ) on Day 14 to AUC (0-inf) on Day 1; results presented are geometric least squares mean ratio and 90% confidence interval.

Two randomized, double-blind, placebo controlled, balanced incomplete block design, crossover studies evaluated the single- and multiple-dose pharmacokinetics of oral cilansetron over a 8 mg to 32 mg dose range. In both studies, subjects were randomized to receive two of three treatments during 2 treatment periods. In one study (12 male and 13 female subjects, aged 23 to 36 years), subjects received 8 mg cilansetron, 16 mg cilansetron, or placebo, while in another study (12 males and 13 females, aged 23 to 41 years), subjects received 16 mg cilansetron, 32 mg cilansetron, or placebo. During each treatment period, subjects received a single dose of their assigned treatment on the first day, followed by a 3-day washout, after which they received the same treatment TID for 5 days.

There was a 5-6 day washout period between treatment periods. Doses on the mornings of pharmacokinetic days were administered under fasted conditions. Serial blood sampling and urine collection was performed up to 12 hours following morning dosing on the first day (single dose) and on the last multiple dosing day of each treatment period, and cilansetron pharmacokinetic parameters were evaluated. The results of these studies are summarized in Table 2.7.2.3.4 and Table 2.7.2.3.5 below.

In both studies, cilansetron was rapidly absorbed following oral administration, with a median Tmax of 1 hour following single and multiple dosing and was rapidly cleared from plasma with a mean half-life of 1.2 to 1.4 hours. No differences were observed in half-life between single and multiple dosing. Comparison of cilansetron AUC between multiple versus single dosing showed a slightly lower than expected exposure with multiple dosing in both studies. Mean AUC(0-τ) (multiple dose) to AUC(0-inf) (single dose) ratios were 81% to 94% over the 8 mg to 32 mg dose range. Intersubject % CVs for AUC and Cmax were again large, ranging from approximately 36-75%, but intrasubject % CVs were lower (23-51%). Renal excretion of cilansetron was negligible following single and multiple dosing at all doses, with less than 1% of the dose excreted unchanged in urine.

TABLE 2.7.2.3.4
Arithmetic mean (% CV) single- and multiple-dose pharmacokinetic
parameters for cilansetron following administration of oral cilansetron
8 mg and 16 mg as single doses and TID for 5 days (Study S.241.1.101)

| | Cilansetron 8 mg | | | |
|---|---|---|---|---|
| | (n = 15) | | Cilansetron 16 mg (n = 16) | |
| | Single | Multiple | Single | Multiple |
| Parameter | Dose | Dose | Dose | Dose |
| AUC | 112 (49) | 88.1 (47) | 229 (72) | 224 (68) |
| (ng · h/mL)^{a} | | | | |
| DN-AUC | 14.0 (49) | 11.0 (47) | 14.3 (72) | 14.0 (68) |
| (ng · h/mL)^{a} | | | | |
| Cmax (ng/mL) | 43.3 (38) | 39.3 (49) | 98.3 (60) | 96.1 (66) |
| Tmax (h)^{b} | 1.00 | 1.00 | 1.00 | 1.00 |
| | (0.67, 1.50) | (0.67, 2.00) | (0.67, 2.00) | (0.67, 2.00) |
| Cmin (ng/mL) | NA | 3.65 (84) | NA | 9.19 (79) |
| T½ (h) | 1.44 (29) | 1.31 (19) | 1.30 (25) | 1.42 (25) |
| % Dose | 0.37 (97) | 0.29 (121) | 0.28 (114) | 0.16 (81) |
| (urine)^{c} | | | | |
| Linearity | NA | 81 (69, 95) | NA | 94 (81, 110) |
| Ratio (%)^{d} | | | | |

NA—not applicable;
% CV - coefficient of variation on arithmetic mean in percent;
DN—dose normalized to a 1 mg dose.
^{a}AUC is AUC (0-inf) for single dose and AUC (0-τ) (i.e. AUC (0-8)) for multiple dose.
^{b}Results presented are median (min, max).
^{c}Percent of dose excreted in urine over a 12-hour interval postdose for single dosing, and over an 8-hour interval postdose for multiple dosing.
^{d}Statistical comparison of AUC (0-τ) on multiple dose day to AUC (0-inf) on single dose day; results presented are geometric least squares mean ratios (90% confidence intervals).

TABLE 2.7.2.3.5
Arithmetic mean (% CV) single- and multiple-dose pharmacokinetic
parameters for cilansetron following administration of oral cilansetron
16 mg and 32 mg as single doses and TID for 5 days
(Study S.241.1.102)

| | Cilansetron 16 mg | | | |
|---|---|---|---|---|
| | (n = 15) | | Cilansetron 32 mg (n = 16) | |
| | Single | Multiple | Single | Multiple |
| Parameter | Dose | Dose | Dose | Dose |
| AUC | 174 (75) | 139 (36) | 451 (61) | 380 (46) |
| (ng · h/mL)^{a} | | | | |
| DN-AUC | 10.9 (75) | 8.69 (36) | 14.1 (61) | 11.9 (46) |
| (ng · h/mL)^{a} | | | | |
| Cmax (ng/mL) | 68.4 (49) | 67.3 (41) | 216 (70) | 175 (55) |
| Tmax (h)^{b} | 1.00 | 1.00 | 1.00 | 1.00 |
| | (0.67, 2.00) | (0.33, 1.50) | (0.67, 2.00) | (0.67, 1.50) |
| Cmin (ng/mL) | NA | 2.83 (111) | NA | 12.0 (149) |
| T½ (h) | 1.35 (30) | 1.29 (32) | 1.22 (27) | 1.24 (29) |
| % Dose | 0.49 (103) | 0.21 (52) | 0.50 (57) | 0.36 (81) |
| (urine)^{c} | | | | |
| Linearity | NA | 90 (76, 107) | NA | 88 (75, 104) |
| Ratio (%)^{d} | | | | |

NA—not applicable;
% CV - coefficient of variation on arithmetic mean in percent;
DN—dose normalized to a 1 mg dose.
^{a}AUC is AUC (0-inf) for single dose and AUC (0-τ) (i.e. AUC (0-8)) for multiple dose.
^{b}Results presented are median (min, max).
^{c}Percent of dose excreted in urine over a 12-hour interval postdose for single dosing, and over an 8-hour interval postdose for multiple dosing.
^{d}Statistical comparison of AUC (0-τ) on multiple dose day to AUC (0-inf) on single dose day; results presented are geometric least squares mean ratios (90% confidence intervals).

The pharmacokinetics of intravenously administered cilansetron have also been evaluated in several studies. In a randomized, double-blind, placebo controlled, rising dose, crossover study in 24 male subjects, aged 23-37 years, the single-dose pharmacokinetics of IV cilansetron were evaluated over a 4 mg to 16 mg dose range. Subjects received, at intervals of 1 week, single doses of 4, 8, and 16 mg IV cilansetron in ascending order and a randomized placebo. Infusion times ranged from 15-30 minutes. All doses were administered under fasted conditions. Serial blood sampling was performed until 12 hours following start of infusion and urine collection was performed up to 24 hours following start of infusion, and cilansetron pharmacokinetic parameters were evaluated. The results of this study showed that cilansetron has a high systemic plasma clearance of about 550-700 mL/min (Table 2.7.2.3.6). On IV dosing, its mean elimination half-life ranged from 1.4 to 1.7 hours, and was independent of dose, demonstrating the absence of nonlinear pharmacokinetics over this dose range. Additionally, the elimination half-life of cilansetron after IV dosing was comparable to the terminal half-life after oral dosing, demonstrating that flip-flop pharmacokinetics do not occur with oral dosing. As seen with oral dosing, renal elimination was negligible (<1% of dose) for all the IV doses studied.

TABLE 2.7.2.3.6
Arithmetic mean (% CV) single-dose pharmacokinetic parameters for
cilansetron following administration of IV rising doses of
4 mg to 16 mg (Study K.241.5004)

| | 4 mg | 8 mg | 16 mg |
|---|---|---|---|
| Parameter | (n = 24) | (n = 22) | (n = 21) |
| AUC (0-inf) (ng · h/mL) | 103 (25) | 254 (31) | 525 (37) |
| DN-AUC (0-inf) (ng · h/mL) | 25.8 (25) | 31.7 (31) | 32.8 (37) |
| AUC (0-last) (ng · h/mL) | 100 (26) | 2 51 (31) | 518 (36) |
| DN-AUC (0-last) (ng · h/mL) | 25.1 (26) | 31.4 (31) | 32.4 (36) |
| T½ (h) | 1.70 (25) | 1.62 (24) | 1.41 (18) |
| CL (mL/min) | 680 (22) | 567 (26) | 564 (30) |
| Vz (L) | 99 (28) | 78 (31) | 68 (34) |
| % Dose (urine) | 0.39 (120) | 0.49 (77) | 0.50 (87) |
| CLr (mL/min) | 2.95 (151) | 2.64 (88) | 2.65 (74) |

DN—dose normalized to a 1 mg dose;
% CV - coefficient of variation on arithmetic mean in percent.

The single- and multiple-dose pharmacokinetics of cilansetron 8 mg administered intravenously have been studied in a randomized, double-blind, placebo controlled, parallel group study in 12 male subjects (10 subjects received cilansetron 8 mg and 2 received placebo, administered as a 30-minute infusion). Subjects received a single dose of their assigned treatment on the first day of the study, followed by a washout of at least 2 days, after which they received their assigned treatment TID for 4 days and a single dose on the morning of the fifth day. Serial blood sampling was performed up to 12 hours following dosing on the single dosing day and up to 30 hours following dosing on the last day of multiple dosing, and urine collection was performed up to 12 hours following dosing on the single dosing day and up to 24 hours following dosing on the last day of multiple dosing, and cilansetron pharmacokinetic parameters were evaluated. Doses on pharmacokinetic days were administered under fasted conditions. The results of this study showed that cilansetron has a high systemic plasma clearance of 800 mL/min (Table 2.7.2.3.7). The mean elimination half-life of cilansetron ranged from 1.7 to 2.1 hours following single and multiple dosing, similar to that seen in other studies with IV and oral cilansetron. Intersubject % CVs for AUC were 31-34% and intrasubject % CV was 18%. Renal elimination was again observed to be negligible (≦1% of dose).

TABLE 2.7.2.3.7
Arithmetic mean (% CV) single- and multiple-dose pharmacokinetic
parameters for cilansetron following IV administration of cilansetron
8 mg as a single dose and TID for 5 days (Study K.241.5005)

| | Single Dose | Multiple Dose |
|---|---|---|
| Parameter | (n = 10) | (n = 9) |
| AUC (ng · h/mL)^{a} | 182 (31) | 226 (34) |
| DN-AUC (ng · h/mL)^{a} | 22.8 (31) | 28.2 (34) |
| Cmin (ng/mL) | NA | 2.90 (44) |
| T½ (h) | 1.70 (47) | 2.05 (55) |
| CL (mL/min) | 792 (28) | NC |
| % Dose (urine)^{b} | 0.44 (70) | 1.11 (108) |
| CLr (mL/min) | 3.29 (64) | 6.37 (88) |
| Linearity Ratio (%)^{c} | NA | 118 (101, 137) |

NC—not calculated;
NA—not applicable;
DN—dose normalized to a 1 mg dose;
% CV - coefficient of variation on arithmetic mean in percent.
Only subjects who completed all single and multiple dosing (n = 9) are included in this summarization
^{a}AUC is AUC (0-inf) for single dose and AUC (0-τ) (i.e. AUC (0-8)) for multiple dose.
^{b}Percent of dose excreted in urine over a 12-hour interval postdose for single dosing, and over an 8-hour interval postdose for multiple dosing.
^{c}Statistical comparison of AUC (0-τ) for multiple dosing to AUC (0-inf) for single dosing; results presented are geometric least squares mean ratio and 90% confidence interval.

On IV administration, cilansetron exhibits a mean steady-state volume of distribution (Vss) of cilansetron of about 56 L, which is slightly higher than total body water, and indicates limited distribution into tissues (Table 2.7.2.3.9).

Cilansetron easily passes biological membranes, probably including the blood-brain barrier. However, cilansetron is a substrate for P-glycoprotein (Table 2.7.2.3.8) and, therefore, brain concentrations are probably kept at a low level at therapeutic concentrations (plasma Cmax of approximately 6-7 ng/mL at a dose of 2 mg TID). Much higher concentrations, above 1000 ng/mL, were required to saturate P-glycoprotein in vitro. The 4-hydroxy metabolites of cilansetron were found to be six times stronger substrates for P-glycoprotein than cilansetron and passed biological membranes more slowly than cilansetron.

TABLE 2.7.2.3.8
In vitro transport of cilansetron and its 4-hydroxy metabolites by human
P-glycoprotein expressed in PK 1 LLC MDR cells (Study K.241.6026)

| Compound | P-gp Factor^{b} | Membrane Passage^{c} |
|---|---|---|
| Cilansetron | 3 | 33% |
| (4S)-hydroxy cilansetron | 17 | 13% |
| (4R)-hydroxy cilansetron | 18 | 14% |
| DU 29373^{a} | 7 | 30% |

^{a}Reference compound (good substrate for P-glycoprotein).
^{b}Ratio of percentages of compound transported from apical to basolateral versus basolateral to apical after 3 hours of incubation.
^{c}Arithmetic mean percentages of compound transported from apical to basolateral versus basolateral to apical after 3 hours of incubation.

Bioavailability

The absolute bioavailability of cilansetron 8 mg oral solution and 8 mg capsule as been evaluated in comparison to an IV solution in a randomized, open-label, single-dose, three-period, crossover study in 18 male subjects, aged 21 to 38 years. Subjects were randomized to receive cilansetron 8 mg IV solution (30-minute infusion), cilansetron 8 mg oral solution, and a cilansetron 8 mg capsule during the three treatment periods, with a 23-day washout between periods. All doses were administered under fasted conditions. Serial blood sampling and urine collection was performed up to 9 hours following each dose and cilansetron pharmacokinetic parameters were evaluated. The results of this study are summarized in Table 2.7.2.3.9 below. The systemic plasma clearance of cilansetron was approximately 600 mL/min, and the absolute bioavailability of an oral solution of cilansetron was 25%. Its low bioavailability, complete absorption from the GI tract, and its 4.5- to 5-fold higher apparent oral clearance in comparison to systemic clearance indicate that cilansetron is a high extraction drug and undergoes significant first pass metabolism on oral administration. Intersubject % CVs were higher for oral (47-73%) than for IV administration (19-20%), reflecting increased variability due to oral absorption. Intrasubject % CVs ranged from 37-47% following oral and IV administration.

TABLE 2.7.2.3.9
Arithmetic mean (% CV) single-dose pharmacokinetic parameters for
cilansetron following administration of cilansetron 8 mg as an IV solution,
oral solution, and oral capsule (Study K.241.5015)

| | | | | Statistical Comparisons | |
|---|---|---|---|---|---|
| | | | | Ratio (90% CI) (%)^{c} | |
| | IV Solution | Oral Solution | Oral Capsule | Oral Soln/ | Oral Capsule/ |
| Parameter | (n = 17) | (n = 17) | (n = 18) | IV Soln | IV Soln |
| AUC(0-inf) (ng · h/mL) | 234 (19) | 73.8 (69) | 66.0 (47) | 26 (20, 34) | 25 (19, 32) |
| AUC(0-last) (ng · h/mL) | 228 (20) | 69.3 (73) | 62.1 (48) | 24 (18, 32) | 23 (18, 31) |
| Cmax (ng/mL) | NA | 39.2 (67) | 31.4 (49) | NA | NA |
| Tmax (h)^{a} | NA | 0.83 (0.33, 1.25) | 1.00 (0.67, 2.50) | NC | NC |
| T½ (h) | 1.33 (22) | 1.47 (21) | 1.28 (22) | NC | NC |
| CL (mL/min) | 590 (18) | NA | NA | NC | NC |
| CL/F (mL/min) | NA | 2648 (60) | 2824 (80) | NC | NC |
| Vas (L) | 55.6 (18) | NA | NA | NC | NC |
| Vz or Vz/F (L)^{b} | 66.7 (24) | 342 (75) | 308 (79) | NC | NC |
| % Dose (urine) | 0.41 (96) | 0.15 (94) | 0.12 (109) | NC | NC |
| CLr (mL/min) | 2.30 (92) | 2.92 (88) | 2.37 (95) | NC | NC |

NC—not calculated;
NA—not applicable;
% CV - coefficient of variation on arithmetic mean in percent.
^{a}Results presented are median (min, max).
^{b}Vz for IV dose and Vz/F for oral doses.
^{c}Results of ANOVA comparisons of log-transformed data (N = 17).

Cilansetron concentrations in this study were also modeled using a standard 2-compartment model for the IV data and a 1-compartment model for the oral data (using TOPFIT 2.0). The results from modeling are compared to the results obtained from non-compartmental analysis in Table 2.7.2.3.10 below, for the oral and IV solution. The results obtained by the two methods are very similar.

TABLE 2.7.2.3.10
Arithmetic mean (SD) single-dose pharmacokinetic parameters for
cilansetron obtained by non-compartmental methods and by modeling,
following administration of cilansetron 8 mg as an IV and oral solution
(Study K.241.5015)

| | IV Solution | | Oral Solution | |
|---|---|---|---|---|
| | Non- | | Non- | |
| Parameter | compartmental | Model^{a} | compartmental | Model^{a} |
| AUC (0-inf) (ng · h/mL) | 234 (45) | 222 (42) | 74 (51) | 69 (49) |
| Cmax (ng/mL) | NA | 149 (29) | 39 (26) | 29 (18) |
| Tmax (h) | NA | 0.55 (0.05) | 0.76 (0.34) | 0.76 (0.40) |
| T½ (h) | 1.33 (0.30) | 1.33 (0.32) | 1.47 (0.30) | 1.14 (0.27) |
| CL (mL/min) | 590 (107) | 619 (108) | NA | NA |
| Vss (L) | 56 (10) | 58 (11) | NA | NA |

NA—not applicable
^{a}Concentrations were fitted using a 2-compartment model for IV and 1-compartment model for oral dosing.

The relative bioavailability of the cilansetron 2 mg tablet was evaluated in comparison to a 2 mg oral solution in a randomized, open-label, single-dose, three-period, crossover study in 12 male and 6 female subjects, aged 21 to 41 years. Additionally, the relative bioavailability of a cilansetron 8 mg capsule was evaluated in comparison to an 8 mg oral solution in a randomized, open-label, single-dose, three-period, crossover study in 18 male subjects, aged 21 to 38 years. In brief, the tablet was found to be bioequivalent to the oral solution with respect to both cilansetron Cmax and AUC. Pharmacokinetic parameters for the 4-hydroxy metabolites were also similar between treatments. In the case of the comparison of capsule to oral solution, cilansetron AUC was found to be comparable between the two treatments and Cmax was only slightly lower (12%) for the capsule than for the solution. Intrasubject % CVs for AUC and Cmax were 19.2% and 24.5%, respectively.

Effect of Food

The effect of a high-fat breakfast on the bioavailability of the cilansetron 2 mg tablet has been evaluated in a randomized, open-label, single-dose, three-period, crossover study in 12 male and 6 female subjects, aged 21 to 41 years. Additionally, the effect of food on the bioavailability of a cilansetron 8 mg capsule has been evaluated in a randomized, open-label, single-dose, two-period, crossover study in 20 male subjects, aged 23 to 35 years. In brief, these studies showed that administration with a high-fat meal caused a small but clinically insignificant increase in the bioavailability of the cilansetron tablet. No change was observed in cilansetron Cmax, but AUC(0-inf) increased by 20% on average under fed conditions. In the case of the 4-hydroxy metabolites, AUC values appeared essentially unchanged and Cmax appeared only slightly lower under fed conditions. For the capsule, a high-fat meal had no effect on the AUC of cilansetron, but produced a small decrease (21% on average) in Cmax.

Diurnal Effects

Diurnal variations in cilansetron pharmacokinetics have been evaluated in a randomized, double-blind, placebo-controlled, 2-way crossover study in 9 male and 9 female healthy subjects, aged 21 to 39 years. Subjects were randomized to receive 2 treatments (cilansetron 16 mg tablet and placebo) during the 2 treatment periods. During each treatment period, subjects received a single dose of their assigned treatment on the first day (Day 1), after which they received the same treatment TID for 4 days (Days 2-5). There was a 10-day washout period between treatment periods. Subjects were fasted for at least 2 hours before and following each dose, and heavy physical activity was restricted. Serial blood samples for the measurement of multiple-dose pharmacokinetics of cilansetron were collected up to 24 hours postdose on Day 5.

Table 2.7.2.3.1 1 presents descriptive statistics for cilansetron AUC and Cmax following each dose on Day 5, and the results of statistical comparisons between parameters measured during the different dosing intervals.

The results of this study showed that cilansetron exposure following TID dosing was identical between the different dosing intervals (mean ratios and 90% confidence intervals were within the 80-125% bioequivalence criterion), but mean Cmax was 30-33% higher following the morning dose than the mid-day and evening doses. The reason for the higher Cmax is not clear but may be due to differences in body posture, physical activity, and/or food intake (since subjects were fasted relative to the morning dose). Tmax was similar during the different dosing intervals. No differences were observed in interindividual variability of cilansetron pharmacokinetics at the different dosing times. Intersubject % CVs for AUC and Cmax were similar over the different time intervals, ranging from 52-64%.

TABLE 2.7.2.3.11
Arithmetic mean (% CV) pharmacokinetic parameters and statistical
comparisons for cilansetron following morning, mid-day, and evening
dosing on Day 5, following oral dosing with 16 mg TID for 4 days
(Study S.241.1.108)

| | Arithmetic Mean (% CV) | | | Statistical Comparisons^{b} | | |
|---|---|---|---|---|---|---|
| | for Dosing Intervals on Day 5 | | | Ratio (90% CI) (%) | | |
| | 0-8 h | 8-16 h | 16-24 h | 0-8 h/8-16 h | 0-8 h/16-24 h | 8-16 h/16-24 h |
| AUC (ng · h/mL) | 214 (64) | 199 (63) | 195 (63) | 106 (99, 115) | 110 (103, 119) | 104 (96, 111) |
| Cmax (ng/h) | 76.9 (52) | 60.8 (58) | 62.4 (59) | 130 (111, 153) | 133 (113, 157) | 102 (87, 120) |
| Tmax (h)^{a} | 1.00 (0.50, 2.00) | 1.25 (0.50, 3.00) | 1.25 (0.50, 3.00) | NC | NC | NC |

NC—not calculated;
% CV - coefficient of variation on arithmetic mean in percent.
^{a}Results presented are median (min, max).
^{b}Geometric least squares mean ratio and 90% confidence interval from ANOVA comparisons of log-transformed data.

Protein Binding

The ex vivo protein binding of cilansetron and its 4-hydroxy metabolites has been measured in serum samples collected in a multiple-dose study in healthy and renally impaired male and female subjects, aged 33-74 years. Subjects were enrolled into 4 groups as follows: 9 subjects in the healthy group (normal renal function, creatinine clearance >82 mL/min), 5 subjects in the mild renal impairment group (creatinine clearance 52 to 78 mL/min), 4 subjects in the moderate renal group (creatinine clearance 32 to 48 mL/min), and 4 subjects in the severe renal group (creatinine clearance <28 mL/min). Healthy subjects were matched to renally-impaired subjects with respect to age, weight, gender, and smoking status. All subjects received oral cilansetron 2 mg TID on Days 1 to 5, and a single dose on the morning of Day 6. Steady-state serum samples were collected at predose (trough samples) and at the approximate time of cilansetron Cmax (1 and 1.5 hours postdose) on Day 6, and were evaluated for protein binding.

Descriptive statistics of cilansetron and metabolite plasma concentrations, the corresponding percents unbound of each analyte, and statistical comparisons of percents unbound between study groups (renally impaired: healthy) are presented in Table 2.7.2.3.12 below. In healthy subjects, cilansetron was 96.0-96.6% protein bound on average, and its metabolites, (4R)-hydroxy and (4S-hydroxy cilansetron, were 72.4-75.3% and 42.0-48.1% protein bound on average, respectively. Extent of binding of all three analytes did not vary with plasma concentrations over the range of concentrations encountered. Additionally, no significant increases or trends toward an increase were observed in percent unbound of cilansetron or its 4-hydroxy metabolites with increasing renal impairment.

TABLE 2.7.2.3.12
Descriptive statistics of plasma concentrations and fractions unbound of
cilansetron and its 4-hydroxy metabolites in healthy and mild to severe
renally impaired subjects following dosing with cilansetron 2 mg TID for 6
days (Study S.241.1.110)

| | | | Arithmetic Mean (% CV) | | Ratio (90% CI) (%)^{a} | |
|---|---|---|---|---|---|---|
| | | | | Fraction | | All Renal Groups |
| | Day 6 | Study | Plasma Conc. | Unbound in | Each Renal Group: | Combined: |
| Analyte | Time Point | Group | (ng/mL) | Serum (%) | Healthy | Healthy |
| Cilansetron | Predose | Healthy | 4.10 (78) | 3.96 (45) | — | — |
| | | Mild | 1.73 (31) | 3.18 (10) | 86 (61, 121) | — |
| | | Moderate | 1.19 (69) | 3.09 (6) | 84 (54, 131) | — |
| | | Severe | 2.95 (37) | 2.75 (30) | 73 (52, 102) | 81 (62, 105) |
| | 1 h | Healthy | 19.5 (43) | 3.51 (37) | — | — |
| | | Mild | 13.3 (27) | 3.13 (26) | 91 (70, 119) | — |
| | | Moderate | 9.61 (83) | 3.55 (19) | 105 (79, 140) | — |
| | | Severe | 23.8 (34) | 2.90 (13) | 87 (65, 115) | 94 (76, 116) |
| | 1.5 h | Healthy | 17.9 (60) | 3.40 (39) | — | — |
| | | Mild | 11.2 (24) | 3.03 (28) | 92 (69, 123) | — |
| | | Moderate | 10.0 (51) | 2.87 (10) | 90 (66, 123) | — |
| | | Severe | 19.1 (34) | 2.59 (13) | 81 (59, 110) | 88 (70, 109) |
| (4R)—OH | Predose | Healthy | 1.56 (24) | 25.3 (11) | — | — |
| | | Mild | 1.68 (24) | 29.6 (30) | 114 (96, 137) | — |
| | | Moderate | 2.06 (40) | 31.0 (21) | 122 (100, 147) | — |
| | | Severe | 4.36 (36) | 32.4 (17) | 128 (105, 155)^{b} | 121 (105, 139)^{b} |
| | 1 h | Healthy | 3.90 (37) | 24.7 (7) | — | — |
| | | Mild | 4.86 (39) | 26.4 (14) | 106 (93, 121) | — |
| | | Moderate | 3.94 (32) | 34.3 (22) | 137 (119, 157)^{b} | — |
| | | Severe | 7.67 (38) | 30.2 (18) | 121 (105, 140)^{b} | 121 (109, 134)^{b} |
| | 1.5 h | Healthy | 3.79 (20) | 27.6 (15) | — | — |
| | | Mild | 3.80 (20) | 26.2 (17) | 95 (83, 109) | — |
| | | Moderate | 4.75 (28) | 28.5 (13) | 104 (90, 120) | — |
| | | Severe | 7.87 (38) | 23.7 (11) | 86 (74, 100) | 95 (85, 105) |
| (4S)—OH | Predose | Healthy | 1.24 (31) | 51.9 (24) | — | — |
| | | Mild | 1.76 (38) | 48.9 (40) | 91 (69, 121) | — |
| | | Moderate | 2.71 (72) | 54.0 (26) | 104 (77, 140) | — |
| | | Severe | 7.00 (35) | 51.4 (30) | 98 (73, 133) | 98 (79, 121) |
| | 1 h | Healthy | 1.60 (26) | 53.6 (20) | — | — |
| | | Mild | 2.26 (43) | 42.8 (7) | 81 (65, 101) | — |
| | | Moderate | 2.74 (58) | 69.3 (45) | 124 (98, 156) | — |
| | | Severe | 8.32 (40) | 56.1 (15) | 106 (84, 133) | 102 (86, 121) |
| | 1.5 h | Healthy | 1.68 (25) | 58.0 (21) | — | — |
| | | Mild | 1.91 (35) | 46.3 (11) | 81 (67, 99) | — |
| | | Moderate | 2.98 (56) | 57.4 (28) | 99 (80, 123) | — |
| | | Severe | 8.57 (32) | 42.4 (9) | 75 (60, 93)^{b} | 84 (72, 99) |

CI—confidence interval;
% CV - coefficient of variation on arithmetic mean in percent.
^{a}Geometric least squares mean ratio and 90% CI from ANOVA comparisons of log-transformed data.
^{b}Statistically significant difference (p < 0.05).

Plasma protein binding was also measured in blood samples collected at 30, 45, and 120 minutes after dosing in a single-dose, crossover study in which cilansetron 8 mg was administered orally (solution and capsule) and intravenously to 18 male subjects. In samples where total and/or free concentrations were measurable, fraction bound was about 96-98%.

The ex vivo protein binding of cilansetron has been measured in serum samples collected in a multiple-dose, parallel group study in 14 healthy and 14 moderately to severely hepatically impaired male and female subjects, aged 41-70 years. Impaired subjects (Child-Pugh score 7 to 11) were matched to healthy subjects by age (+2 years), sex, menstrual status, and smoking history status. All subjects received oral cilansetron 8 mg TID on Days 1 to 5, and a single dose on the morning of Day 6. Steady state serum samples for measurement of cilansetron protein binding were collected at predose, and 1 and 1.5 hours postdose on Day 6. Though mean percents bound were slightly lower than that observed in other studies, there were no differences in binding between impaired and healthy subjects (average percent bound of 93% in healthy subjects and 94.5% in impaired subjects) (Table 2.7.2.3.23).

Protein binding was also evaluated in an in vitro study in human, mouse, rat, and dog plasma using equilibrium dialysis (12-14K molecular weight cut-off membrane). This study measured the binding of (¹⁴C)-cilansetron in plasma at nominal concentrations (30, 90, and 300 ng/mL) which are much higher than those encountered in vivo in humans. This study also measured the binding of (¹⁴C)-cilansetron to purified human serum albumin at cilansetron concentrations ranging from 30-3000 ng/mL and to purified human alpha-1 acid glycoprotein (AAG) at concentrations ranging from 30-10000 ng/mL. Additionally, the effect of warfarin, at a nominal concentration of 10 μg/mL, on the binding of (¹⁴C)-cilansetron to human plasma proteins was determined at cilansetron nominal concentrations of 30, 300, and 3000 ng/mL.

In spite of the higher than therapeutic cilansetron concentrations evaluated in this in vitro study, mean protein binding in human plasma was similar (95.8%) to that seen in the ex vivo studies and was independent of concentration over the concentration range studied (30-300 ng/mL). Plasma binding in humans was higher than in mouse, rat, and dog, which was 83.3%, 88.2%, and 86.5% on average, respectively. The results of this study also showed that cilansetron bound to human albumin to an extent of 65.9%, and its binding was non-saturable and independent of concentration over the concentration range studied. Binding of cilansetron (at concentrations of 30-10000 ng/mL) to human AAG was saturable at high concentrations, with an affinity constant (Ka) of 0.56 μM⁻¹ (apparent binding sites of 0.6). Binding to AAG was around 90% at the lower concentrations but gradually decreased to around 65% due to binding saturation above concentrations of 900 ng/mL. Since the Cmax at the proposed 2 mg TID dose is around 6-7 ng/mL, saturation of AAG is not expected to occur under clinical conditions. This is supported by the lack of concentration dependence observed in plasma protein binding in this in vitro study or the ex vivo studies.

Evaluation of the effect of warfarin at a concentration of 10 μg/mL (notably higher than therapeutic concentrations of approximately 2.2±0.41 μg/mL) showed no binding displacement of cilansetron at concentrations of 30, 300, and 3000 ng/mL. Cilansetron was 93-96% protein bound in the absence of warfarin and 94-96% bound in the presence of warfarin over these concentrations. The effect of cilansetron on the binding of warfarin was not evaluated.

Dose Proportionality

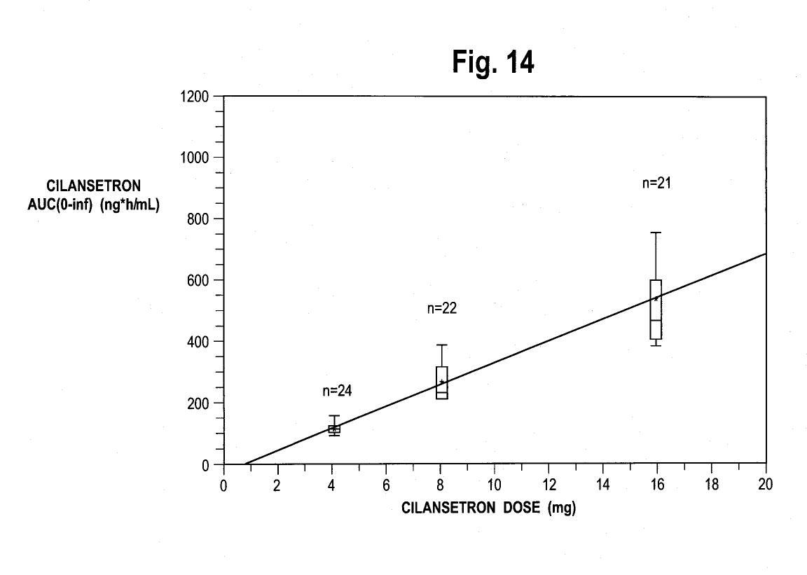

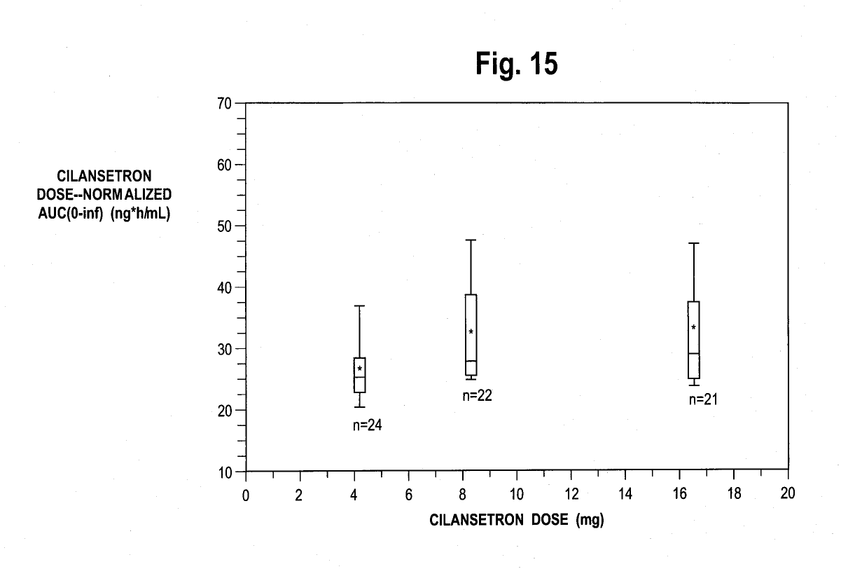

In a randomized, double-blind, placebo controlled, rising dose, crossover study, 24 male subjects (aged 23-37 years) received single doses of 4, 8, and 16 mg IV cilansetron in ascending order and a randomized placebo, infused over 15-30 minutes. All doses were administered under fasted conditions. FIG. 14 and FIG. 15 present box plots of cilansetron AUC(0-inf) and dose-normalized AUC(0-inf) values (normalized to a 1 mg dose), respectively, and Table 2.7.2.3.13 presents descriptive statistics for the results of statistical pairwise comparisons of dose-normalized parameters between doses. The data show that cilansetron exposure was generally linear over the 4-16 mg dose range, indicating that the distribution and elimination of cilansetron are linear over this range.

TABLE 2.7.2.3.13
Arithmetic mean (% CV) pharmacokinetic parameters for cilansetron and
results of statistical comparisons between doses following administration
of single IV rising doses of 4 mg to 16 mg (Study K.241.5004)

| | | | | Statistical Comparisons^{a} | | |
|---|---|---|---|---|---|---|
| | Arith. Mean (% CV) | | | Ratio (90% CI) (%) | | |
| | 4 mg | 8 mg | 16 mg | | 16 mg/ | |
| Parameter | (n = 24) | (n = 22) | (n = 21) | 8 mg/4 mg | 8 mg | 16 mg/4 mg |
| DN-AUC(0-inf) | 25.8 (25) | 31.7 (31) | 32.8 (37) | 121 | 102 | 124 |
| (ng · h/mL) | | | | (112, 131) | (94, 111) | (114, 134) |
| DN-AUC(0-last) | 25.1 (26) | 31.4 (31) | 32.4 (36) | 123 | 102 | 126 |
| (ng · h/mL) | | | | (114, 134) | (94, 111) | (116, 137) |
| T½ (h) | 1.70 (25) | 1.62 (24) | 1.41 (18) | NC | NC | NC |
| CL (mL/min) | 680 (22) | 567 (26) | 564 (30) | NC | NC | NC |

DN - dose normalized to a 1 mg dose;
NC—not calculated;
% CV - coefficient of variation on arithmetic mean in percent.
^{a}Results of ANOVA comparisons of log-transformed data (n = 21).

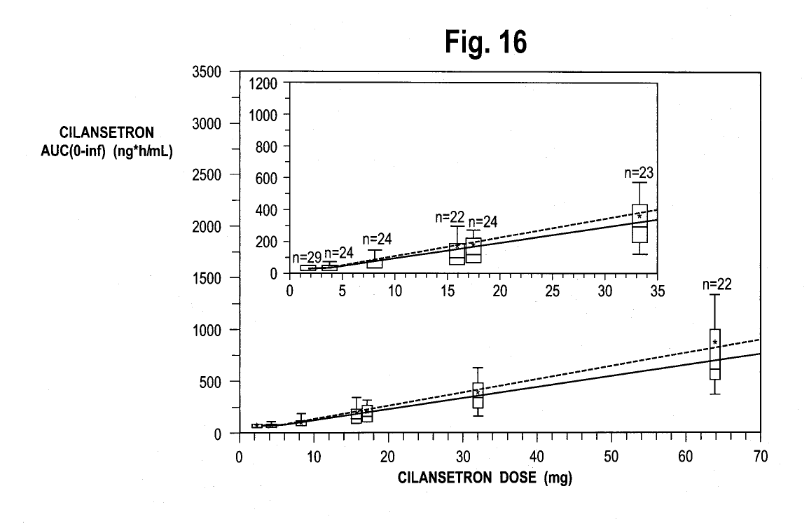

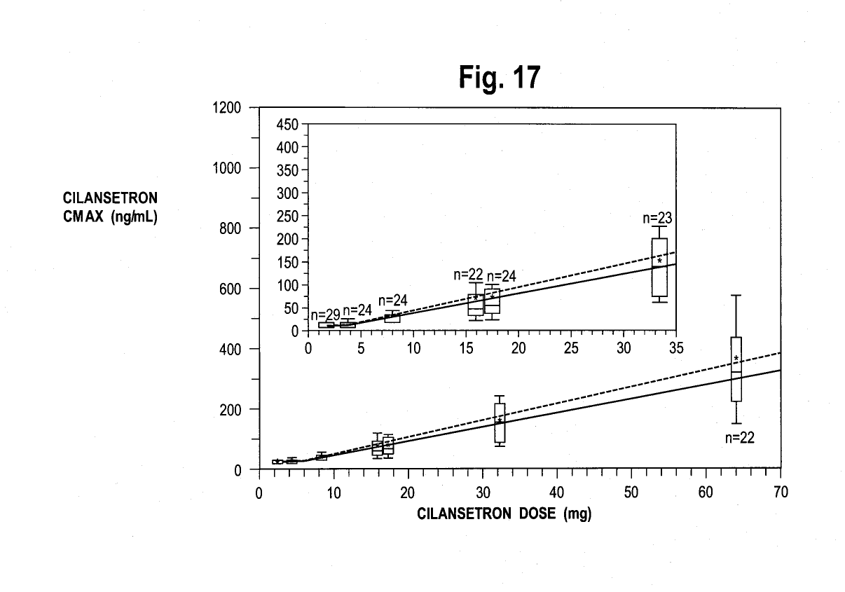

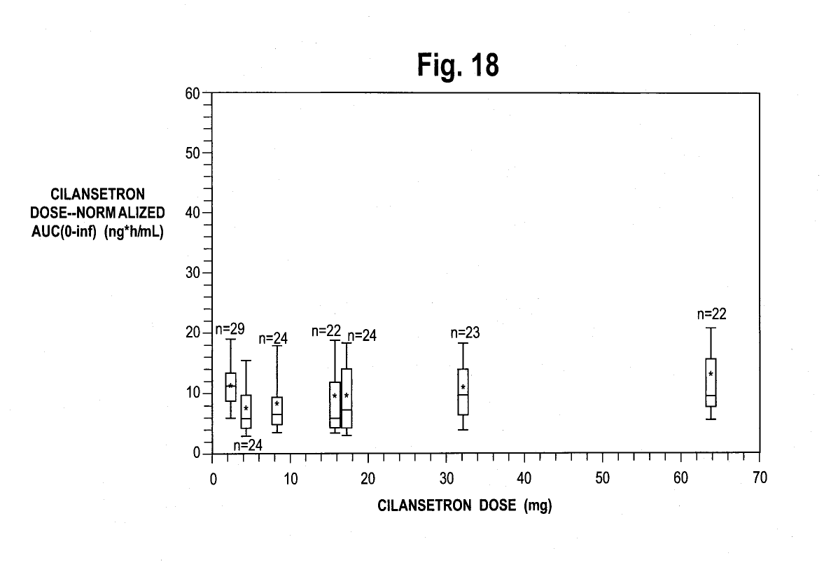

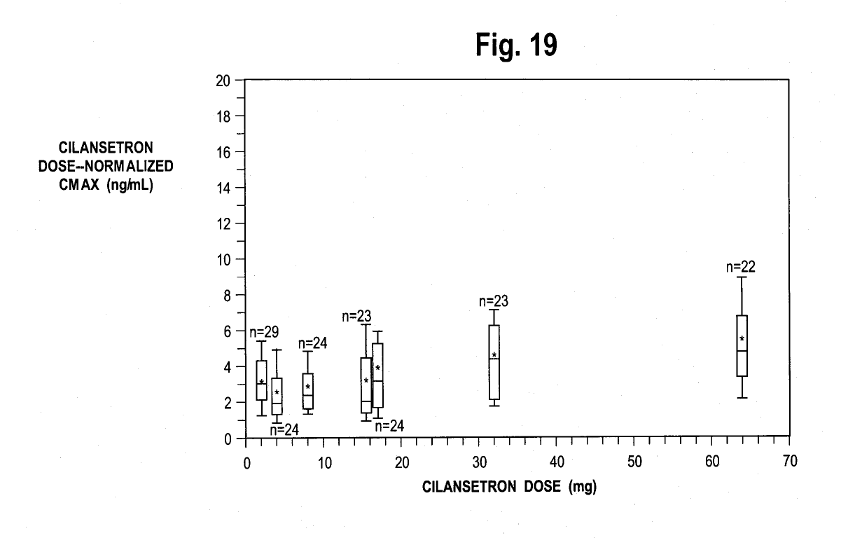

Data for the evaluation of dose proportionality of oral cilansetron is available from three studies which have evaluated the pharmacokinetics of single doses over a 2 mg to 64 mg dose range. Two randomized, double-blind, placebo controlled, rising dose, crossover studies, in 24 male subjects each, evaluated the single dose pharmacokinetics of oral cilansetron (capsules) over a 4-16 mg and 16-64 mg dose range, respectively. In each study, subjects received, at intervals of ≧1 week, single oral doses of cilansetron in ascending order and a randomized placebo. All doses were administered under fasted conditions. A third open-label, randomized, parallel-group study in 36 male and female subjects, evaluated the single dose pharmacokinetics of a cilansetron 2 mg tablet administered under fed conditions. FIG. 16 to FIG. 19 present box plots of cilansetron AUC(0-inf) and Cmax from these three studies (males only), and corresponding dose-normalized values (normalized to a 1 mg dose). Data from two different formulations (tablet and capsule) under different conditions of food intake (fed and fasted) have been combined in these plots, since previous studies have shown that formulation and food intake do not have a clinically significant effect on the bioavailability of cilansetron. Table 2.7.2.3.14 presents descriptive statistics of cilansetron dose-normalized AUC and Cmax in these studies, and the results of statistical comparisons between doses. These data demonstrate that the pharmacokinetics of cilansetron are approximately dose proportional over a dose range of 2-32 mg, with a slightly greater than proportional increase (approximately 30%) at the higher dose (64 mg).

TABLE 2.7.2.3.14
Arithmetic mean (% CV) pharmacokinetic parameters for cilansetron and
results of statistical comparisons between doses following administration
of single oral rising doses of 4 mg to 64 mg (Studies K.241.5001 and
K.241.5002)

| | | | | Statistical Comparisons^{a} | | |
|---|---|---|---|---|---|---|
| Parameter | Arith. Mean (% CV) | | | Ratio (90% CI) (%) | | |
| Study K.241.5001 | 4 mg | 8 mg | 16 mg | 8 mg/4 mg | 16 mg/8 mg | 16 mg/4 mg |
| DN-AUC(0-inf) | 6.67 (70) | 7.43 (77) | 8.63 (97) | 109 | 108 | 117 |
| (ng · h/mL) | | | | (93, 126) | (93, 126) | (100, 137) |
| DN-AUC(0-last) | 5.80 (78) | 6.94 (81) | 8.06 (103) | 120 | 110 | 132 |
| (ng · h/mL) | | | | (101, 142) | (92, 131) | (111, 157) |
| DN-Cmax (ng/mL) | 2.47 (68) | 2.76 (65) | 3.12 (85) | 115 | 107 | 122 |
| | | | | (98, 135) | (91, 125) | (104, 144) |
| T½ (h) | 1.56 (24) | 1.47 (30) | 1.34 (28) | NC | NC | NC |
| Study K.241.5002 | 16 mg | 32 mg | 64 mg | 32 mg/16 mg | 64 mg/32 mg | 64 mg/16 mg |
| DN-AUC(0-inf) | 8.83 (79) | 10.3 (72) | 12.7 (80) | 127 | 125 | 158 |
| (ng · h/mL) | | | | (114, 141) | (113, 139) | (143, 176) |
| DN-AUC(0-last) | 8.50 (83) | 10.1 (74) | 12.6 (81) | 132 | 127 | 168 |
| (ng · h/mL) | | | | (118, 147) | (114, 142) | (150, 187) |
| DN-Cmax (ng/mL) | 3.80 (77) | 4.46 (62) | 5.45 (64) | 127 | 129 | 164 |
| | | | | (112, 144) | (113, 146) | (144, 186) |
| T½ (h) | 1.23 (26) | 1.28 (22) | 1.30 (25) | NC | NC | NC |

DN - dose normalized to a 1 mg dose;
NC—not calculated;
% CV - coefficient of variation on arithmetic mean in percent.
^{a}Results of ANOVA comparisons of log-transformed data (n = 24 for Study K.241.5001 and K.241.5002)

Data for the evaluation of dose proportionality of oral cilansetron following multiple dosing is available from four studies which have evaluated the pharmacokinetics of TID dosing over a 2 mg to 32 mg dose range. In a randomized, double-blind, placebo controlled, parallel group study, 14 male subjects received 4 mg cilansetron TID and 14 received 8 mg cilansetron TID for 6 days and a single dose on the morning of the seventh day. In two randomized, double-blind, placebo controlled, balanced incomplete block design, crossover studies, subjects were randomized to receive two of three treatments TID for 5 days during 2 treatment periods, as follows: 12 male and 13 female subjects received 8 mg cilansetron, 16 mg cilansetron, or placebo in one study, and, in another study, 12 male and 13 female subjects received 16 mg cilansetron, 32 mg cilansetron, or placebo. There was a 5-6 day washout period between treatment periods in the two studies. In a fourth open-label, randomized, parallel-group study, 36 male and female subjects received cilansetron alone (2 mg tablet TID) for 6 days. The pharmacokinetics of cilansetron were evaluated on the last treatment day of each study.

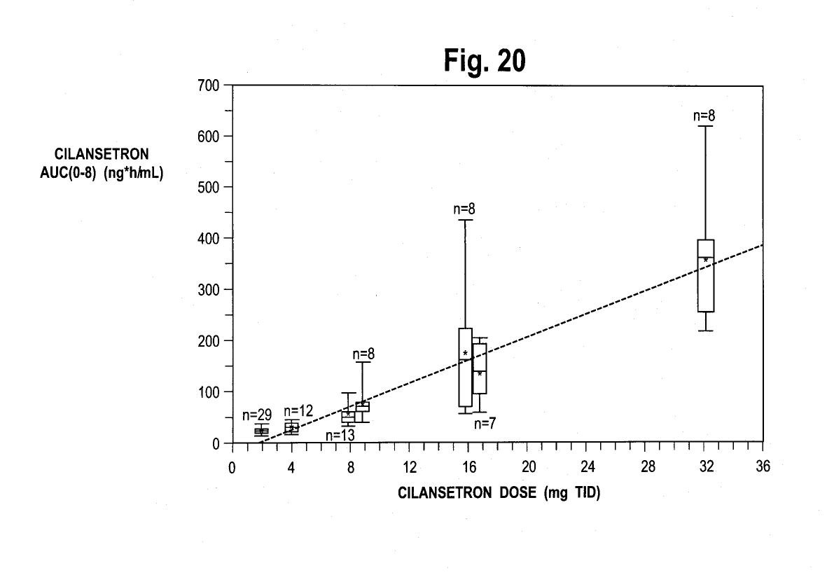

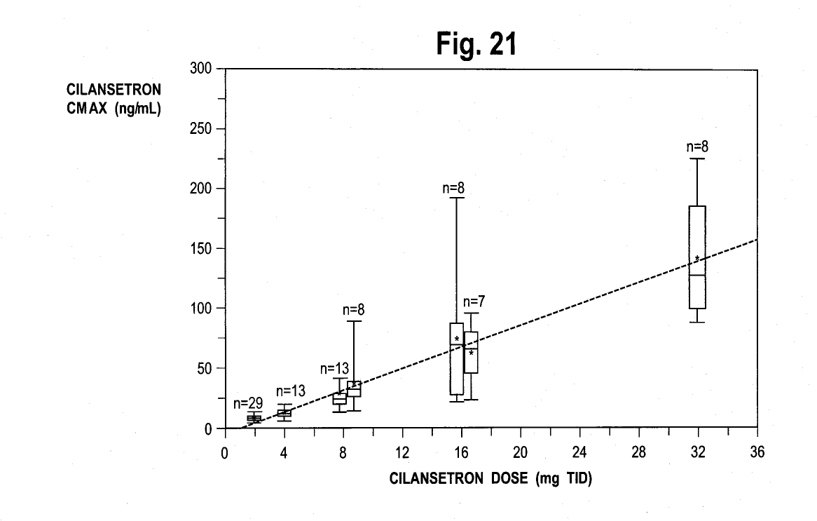

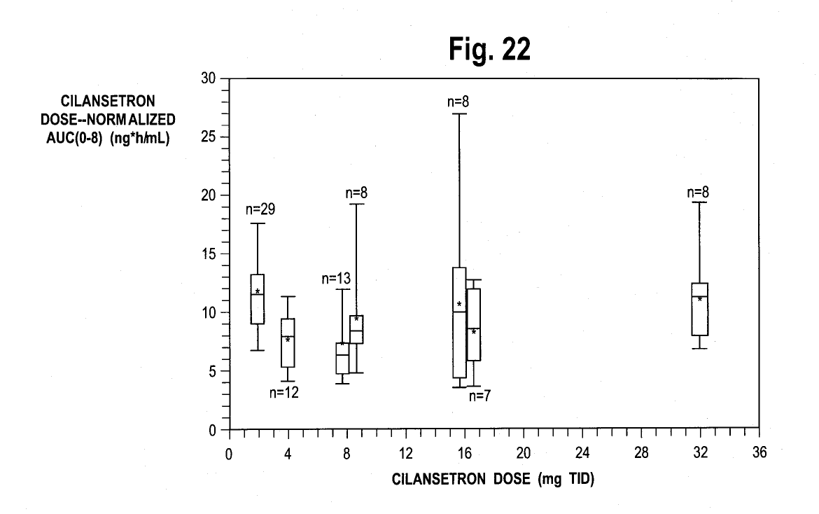

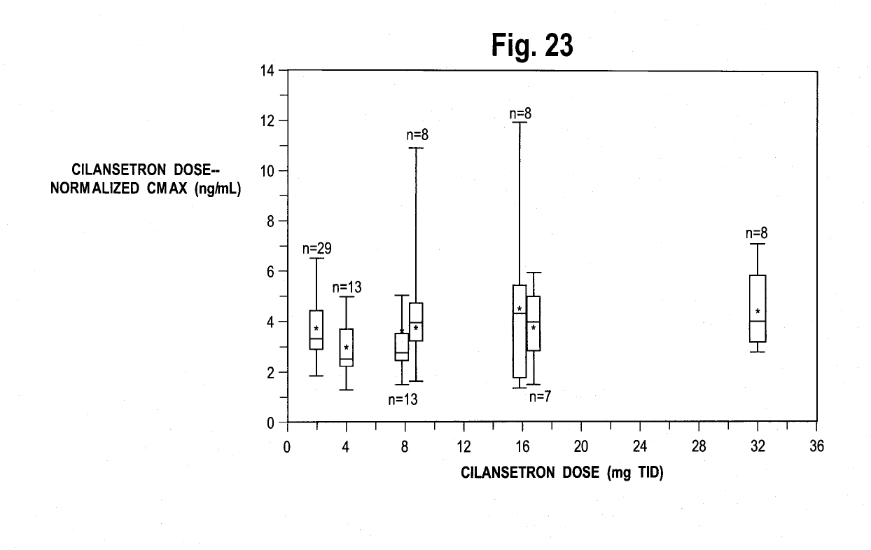

FIG. 20, FIG. 21, FIG. 22, and FIG. 23 present box plots of cilansetron AUC(0-τ) and Cmax from these four studies (males only), and corresponding dose-normalized values (normalized to a 1 mg dose). As in the case of single dosing, data from two different formulations (tablet and capsule) under different conditions of food intake (fed and fasted) have been combined in these plots because of the absence of significant formulation or food effects on the bioavailability of cilansetron. The data in these plots show that the pharmacokinetics of cilansetron are dose proportional over a multiple-dose range of 2 mg to 32 mg.

Metabolism and Excretion

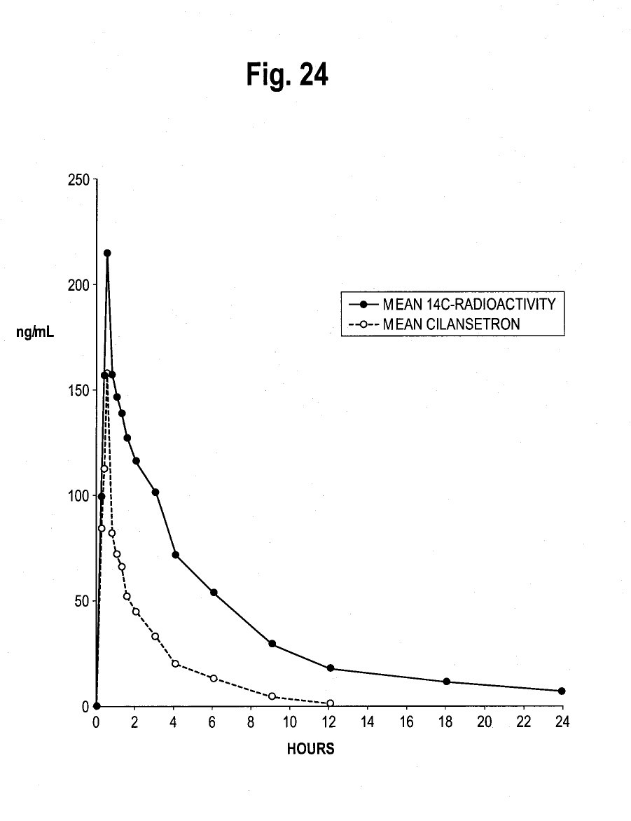

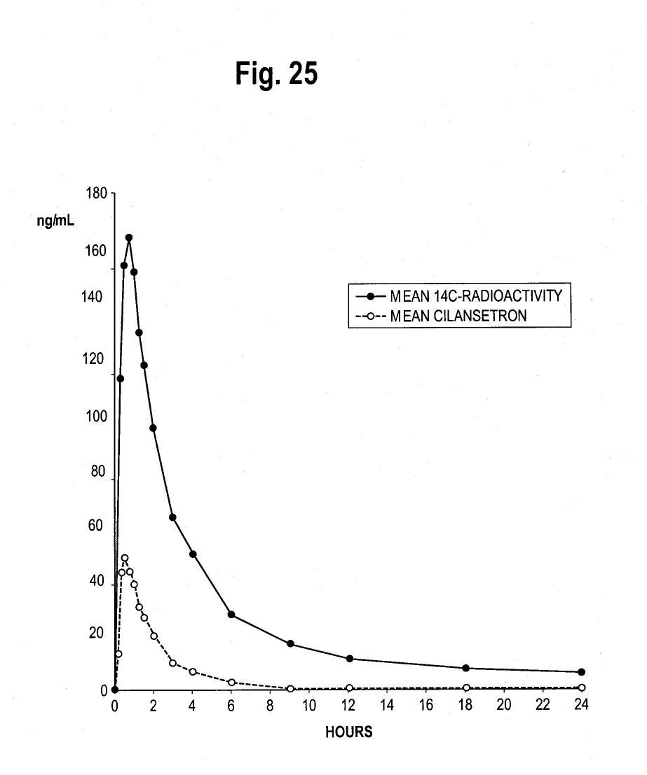

The extent of absorption, distribution, metabolism, and elimination of cilansetron has been examined in an open-label, randomized, two period, crossover, mass balance study in 6 healthy male subjects who were treated with a single oral and single N dose (30-minute infusion) of 8 mg ¹⁴C-labeled (50 K μCi) cilansetron, with a washout of at least 2 weeks between periods. The ¹⁴C-cilansetron was appropriately labeled in the aromatic ring and the label was therefore not metabolically labile. Serial blood samples, urine collection, and fecal collection were performed up to 144 hours following each dose. ¹⁴C-radioactivity was measured in plasma, whole blood, urine, and feces, and unchanged cilansetron was measured in plasma and urine. Identification of metabolites of cilansetron was performed in plasma, urine, and feces. The results of this study are presented in Table 2.7.2.3.15 and the mean concentration-time plots for ¹⁴C-radioactivity and unchanged cilansetron are shown in FIG. 24 and FIG. 25.

TABLE 2.7.2.3.15
Arithmetic mean (SD) single-dose pharmacokinetic parameters for total
radioactivity and unchanged cilansetron following administration of
14C-labeled cilansetron 8 mg as IV and oral solutions to 6 subjects
(Study S.241.1.103)

| | ¹⁴C-radioactivity | | Unchanged Cilansetron | |
|---|---|---|---|---|
| Parameter | Oral | IV | Oral | IV |
| AUC (0-inf) (ng[-equiv] · h/mL)^{a} | 1047 (248) | 1217 (299) | 98.2 (55.9) | 209 (36) |
| AUC (0-last) (ng[-equiv] · h/mL)^{a} | 908 (244) | 999 (223) | 91.4 (52.2) | 200 (35) |
| Cmax (ng[-equiv]/mL)^{a} | 171 (63) | NA | 52.9 (36.4) | NA |
| Tmax (h)^{b} | 0.75 | NA | 0.51 | NA |
| | (0.35, 1.00) | | (0.33, 0.75) | |
| T½ (h) | 73.9 (20.6) | 107 (35) | 1.23 (0.46) | 1.51 (0.32) |
| CL or CL/F (plasma) (mL/min)^{c} | 133 (29) | 118 (42) | 1815 (1195) | 655 (127) |
| CL or CL/F (blood) (mL/min)^{c} | NC | NC | 2857 (1912) | 1041 (212) |
| Vz or Vz/F (L)^{c} | 836 (275) | 1001 (162) | 155 (38) | 85.3 (21.9) |
| Absolute Bioavailability (%) | 89.3 (22.3) | NA | 48.7 (29.7) | NA |
| Recovery (%) | | | | |
| Renal | 63.3 (2.2) | 63.5 (2.3) | 0.31 (0.26) | 0.36 (0.16) |
| Fecal | 27.5 (3.2) | 28.9 (1.8) | NC | NC |
| Total | 90.8 (2.2) | 92.3 (1.5) | | |
| Radioactivity in blood cells (%) | 10.5 (5.7) | 9.5 (4.1) | | |

NC—not calculated;
NA—not applicable.
^{a}Units are expressed in terms of ng for unchanged cilansetron and ng-equivalents for radioactivity.
^{b}Results presented are median (min, max).
^{c}CL and Vz for IV dose and CL/F and vz/F for oral doses.

The results of this study showed that cilansetron is completely absorbed from the gastrointestinal (GI) tract after oral dosing (mean absolute bioavailability of total radioactivity of 89%, range 58-124%). The absorption calculated from the percentage renal recovery is 99.8%±3.9%.

The average absolute bioavailability of unchanged cilansetron was 49% (range 17-94%) in this study, which was higher than that measured in another study (bioavailability of 25%), but this difference is likely to be attributable to the small number of subjects used in this mass balance study and the intrinsically high variability associated with cilansetron pharmacokinetics. Since cilansetron is completely absorbed from the GI tract, its low oral bioavailability can be attributed to extensive first pass metabolism following oral dosing, which is also evident in its relatively high apparent oral clearance (CUF) following oral dosing, in comparison to systemic clearance.

The mean elimination half-life of cilansetron was 1.2 to 1.5 hours following oral and IV dosing, which was comparable to estimates in other studies. However, the half-life for disappearance of total radioactivity from plasma was considerably longer, with mean values of 74 and 107 hours for oral and IV dosing, indicating a longer retention time for metabolites. Partitioning into red blood cells was low, with only about 10% of the total radioactivity present in whole blood being contained in blood cells and the remainder in plasma.

After both oral and IV administration, about 28% of the administered dose was recovered in feces and 63% was recovered in urine (<1% as unchanged drug) over a period of 144 hours, with ≧87% of the excreted amount in urine being excreted within 24 hours of dosing. The major metabolite peaks observed in plasma, urine, and feces were the 4-hydroxy metabolites of cilansetron. Biliary excretion of cilansetron and its metabolites in humans is low, since only 28% of the dose is recovered in feces.

Cilansetron and metabolite profiles in plasma, urine, and feces were evaluated using HPLC separation followed by determination of radioactivity using scintillation counting. Cilansetron, (4R)-hydroxy cilansetron, and (4S)-hydroxy cilansetron accounted for 29.3%, 25.7%, and 13.8% of the total radioactivity, respectively, in plasma at 6 hours after oral administration, and for 46.2%, 20.0% and 14.1%3 of the total radioactivity in plasma at 3 hours after IV administration. The cumulative percent of cilansetron and 4-hydroxy metabolites excreted in urine over a 48-hour interval following dosing was 0.5% and 32.8% of the dose, respectively, following oral dosing, and 1.1% and 31.9% of the dose following IV dosing. In the case of fecal excretion over a 96-hour postdose, cilansetron, (4R)-hydroxy cilansetron, and (4S)-hydroxy cilansetron accounted for 1.2%, 6.9%, and 4.6% of the dose following oral administration, and 1.3%, 5.1%, and 11.3% of the dose following IV administration.

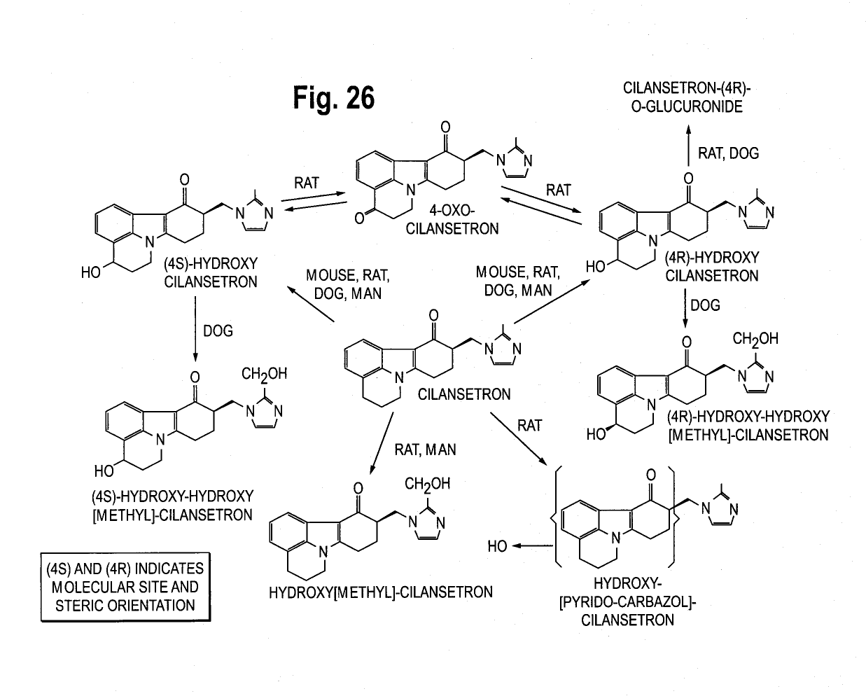

The oxidative metabolism of cilansetron, as mediated by cytochrome P450, flavin-monoxygenase, and mono-aminoxidase, has been evaluated in several in vitro studies using human hepatic microsomes and recombinant CYP isozymes. The results of these studies, which utilized high cilansetron concentrations ranging from 20-100 μM (approximately 6-32 μg/mL), suggested that CYP3A4, CYP2D6, CYP1A1, CYP1A2, and CYP2C19 were involved in the metabolism of cilansetron, with the largest contribution coming from CYP3A4 under these in vitro conditions. The major metabolic route in humans is oxidation of the tetrahydropyridine ring, leading to the formation of the active metabolites, (4R)-hydroxy and (4S)-hydroxy cilansetron, both of which have much lower activity than cilansetron. (4R)-hydroxy and (4S)-hydroxy cilansetron bind to the human 5-HT3 receptor in the nanomolar range and exhibit pKi values of 8.5 and 8.0, respectively, compared to cilansetron which exhibits subnanomolar affinities to the human 5-HT3 receptor with a pKi value of 9.4. The ratio of formation of (4R)-hydroxy to (4S)-hydroxy cilansetron in vitro was found to be approximately 1.0 for humans. (4R)-hydroxy cilansetron undergoes further metabolism by CYP2D6 and CYP3A4 and (4S)-hydroxy cilansetron was mainly metabolized by CYP3A4. Seventeen other hydroxylated metabolites of cilansetron were formed in vitro, but only in very small quantities. FIG. 26 below presents the major metabolic routes for cilansetron that have been identified in humans, mouse, rat, and dog.

The influence of CYP2D6 gene polymorphism on the pharmacokinetics of cilansetron has been evaluated in blood samples collected from various single- and multiple-dose Phase I clinical studies of orally or intravenously administered cilansetron. Of a total of 179 healthy male and female subjects who participated in studies, 8 subjects were poor metabolizers with respect to CYP2D6. Cilansetron plasma concentrations and pharmacokinetic parameters (AUC, Cmax, and half-life) were not higher in these subjects than in extensive metabolizers.

The CYP mRNA induction potentials of cilansetron and its two main metabolites, (4S)-hydroxy and (4R)-hydroxy cilansetron, were determined in human hepatocytes and compared to three model inducers, 3-methylcholantrene, phenobarbital, and rifampin. Cilansetron, but not its metabolites, had a weak inducer effect on the CYPIA family (approximately 2% that of 3-methylcholantrene), which is considered negligible. Cilansetron and its metabolites increased CYP2C19 to a level that was about half of that caused by phenobarbital. Phenobarbital is a moderate inducer of CYP2C19 in vitro but is reportedly inactive in inducing this enzyme in humans in vitro. Comparison with the other model inducer used, rifampin, gave variable results, suggesting that the significance of any findings was doubtful. Cilansetron and its two metabolites did not induce CYP3A4 mRNA but doubled CYP3A5 mRNA, which was similar to the effect seen with the model inducers 3-methylcholantrene and phenobarbital. Since CYP3A5 is a minor isoform in human liver, shares substrate specificity with CYP3A4, and has a low activity compared to that of CYP3A4, limited variations in CYP3A5 activity are unlikely to cause major changes in the overall CYP3A activity. The lack of a significant inductive effect on CYP3A by cilansetron is also supported by the results of a clinical study which show that concomitantly administered cilansetron does not affect the pharmacokinetics or pharmacodynamics of the CYP3A substrate, Ortho Tri-Cyclen. Cilansetron and its 4-hydroxy metabolites did not show an inductive effect on other CYP isozymes (CYP2A6, CYP2B6, CYP2C9, CYP2D6, and CYP2EI).

In an in vitro study utilizing human hepatic microsomes (K.241.6013, Left, Vol.9 Righ) to evaluate the potential for cilansetron to inhibit cytochrome P450 enzymes, cilansetron showed only weak inhibition (1C50 of 60 μM, which is >3000× Cmax at the proposed dose of 2 mg TID) of 7-ethoxyresorufin O-deethylation (a marker of CYP1A2 activity) and no inhibition of any other cytochrome P450 enzyme activity, suggesting that potential drug interactions due to inhibition of CYP enzymes by cilansetron are unlikely. In another study, (4R)-hydroxy and (4S)-hydroxy cilansetron were shown to have no significant inhibitory effect on any cytochrome P450 enzyme (IC50>100 μM).

An in vitro study using human hepatic microsomes evaluated the effect of some enzyme inhibitors on the metabolism of cilansetron (100 μM). The inhibitors tested were troleandomycin (10 μM, CYP3A4 inhibitor) and ketoconazole at lower (1 μM, CYP3A4 inhibitor) and higher (25 μM, general inhibitor) concentrations. On average, troleandomycin (10 μM) and ketoconazole (1 μM) inhibited about 70% of cilansetron metabolism while ketoconazole (25 μM) inhibited 93% of its metabolism. Troleandomycin (10 μM) and ketoconazole (1 μM) inhibited 34% and 47%, respectively, of the formation of the 4-hydroxy metabolites while ketoconazole (25 μM) inhibited 69% of formation.

Since in vitro studies have indicated that cilansetron is metabolized by CYP3A4, CYP1A, and CYP2D6, a clinical study was conducted to evaluate the effect of ketoconazole (CYP3A4 inhibitor), fluvoxamine (inhibitor of CYP1A2, but also of CYP2C9, CYP2C19, and CYP3A), and paroxetine (CYP2D6 inhibitor) on the pharmacokinetics of cilansetron 2 mg TID. In brief, the results showed that concomitant administration of fluvoxamine had a profound effect on the pharmacokinetics of cilansetron, causing a mean 6.6-fold and 3.7-fold increase in AUC and Cmax, respectively. Some accompanying decrease in exposure was observed for (4R)-hydroxy cilansetron (mean AUC and Cmax decreased by 24 and 40%) but no change was observed for (4S)-hydroxy cilansetron. Concomitant administration of ketoconazole caused some increase in exposure to cilansetron and its 4-hydroxy metabolites. Mean cilansetron AUC and Cmax increased by 40% and 51%, mean (4R)-hydroxy cilansetron AUC and Cmax increased by 76% and 109%, and (4S)-hydroxy cilansetron AUC and Cmax increased by 63% and 74%, respectively. Concomitant administration of paroxetine had a small but clinically insignificant effect on the pharmacokinetics of cilansetron. Mean cilansetron AUC increased by 20% and mean Cmax by 36%. Mean (4R)-hydroxy cilansetron AUC and Cmax increased by 16% and 27%, respectively, but there was no change in the pharmacokinetics of (4S)-hydroxy cilansetron. One subject receiving paroxetine was identified by genotyping as a poor metabolizer for CYP2D6 and another was identified as a poor metabolizer for CYP2C 19. No difference in cilansetron pharmacokinetics was observed in the poor metabolizer for CYP2D6 but an approximate 2-fold increase was observed in AUC(0-τ) and Cmax in the poor metabolizer for CYP2C19, following coadministration of paroxetine.

In line with the results of in vitro studies, which suggested that CYP3A4, CYP2D6, CYP1A1, CYP1A2, and CYP2CI9 may be involved in the metabolism of cilansetron, this clinical study showed that cilansetron pharmacokinetics are affected during coadministration with fluvoxamine (inhibitor of CYPIA2 and other CYP enzymes such as CYP2C19) and with ketoconazole (CYP3A4 inhibitor). This apparent discrepancy may be due to the inhibitory effect of fluvoxamine on multiple enzymes, differences in expressiodactivity of these enzymes in vitro compared to in vivo, or the use of cilansetron concentrations (20-100 μM) in the in vitro studies which were much higher than therapeutic concentrations (Cmax of 6-7 ng/mL for 2 mg TID dose).

Chiral Inversion

Chiral inversion of cilansetron to its distomer [(+)-(10S) isomer], has been evaluated in 72 human plasma samples collected from a total of 36 subjects after oral administration of cilansetron 2 mg as a single dose or TID. It has also been evaluated in 166 human plasma samples collected after single-dose oral or IV administration of cilansetron. It was concluded that no in vivo chiral inverson of cilansetron to KC9945 occurs in humans.

Effect of Ape

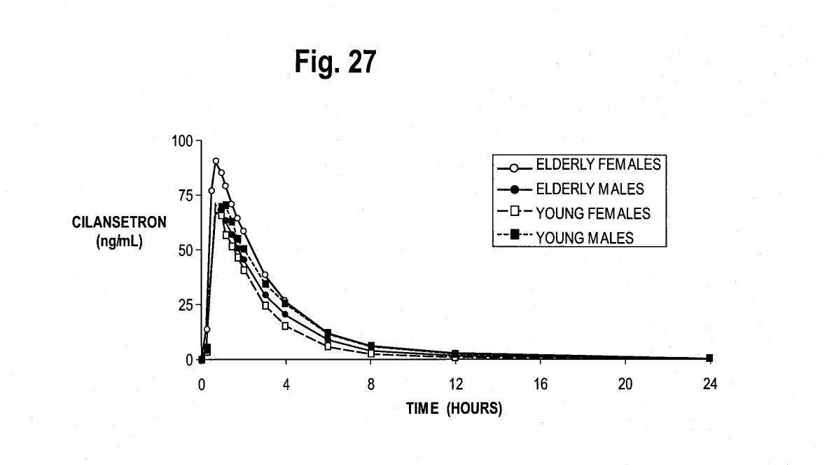

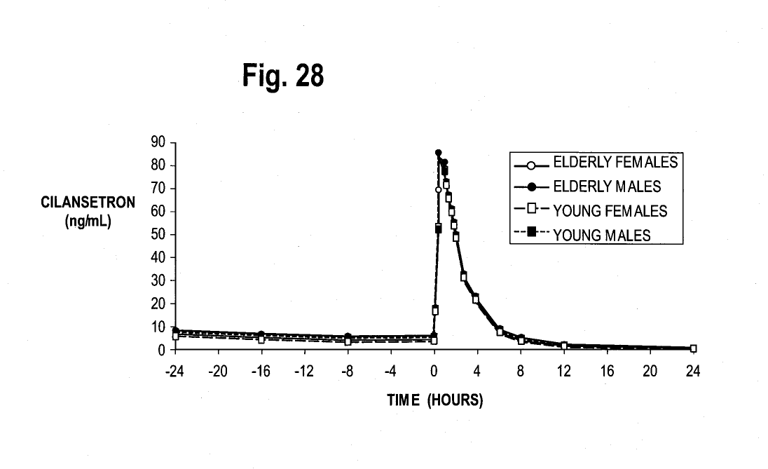

The effect of age on the pharmacokinetics of cilansetron was evaluated in an open-label, parallel group study in 16 male (8 young and 8 elderly) and 16 female (8 young and 8 elderly) subjects. Subjects were treated with a single oral dose of cilansetron 8 mg (tablet) on Day 1, followed by 8 mg TID for 5 days (Days 2-6) and a single 8 mg dose on the morning of Day 7. Young subjects ranged in age from 19-45 years, and elderly from 66-79 years. Serial blood samples were collected up to 24 hours postdose on Days 1 and 7, and cilansetron pharmacokinetic parameters were calculated. Doses on pharmacokinetic sampling days were administered under fasted conditions. The results are presented in Table 2.7.2.3.16, and mean cilansetron concentration-time profiles by age and gender on Days 1 and 7 are presented in FIG. 27 and FIG. 28. No statistically significant differences (p>0.05) were observed in any single- or multiple-dose pharmacokinetic parameter, including AUC and Cmax, between young and elderly subjects, except for a small increase in half-life in elderly compared young subjects.

TABLE 2.7.2.3.16
Arithmetic mean (% CV) single- and multiple-dose pharmacokinetic
parameters for cilansetron in young and elderly male and female subjects
following oral administration of cilansetron 8 mg single dose and TID for
5 days (Study S.241.1.105)

| | Elderly | | Young | |
|---|---|---|---|---|
| Parameter | Females (n = 8) | Males (n = 8) | Females (n = 8) | Males (n = 8) |
| Single Dose (Day 1) | | | | |
| AUC (0-inf) (ng · h/mL) | 289 (34) | 228 (53) | 178 (42) | 252 (123) |
| AUC (0-τ) (ng · h/mL) | 263 (33) | 196 (50) | 172 (38) | 223 (113) |
| Cmax (ng/mL) | 95.1 (44) | 58.8 (46) | 72.9 (34) | 77.5 (90) |
| Tmax (h)^{a} | 0.88 (0.50, 1.25) | 1.31 (0.75, 2.00)^{b} | 0.84 (0.75, 1.25) | 1.00 (0.75, 1.25) |
| T½ (h) | 2.94 (24)^{c} | 3.63 (20)^{c} | 2.72 (44)^{c} | 2.30 (46)^{c} |
| CL/F (L/h) | 31.9 (46) | 52.5 (92) | 55.3 (58) | 101 (136) |
| Vz/F (L) | 124 (24) | 243 (60) | 178 (21) | 231 (102) |
| Multiple Dose (Day 7) | | | | |
| AUC (0-τ) (ng · h/mL) | 247 (27) | 249 (61) | 206 (57) | 198 (77) |
| Cmax (ng/mL) | 99.0 (26) | 71.9 (49) | 78.2 (49) | 73.0 (59) |
| Tmax (h)^{a} | 0.78 (0.50, 1.00) | 1.17 (0.75, 1.75)^{b} | 1.00 (0.75, 2.00) | 0.91 (0.50, 1.50) |
| T½ (h) | 4.45 (12) | 5.05 (24) | 5.46 (21) | 5.15 (31) |
| CL/F (L/h) | 34.4 (26) | 50.6 (87) | 52.0 (54) | 67.6 (92) |
| Vz/F (L) | 218 (24) | 417 (110) | 404 (72) | 416 (46) |
| Accumulation Ratio (AUC) | 1.01 (32) | 1.27 (33) | 1.26 (55) | 1.25 (48) |
| Accumulation Ratio (Cmax) | 1.14 (32) | 1.26 (34) | 1.11 (43) | 1.21 (44) |

Note:
Log-transformed AUC and Cmax were tested for age (elderly versus young) and gender differences using ANOVA.
Tmax (untransformed) was tested using non-parametric analysis.
^{a}Results presented are median (min, max).
^{b}Significantly different from elderly females (p < 0.05).
^{c}Statistically significant age effect (p < 0.05).

The effect of age on the pharmacokinetics of cilansetron and its 4-hydroxy metabolites was also evaluated in a population pharmacokinetic analysis of a Phase II study in non-constipated IBS patients being treated with 1 mg, 2 mg, 8 mg, or 16 mg cilansetron or placebo TID for 12 weeks. Age did not significantly affect the pharmacokinetics of cilansetron or its metabolites.

Effect of Gender

In an open-label, parallel group study, 16 male (8 young and 8 elderly) and 16 female (8 young and 8 elderly) subjects were treated with a single oral dose of cilansetron 8 mg (tablet) on Day 1, followed by 8 mg TID for 5 days (Days 2-6) and a single 8 mg dose on the morning of Day 7. Young subjects ranged in age from 19-45 years, and elderly from 66-79 years. Serial blood samples were collected up to 24 hours postdose on Days 1 and 7, and cilansetron pharmacokinetic parameters were evaluated. Doses on pharmacokinetic sampling days were administered under fasted conditions. The results are presented in Table 2.7.2.3.16, and mean cilansetron concentration-time profiles on Days 1 and 7 by age and gender are presented in FIG. 27 and FIG. 28. No statistically significant differences (p>0.05) or even trends were observed in single- or multiple-dose pharmacokinetic parameters between males or females, except for single-dose Tmax which showed a small increase in elderly males compared to elderly females.

In two randomized, double-blind, placebo controlled, balanced incomplete block design, crossover studies evaluating the single- and multiple-dose pharmacokinetics of oral cilansetron over a 8 mg to 32 mg dose range, subjects were randomized to receive two of three treatments during 2 treatment periods. In Study S.241.1.101; (12 male and 13 female subjects, aged 23 to 36 years), subjects received 8 mg cilansetron, 16 mg cilansetron, or placebo, while in Study S.24 1.1.102; (12 males and 13 females, aged 23 to 41 years), subjects received 16 mg cilansetron, 32 mg cilansetron, or placebo. During each treatment period, subjects received a single dose of their assigned treatment on the first day, followed by a 3-day washout, after which they received the same treatment TID for 5 days. There was a 5-6 day washout period between treatment periods. Doses on the mornings of pharmacokinetic days were administered under fasted conditions. Serial blood sampling and urine collection was performed up to 12 hours following morning dosing on the first day (single dose) and on the last multiple dosing day of each treatment period, and cilansetron pharmacokinetic parameters were evaluated.

The results of these studies are summarized in Table 2.7.2.3.17 and Table 2.7.2.3.18 below. In both studies, no differences were observed in Tmax between males and females following single or multiple dosing. In Study S.241.1.101, mean cilansetron exposure (AUC and Cmax) was 16-46% lower in males than in females following both single and multiple dosing. However in Study S.241.1.102, cilansetron exposure varied between the sexes, ranging from a 40% lower exposure to a 72% higher exposure in males than in females (based on mean AUC and Cmax). In both studies, mean AUC(0-T) (multiple dose) to AUC (0-inf) (single dose) ratios were variable (69-115%), but showed no clear differences between males and females. Coefficients of variation for AUC and Cmax were large, ranging from approximately 28-81% across both studies, and did not show any differences between males and females. Renal excretion of cilansetron was negligible following single and multiple dosing in both genders at all doses, with less than 1% of the dose excreted unchanged in urine.

TABLE 2.7.2.3.17
Arithmetic mean (% CV) single- and multiple-dose pharmacokinetic
parameters for cilansetron following administration of oral cilansetron
8 mg and 16 mg as single doses and TID for 5 days (Study S.241.1.101)

| | | Cilansetron 8 mg | | Cilansetron 16 mg | |
|---|---|---|---|---|---|
| | | Single | Multiple | Single | Multiple |
| Group | Parameter | Dose | Dose | Dose | Dose |
| Females | AUC (ng · h/ml.)^{a} | 138 (48) | 104 (44) | 260 (81) | 275 (61) |
| (n = 7-8) | DN-AUC (ng · h/mL)^{a} | 17.2 (48) | 13.0 (44) | 16.3 (81) | 17.2 (61) |
| | Cmax (ng/mL) | 52.2 (35) | 43.2 (37) | 110 (65) | 121 (53) |
| | Tmax (h)^{b} | 1.00 (0.67, 1.50) | 1.00 (1.00, 1.50) | 1.00 (0.67, 2.00) | 0.84 (0.67, 2.00) |
| | Cmin (ng/mL) | NA | 4.56 (91) | NA | 8.66 (77) |
| | T½ (h) | 1.45 (36) | 1.41 (16) | 1.29 (34) | 1.45 (32) |
| | % Dose (urine)^{c} | 0.55 (82) | 0.45 (97) | 0.42 (100) | 0.20 (72) |
| | Linearity Ratio (%)^{d} | NA | 80 (61, 107) | NA | 115 (89, 147) |
| Males | AUC (ng · h/mL)^{a} | 89.9 (36) | 74.5 (46) | 199 (54) | 172 (73) |
| (n = 8) | DN-AUC (ng · h/mL)^{a} | 11.2 (36) | 9.32 (46) | 12.4 (54) | 10.8 (73) |
| | Cmax (ng/mL) | 35.5 (28) | 35.8 (62) | 86.5 (52) | 71.4 (78) |
| | Tmax (h)^{b} | 1.00 (0.67, 1.50) | 1.00 (0.67, 2.00) | 1.00 (0.67, 2.00) | 1.00 (0.67, 1.50) |
| | Cmin (ng/mL) | NA | 2.85 (50) | NA | 9.71 (85) |
| | T½ (h) | 1.44 (24) | 1.22 (20) | 1.30 (15) | 1.39 (17) |
| | % Dose (urine)^{c} | 0.21 (65) | 0.14 (107) | 0.15 (67) | 0.11 (79) |
| | Linearity Ratio (%)^{d} | NA | 81 (68, 97) | NA | 77 (65, 93) |
| Male/ | AUC Ratio (90% CI) (%)^{e} | 81 (46, 145) | 84 (46, 154) | 82 (47, 142) | 58 (32, 103) |
| Female | Cmax Ratio (90% CI) (%)^{e} | 77 (46, 128) | 82 (42, 160) | 79 (49, 130) | 54 (29, 103) |

NA—not applicable;
% CV - coefficient of variation on arithmetic mean in percent;
DN - dose normalized to a 1 mg dose.
^{a}AUC is AUC(0-inf) for single dose and AUC(0-τ) (i.e. AUC(0-8)) for multiple dose.
^{b}Results presented are median (min, max).
^{c}Percent of dose excreted in urine over a 12-hour interval postdose for single dosing, and over an 8-hour interval postdose for multiple dosing.
^{d}Statistical comparison of AUC(0-τ) on multiple dose day to AUC(0-inf) on single dose day; results presented are geometric least squares mean ratios (90% confidence intervals).
^{e}Statistical comparison of AUC values and Cmax values between males versus females; results presented are geometric least squares mean ratios (90% confidence intervals).

TABLE 2.7.2.3.18
Arithmetic mean (% CV) single- and multiple-dose pharmacokinetic
parameters for cilansetron following administration of oral cilansetron
16 mg and 32 mg as single doses and TID for 5 days (Study S.241.1.102)

| | | Cilansetron 16 mg | | Cilansetron 32 mg | |
|---|---|---|---|---|---|
| | | Single | Multiple | Single | Multiple |
| Group | Parameter | Dose | Dose | Dose | Dose |
| Females | AUC (ng · h/mL)^{a} | 130 (46) | 143 (35) | 433 (52) | 405 (54) |
| (n = 8) | DN-AUC (ng · h/mL)^{a} | 8.14 (46) | 8.95 (35) | 13.5 (52) | 12.7 (54) |
| | Cmax (ng/mL) | 65.1 (41) | 74.5 (40) | 270 (65) | 210 (56) |
| | Tmax (h)^{b} | 1.00 (1.00, 1.50) | 1.00 (0.67, 1.50) | 1.00 (0.67, 1.00) | 1.00 (0.67, 1.50) |
| | Cmin (ng/mL) | NA | 2.19 (149) | NA | 12.5 (196) |
| | T½ (h) | 1.13 (17) | 1.11 (31) | 1.01 (14) | 1.08 (23) |
| | % Dose (urine)^{c} | 0.35 (70) | 0.21 (59) | 0.36 (54) | 0.22 (68) |
| | Linearity Ratio (%)^{d} | NA | 114 (99, 131) | NA | 91 (79, 104) |
| Males | AUC (ng · h/mL)^{a} | 224 (78) | 133 (39) | 469 (71) | 354 (36) |
| (n = 7-8) | DN-AUC (ng · h/mL)^{a} | 14.0 (78) | 8.33 (39) | 14.7 (71) | 11.1 (36) |
| | Cmax (ng/mL) | 72.2 (57) | 59.0 (40) | 161 (67) | 140 (39) |
| | Tmax (h)^{b} | 1.00 (0.67, 2.00) | 0.67 (0.33, 1.50) | 1.50 (1.00, 2.00) | 1.00 (0.67, 1.50) |
| | Cmin (ng/mL) | NA | 3.56 (86) | NA | 11.6 (80) |
| | T½ (h) | 1.61 (27) | 1.50 (27) | 1.44 (22) | 1.40 (29) |
| | % Dose (urine)^{c} | 0.63 (108) | 0.21 (47) | 0.65 (47) | 0.49 (69) |
| | Linearity Ratio (%)^{d} | NA | 69 (51, 93) | NA | 85 (64, 114) |

NA—not applicable;
% CV - coefficient of variation on arithmetic mean in percent;
DN - dose normalized to a 1 mg dose.
^{a}AUC is AUC(0-inf) for single dose and AUC(0-τ) (i.e. AUC(0-8)) for multiple dose.
^{b}Results presented are median (min, max).
^{c}Percent of dose excreted in urine over a 12-hour interval postdose for single dosing, and over an 8-hour interval postdose for multiple dosing.
^{d}Statistical comparison of AUC(0-τ) on multiple dose day to AUC(0-inf) on single dose day; results presented are geometric least squares mean ratios (90% confidence intervals).

In a single-dose, crossover study conducted to evaluate the potential for a drug-drug interaction between liquid Maalox and cilansetron, 12 male and 12-female subjects (aged 19-39 years) received single doses of cilansetron 16 mg alone and cilansetron 16 mg +Maalox in a crossover fashion under fasted conditions. Blood samples for measurement of plasma cilansetron pharmacokinetics were collected up to 24 hours postdose following each treatment. Table 2.7.2.3.19 below presents cilansetron pharmacokinetic parameters for male and female subjects following treatment with cilansetron alone. A statistically significant gender effect was observed in this study (p<0.05). Based on mean values, cilansetron AUC and Cmax were 56% lower in males than in females, though no difference was observed in Tmax. This was accompanied by a 41% lower mean half-life and an almost 2-fold higher apparent oral clearance in males than in females.

TABLE 2.7.2.3.19
Arithmetic mean (% CV) cilansetron pharmacokinetic
parameters in male and female subjects following oral
treatment with cilansetron 16 mg alone
(Study S.241.1.106).

| | Females | Males |
|---|---|---|
| Parameter | (n = 12) | (n = 12) |
| AUC (0-inf) (ng · h/mL) | 361 (52) | 159 (41) |
| AUC (0-last) (ng · h/mL) | 360 (52) | 159 (41) |
| Cmax (ng/mL) | 137 (39) | 63.1 (39) |
| Tmax (h)^{a} | 1.00 (0.75, 1.25) | 1.00 (0.75, 1.25) |
| T½ (h) | 2.97 (46) | 1.75 (32) |
| CL/F (L/h) | 60.2 (64) | 115 (39) |
| Vz/F (L) | 202 (31) | 276 (32) |

% CV - coefficient of variation on arithmetic mean in percent
^{a}Results presented are median (min, max).

The effect of gender on the pharmacokinetics of cilansetron and its 4-hydroxy metabolites was also evaluated in a population pharmacokinetic analysis of a Phase I1 study in non-constipated IBS patients being treated with 1 mg, 2 mg, 8 mg, or 16 mg cilansetron or placebo TID for 12 weeks. Gender was not found to have a significant effect on the pharmacokinetics of cilansetron or its metabolites.

Effect of Renal Impairment

The effect of renal impairment on the pharmacokinetics of cilansetron was evaluated in a study excluding subjects with end-stage renal disease, and utilizing a single-center, open-label, multiple-dose, parallel group study in 13 male and 9 female subjects, aged 33-74 years. Subjects were enrolled into 4 groups as follows: 9 subjects in the healthy group (normal renal function, creatinine clearance >82 mL/min), 5 subjects in the mild renal impairment group (creatinine clearance 52 to 78 mL/min), 4 subjects in the moderate renal group (creatinine clearance 32 to 48 mL/min), and 4 subjects in the severe renal group (creatinine clearance <28 mL/min). Healthy subjects were matched to renally-impaired subjects with respect to age, weight, gender, and smoking status. All subjects received oral cilansetron 2 mg TID on Days 1 to 5, and a single dose on the morning of Day 6. Blood samples for measurement of plasma pharmacokinetics of cilansetron and its 4-hydroxy metabolites were collected up to 24 hours following dosing on Day 6, and log-transformed pharmacokinetic parameters for these analytes were statistically compared between study groups. Serum concentrations of unbound cilansetron and its 4-hydroxy metabolites were also measured prior to dosing and at 1 and 1.5 hours postdose on Day 6.

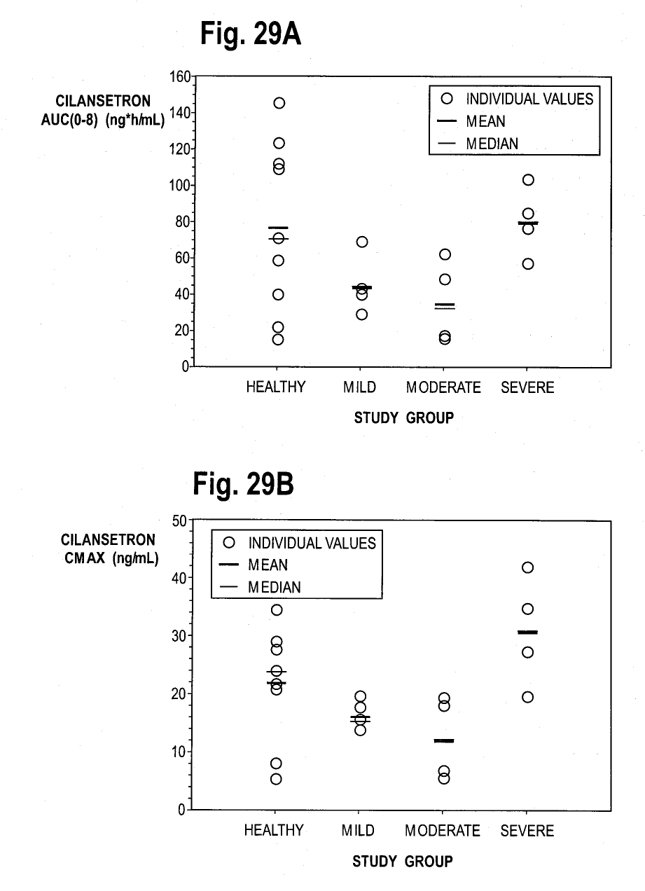

Table 2.7.2.3.20 presents descriptive statistics for the pharmacokinetic parameters of cilansetron and its 4-hydroxy metabolites by study group on Day 6, and FIGS. 29a-b.

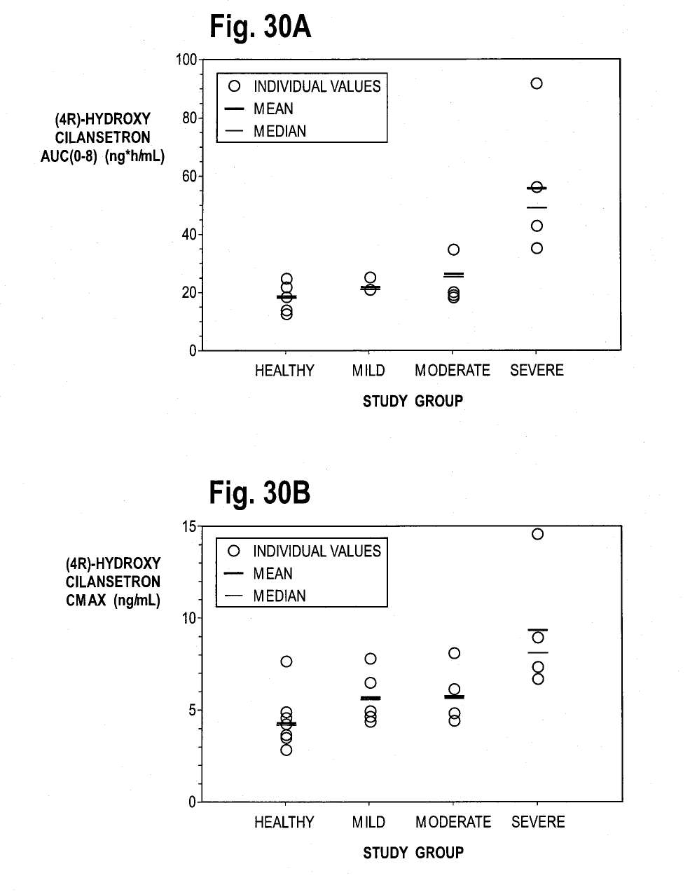

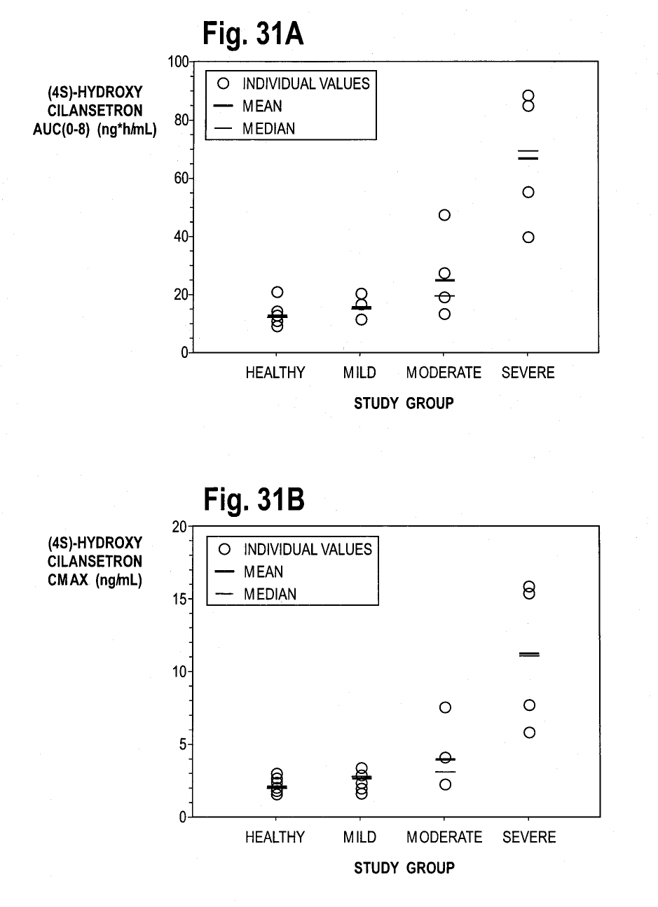

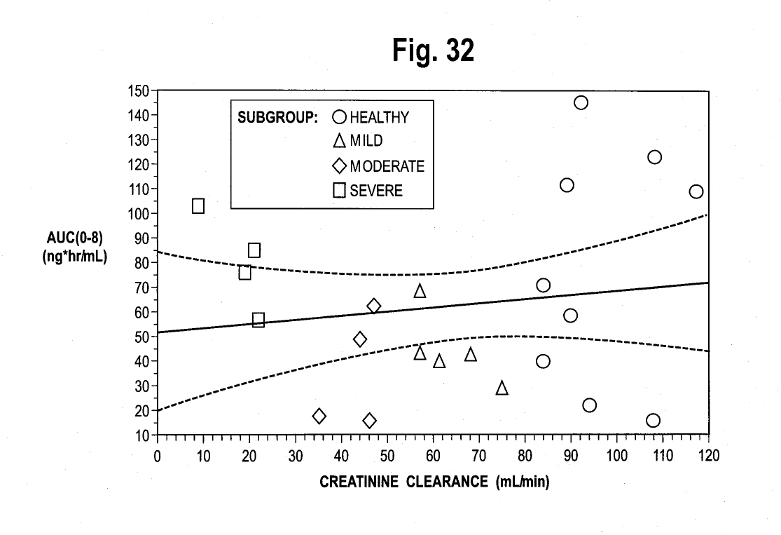

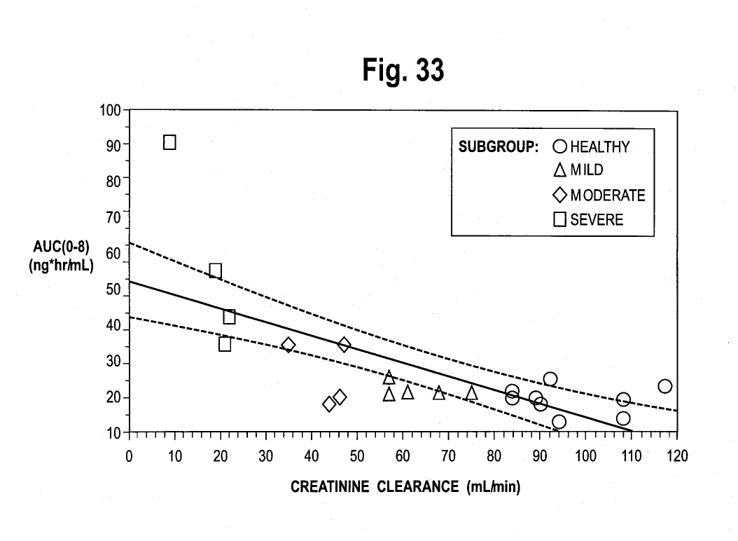

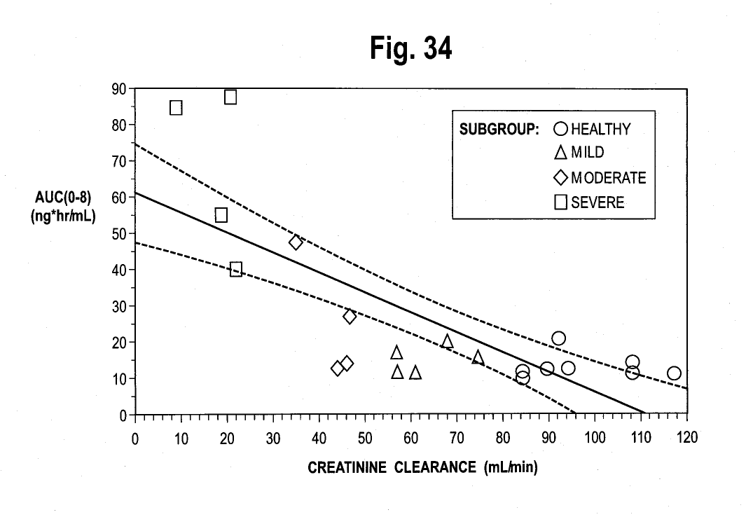

FIGS. 30a-b, and FIGS. 31a-b present individual, mean, and median AUC(0-τ) and Cmax values on Day 6. Table 2.7.2.3.2 1 presents the results of statistical comparisons of pharmacokinetic parameters between study groups (renally impaired to healthy) for cilansetron and its 4-hydroxy metabolites. FIG. 32, FIG. 33, and FIG. 34 show the results of linear regressions of AUC(0-τ) of cilansetron and its 4-hydroxy metabolites versus creatinine clearance. The results of this study showed that the steady-state pharmacokinetics of cilansetron were not significantly affected by renal impairment. Though variability was high [intrasubject % CV of 24-66% for AUC(0-T) and 15-59% for Cmax] resulting in large differences in means between groups, no significant changes or trends were observed in cilansetron AUC(0-τ) or Cmax with increasing renal impairment. Additionally, regression analyses of cilansetron AUC(0-τ) and Cmax versus creatinine clearance showed no correlations between these parameters. No significant differences were observed between study groups for Tmax or half-life.

Unlike the parent drug, the steady-state pharmacokinetics of the two 4-hydroxy metabolites were significantly affected by renal impairment and were especially pronounced with severe impairment [approximate 2- to 3- fold increase for (4R)-hydroxy cilansetron and 5-fold increase for (4S)-hydroxy cilansetron]. For (4R)-hydroxy cilansetron, mean AUC(0-τ) increased by 18%, 37%, and 184%, and mean Cmax increased by 34%, 37%, and 115% with mild, moderate, and severe impairment, respectively, compared to healthy controls. For (4S)-hydroxy cilansetron, geometric mean AUC(0-τ) increased by i9%, 74%, and 407%, and mean Cmax increased by 29%, 71%, and 385% with mild, moderate, and severe impairment, respectively, compared to healthy controls. No significant change was observed in Tmax for either metabolite. Linear regressions of AUC(0-τ) and Cmax for both metabolites versus creatinine clearance showed a clear increase in these parameters with decreasing creatinine clearance. Half-lives for both metabolites tended to increase with increasing renal impairment and were almost doubled with severe impairment, further supporting the hypothesis that the increased exposure of these metabolites is a result of decreased renal elimination.

No significant increases or trends toward an increase were observed in percent free (unbound) cilansetron or its 4-hydroxy metabolites with increasing renal impairment at predose, 1 hour, or 1.5 hours postdose on Day 6 (Table 2.7.2.3.12).

TABLE 2.7.2.3.20
Arithmetic mean (% CV) pharmacokinetic parameters for cilansetron and
its 4-hydroxy metabolites on Day 6 in healthy and renally impaired
subjects following dosing with cilansetron 2 mg TID (Study S.241.1.110)

| | | | Mild | Moderate | Severe |
|---|---|---|---|---|---|
| | | Healthy | Impairment | Impairment | Impairment |
| Analyte | Parameter | (n = 9) | (n = 5) | (n = 4) | (n = 4) |
| Cilansetron | AUC(0-τ) (ng · h/mL) | 77.0 (61) | 44.5 (33) | 35.5 (66) | 80.3 (24) |
| | Cmax (ng/mL) | 22.0 (44) | 16.0 (15) | 12.3 (59) | 30.8 (31) |
| | Tmax (h)^{a} | 1.00 (0.50, 1.52) | 1.25 (0.75, 1.75) | 1.13 (0.75, 1.75) | 0.76 (0.53, 1.00) |
| | Cmin (ng/mL)^{b} | 4.10 (78) | 1.73 (31) | 1.19 (69) | 2.95 (37) |
| | T½ (h) | 2.85 (42) | 2.29 (16) | 1.80 (31) | 2.39 (27) |
| | CL/F (mL/min) | 762 (93) | 808 (30) | 1365 (64) | 435 (26) |
| | Vz/F (L) | 150 (63) | 158 (30) | 182 (42) | 90.4 (35) |
| (4R)—OH | AUC(0-τ) (ng · h/mL) | 18.7 (21) | 21.6 (9) | 26.3 (34) | 55.8 (45) |
| | Cmax (ng/mL) | 4.30 (33) | 5.66 (25) | 5.83 (28) | 9.35 (39) |
| | Tmax (h)^{a} | 1.25 (0.75, 1.52) | 1.25 (0.75, 1.50) | 1.25 (0.75, 2.07) | 1.50 (1.00, 2.02) |
| | Cmin (ng/mL)^{b} | 1.56 (24) | 1.68 (24) | 2.06 (40) | 4.36 (36) |
| | T½ (h) | 7.05 (21) | 7.93 (38) | 9.32 (36) | 13.9 (30) |
| (4S)—OH | AUC(0-τ) (ng · h/mL) | 12.9 (25) | 15.3 (25) | 25.3 (64) | 67.0 (35) |
| | Cmax (ng/mL) | 2.15 (21) | 2.77 (22) | 4.10 (60) | 11.2 (46) |
| | Tmax (h)^{a} | 2.00 (0.50, 4.00) | 2.00 (1.00, 3.03) | 1.75 (1.75, 3.00) | 1.40 (0.75, 3.00) |
| | Cmin (ng/mL)^{b} | 1.24 (31) | 1.76 (38) | 2.71 (72) | 7.00 (35) |
| | T½ (h) | 8.68 (29) | 9.31 (24) | 12.4 (28) | 16.7 (45) |

% CV - coefficient of variation on arithmetic mean in percent.
^{a}Results presented are median (min, max).
^{b}Day 6 morning predose value.

TABLE 2.7.2.3.21
Results of statistical comparisons of pharmacokinetic parameters for
cilansetron and its 4-hydroxy metabolites on Day 6 between healthy and
renally impaired subjects following dosing with cilansetron 2 mg TID
(Study S.241.1.110)

| | | Geometric Least Squares Mean Ratio (90% CI) (%)^{a} | | | |
|---|---|---|---|---|---|
| | | Mild/ | Moderate/ | Severe/ | Renal Combined/ |
| Analyte | Parameter | Healthy | Heathy | Healthy | Healthy |
| Cilansetron | AUC(0-τ) | 71 (38, 132) | 49 (25, 95) | 130 (66, 254) | 76 (47, 124) |
| | Cmax | 83 (50, 140) | 55 (32, 96) | 156 (89, 272) | 90 (60, 134) |
| | T½ | 85 (63, 114) | 65 (47, 89)* | 87 (64, 120) | 78 (62, 99) |
| | CL/F | 141 (76, 264) | 206 (105, 403) | 77 (39, 151) | 131 (81, 213) |
| (4R)—OH | AUC(0-τ) | 118 (90, 153) | 137 (104, 182) | 284 (215, 377)* | 166 (136, 204)* |
| | Cmax | 134 (101, 177) | 137 (102, 185) | 215 (159, 290)* | 158 (127, 196)* |
| | T½ | 109 (84, 142) | 129 (97, 171) | 195 (147, 259)* | 140 (114, 172)* |
| (4S)—OH | AUC(0-τ) | 119 (85, 168) | 174 (120, 251)* | 507 (351, 733)* | 219 (168, 286)* |
| | Cmax | 129 (91, 181) | 171 (118, 248)* | 485 (335, 702)* | 220 (168, 288)* |
| | T½ | 108 (81, 144) | 142 (104, 193) | 186 (136, 254)* | 142 (113, 177)* |

CI—confidence interval.
*Statistically significant difference (p < 0.05).
^{a}Results of ANOVA comparisons of log-transformed data.

The effect of renal function on cilansetron pharmacokinetics was also evaluated in a population pharmacokinetic analysis of cilansetron and 4-hydroxy metabolite concentrations from a Phase II study in non-constipated IBS patients being treated with 1 mg, 2 mg, 8 mg, or 16 mg cilansetron or placebo TID for 12 weeks. Cilansetron clearance was not found to be related to serum creatinine level or creatinine clearance. In the case of the 4-hydroxy metabolites, however, creatinine clearance was found to be a covariate for the population pharmacokinetic model of both metabolites. The model showed that as creatinine clearance increased by 1 mL/min, the apparent oral clearance increased by 0.447 L/h and 0.883 L/h for (4R)-hydroxy and (4S)-hydroxy cilansetron, respectively.

Increases in AUC(0-τ) of the two metabolites at levels of renal impairment comparable to that of the renal impairment study were predicted from NONMEM simulations utilizing the final model developed in the population pharmacokinetic analysis. The results of these simulations are compared to the results obtained from Study S.241.1.110 in Table 2.7.2.3.22 below. The effect on AUC(0-τ) predicted by the population model for mild impairment was similar to that observed in Study S.241.1.110 for both (4R)-hydroxy and (4S)-hydroxy cilansetron. The predicted effect was much smaller than the observed effect for the moderate and severe impairment groups, but this is probably due to the fact that the study population used for the population analysis consisted mainly of subjects with normal renal status (64%) and mild impairment (34%), with only 2% of subjects showing moderate impairment and no subjects having severe impairment.

TABLE 2.7.2.3.22
Observed mean changes in AUC(0-τ) of (4R)-hydroxy and (4S)-hydroxy
cilansetron with renal impairment compared to simulated changes from a
covariate population pharmacokinetic model at the same levels of
impairment (Studies S.241.1.110 and S.241.2.112)

| | | % Increase in AUC(0-τ) compared to Healthy Subjects | | | |
|---|---|---|---|---|---|
| | | (4R)—OH Cilansetron | | (4S)—OH-Cilansetron | |
| Renal | CLcr^{a} | Observed^{b} | Simulated^{c} | Observed^{b} | Simulated^{c} |
| Function | (mL/min) | (Study S.241.1.110) | (NONMEM) | (Study S.241.1.110) | (NONMEM) |
| Healthy | 96.2 | — | — | — | — |
| Mild | 63.6 | 18 | 11.8 | 19 | 14.3 |
| Moderate | 43.0 | 37 | 20.8 | 74 | 25.7 |
| Severe | 17.8 | 184 | 34.0 | 407 | 43.1 |
| ^{a}CLcr values are arithmetic mean values from Study S.241.1.110. These mean values were used in the NONMEM simulation. | | | | | |
| ^{b}Observed % increase, based on the geometric least squares mean ratios from statistical comparisons of study groups in Study S.241.1.110. | | | | | |
| ^{c}These simulations are done in support of this submission and are not reported in the study report. The details of this NONMEM simulation are as follows: Two groups of 4 subjects were simulated - one group per subpopulation of subjects based on Kn. Within each group, there was one subject with each of the CLcr values shown above. Steady-state (4R)—OH and (4S)—OH cilansetron concentrations were calculated using NONMEM, based on the respective population PK parameter estimates | | | | | |
| # and assuming a TID dose of 2 mg. Predicted concentration versus time data were then used to calculate AUC(0-τ). There were no differences in AUC between the 2 simulated groups for a given level of renal function. The outputs for these simulations are on file at Solvay Pharmaceuticals. | | | | | |

Effect of Hepatic Impairment

The effect of hepatic impairment on the pharmacokinetics of cilansetron was evaluated in a multiple-dose, parallel group study in 14 healthy and 14 moderately to severely hepatically impaired male and female subjects, aged 41-70 years. Impaired subjects (Child-Pugh score 7 to 11) were matched to healthy subjects by age (±2 years), sex, menstrual status, and smoking history status. All subjects received oral cilansetron 8 mg TID on Days 1 to 5, and a single dose on the morning of Day 6. Blood samples for measurement of plasma pharmacokinetics of cilansetron and its 4-hydroxy metabolites were collected up to 24 hours following dosing on Day 6, and log-transformed AUC(0-τ) and Cmax for these analytes were statistically compared between study groups. Blood samples were also collected at predose on Day 1, and at predose, 1 hour and 1.5 hours' postdose on Day 6, for measurement of-cilansetron protein binding in serum.

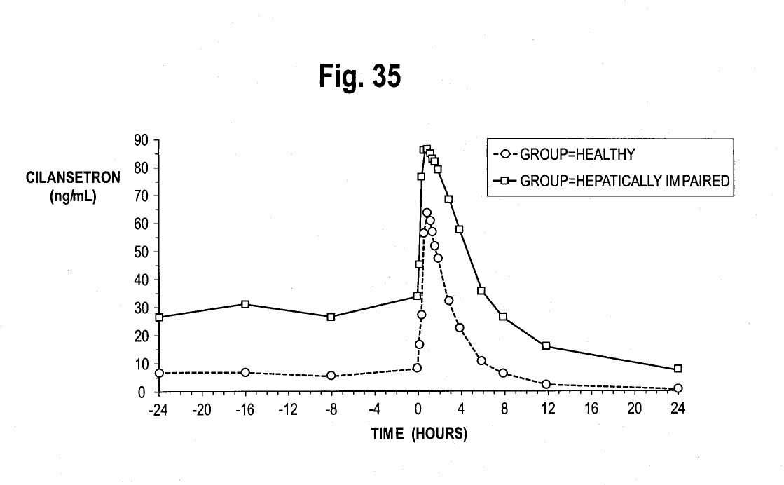

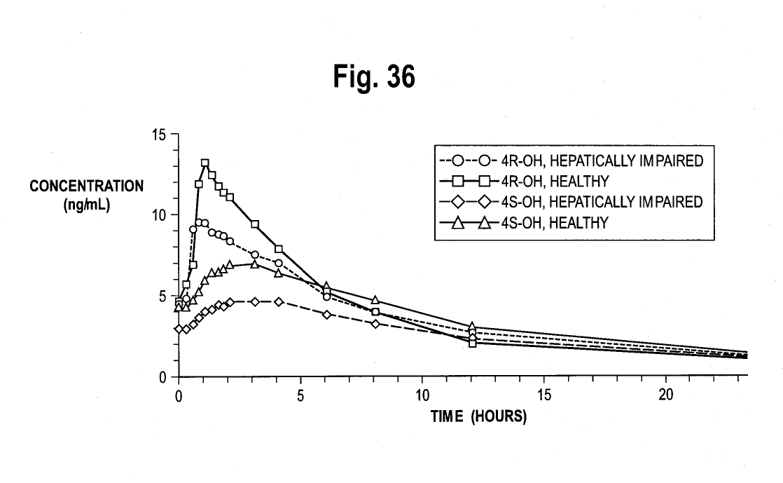

FIG. 35 and FIG. 36 present mean plasma concentration-time profiles for cilansetron and its 4-hydroxy metabolites on Day 6, in healthy and hepatically impaired subjects. Table 2.7.2.3.23 presents descriptive statistics for the pharmacokinetic parameters of cilansetron and its 4-hydroxy metabolites, and the results of statistical comparisons between study groups (hepatically impaired versus healthy). Mean estimates of cilansetron pharmacokinetic parameters in healthy subjects, including AUC(0-τ), Cmax, half-life, and percent unbound, were higher in this study than in some other studies using a similar dose (cilansetron 8 mg TID). Nevertheless, the results of this study showed that cilansetron clearance was decreased with hepatic impairment, resulting in an 88% mean increase in cilansetron AUC(0-τ) a 22% increase in Cmax, and a 2-fold increase in halflife in impaired compared to healthy subjects. Plasma protein binding did not show any notable difference between impaired and healthy subjects. Consistent with the decrease in cilansetron clearance with hepatic impairment, the extent of formation of the 4-hydroxy metabolites of cilansetron was also decreased. On average, (4R)-hydroxy cilansetron AUC(0-τ) and Cmax decreased by 14% and 30% respectively, and (4S)-hydroxy cilansetron AUC(0-τ) and Cmax decreased by 32% and 36% respectively, in impaired compared to healthy subjects.

The relationship between hepatic function (total bilirubin, albumin, prothrombin time, Child- Pugh scores, encephalopathy, and ascites) and body weight adjusted and unadjusted AUC, CLE, and Vz/F for cilansetron was evaluated using correlation analyses. Statistically significant correlations (p<0.05) were found between the hepatic parameters, bilirubin, albumin, and prothrombin time, and AUC and/or CL/F. Particularly strong negative correlations were observed between albumin and cilansetron AUC or CIJF (r>0.7).

TABLE 2.7.2.3.23
Arithmetic mean (% CV) pharmacokinetic parameters for cilansetron and
its 4-hydroxy metabolites in hepatically impaired and healthy subjects and
results of statistical comparisons between groups following oral treatment
with cilansetron 8 mg TID (Study S.241.1.107)

| | | | | Statistical | | |
|---|---|---|---|---|---|---|
| | | Arith. Mean (% CV) | | Comparison^{b} | | |
| | | Hepatically | Healthy | Geom. LS Means | | |
| Analyte | Parameter | Impaired (N = 13) | (N = 13) | Impaired | Healthy | Ratio^{c} |
| Cilansetron | AUC(0-τ) (ng · h/mL) | 444 (59) | 208 (54) | 334 | 178 | 1.88 |
| | Cmax (ng/mL) | 94.4 (43) | 75.2 (40) | 83.9 | 68.7 | 1.22 |
| | Tmax (h) | 0.75 (0.50, 1.75)^{a} | 1.00 (0.75, 1.75)^{a} | NC | NC | NC |
| | Cmin (ng/mL) | 26.3 (75) | 5.47 (88) | 13.7 | 3.63 | 3.78* |
| | T½ (h) | 8.16 (51) | 4.37 (25) | NC | NC | NC |
| | CL/F (L/h) | 38.5 (118) | 55.0 (74) | NC | NC | NC |
| | Vz/F (L) | 388 (123) | 303 (45) | NC | NC | NC |
| | % free in serum | 5.5 (23) | 7.0 (73) | NC | NC | NC |
| (4R)—OH | AUC(0-τ) (ng · h/mL) | 53.7 (40) | 62.7 (32) | 49.4 | 57.4 | 0.86 |
| | Cmax (ng/mL) | 11.1 (62) | 14.1 (28) | 9.30 | 13.2 | 0.70 |
| | Tmax (h) | 1.50 (0.50, 3.00) | 1.00 (0.75, 2.00) | NC | NC | NC |
| | T½ (h) | 10.0 (41) | 6.38 (18) | NC | NC | NC |
| (4S)—OH | AUC(0-τ) (ng · h/mL) | 33.8 (57) | 47.9 (36) | 29.1 | 42.5 | 0.68 |
| | Cmax (ng/mL) | 5.17 (59) | 7.57 (33) | 4.39 | 6.89 | 0.64 |
| | Tmax (h) | 3.00 (1.25, 4.00) | 3.00 (1.25, 4.00) | NC | NC | NC |
| | T½ (h) | 9.41 (30) | 7.65 (17) | NC | NC | NC |

NC—not calculated;
Geom. LS mean - geometric least squares mean;
% CV - coefficient of variation on arithmetic mean in percent;
AUC(0-τ) - AUC(0-8)
^{a}Results presented are median (min, max).
^{b}Results of ANOVA comparisons of log-transformed data.
^{c}Geometric least squares mean ratio (impaired/healthy).
*Statistically significant (p < 0.05).

The effect of concomitantly administered antacid (liquid,Maalox) on the pharmacokinetics of cilansetron has been evaluated in a single-dose, crossover study in 24 healthy, male and female subjects, aged 19 to 39 years. Subjects received the following two treatments during each of the study periods in a crossover fashion: a single oral dose of cilansetron 16 mg tablet (reference treatment); a single oral dose of cilansetron 16 mg tablet +20 mL oral Maalox liquid administered every 4 hours (test treatment; 4 doses of Maalox in total; cilansetron was administered 5 minutes after the first dose of Maalox). Both cilansetron treatments were administered under fasted conditions. Blood samples for measurement of plasma cilansetron pharmacokinetics were collected up to 24 hours postdose following each treatment, and log-transformed cilansetron AUC(0-last), AUC(0-inf), and Cmax values were statistically compared between treatments.

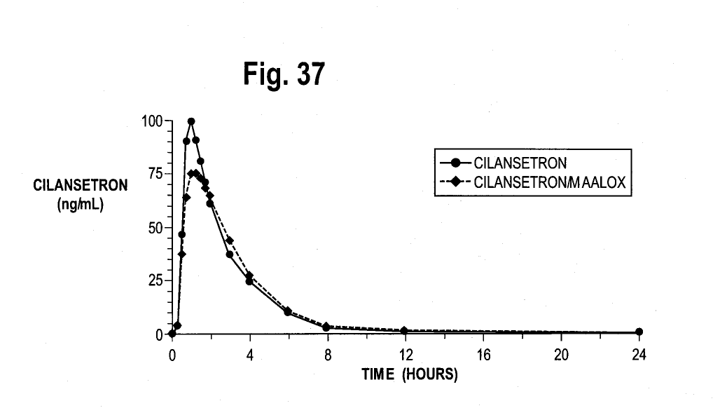

FIG. 37 presents the mean plasma cilansetron concentration- time profile following each treatment, and Table 2.7.2.3.24 presents descriptive statistics for cilansetron pharmacokinetic parameters and the results of statistical comparisons between treatments. The results of this study show that concomitant administration of Maalox has no significant effect on the extent of exposure [AUC(O-last) and AUC(0-inf)] of cilansetron. A small decrease (13% mean decrease) was observed in cilansetron Cmax following concomitant administration, but this decrease is not considered to be clinically significant.

TABLE 2.7.2.3.24
Arithmetic mean (% CV) cilansetron pharmacokinetic parameters and
results of statistical comparisons between treatments following oral
treatment with cilansetron 16 mg alone or in combination with Maalox
liquid (Study S.241.1.106)

| | | | Statistical | |
|---|---|---|---|---|
| | | | Comparisons | |
| | Arith. Mean (% CV) | | (Treatment B/A)^{b} | |
| Parameter | Treatment B (N = 24) | Treatment A (N = 23) | Ratio (%) | 90% CI |
| AUC (0-last) (ng · h/mL) | 259 (63) | 264 (66) | 100 | 93, 108 |
| AUC (0-inf) (ng · h/mL) | 260 (63) | 264 (66) | 100 | 93, 108 |
| Cmax (ng/mL) | 88.0 (57) | 102 (55) | 87 | 79, 96 |
| Tmax (h)^{a} | 1.00 (0.75, 2.00)^{a} | 1.00 (0.75, 1.25)^{a} | NC | NC |
| T½ (h) | 2.51 (47) | 2.39 (51) | NC | NC |
| CL/F (L/h) | 83.5 (52) | 86.7 (57) | NC | NC |
| Vz/F (L) | 253 (40) | 237 (35) | NC | NC |

Treatment A: Cilansetron 16 mg tablet;
Treatment B: Cilansetron 16 mg tablet + 4 × 20 mL Maalox liquid.
NC—not calculated;
% CV - coefficient of variation on arithmetic mean in percent.
^{a}Results presented are median (min, max).
^{b}Geometric least squares mean ratio and 90% confidence interval from ANOVA comparisons of log-transformed data.

The effects of concomitant administration of cilansetron and a commonly used oral contraceptive, Ortho Tri-Cyclen (ORC), on the pharmacokinetics and pharmacodynamics of ORC and the pharmacokinetics of cilansetron has been evaluated in a randomized, double-blind, placebo-controlled, multiple-dose, 2-period, crossover study in 20 healthy, female subjects, aged 19 to 42 years. Subjects received the following two treatments during each of two 28-day study periods (2 ORC cycles) in a randomized, crossover fashion: Treatment B (reference treatment)—ORC on Days 1-28 (standard ORC cycle)+placebo TID on Days 14-20 and a single dose of placebo on Day 2 1; Treatment A (test treatment)—ORC on Days 1-28 (standard ORC cycle)+cilansetron 2 mg TID on Days 14-20 and a single dose of cilansetron 2 mg on Day 21. Serial blood samples for the measurement of the plasma pharmacokinetics of ethinyl estradiol (EE), norgestimate, (NGM), and the major metabolites of NGM (17-deacetyl-norgestimate [17-d-NGM] and norgestrel [NGL]) were collected following dosing on Day 2 1 of each period. Log-transformed AUC(0-τ) (estimated over the 24-hour dosing interval) and Cmax for EE and 17-d-NGM on Day 21 were compared statistically between treatments. For evaluation of the effect of concomitant cilansetron on the pharamacodynamics of ORC, blood samples were collected prior to morning dosing on Days 18, 19, 20, 26, 27, and 28 for measurement of serum progesterone (PG), luteinizing hormone (LH), and follicle stimulating hormone (FSH) concentrations, and untransformed data were compared statistically between treatments. Blood samples were also collected at 2, 4, 8, and 24 hours postdose on Day 21 for measurement of concentrations of cilansetron and its metabolites, (4R)-hydroxy and (4S)-hydroxy cilansetron.

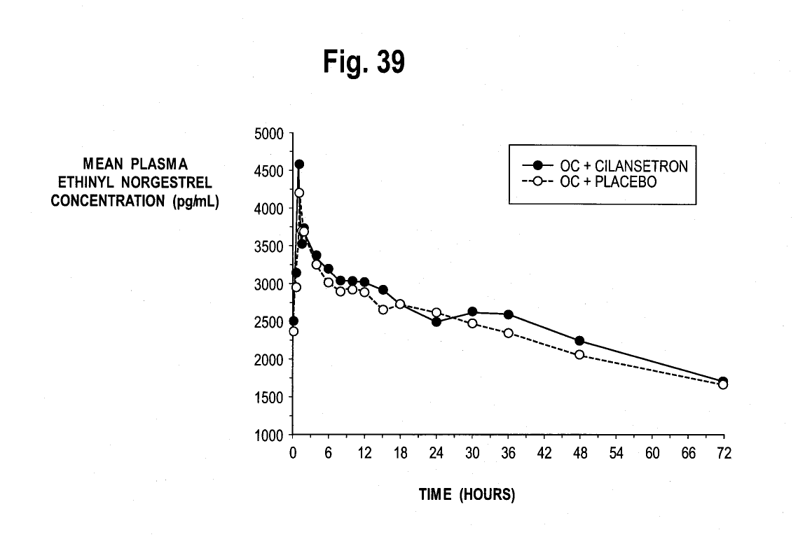

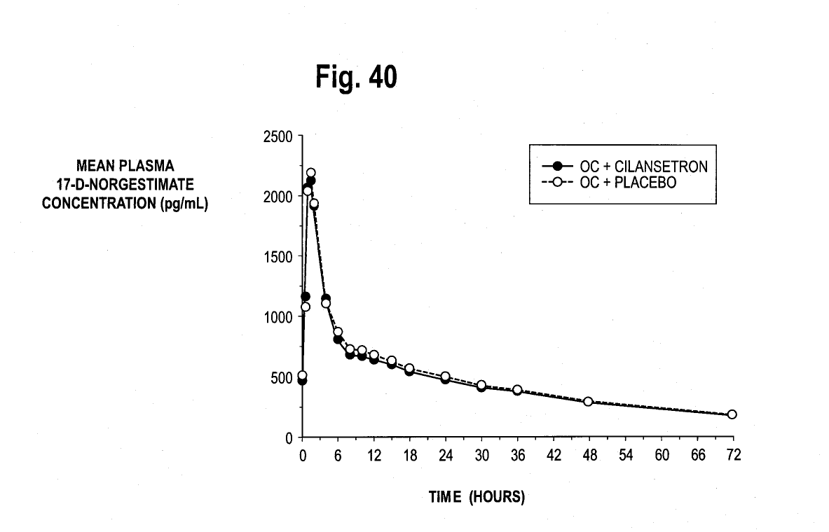

FIG. 38, FIG. 39, and FIG. 40 present mean plasma concentration-time profiles for EE, NGL, and 17-d-NGM on Day 21 following each treatment. Insufficient quantifiable NGM concentrations were available to evaluate the pharmacokinetics of this analyte. Steady state was shown to be achieved for both EE and 17-d-NGM by Day 21. Table 2.7.2.3.25 presents descriptive statistics for EE, NGL, and 17-d-NGM pharmacokinetic parameters and the results of statistical comparisons between treatments for EE and 17-d-NGM. The results of this study showed that concomitant administration of cilansetron does not affect the steady-state pharmacokinetics of EE or 17-d-NGM. A small mean decrease (10% and 8%, respectively) was observed in extent of exposure [AUC(0-τ)] and Cmax of EE, but the 90% confidence intervals for both parameters were within a range of 80-125% demonstrating that this difference was not significant. No differences were observed in AUC(0-τ) and Cmax of 17-d-NGM. Additionally, the mean concentration-time profiles of NGL were similar, showing that NGL pharmacokinetics were not affected.

TABLE 2.7.2.3.25
Arithmetic mean (SD) ethinyl estradiol, morgestrel, and 17-deacetyl-
norgestimate pharmacokinetic parameters and results of statistical
comparisons between treatments, following oral treatment with Ortho Tri-
Cyclen alone or in combination with cilansetron 2 mg TID (Study S.241.1.104)

| | | | | Statistical | |
|---|---|---|---|---|---|
| | | | | Comparisons | |
| | | Arith. Mean (SD)^{a} | | (Treatment A/B)^{c} | |
| Analyte | Parameter | ORC + Cilansetron | ORC + Placebo | Ratio (%) | 90% CI |
| EE | AUC(0-τ) (ng · h/mL) | 1.21 (0.55) | 1.32 (0.57) | 90 | 86, 95 |
| | Cmax (ng/mL) | 0.144 (0.060) | 0.156 (0.061) | 92 | 85, 100 |
| | Tmax (h) | 1.25 (1.00, 2.00)^{b} | 1.50 (1.00, 2.00)^{b} | NC | −0.5, 0.0 |
| | Cmin (ng/mL) | 0.022 (0.013) | 0.024 (0.013) | NC | NC |
| | T½ (h) | 20.9 (5.07) | 21.2 (6.07) | NC | NC |
| NGL | AUC(0-τ) (ng · h/mL) | 76.4 (31.0) | 69.7 (34.2) | NC | NC |
| | Cmax (ng/mL) | 5.21 (4.52) | 4.53 (3.87) | NC | NC |
| | Tmax (h) | 2.00 (1.00, 18.00) | 1.75 (1.00, 2.00) | NC | NC |
| | Cmin (ng/mL) | 2.45 (1.01) | 2.34 (1.19) | NC | NC |
| | T½ (h) | 55.8 (23.4) | 67.6 (29.7) | NC | NC |
| 17-d-NGM | AUC(0-τ) (ng · h/mL) | 18.9 (5.8) | 19.5 (6.1) | 97 | 91, 103 |
| | Cmax (ng/mL) | 2.33 (0.89) | 2.29 (0.79) | 102 | 90, 115 |
| | Tmax (h) | 1.50 (1.00, 2.00)^{b} | 1.50 (1.00, 2.00)^{b} | NC | −0.5, 0.0^{d} |
| | Cmin (ng/mL) | 0.454 (0.163) | 0.479 (0.163) | NC | NC |
| | T½ (h) | 33.9 (10.6) | 34.8 (11.9) | NC | NC |

NC—not calculated;
AUC(0-τ) - AUC(0-8);
ORC - Ortho Tri-Cyclen;
EE—ethinyl estradiol;
NGL—norgestrel;
17-d-NGM - 17-deacetyl-norgestimate.
^{a}Mean (SD) values are based on 16 subjects who completed both treatments, except for NGL where it is based on 14-16 subjects.
^{b}Results presented are median (min, max).
^{c}Geometric least squares mean ratios and 90% confidence intervals from ANOVA comparisons of log-transformed data; Treatment A: ORC + cilansetron; Treatment B: ORC + placebo.
^{d}90% CI from nonparameteric analysis of Tmax.

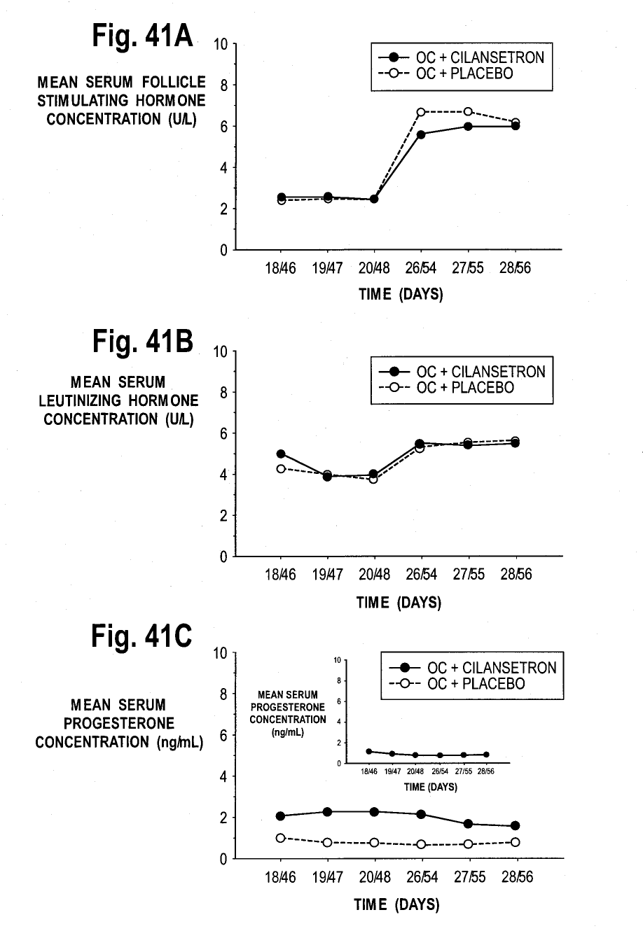

FIGS. 41a-c presents mean predose serum concentrations of PG, LH, and FSH on Days 18, 19, 20, 26, 27, and 28 following treatment with ORC+cilansetron or ORC+placebo. Mean concentrations of FSH and LH were generally superimposable between treatments. Mean concentrations of PG were slightly higher for ORC+cilansetron, but this was attributable to elevated levels in one subject, as demonstrated by the inset plot in FIGS. 41a-c which shows superimposable mean concentrations of PG when the subject was excluded. No statistically significant differences were found between treatments for any of the three analytes.

Since cilansetron has been shown to undergo metabolism by CYP3A4, CYPIA, and CYP2D6, the potential for inhibitors of these enzymes to affect the metabolism of cilansetron was evaluated using ketoconazole (CYP3A4 inhibitor), fluvoxamine (inhibitor of CYP1A2, but also of CYP2C 19, CYP2C9, and CYP3A), and paroxetine (CYP2D6 inhibitor). In an open-label, randomized, parallel-group study in three groups of 12 healthy, male and female subjects (aged 19 to 45 years), subjects received a 2 mg single dose of cilansetron on Day 1, 2 mg TID on Days 2-13, and a 2 mg single dose on Day 14. Additionally, subjects in one group received fluvoxamine (Fevarin®) 50 mg QD on Days 8-14, the second group received paroxetine (Seroxat®) 20 mg QD on Days 8-14, and the third group received ketoconazole (Nizoral®) 200 mg BID on Days 12-14. All morning doses of cilansetron and enzyme inhibitors were administered immediately after a standard breakfast. Serial blood samples for the measurement of steady-state pharmacokinetics of cilansetron and its 4-hydroxy metabolites were collected on Day 7 (cilansetron alone) and Day 14 (cilansetron+enzyme inhibitor) for each-of the three groups. For each treatment group, log-transformed cilansetron AUC(0-τ) and Cmax values on Day 14 (cilansetron+enzyme inhibitor) were compared statistically versus values on Day 7 (cilansetron alone).

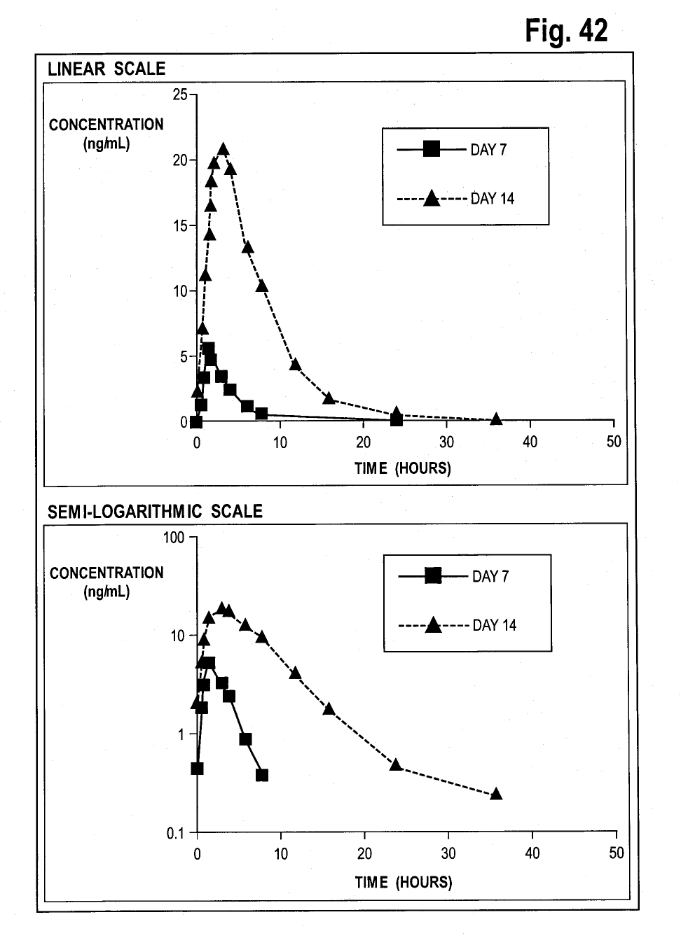

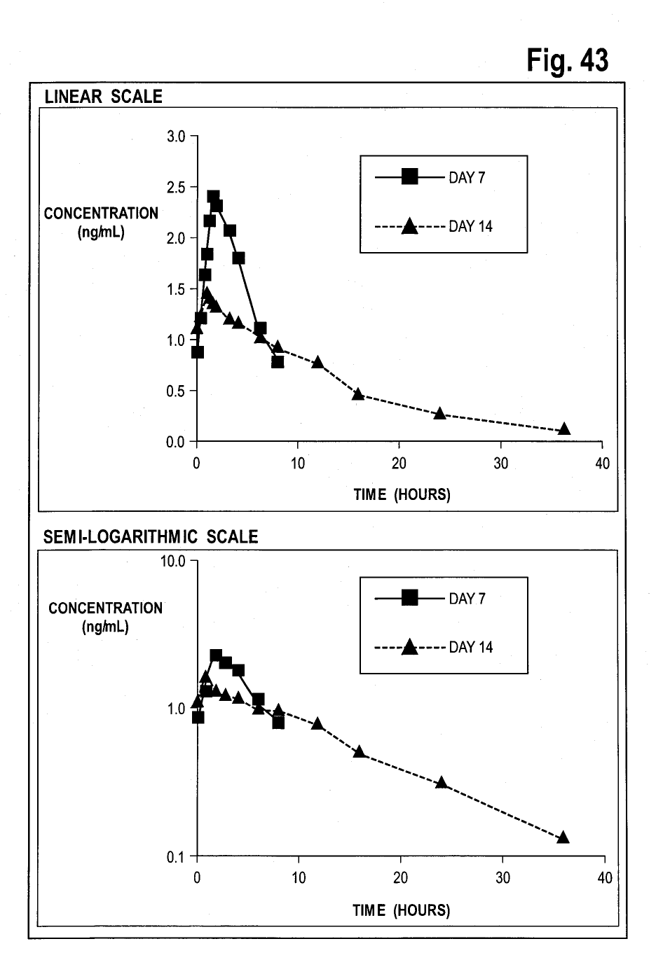

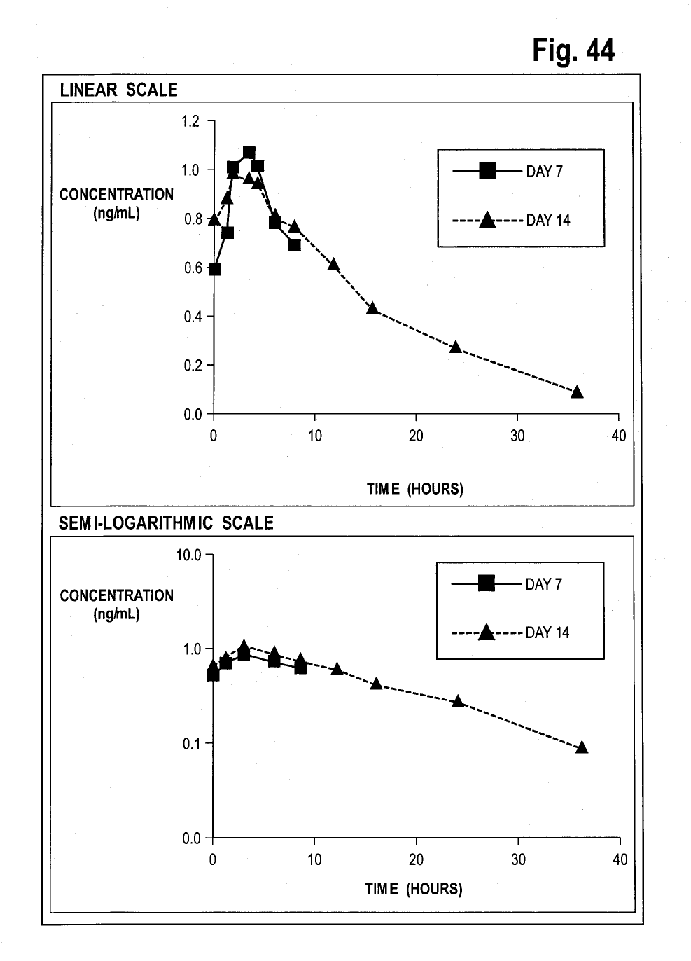

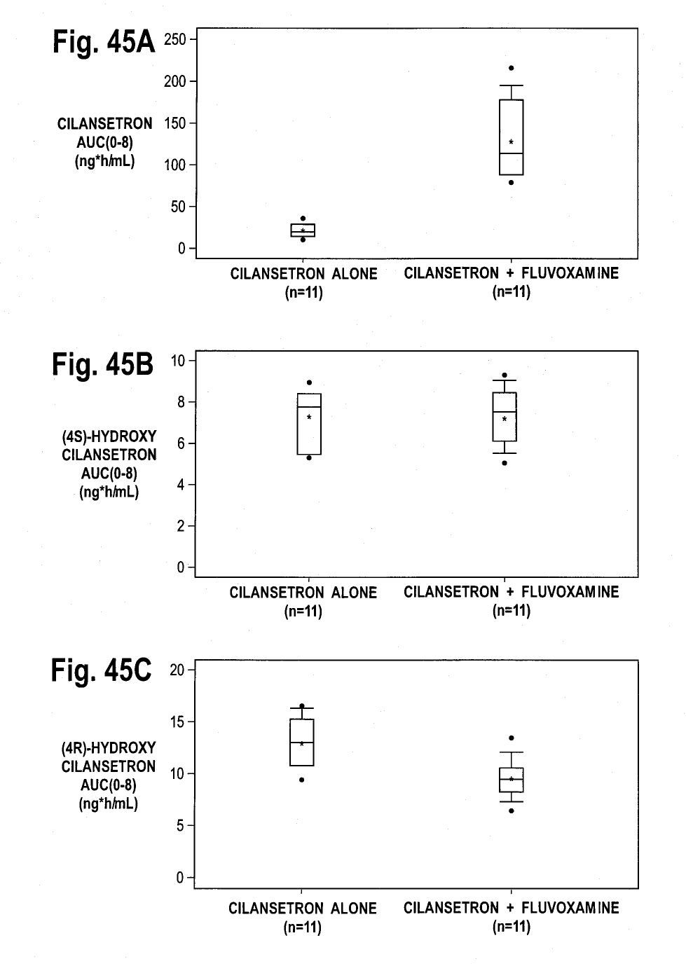

FIG. 42, FIG. 43, and FIG. 44 present geometric mean steady-state plasma concentration-time profiles for cilansetron and its 4-hydroxy metabolites in the absence and presence of concomitant fluvoxamine. Table 2.7.2.3.26 presents descriptive statistics for the pharmacokinetic parameters of cilansetron and its 4-hydroxy metabolites, and the results of statistical comparisons between treatments (cilansetron+fluvoxamine versus cilansetron alone), and FIGS. 45a-c shows the distribution of AUC(0-τ) values for the two treatments. The results showed that coadministration of fluvoxamine produced a large increase in exposure to cilansetron. Mean cilansetron AUC(0-τ) and Cmax increased by 6.6-fold and 3.7-fold, respectively, in the presence of concomitant fluvoxamine. Some corresponding decrease in exposure was observed for its metabolite, (4R)-hydroxy cilansetron, with mean AUC(0-τ) and Cmax decreasing by 24 and 40%, respectively, but no change was observed for (4S)-hydroxy cilansetron.

TABLE 2.7.2.3.26
Arithmetic mean (% CV) steady-state pharmacokinetic parameters for
cilansetron and its 4-hydroxy metabolites and results of statistical
comparisons between treatments following oral treatment with cilansetron
2 mg TID alone or in combination with fluvoxamine 50 mg once daily
(Study S.241.1.109)

| | | Arith. Mean (% CV) | | Statistical | |
|---|---|---|---|---|---|
| | | Cilansetron + | Cilansetron | Comparisons | |
| | | Fluvoxamine | Alone | (Treatment B/A)^{b} | |
| Analyte | Parameter | (N = 11) | (N = 11) | Ratio (%) | 90% CI |
| Cilansetron | AUC(0-τ) (ng · h/mL) | 127 (36) | 20.8 (36) | 656 | 555, 776 |
| | Cmax (ng/mL) | 22.8 (33) | 6.45 (29) | 367 | 308, 439 |
| | Tmax (h) | 3.00 (1.75, 4.00)^{a} | 1.25 (0.75, 4.00)^{a} | NC | NC |
| (4R)—OH | AUC(0-τ) (ng · h/mL) | 9.57 (21) | 12.9 (19) | 76 | 70.83 |
| | Cmax (ng/mL) | 1.63 (22) | 2.74 (18) | 60 | 53, 68 |
| | Tmax (h) | 0.75 (0.0, 1.25)^{a} | 1.75 (0.75, 4.00)^{a} | NC | NC |
| (4S)—OH | AUC(0-τ) (ng · h/mL) | 7.17 (20) | 7.29 (19) | 101 | 91, 118 |
| | Cmax (ng/mL) | 1.07 (23) | 1.17 (17) | 93 | 84, 103 |
| | Tmax (h) | 2.00 (0.0, 16.0)^{a} | 3.00 (1.80, 4.00)^{a} | NC | NC |

NC—not calculated;
% CV - coefficient of variation on arithmetic mean in percent;
AUC(0-τ) - AUC(0-8)
^{a}Results presented are median (min, max).
^{b}Results of ANOVA comparisons of log-transformed data; Treatment B: cilansetron + fluvoxamine, Treatment A: cilansetron alone.

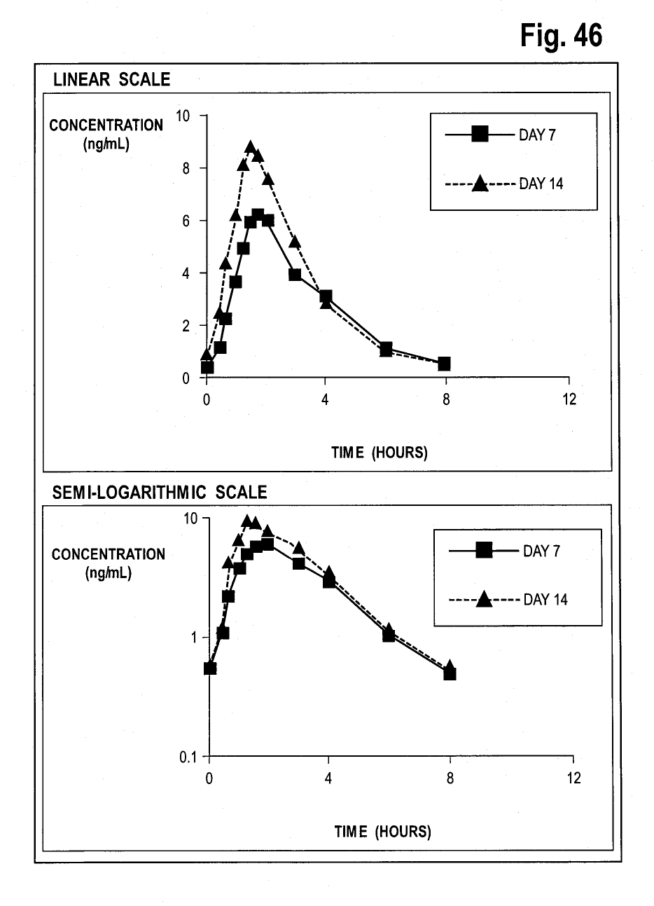

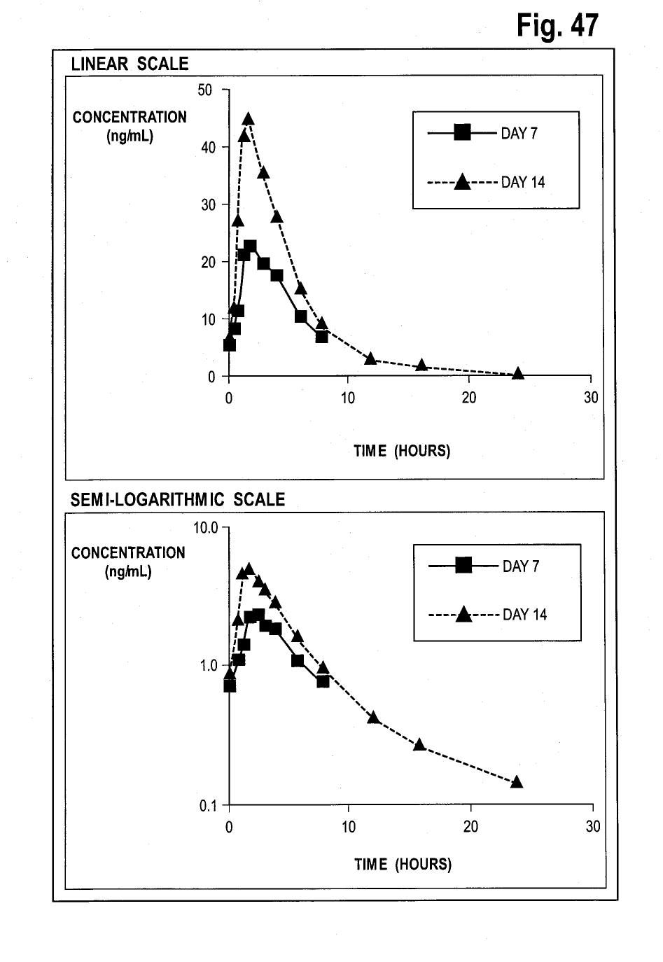

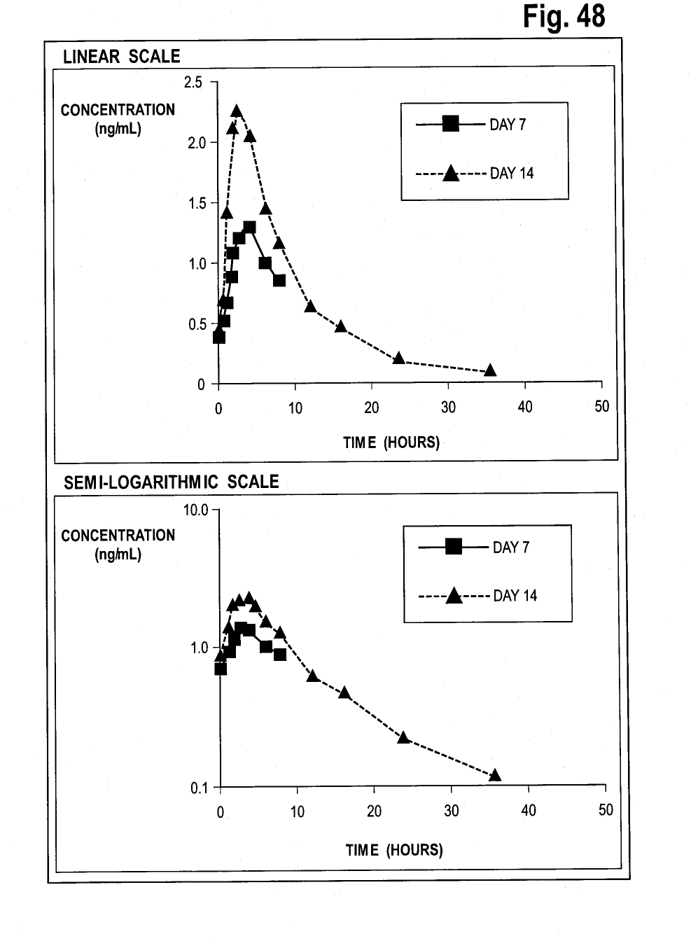

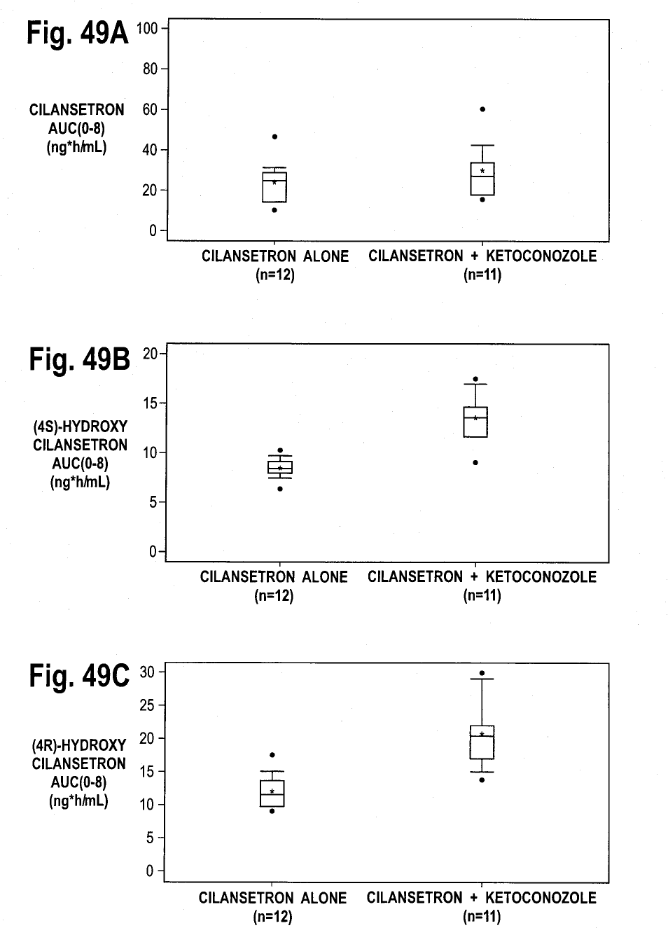

FIG. 46, FIG. 47, and FIG. 48 present geometric mean steady-state plasma concentration-time profiles for cilansetron and its Chydroxy metabolites in the absence and presence of concomitant ketoconazole. Table 2.7.2.3.27 presents descriptive statistics for the pharmacokinetic parameters of cilansetron and its 4-hydroxy metabolites, and the results of statistical comparisons between treatments (cilansetron+ketoconazole versus cilansetron alone), and FIGS. 49a-c presents the distribution of AUC(0-τ) values for the two treatments. The results showed that coadministration of ketoconazole caused an increase in exposure to cilansetron and its 4-hydroxy metabolites. Mean cilansetron AUC(0-τ) and Cmax increased by 40% and 51%, respectively. Similarly, mean (4R)-hydroxy cilansetron AUC(0-T) and Cmax increased by 76% and 109%, respectively, and (4S)-hydroxy cilansetron AUC(0-T) and Cmax increased by 63% and 74%, respectively.

One subject in the cilansetron+ketoconazole study group was identified by genotyping as an ultrarapid metabolizer for CYP2D6. No difference in cilansetron pharmacokinetics was observed in this subject.

TABLE 2.7.2.3.27
Arithmetic mean (% CV) steady-state pharmacokinetic parameters for
cilansetron and its 4-hydroxy metabolites and results of statistical
comparisons between treatments following oral treatment with cilansetron
2 mg TID alone or in combination with ketoconazole 200 mg twice daily
(Study S.241.1.109)

| | | Arith. Mean (% CV) | | Statistical | |
|---|---|---|---|---|---|
| | | Cilansetron + | Cilansetron | Comparisons | |
| | | Ketoconazole | Alone | (Treatment B/A)^{b} | |
| Analyte | Parameter | (N = 11) | (N = 12) | Ratio (%) | 90% CI |
| Cilansetron | AUC(0-τ) (ng · h/mL) | 29.6 (43) | 23.7 (48) | 140 | 118, 165 |
| | Cmax (ng/mL) | 10.7 (45) | 7.99 (60) | 151 | 127, 181 |
| | Tmax (h) | 1.25 (0.75, 2.00)^{a} | 1.50 (0.75, 4.00)^{a} | NC | NC |
| (4R)—OH | AUC(0-τ) (ng · h/mL) | 20.8 (24) | 12.2 (20) | 176 | 161, 192 |
| | Cmax (ng/mL) | 5.11 (28) | 2.48 (22) | 209 | 184, 238 |
| | Tmax (h) | 1.50 (0.75, 2.00)^{a} | 2.00 (1.25, 4.00)^{a} | NC | NC |
| (4S)—OH | AUC(0-τ) (ng · h/mL) | 13.5 (18) | 8.42 (12) | 163 | 147, 180 |
| | Cmax (ng/mL) | 2.29 (17) | 1.35 (12) | 174 | 158, 192 |
| | Tmax (h) | 2.00 (1.75, 4.00)^{a} | 3.00 (1.75, 4.00)^{a} | NC | NC |

NC—not calculated;
% CV - coefficient of variation on arithmetic mean in percent;
AUC(0-τ) - AUC(0-8)
^{a}Results presented are median (min, max).
^{b}Results of ANOVA comparisons of log-transformed data; Treatment B: cilansetron + ketoconazole, Treatment A: cilansetron alone.

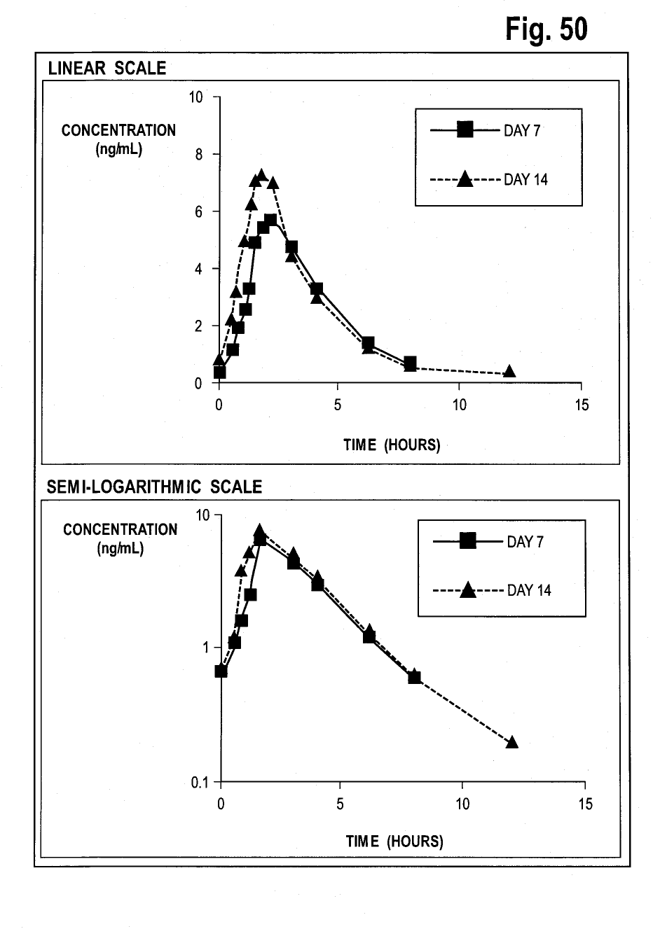

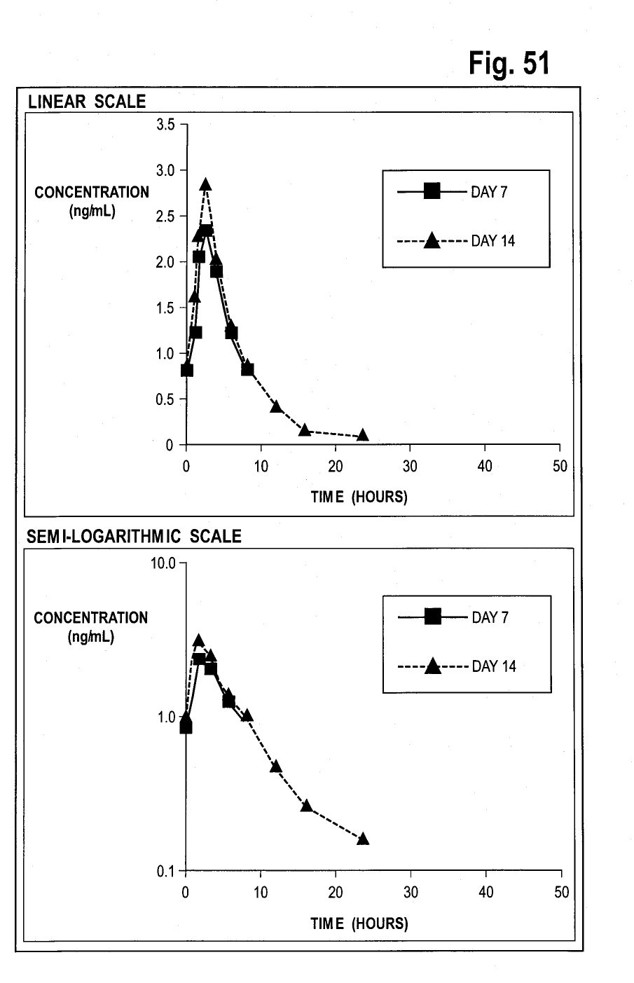

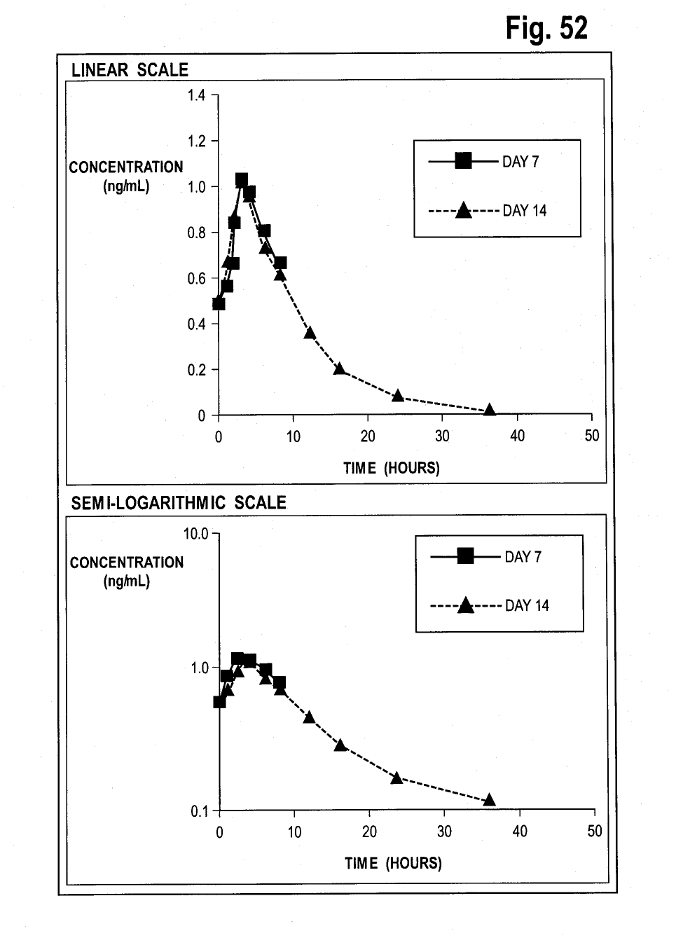

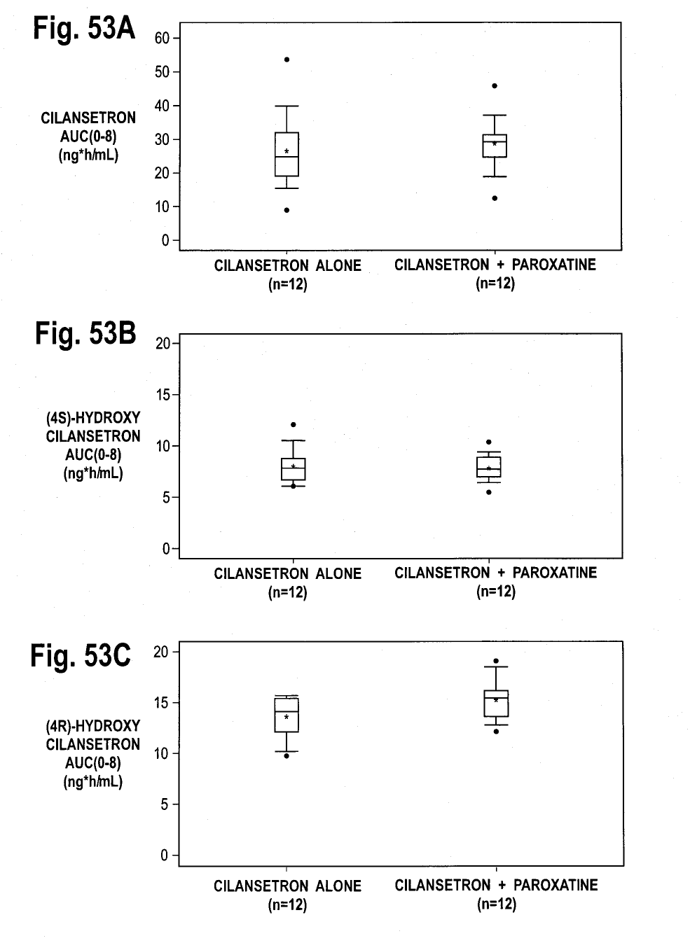

FIG. 50, FIG. 51, and FIG. 52 present geometric mean steady-state plasma concentration-time profiles for cilansetron and its 4-hydroxy metabolites in the absence and presence of concomitant paroxetine. Table 2.7.2.3.28 presents descriptive statistics for the pharmacokinetic parameters of cilansetron and its 4-hydroxy metabolites, and the results of statistical comparisons between treatments (cilansetron+paroxetine versus cilansetron alone), and FIGS. 53a-c shows the distribution of AUC(0-τ) values for the two treatments. The results showed that coadministration of paroxetine produced a small increase in exposure to ilansetron. Mean cilansetron AUC(0-τ) increased by 20% and mean Cmax by 36%. Mean (4R)-hydroxy cilansetron AUC(0-τ) and Cmax increased by 16% and 27%, respectively, but there was no change in exposure to (4S)-hydroxy cilansetron. These increases in exposure are not clinically significant.

One subject in the cilansetron+paroxetine study group was identified by genotyping as a poor metabolizer for CYP2D6 and another was identified as a poor metabolizer for CYP2C19. No difference in cilansetron pharmacokinetics was observed in the poor metabolizer for CYP2D6 but an approximate 2-fold increase was observed in AUC(0-τ) and Cmax in the poor metabolizer for CYP2C19, following coadministration of paroxetine.

TABLE 2.7.2.3.28
Arithmetic mean (% CV) steady-state pharmacokinetic parameters for
cilansetron and its 4-hydroxy metabolites and results of statistical
comparisons between treatments following oral treatment with cilansetron
2 mg TID alone or in combination with paroxetine 20 mg QD
(Study S.241.1.109)

| | | Arith. Mean (% CV) | | Statistical | |
|---|---|---|---|---|---|
| | | Cilansetron + | Cilansetron | Comparisons | |
| | | Paroxetine | Alone | (Treatment B/A)^{b} | |
| Analyte | Parameter | (N = 12) | (N = 12) | Ratio (%) | 90% CI |
| Cilansetron | AUC(0-τ) (ng · h/mL) | 27.4 (34) | 25.8 (50) | 120 | 102, 142 |
| | Cmax (ng/mL) | 9.99 (40) | 8.11 (53) | 136 | 114, 162 |
| | Tmax (h) | 1.50 (0.75, 3.00)^{a} | 2.00 (0.50, 4.00)^{a} | NC | NC |
| (4R)—OH | AUC(0-τ) (ng · h/mL) | 15.2 (13) | 13.5 (16) | 116 | 107, 127 |
| | Cmax (ng/mL) | 3.41 (19) | 2.76 (25) | 127 | 112, 143 |
| | Tmax (h) | 1.50 (0.75, 3.00)^{a} | 2.50 (0.75, 4.00)^{a} | NC | NC |
| (4S)—OH | AUC(0-τ) (ng · h/mL) | 7.62 (18) | 7.97 (21) | 99 | 90, 109 |
| | Cmax (ng/mL) | 1.23 (19) | 1.22 (21) | 105 | 95, 115 |
| | Tmax (h) | 3.00 (1.50, 6.00)^{a} | 3.00 (1.75, 4.00)^{a} | NC | NC |

NC—not calculated;
% CV - coefficient of variation on arithmetic mean in percent;
AUC(0-τ) - AUC(0-8)
^{a}Results presented are median (min, max).
^{b}Results of ANOVA comparisons of log-transformed data; Treatment B: cilansetron + paroxetine, Treatment A: cilansetron alone.

Several in vitro studies have been also conducted to evaluate the potential for-drug interactions with cilansetron. In an in vitro study utilizing human hepatic microsomes to evaluate the inhibitory potential of cilansetron on cytochrome P450 enzymes, cilansetron showed only weak inhibition (IC50 of 60 μM, which is >3000× Cmax at the proposed dose of 2 mg TID) of 7-ethoxyresorufin O-deethylation (a marker of CYP1A2 activity) and no inhibition of any other cytochrome P450 enzyme activity, suggesting that potential drug interactions due to inhibition of CYP enzymes by cilansetron are unlikely. In another study, (4R)-hydroxy and (4S)-hydroxy cilansetron were shown to have no significant inhibitory effect on any cytochrome P450 enzyme (IC50s >100 μM). An in vitro study using human hepatic microsomes evaluated the effect of some enzyme inhibitors on the metabolism of cilansetron (100 μM). The inhibitors tested were troleandomycin (10 μM, CYP3A4 inhibitor) and ketoconazole at lower (1 μM, CYP3A4 inhibitor) and higher (25 μM, general inhibitor) concentrations. On average, troleandomycin (10 μM) and ketoconazole (1 μM) inhibited about 70% of cilansetron metabolism while ketoconazole (25 μM) inhibited 93% of its metabolism. Troleandomycin (10 μM) and ketoconazole (1 μM) inhibited 34% and 47%, respectively, of the formation of the 4-hydroxy metabolites while ketoconazole (25 μM) inhibited 69% of formation.

The inductive effect of cilansetron and its 4-hydroxy metabolites on various cytochrome P450 enzymes was evaluated in cultured human hepatocytes using an RT-PCR assay. The results showed that cilansetron is a very weak inducer of CYPIA (2% that of 3-methylcholantrene), but this effect is considered negligible. Its 4-hydroxy metabolites have no effect on this enzyme. Cilansetron and its 4-hydroxy metabolites increased CYP2C19 and this effect was about half of that caused by phenobarbital. Phenobarbital is a moderate inducer of CYP2C19 in vitro but is reportedly inactive in inducing this enzyme in humans in vivo. Comparison with the other model inducer used, rifampin, gave variable results indicating that the significance of any findings was doubtful. Cilansetron and its two metabolites did not induce CYP3A4 mRNA but doubled CYP3A5 mRNA, which was similar to the effect seen with the model inducers 3-methylcholantrene and phenobarbital. Since CYP3A5 is a minor isoform in human liver, it shares substrate specificity with CYP3A4, and its activity is low compared to that of CYP3A4, limited variations in CYP3A5 activity may not cause major changes in the overall CYP3A activity. The lack of a significant inductive effect on CYP3A by cilansetron is also supported by the results of the clinical interaction study with Ortho Tri-Cyclen, which shows that concomitantly administered cilansetron does not affect the pharmacokinetics or pharmacodynamics of this CYP3A substrate.

The potential for a protein binding interaction effect of warfarin on cilansetron was investigated in an in vitro study in plasma using equilibrium dialysis. The effect of warfarin, at a nominal concentration of 10 μg/mL (notably higher than therapeutic concentrations of approximately 2.2±0.4 1 μg/mL) on the binding of ¹⁴C-labeled cilansetron to human plasma proteins was determined at cilansetron nominal concentrations of 30, 300, and 3000 ng/mL. No effect of warfarin on the binding of cilansetron was observed.

Population Pharmacokinetics and Pharmacodynamics

Pharmacokinetics in the Target Population

The pharmacokinetics and pharmacodynamics of cilansetron following multiple oral administration in non-constipated IBS patients have been examined in a population pharmacokinetic (NONMEM) analysis of a Phase II trial. The study was a double-blind, placebo-controlled, randomized, multi-center, parallel-group, dose ranging study to investigate the Efficacy and safety of four different doses of cilansetron (1 mg, 2 mg, 8 mg, and 16 mg TID) in patients with established IBS (non-constipated patients who suffered from IBS according to the Rome Criteria and were not bothered by constipation for >25% of their IBS episodes). The study was to be performed in approximately 50 centers. A sufficient number of patients were to be recruited to insure that at least 88 patients per treatment group were evaluable for efficacy and to get a total of 440 completers. Subjects had an assessment at the beginning (Visit 1; pre-study) and end (Visit 2; baseline) of an 18-day drugfree run-in period, after which they were randomized to a study treatment for a period of 12 weeks. There were interim assessments at the end of the 4^{th} week (Visit 3; Day 29) and 8^{th} week (Visit 4; Day 57) and a final assessment at the end of the 12th week (Visit 5; poststudy). Single blood samples for population pharmacokinetic analyses were drawn at Visits 3, 4, 5 or early termination, at 3 1 of the participating sites. With the exception of Visit 3, the timing of the blood draw in relation to the previous dose was not specified for any of the samples collected, but the time of sampling and the time of last dosing was recorded for each sample collected. For Visit 3, the blood sample had to be taken between 15 minutes and 2 hours of the morning or midday dose.

Various covariates were screened during development of the population pharmacokinetic model, including age, height, weight, body surface area, body mass index, race, gender, smoking status, serum creatinine levels, serum creatinine clearance, total bilirubin levels, cilansetron dose, and concomitant medication usage. Some efficacy and safety parameters were also evaluated in this population analysis using correlation analyses. Efficacy parameters evaluated included overall relief of IBS symptoms (Responder or Non-Responder), intensity of abdominal pain, number of stools and stool consistency, symptoms of abdominal and rectal discomfort (straining, urgency, feeling of incomplete defecation, passage of mucus, bloating, and feeling of abdominal distension), and upper GI symptoms at Visit 5 (vomiting, heartburn, early satiety, postprandial fullness, sensation of prolonged digestion, and nausea). Safety parameters evaluated included the top ten most frequently observed adverse events (constipation, flatulence, nausea, abdominal pain, headache, dyspepsia, infection, gastrointestinal disorders, anorexia, and diarrhea).

The population analysis included data from 160 patients (63 males and 97 females) with a median age of 45.5 years (range: 20-81 years), median weight of 78 kg (range: 47-121 kg), and median height of 170 cm (range: 147-196 cm). More than 90% of the patients were White and 18.8% of the patients were considered smokers. The demographics of the patient population in this analysis were comparable to the overall study population. A total of 392 quantifiable concentrations for cilansetron, 393 quantifiable concentrations for (4S)-hydroxy cilansetron, and 397 quantifiable concentrations for (4R)-hydroxy cilansetron from 160 patients were included in the NONMEM datasets for population-analyses.

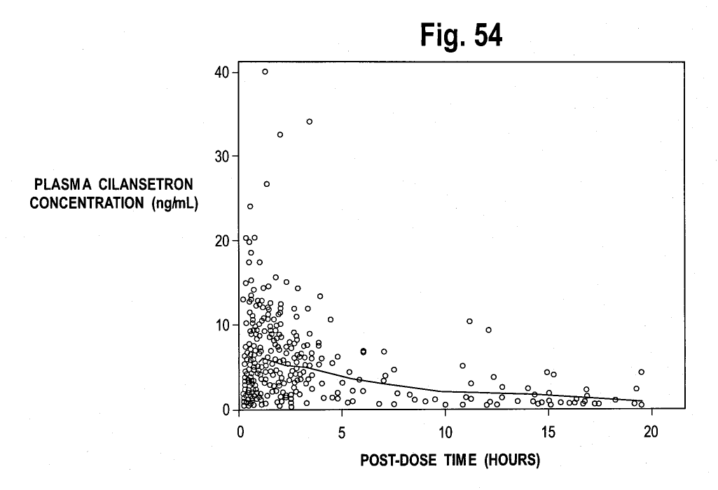

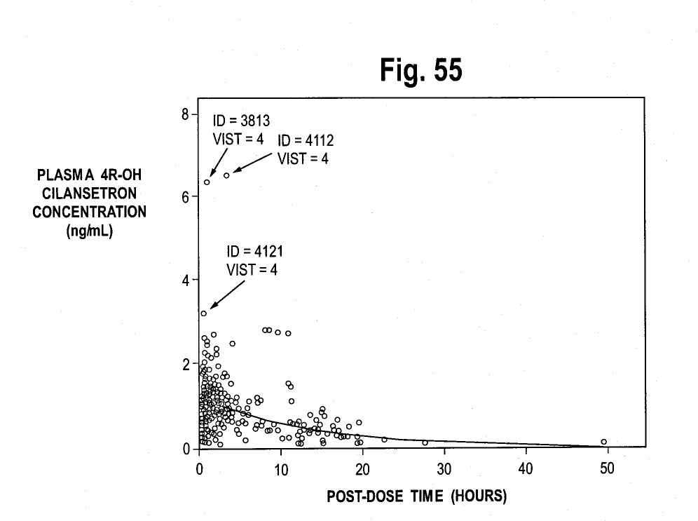

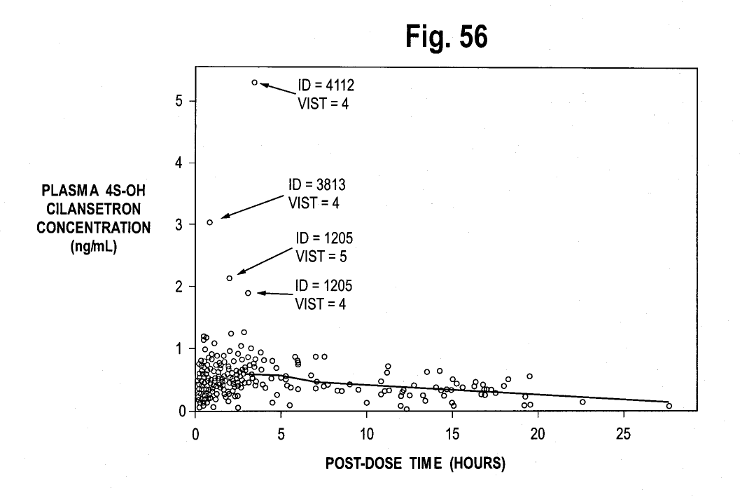

FIG. 54, FIG. 55, and FIG. 56 present mean steady-state plasma concentration-time profiles for cilansetron and its 4-hydroxy metabolites, and Table 2.7.2.3.29 and Table 2.7.2.3.30 present descriptive statistics for the estimated pharmacokinetic parameters of cilansetron and its 4-hydroxy metabolites from the final covariate population pharmacokinetic model. The cilansetron concentration data were best described by a one-compartment model with first-order input, an absorption lag time, and first-order elimination. A log-normal distribution was used to describe the variability of clearance, volume of distribution and absorption rate constant between patients. The residual error was best described by an exponential model. Use of a one compartment model is in agreement with pharmacokinetic data obtained from Phase I studies in healthy subjects. The population estimates of model parameters (apparent clearance, apparent volume of distribution, half-life, and predicted AUC and Cmax at steady state) were also similar to estimates in healthy subjects.

TABLE 2.7.2.3.29
Arithmetic mean (SD) pharmacokinetic parameters for cilansetron and its
4-hydroxy metabolites based on Bayesian estimates from a covariate
population pharmacokinetic model, from a Phase II study of cilansetron
1 mg, 2 mg, 8 mg, or 16 mg TID in IBS patients for 12 weeks (Study S.241.2.112)

| | | 1 mg TID | 2 mg TID | 8 mg TID | 16 mg TID |
|---|---|---|---|---|---|
| Analyte | Parameter | (n = 50-52) | (n = 27-28) | (n = 37-38) | (n = 40-41) |
| Cilansetron | CL/F (L/h) | 28.3 (14.5) | 34.1 (16.0) | 34.2 (22.2) | 37.3 (21.4) |
| | V/F (L) | 159 (65) | 178 (60) | 170 (65) | 188 (61) |
| | Ka (h-1) | 2.92 (0.59) | 2.98 (0.67) | 3.16 (0.69) | 3.06 (0.69) |
| | T½ (h) | 4.76 (2.74) | 4.30 (2.44) | 4.50 (2.62) | 4.50 (2.82) |
| (4R)—OH | CL/F (L/h) | 234 (56) | 232 (52) | 229 (53) | 232 (62) |
| | T½ (h) | 9.55 (2.25) | 9.56 (2.02) | 9.79 (2.40) | 9.80 (2.55) |
| (4S)—OH | CL/F (L/h) | 140 (30) | 136 (26) | 134 (27) | 142 (27) |
| | T½ (h) | 5.42 (1.19) | 5.55 (1.14) | 5.64 (1.10) | 5.31 (1.06) |

TABLE 2.7.2.3.30
Arithmetic mean (SD) predicted steady-state AUC(0-τ) and Cmax values
for cilansetron and its 4-hydroxy metabolites from a covariate population
pharmacokinetic model, for a Phase II study of cilansetron 1 mg, 2 mg,
8 mg, or 16 mg TID in IBS patients for 12 weeks (Study S.241.2.112)

| | | 1 mg TID | 2 mg TID | 8 mg TID | 16 mg TID |
|---|---|---|---|---|---|
| Analyte | Parameter | (n = 50-52) | (n = 27-28) | (n = 37-38) | (n = 40-41) |
| Cilansetron | AUC(0-τ) (ng · h/mL) | 41.8 (28.8) | 63.9 (33.3) | 301 (194) | 503 (290) |
| | Cmax (ng/mL) | 7.92 (4.40) | 12.7 (5.4) | 58.0 (30.3) | 98.5 (44.7) |
| (4R)—OH | AUC(0-τ) (ng · h/mL) | 6.42 (1.40) | 13.0 (2.64) | 53.0 (10.1) | 98.6 (18.8) |
| | Cmax (ng/mL) | 1.05 (0.19) | 2.17 (0.37) | 8.56 (1.70) | 16.7 (3.0) |
| (4S)—OH | AUC(0-τ) (ng · h/mL) | 3.85 (0.92) | 7.68 (1.59) | 31.6 (7.68) | 63.2 (16.5) |
| | Cmax (ng/mL) | 0.546 (0.105) | 1.10 (0.20) | 4.46 (1.04) | 9.03 (2.03) |

Dose was not found to be a covariate in the population model, indicating that the pharmacokinetic parameters CLIF and VIF of cilansetron in non-constipated IBS patients are dose-independent in the dose range of 1 to 16 mg TID. Overall, plasma concentrations of cilansetron and its Chydroxy metabolites increased with dose over this dose range. This conclusion is consistent with observations in Phase I studies in healthy subjects under similar dose regimens.

In some Phase I studies, it has been observed that female subjects tended to have higher concentrations of cilansetron in plasma when compared to males after single and repeated oral administration. Also, age was found to have some effect on cilansetron clearance and half-life. However, neither a gender nor age effect was obtained in this population analysis, which could be due to the relatively large inter- and intra-subject variability of the disposition of cilansetron in the study population.

Cilansetron is extensively metabolized. The population analysis showed that cilansetron clearance was not related to serum creatinine level or creatinine clearance. This is consistent with the fact that renal elimination of cilansetron is negligible (<1% of unchanged drug in urine).

Covariate analysis revealed that the presence of concomitant medications and smoking status had a statistically significant_influpencepon the apparent oral clearance of cilansetron. Apparent oral-clearance decreased by 38.6% in the presence of concomitant medications; however a specific group of concomitant medications contributing to such reduction could not be identified at this stage. In a drug-drug interaction trial in healthy subjects, substantial increases in plasma concentrations of cilansetron, as evidenced by increases in both Cmax and AUC(0-τ) were observed after concomitant administration with fluvoxamine (an inhibitor of CYP1A2 and other CYP enzymes). However, the influence of fluvoxamine, or any other CYP1A2 inhibitors or inducers, could not be identified in this population analysis due to a limited number of patients taking this type of concomitant medications. It has been well established that cigarette smoking can lead to induction of CYP1A2 isozymes. Smoking was found to be a significant covariate of apparent clearance for cilansetron in this population analysis. Apparent oral clearance increased by 32.2% in patients who were considered smokers. This is in agreement with the observed effect of the CYP1A2 modulator drug, fluvoxamine, on the elimination of cilansetron.

Exposure to the two 4-hydroxy metabolites of cilansetron was lower than the parent drug following an oral-dose. The structural and covariate models that best described the concentration profiles of (4R)-hydroxy and (4s)-hydroxy cilansetron after oral doses of cilansetron were the same for both metabolites; namely a one-compartment model with first-order input and serum creatinine clearance as the predictor of apparent clearance. Inter-subject variability was modeled for apparent clearance assuming a log-normal distribution and residual error was best described by an exponential model. The exposure to the R-enantiomer was slightly higher than the S-enantiomer, in patients enrolled in this trial. The identification of creatinine clearance as a covariate to the population pharmacokinetic model of the 4-hydroxy metabolites is in agreement with the fact that these two metabolites are primarily excreted in the urine. As creatinine clearance increased by 1 mL/min, the apparent oral clearance increased by 0.447 L/h and 0.883 L/h for (4R)-hydroxy and (4S)-hydroxy cilansetron, respectively.

Increases in AUC(0-τ) of the 4-hydroxy metabolites at levels of renal impairment comparable to that of the renal impairment study were predicted from NONMEM simulations utilizing the population pharmacokinetic model. The effect on AUC(0-τ) predicted by the population model for mild impairment was similar to that observed in Study S.241.1.110 for both (4R)-hydroxy and (4S)-hydroxy cilansetron (Table 2.7.2.3.22). The predicted effect was much smaller than the observed effect for the moderate and severe impairment groups, but this is probably due to the fact that the study population used for the population analysis consisted mainly of subject with normal renal status (64%) and mild impairment (34%), with only 2% of subjects showing moderate impairment and no subjects having severe impairment.

A statistically significant negative correlation was found between exposure (AUC and Cmax at steady state) and some efficacy parameters including change in stool consistency (adjusted for baseline) and the experience of straining. Patients with higher exposure to cilansetron and/or its 4-hydroxy metabolites were more likely to have lower stool consistency scores (a tendency for harder stools) than patients with lower exposure. From linear regression, an increase of 600 ng*h/mL in AUC or 100 ng/mL in Cmax of cilansetron correlated with a 0.5 point decrease in the stool consistency score. In addition, patients with higher exposure to cilansetron were more likely to experience straining. From this model, when AUC of cilansetron reached 360 ng*h/mL or Cmax reached 65 ng/mL, IBS patients were observed to have a 50% chance of experiencing straining.

Some difference in exposure-response relationships was observed for the 4-hydroxy metabolites. A statistically significant correlation was found between exposure (AUC and Cmax at steady state) to (4R)-hydroxy cilansetron and the reporting of dyspepsia as an adverse event. No such correlation was identified for (4s)-hydroxy cilansetron. A statistically significant negative correlation was also found between experiencing gastrointestinal (GI) disorders and exposure to cilansetron or its 4-hydroxy metabolites.

The pharmacokinetics of cilansetron have also been evaluated in another clinical study in EBS patients. In a Phase III, double-blind, placebo-controlled, randomized, multicenter study in nonconstipated IBS patients who were treated with cilansetron 16 mg or placebo TID for 12 weeks, blood samples were collected during periodic study visits in a subset of patients for the evaluation of the pharmacokinetics of cilansetron and its 4-hydroxy metabolites. This study was terminated prematurely sb pharmacokinetic analysis was not performed. However, Table 2.7.2.3.3 1 below summarizes cilansetron and 4-hydroxy metabolite concentrations in samples collected during the study.

TABLE 2.7.2.3.31
Summary Statistics of plasma concentrations of cilansetron and its
4-hydroxy metabolites in IBS patients during oral treatment with
cilansetron 16 mg TID for 12 weeks (Study S.241.3.001)

| | | | 4(R)—OH- | 4(S)—OH- |
|---|---|---|---|---|
| | | Cilansetron | Cilansetron | Cilansetron |
| Visit | Statistic | (ng/mL) | (ng/mL) | (ng/mL) |
| 3 | N | 64 | 63 | 63 |
| (Day 29-31) | Mean | 57.02 | 10.75 | 6.94 |
| | SD | 73.05 | 4.17 | 8.93 |
| 5 | N | 11 | 12 | 12 |
| (Day 83-87) | Mean | 29.0 | 8.78 | 5.67 |
| | SD | 48.91 | 15.16 | 5.19 |

Pharmacodynamics
Effects on Gastrointestinal Function
Effect on Visceral Perception

The effect of cilansetron on visceral perception has been studied in 3 clinical studies in healthy subjects and 1 study in IBS patients.

The effect of cilansetron on esophageal visceral perception was evaluated in a randomized, double-blind, placebo-controlled, 3-way crossover study in 12 male healthy subjects (FIT population), aged 32-62 years. Subjects received placebo, cilansetron 4 mg, or cilansetron 8 mg administered orally TID for 7 days during each of the 3 periods, with a 7-day washout between periods. Esophageal distension tests were done one hour after administration of the second dose on Day 7 of each treatment. A manually inflated intraesophageal balloon was used to produce distensions (successive 1 mL increases, up to a sensation of abdominal pain or a maximum balloon volume of 20 mL). The primary efficacy parameter was the distension volume inducing esophageal pain. Secondary parameters were the distension volumes and pressures leading to first esophageal sensation. The pain threshold and other secondary parameters for each of the cilansetron doses was compared to placebo using a one-sided Dunnett t-test.

The results of the analysis are summarized in Table 2.7.2.3.32. A small mean increase in the pain perception threshold volume and pressure was observed with the cilansetron treatments compared to placebo, but the differences were not statistically significant. Similarly, no significant differences were found in the volume or pressure for the threshold of sensation perception.

TABLE 2.7.2.3.32
Arithmetic mean (SD) measures of esophageal visceral perception
following administration of oral doses of cilansetron 4 mg, 8 mg, or
placebo TID for 7 days in healthy subjects (Study K.241.5009)

| Absolute Values | | | Differences from Placebo^{a} | |
|---|---|---|---|---|
| 4 mg | 8 mg | Placebo | 4 mg | 8 mg |
| (n = 12) | (n = 12) | (n = 12) | (n = 12) | (n = 12) |

Volume (mL) first producing esophageal pain

| | | | | |
|---|---|---|---|---|
| 14.6 (4.3) | 13.9 (5.8) | 13.2 (5.8) | 1.4 (4.9) | 0.8 (3.6) |

Volume (mL) first producing esophageal sensation

| | | | | |
|---|---|---|---|---|
| 5.3 (3.5) | 5.2 (3.6) | 5.7 (3.4) | −0.3 (4.8) | −0.5 (1.8) |

Pressure (mmHg) first producing esophageal pain

| | | | | |
|---|---|---|---|---|
| 42.3 (11.3) | 41.0 (12.5) | 39.8 (16.8) | 3.0 (24.0) | 1.3 (22.5) |

Pressure (mmHg) first producing esophageal sensation

| | | | | |
|---|---|---|---|---|
| 27.4 (9.6) | 28.4 (10.9) | 30.7 (15.4) | −2.5 (16.4) | −2.5 (6.9) |

^{a}No statistically significant differences (p > 0.05) were found for any parameter, in comparisons of cilansetron treatments to placebo using a one-sided Dunnett t-test.

The effect of cilansetron on gastric perception was evaluated in a randomized, double-blind, placebo-controlled, parallel-group study in 44 male and female healthy subjects (IT population; 22 subjects received placebo and 22 received 8 mg cilansetron), aged 19-43 years. Subjects received cilansetron 8 mg TID orally for 7 days and gastric distension tests were done at baseline (Day 1, prior to dosing) and poststudy (Day 8). An electronic barostat was used to produce isobarometric distensions (starting from 2 mmHg, progressive distensions in 2 mmHg increments were performed every 2 minutes until gastric pain occurred or an upper limit of 20 mmHg was reached) and isovolumetric distensions (starting from an initial volume of 200 mL, progressive distensions in 100 mL increments were performed every 2 minutes until gastric pain occurred or an upper limit of 1100 mL was reached). The primary efficacy parameter was the difference in pain threshold from baseline to poststudy, measured by the amount of distension pressure inducing gastric pain. Secondary parameters were the mean distension pressure/volume inducing the first feelings of gastric sensation and gastric discomfort, and changes in bowel habits over the treatment period. A 2-sample t-test was used to compare baseline-adjusted pain threshold and other secondary efficacy parameters between cilansetron and placebo treatments.

The results are summarized in Table 2.7.2.3.33. A statistically significant increase was observed in all sensitivity thresholds (pain, first sensation, and discomfort) with cilansetron 8 mg TID, showing that cilansetron decreased the gastric visceral sensitivity in healthy subjects compared to placebo. Following treatment with cilansetron, the mean gastric pain threshold was raised by 1.9 mmHg compared to baseline and following placebo it was lowered by 0.9 mmHg, resulting in a 2.8 mmHg mean increase (p<0.001) for cilansetron compared to placebo. The gastric sensation threshold was raised by 0.9 mmHg on cilansetron and lowered by 1.1 mmHg on placebo, with a resultant treatment increase of 2.1 mmHg (p=0.002). The gastric discomfort threshold was raised by 0.9 mmHg on cilansetron and lowered by 1.3 mmHg on placebo, with a treatment increase of 2.2 mmHg (p=0.001). The volumes at the different thresholds were comparable between cilansetron and placebo indicating that the different thresholds are not caused by differences in accommodation status. The mean volume required to reach the gastric pain threshold was raised after both cilansetron (by 77.3 mL) and placebo (by 95.2 mL), and the difference between the treatments of −18.0 mL was not statistically significant (p=0.600).

TABLE 2.7.2.3.33
Arithmetic mean (SD) measures of gastric visceral perception and results
of statistical comparisons between treatments following administration of
cilansetron 8 mg or placebo TID orally for 7 days in healthy subjects
(Study S.241.2.104)

| Arithmetic Mean (Standard Deviation) | | | | | | Statistical |
|---|---|---|---|---|---|---|
| Cilansetron 8 mg TID (n = 22) | | | Placebo TID (n = 22) | | | Comparison^{a} |
| | | Change from | | | Change from | Difference |
| Baseline | Poststudy | Baseline | Baseline | Poststudy | Baseline | (95% CI) |

Pressure (mmHg) first producing gastric pain

| | | | | | | |
|---|---|---|---|---|---|---|
| 14.2 (2.6) | 15.4 (2.6) | 1.9 (2.3) | 16.8 (2.4) | 15.6 (2.9) | −0.9 (1.8) | 2.8 (1.44, 4.14)* |

Volume (mL) first producing gastric pain

| | | | | | | |
|---|---|---|---|---|---|---|
| 686 (113) | 764 (140) | 77 (119) | 682 (126) | 767 (107) | 95 (102) | −18 (−87, 51) |

Pressure (mmHg) first producing gastric sensation

| | | | | | | |
|---|---|---|---|---|---|---|
| 8.0 (2.2) | 8.9 (2.7) | 0.9 (1.6) | 9.5 (2.8) | 8.6 (2.1) | −1.1 (2.4) | 2.1 (0.8, 3.3)* |

Pressure (mmHg) first producing gastric discomfort

| | | | | | | |
|---|---|---|---|---|---|---|
| 11.0 (2.1) | 11.9 (2.7) | 0.9 (1.9) | 12.6 (2.3) | 11.3 (1.8) | −1.3 (1.9) | 2.2 (1.0, 3.4)* |

CI—confidence interval;
*Statistically significant (p < 0.01).
^{a}Results of a 2-sample t-test to compare cilansetron versus placebo using baseline-adjusted values.

The effect of cilansetron on colonic visceral perception was evaluated in a randomized, doubleblind, placebo-controlled, 2-way crossover study in 16 male and female IBS patients (ITT population), aged 28-66 years. Subjects received single IV doses of placebo or cilansetron 16 mg as 30-minute infusions on two consecutive study days, with no washout between the study days. Colonic distension tests were performed just prior to dosing and after completion of the infusion during each treatment. An electronic barostat was used to produce distensions (successive 4 mmHg increases every 5 minutes, up to a sensation of abdominal pain or a maximum of 40 mmHg). The primary efficacy parameter was the mean distension pressure inducing a sensation of abdominal pain. Secondary effect parameters were the mean distension pressure inducing a sensation of abdominal discomfort, and the response rate, defined as the number of patients experiencing no abdominal pain at the maximum pressure of 40 mmHg. The primary and secondary parameters were compared between cilansetron and placebo treatments using an ANOVA.

The results of the analysis are summarized in Table 2.7.2.3.34. No statistically significant differences were found in mean distension pressures leading to abdominal pain or abdominal sensation, for cilansetron 16 mg compared to placebo. Additionally, only 1 of 12 subjects met the definition of a responder during the study (no abdominal pain at a pressure of 40 mmHg, following cilansetron treatment).

TABLE 2.7.2.3.34
Arithmetic mean (SD) measures of colonic visceral perception
and results of statistical comparisons following administration
of single IV doses of cilansetron 16 mg or placebo in IBS
patients (Study K.241.5016)

| Difference Postdose^{a} | | Statistical Comparison to Placebo^{c} | | |
|---|---|---|---|---|
| 16 mg | Placebo | Mean | 95% Confidence Interval | |
| (n = 12)^{b} | (n = 12)^{b} | Difference | Lower Limit | Upper Limit |

Pressure (mmHg) inducing abdominal pain

| | | | | |
|---|---|---|---|---|
| 3.67 (4.66) | 5.00 (4.55) | −1.33 | −5.89 | 3.22 |

Pressure (mmHg) inducing abdominal sensation

| | | | | |
|---|---|---|---|---|
| 2.67 (3.55) | 3.33 (3.75) | −0.67 | −4.76 | 3.43 |

^{a}Difference between postdose and predose values.
^{b}Only those subjects who completed both treatments (n = 12) are summarized.
^{c}ANOVA comparison of the cilansetron treatment to placebo. No statistically significant differences (p > 0.05) were found for any parameter.

The effect of cilansetron on rectal visceral perception was evaluated in a randomized, doubleblind, placebo-controlled, 4-way crossover study in 12 male healthy subjects (ITT population), aged 18-26 years. Subjects received single IV doses of placebo or cilansetron 4 mg, 8 mg, or 16 mg as 30-minute infusions during each of the periods, with a 14-day washout between periods. Rectal distenision tests were done during each treatment, immediately following the completion of each infusion. An electronic barostat was used to produce isobarometric distensions (successive 4 mmHg increases every 5 minutes, up to a sensation of abdominal pain or a maximum of 44 mmHg). The primary efficacy parameter was the distension pressure required to induce a sensation of abdominal pain. Secondary parameters were the distension pressure at the first feeling of abdominal/rectal sensation and the first feeling of need to defecate, and the response rate (defined as the number of subjects who experienced no abdominal pain at the maximum pressure of 44 mmHg). The pain threshold for each of the three cilansetron doses was compared to placebo using a one-sided Dunnett t-test.

The results of the analysis are summarized in Table 2.7.2.3.35. A mean elevation of the pain threshold was observed for all cilansetron doses compared to placebo, but the data were highly variable and consequently, no statistically significant differences were found between cilansetron and placebo. An evaluation of the dose-response curve for the abdominal pain threshold showed no linearity, with the strongest elevation of the pain threshold being obtained at the 8 mg rather than the 16 mg cilansetron dose. Additionally, no statistically significant differences were found between the cilansetron treatments and placebo for the threshold of abdominal sensation or need to defecate.

TABLE 2.7.2.3.35
Arithmetic mean (SD) measures of rectal visceral perception following
administration of single IV doses of cilansetron 4 mg, 8 mg, or 16 mg, or
placebo in healthy subjects (Study K.241.5007)

| Absolute Values | | | | Differences from Placebo^{a} | | |
|---|---|---|---|---|---|---|
| 4 mg | 8 mg | 16 mg | Placebo | 4 mg | 8 mg | 16 mg |
| (n = 12) | (n = 12) | (n = 12) | (n = 12) | (n = 12) | (n = 12) | (n = 12) |

Pressure (mmHg) first producing abdominal pain

| | | | | | | |
|---|---|---|---|---|---|---|
| 31.0 (6.6) | 32.3 (6.5) | 31.7 (6.9) | 30.3 (5.8) | 0.67 (6.34) | 2.00 (7.34) | 1.33 (6.46) |

Volume (mL) first producing abdominal pain

| | | | | | | |
|---|---|---|---|---|---|---|
| 310 (74) | 297 (63) | 313 (71) | 298 (53) | 12.7 (43.8) | −0.83 (48.3) | 15.8 (34.2) |

Pressure (mmHg) first producing feeling of abdominal sensation

| | | | | | | |
|---|---|---|---|---|---|---|
| 12.7 (4.8) | 12.0 (3.0) | 12.7 (3.8) | 13.7 (2.7) | −1.00 (4.22) | −1.67 (3.60) | −1.00 (4.22) |

Volume (mL) first producing feeling of abdominal sensation

| | | | | | | |
|---|---|---|---|---|---|---|
| 132 (78) | 133 (48) | 138 (90) | 152 (39) | −20.0 (76.7) | −19.2 (54.9) | −14.2 (94.1) |

Pressure (mmHg) first producing feeling of need to defecate

| | | | | | | |
|---|---|---|---|---|---|---|
| 20.3 (3.6) | 20.3 (2.7) | 20.0 (4.5) | 21.0 (3.0) | −0.67 (2.87) | −0.67 (3.34) | −1.00 (4.22) |

Volume (mL) first producing feeling of need to defecate

| | | | | | | |
|---|---|---|---|---|---|---|
| 233 (76) | 232 (52) | 238 (73) | 242 (51) | −8.75 (51.2) | −10.0 (58.5) | −3.75 (58.7) |

^{a}No statistically significant differences (p > 0.05) were found for any parameter, in comparisons of cilansetron treatments to placebo using a one-sided Dunnett t-test.

Four additional clinical studies were conducted in patients with IBS, dyspepsia, or non-cardiac chest pain to evaluate the effect of cilansetron on visceral perception but these studies were prematurely terminated or stopped due to low recruitment, so no conclusions could be drawn from these studies.

Effects on Esophageal Motility, Gastric Emptying Time, Colonic Motility, and Colonic Transit Time

The effect of cilansetron on esophageal motility was evaluated in a randomized, double-blind, placebo-controlled, 3-way crossover study in 12 male healthy subjects (ITT population), aged 32-62 years. Subjects received placebo, cilansetron 4 mg, or cilansetron 8 mg administered orally TID for 7 days during each of the 3 periods, with a 7-day washout between periods. Before conduct of esophageal distension tests using an intraesophageal balloon, primary esophageal motility was induced by at least 5 wet swallows of 5 mL water and 21.0 (3.0) manometrically registered. Amplitude and duration of esophageal contractions were measured in 3 channels proximal and distal to the balloon and the velocity of propagation of contractions was −0.67 (2.87) measured over the 2 inter-channel intervals. The results are summarized in the Table 2.7.2.3.36 below. The mean contraction amplitude was slightly reduced with cilansetron at both doses and at each measurement point compared to placebo, and a dose dependent trend was observed. No such tendencies were observed in duration. For velocity of propagation, a slight trend towards −0.67 (3.34) reduced velocity with increasing cilansetron dose was found in the 7-2 cm interval but not in the 17-7 cm interval, and there was no effects seen on onset in either interval.

TABLE 2.7.2.3.36
Arithmetic mean (SD) for esophageal motility parameters following
administration of cilansetron 4 mg, 8 mg, or placebo orally TID for 7 days
in healthy subjects (Study K.241.5009)

| | Channel/ | Arithmetic Mean (Standard Deviation) | | | | |
|---|---|---|---|---|---|---|
| | Inter- | | Cilansetron 4 mg | | Cilansetron 8 mg | |
| | Channel | | Absolute | Change from | Absolute | Change from |
| Parameter | Intervals | Placebo | Values | Placebo | Values | Placebo |

Contraction Force

| | | | | | | | |
|---|---|---|---|---|---|---|---|
| Amplitude | 17 | cm | 66.8 (20.8) | 61.5 (20.5) | −5.3 (18.0) | 59.3 (13.3) | −7.5 (17.5) |
| (mmHg) | 7 | cm | 88.3 (33.3) | 84.7 (36.1) | −3.6 (16.9) | 85.4 (28.6) | −2.8 (19.4) |
| | 2 | cm | 92.6 (37.0) | 88.3 (38.6) | −4.2 (15.1) | 85.5 (26.1) | −7.1 (34.1) |
| Duration | 17 | cm | 2.9 (0.9) | 3.0 (1.3) | 0.1 (0.8) | 2.9 (1.2) | −0.0 (1.0) |
| (sec) | 7 | cm | 4.2 (1.6) | 3.7 (1.2) | −0.5 (1.5) | 4.0 (1.0) | −0.2 (1.5) |
| | 2 | cm | 4.6 (1.3) | 4.5 (1.5) | −0.0 (1.4) | 5.0 (1.6) | 0.4 (1.5) |

Velocity of Propagation (cm/sec)

| | | | | | | | |
|---|---|---|---|---|---|---|---|
| Onset | 17-7 | cm | 2.3 (0.5) | 2.3 (0.6) | 0.0 (0.6) | 2.3 (0.6) | −0.0 (0.4) |
| | 7-2 | cm | 3.3 (1.3) | 3.7 (2.0) | 0.4 (1.3) | 3.5 (2.0) | 0.2 (1.4) |
| Peak | 17-7 | cm | 2.7 (0.4) | 2.8 (0.6) | 0.1 (0.4) | 2.7 (0.6) | 0.0 (0.4) |
| | 7-2 | cm | 3.8 (1.3) | 3.6 (1.5) | −0.2 (1.3) | 3.3 (1.3) | −0.5 (1.2) |

The effect of cilansetron on gastric emptying time and colonic transit time has been studied in 1 clinical study in healthy young subjects and 2 studies in elderly subjects. For measurement of gastric emptying time, breath samples for the determination of the ¹²C/¹³C ratio were taken on the last day of each treatment period immediately after ingestion of a ¹³Clabeled meal. The patients' breath was collected in separate 15-minute intervals, for four hours in total. ¹³CO₂ measurements were performed with an isotope ratio mass spectrometer. The half-life (TK) and lag-time (Tlag), which describe the duration of gastric emptying, were used as the primary parameters to evaluate gastric emptying time. In all 3 studies, colonic transit time was measured by administration of radio-opaque markers for the last six days of each treatment period, followed by a single abdominal X-ray image at 24 hours after ingestion of the last set of markers. The numbers of markers within the colon were used to calculate segmental (right colon, left colon, and rectosigmoid) and total colonic transit time.

Study K.241.5013 was a randomized, double-blind, placebo-controlled, 3-way crossover study in 12 healthy male subjects (ITT population), aged 20-27 years. Subjects received cilansetron 4 mg, cilansetron 8 mg, or placebo orally TID for 14 days during each of the treatment periods, with a 14-day washout between periods. The primary efficacy parameter was the total mean colonic transit time during each treatment and the secondary parameters were the segmental colonic transit times and the gastric emptying measurements. A one-sided Dunnett t-test procedure was used to compare efficacy parameters between the cilansetron and placebo treatments.

The results of this study are summarized in Table 2.7.2.3.37 and Table 2.7.2.3.38. Both total and segmental colonic transit times showed a trend towards a dose-related increase with cilansetron in comparison to placebo. A near-statistically significant increase was observed in total colonic transit time for the cilansetron 8 mg treatment compared to placebo (p=0.032, but null hypothesis not rejected), but this could not be confirmed due to the large variability in the data. Mean gastric emptying times were also higher for the cilansetron treatments compared to placebo, but differences were not statistically significant.

TABLE 2.7.2.3.37
Arithmetic mean (SD) colonic transit times following administration of
cilansetron 4 mg, 8 mg, or placebo TID orally for 14 days in healthy
subjects (Study K.241.5012)

| | Transit Times (hours) | | | |
|---|---|---|---|---|
| | Colonic Segments | | | Colon |
| Treatment | Right | Left | Rectosigmold | Total |
| Placebo (n = 12) | 7.6 (7.0) | 20.4 (10.7) | 14.2 (13.9) | 42.2 (18.0) |
| Cilansetron 4 mg (n = 12) | 9.4 (9.5) | 24.2 (12.9) | 15.8 (13.5) | 49.4 (24.6)^{a} |
| Cilansetron 8 mg (n = 12) | 11.8 (13.9) | 25.8 (16.7) | 16.2 (11.9) | 53.8 (28.5)^{a} |

^{a}Cilansetron treatments were compared to placebo using a one-sided Dunnett t-test. Null hypothesis was not rejected for either cilansetron dose.

TABLE 2.7.2.3.38
Arithmetic mean (SD) parameters for gastric emptying time following
administration of cilansetron 4 mg, 8 mg, or placebo TID orally for
14 days in healthy subjects (Study K.241.5012)

| Treatment | T½ (hours)^{a} | Tlag (hours)^{a} |
|---|---|---|
| Placebo (n = 12) | 2.29 (0.26) | 1.58 (0.19) |
| Cilansetron 4 mg (n = 12) | 2.46 (0.35) | 1.70 (0.22) |
| Cilansetron 8 mg (n = 12) | 2.38 (0.42) | 1.63 (0.30) |

^{a}No statistically significant differences (p > 0.05) were found for comparisons of cilansetron treatments to placebo, using a one-sided Dunnett t-test.

Studies K.241.5013 and K.241.5014 were randomized, double-blind, placebo-controlled, 2-way crossover studies in 8 healthy male and female elderly subjects, each (ITT population, aged 64-83 years in K.241.5013 and 66-84 years in K.241.5014). Elderly subjects were selected for these studies because they commonly suffer from constipation and it was of interest whether cilansetron would prolong gastrointestinal transit times in this population. Subjects received cilansetron 4 mg or placebo in Study K.241.5013 and cilansetron 8 mg or placebo in Study K.241.5014. Treatments were administered orally TID for 14 days during each period, with a 14-day washout between periods. The primary efficacy parameter was the total mean colonic transit time during each treatment and the secondary parameters were the segmental colonic transit times and the gastric emptying measurements. One-sided t-tests were used to compare efficacy parameters between the cilansetron and placebo treatments.

The results of these studies are summarized in Table 2.7.2.3.39 and Table 2.7.2.3.40. For the 4 mg cilansetron treatment, mean total colonic transit time was higher for cilansetron than for placebo, but a statistically significant difference was not obtained due to the high variability observed in the data. However, for the 8 mg cilansetron treatment, a statistically significant increase in total colonic transit time was observed for cilansetron compared to placebo p=0.033). No single part of-the colon was responsible for the increased total colonic transit time with either dose since all three segments (right, left, rectosigmoid) generally showed a similar tendency, although statistical significance was not reached for any of the individual segments. A small but non-significant increase in gastric emptying time (T % and Tlag) was observed with cilansetron treatment compared to placebo for the 4 mg dose. With the 8 mg cilansetron treatment, a significant increase in gastric emptying time was observed for cilansetron compared to placebo (significant increase in Tlag [p=0.039] and near-significant increase in T1/2 [p=0.055]).

TABLE 2.7.2.3.39
Arithmetic mean (SD) colonic transit times following administration of
cilansetron (4 mg or 8 mg) or placebo TID orally for 14 days in healthy,
elderly subjects (Studies K.241.5013 and K.241.5014)

| | Transit Times (hours) | | | |
|---|---|---|---|---|
| | Colonic Segments^{a} | | | |
| Treatment | Right | Left | Rectosigmold | Colon Total^{a} |
| Study K.241.5013 | | | | |
| Placebo (n = 8) | 21.9 (12.5) | 19.8 (18.4) | 8.1 (6.8) | 49.8 (33.3) |
| Cilansetron 4 mg (n = 8) | 27.3 (29.2) | 21.9 (16.0) | 6.3 (8.3) | 55.5 (41.3) |
| Study K.241.5014 | | | | |
| Placebo (n = 8) | 22.6 (27.2) | 19.1 (17.2) | 14.4 (17.3) | 56.1 (52.9) |
| Cilansetron 8 mg (n = 8) | 31.4 (21.0) | 31.5 (30.5) | 16.7 (25.2) | 79.8 (49.8)* |

^{a}Cilansetron treatment compared to placebo using a one-sided t-test.
*Statistically significant difference (p < 0.05)

TABLE 2.7.2.3.40
Arithmetic mean (SD) parameters for gastric emptying time following
administration of cilansetron (4 mg or 8 mg) or placebo TID orally for 14
days in healthy, elderly subjects (Studies K.241.5013 and K.241.5014)

| Treatment | T½ (min)^{a} | Tlag (min)^{a} |
|---|---|---|
| Study K.241.5013 | | |
| Placebo (n = 8) | 169 (36) | 111 (18) |
| Cilansetron 4 mg (n = 8) | 183 (32) | 122 (24) |
| Study K.241.5014 | | |
| Placebo (n = 8) | 144 (24) | 95.0 (15.4) |
| Cilansetron 8 mg (n = 8) | 166 (18) | 114 (18)* |

^{a}Cilansetron treatment compared to placebo using a one-sided t-test.
*Statistically significant difference (p < 0.05)

One additional clinical study was conducted in IBS patients to evaluate the effect of cilansetron on colonic transit time but this study was stopped due to low recruitment, so no conclusions could be drawn from this'study.

The effect of cilansetron on colonic motility was evaluated in a randomized, double-blind, placebo-controlled, 3-way crossover study in 12 healthy male and female subjects (ITT population), aged 24-34 years. Subjects received cilansetron 4 mg, cilansetron 8 mg, or placebo orally TID for 7 days during each of the treatment periods, with a 7-day washout between periods. Colonic motility assessments were done on the last day of each treatment period, around the time of the last dose. Subjects were fasted for 12 hours prior to start of colonic assessments. Colonic motor activity was recorded from 30 minutes prior to dosing until 240 minutes postdose. A high-calorie meal was administered at 1 hour postdose and 1 mg neostigmine was administered intramuscularly at 2.5 hours postdose. The main objective of the assessments was to establish whether cilansetron was able to reduce colonic motor activity after meal ingestion and after neostigmine injection. The main parameter of interest was the motility index, which was measured in intervals of ten minutes for a total period of 150 minutes postprandially. The time intervals of major interest were the 30-minute interval after start of meal ingestion (9 1-120 minutes) and the 30-minute interval after neostigmine injection (181-210 minutes). An ANOVA was used to compare the motility indices between treatments.

Results of the motility index assessments are summarized in Table 2.7.2.3.41. After meal ingestion, there was no statistically significant difference in motility index between placebo and cilansetron 4 mg p=1.00) or cilansetron 8 mg p=1.00). However, following administration of neostigmine injection, there was a trend towards an increase in motility index following both doses, and a trend towards a statistically significant increase for cilansetron 4 mg p=0.06) but not for cilansetron 8 mg when compared with placebo. Cilansetron also tended to increase the number, amplitude and duration of contractions after neostigmine and to increase the number of contractions after meal ingestion.

TABLE 2.7.2.3.41
Arithmetic mean (SD) for colonic motility index following
administration of cilansetron 4 mg, 8 mg, or placebo orally
TID for 7 days in healthy subjects (Study S.241.2.106)

| | | | | Comparison to | |
|---|---|---|---|---|---|
| | | | | Ratio Placebo: | |
| | Arithmetic Mean (SD) | | | (95% CI) (%)^{a} | |
| Measurement | 4 mg | 8 mg | Placebo | 4 mg | 8 mg |
| Interval | (n = 12) | (n = 12) | (n = 12) | (n = 12) | (n = 12) |
| After Meal | 20358 | 23478 | 19931 | 101 | 106 |
| Ingestion | (12818) | (16168) | (10644) | (68, 150) | (71, 157) |
| After Neostigmine | 50580 | 48691 | 24992 | 192 | 162 |
| | (36028) | (37140) | (12879) | (114, 321) | (97, 271) |

^{a}Comparison between cilansetron treatments and placebo using ANOVA.

Effect on Nausea and Vomiting

The effect of cilansetron on nausea and vomiting has been studied in 2 clinical studies in cancer patients. Both studies were randomized, singleblind, positive-controlled, parallel-group, pilot studies in male and female patients with malignant tumors, to investigate the-effect of cilansetron on cytostatic-induced nausea and vomiting. In both studies, patients were on their first cycle of chemotherapy and were being treated with cisplatin (275 mg/m2 as a single dose in Study S.24 1.2.10 1 and 220 mg/m2/day for 5 days in Study K.24 1 S O 17). Patients in Study S.24 1.2.10 1 were aged 40-74 years (ITT population=16) and patients in Study K.24 1 S O 17 were aged 39-74 years (ITT population=19). In both studies, patients were randomized to receive either N cilansetron 8 mg TID or IV ondansetron 8 mg TID for 1 day (Study S.241.2.101) or 5 days (Study K.2415017), corresponding to the day(s) of their cisplatin administration. If nausea and vomiting were completely controlled on a treatment day, the second and/or third doses of cilansetron or ondansetron could be omitted on that day. The primary efficacy parameter in both studies was the combination of the occurrence of vomiting and the severity of nausea, with a responder being defined as a patient with no vomiting and not more than mild nausea during the treatment day(s).

Secondary parameters were the severity of nausea, number of vomiting episodes, and global well-being assessments. Since subject numbers were small in both studies, only descriptive statistics were used to compare treatments and no inferential statistics were performed.

Both studies were pilot studies with small sample sizes, and therefore no definite conclusions could be drawn. Additionally, the dosing regimens of cisplatin differed between the 2 studies, which could lead to differing profiles of nausea and vomiting. However, the results suggested that the response rate of cilansetron was similar to or lower than ondansetron. In a first study, the response rate of cilansetron (2/12 [16.7%]) was similar to that of ondansetron (114 [25.0%]), while in another study, the response rate of cilansetron (3/14 [2 1.4%]) was lower than for ondansetron (315 [60.0%]). Similarly, in the first study, the severity of nausea and the number of vomiting episodes did not show relevant differences over time, nor clear differences between the cilansetron and ondansetron groups, but in Study K.241.5017, the maximum severity of nausea and total number of vomiting episodes were higher for the cilansetron group compared to the ondansetron group. In both studies, the investigation of the patient's and investigator's global well-being assessment did not show clear differences between the treatment groups.

Effects on Electrocardiogram

The effect of cilansetron on ECG parameters, particularly the QTc interval, has been evaluated at a cilansetron dose that is 8-fold higher than that planned for clinical use. In a randomized, double-blind, placebo-controlled, 2-way crossover study in 9 male and 9 female healthy subjects (IT population, aged 21-39 years), subjects were randomized to receive 2 treatments (cilansetron 16 mg tablet and placebo) during the 2 treatment periods. During each treatment period, subjects received a single dose of their assigned treatment on the first day (Day 1), after which they received the same treatment TID for 4 days (Days 2-5). There was a 10-day washout period between treatment periods. Subjects were fasted for at least 2 hours before and following each dose, and heavy physical activity was restricted. Serial 12-lead ECGs was recorded up to 24 hours following dosing on Day 1 (single dose) and on the last day (Day 5) of each treatment period. Heart rate and ECG intervals (RR, PR, QRS, and QT) were measured electronically, but were manually verified and corrected if needed by the investigator. QT intervals were corrected (QTc) electronically for heart rate using the Bazett's correction. Mean and maximum QTc intervals were calculated over different time intervals postdose and were compared between treatments using an ANOVA accounting for sequence, gender, subject within sequence and gender, period, and treatment effects.

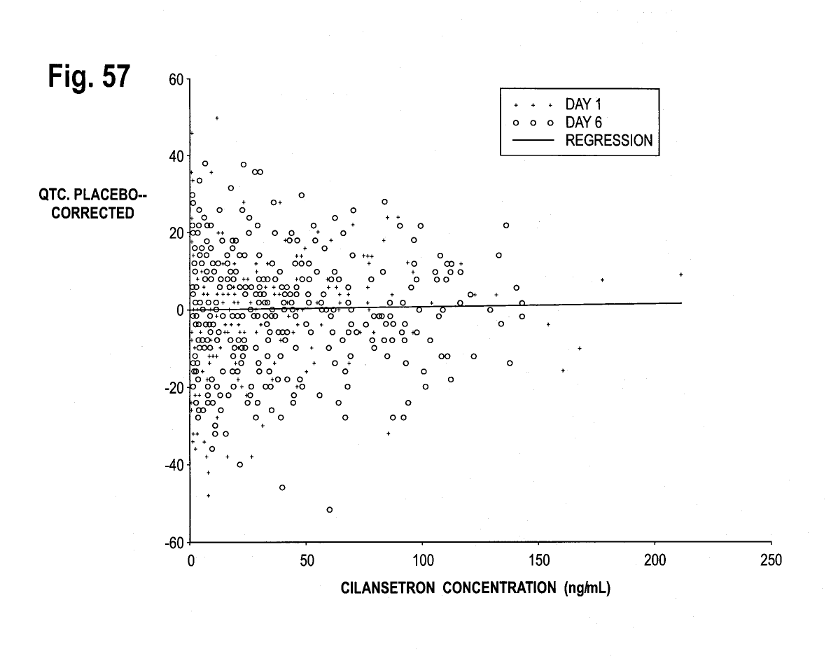

The results of the QTc evaluation are summarized in Table 2.7.2.3.42. Cilansetron has no effect p>0.05) on the mean or maximum QTc interval following single or multiple dosing. In accordance with the generally higher QTc intervals in females compared to males in the general population, slightly higher mean and maximum QTc intervals were observed in females than in males in this study, resulting in a significant gender effect (p<0.10) for statistical comparisons of some QTc parameters. During the entire study, only 6 occurrences of a QTc interval greater than 450 ms were observed and the frequency was comparable between treatments (3 occurrences following each treatment). No QTc values greater than 500 ms were observed. A comparison of individual mean QTc values on Days 1 and 5 showed no individual increases above 30 ms. No correlation was observed between placebo-corrected QTc intervals and cilansetron concentrations following single or multiple dosing (FIG. 57).

TABLE 2.7.2.3.42
Arithmetic mean (% CV) of QTc intervals (Bazett's correction) and results
of statistical comparisons between treatments following administration of
cilansetron 16 mg and placebo orally as a single dose and TID for 4 days in
healthy subjects (Study S.241.1.108)

| | Arithmetic Mean (% CV) | | | | | |
|---|---|---|---|---|---|---|
| | Day 1 (Single dose) | | Day 5 (Multiple Dose) | | Statistical Comparison^{c} | |
| | Cilansetron | Placebo | Cilansetron | Placebo | Mean Diff (90% CI) (ms) | |
| QTc Parameters^{a} | (n = 18) | (n = 18) | (n = 18) | (n = 18) | Day 1 | Day 5 |
| Mean QTc(0-24) (ms) | | | | | | |
| All | 407 (3) | 408 (3) | 407 (3) | 407 (3) | NC | −0.2 (−2.3, 1.8) |
| Females | NC | NC | 412 (3) | 412 (3) | NC | NC |
| Males | NC | NC | 401 (2) | 402 (3) | NC | NC |
| Max QTc(0-24) (ms) | 425 (4) | 429 (3) | 429 (3) | 428 (3) | NC | 0.9 (−2.4, 4.1) |
| Tmax(0-24) (h)^{b} | 3 (0.24) | 3 (0.12) | 17 (0.22) | 17 (1.24) | NC | NC |
| Mean QTc(0-8) (ms) | 410 (4) | 412 (2) | 406 (3) | 405 (3) | −2.5 (−6.8, 1.8) | 0.9 (−1.8, 3.5) |
| Mean QTc(8-16) (ms) | NA | NA | 404 (3) | 405 (3) | NA | −0.8 (−4.4, 2.7) |
| Mean QTc(16-24) (ms) | NA | NA | 410 (3) | 411 (4) | NA | −0.7 (−2.7, 1.3) |

NA—not applicable;
NC—not calculated;
% CV - coefficient of variation on arithmetic mean in percent.
^{a}QTc using Bazett's correction.
^{b}Results presented are median (min, max).
^{c}Least squares mean differences and 90% confidence intervals from ANOVA comparison of the cilansetron treatment to placebo. No statistically significant differences (p < 0.05) were found for any parameter.

Clinical Efficacy

Three double-blind, placebo-controlled, randomized, multicenter, multinational, parallel-group studies were performed to investigate the safety and Efficacy of cilansetron 2 mg TID in diarrhea-predominant IBS subjects over 12 week studies (S241.3.006 and S241.3.011) or 26 week studies (S241.3.009). In Study S241.3.011, the 12-week treatment period was followed by a four-week rerandomized treatment period. Data from the three efficacy studies are presented based on the individual clinical study reports presented below.

Moreover, Phase 11, double-blind, placebo-controlled, randomized, multicenter, multinational, parallel-group dose-ranging studies were performed to investigate the efficacy and safety of cilansetron in non-constipated EBS subjects over 12 weeks of treatment. Supporting efficacy data from these two studies relevant to dosing recommendations are presented based on the individual CSRs below.

Summary of Results of Individual Studies

A tabular listing of all studies that provided information relevant to the efficacy of cilansetron is presented in Table 2.7.3:2.

TABLE 2.7.3: 2
Results of Efficacy Studies

| | | | | | |
|---|---|---|---|---|---|
| | | | P-Value | | |
| | Overall | | for Number | P-Value | |
| | Number | | of Months a | for Overall | |
| | Randomized/ | Weekly Diary Questions | Subject was a | Responder | Primary and Main |
| Study | Completed | on Adequate Relief | Responder^{a,b} | Rate^{a,b} | Secondary Endpoints |

Pivotal Efficacy Studies

| | | | | | |
|---|---|---|---|---|---|
| S241.3.006 | 711/587 | IBS symptoms | <0.001** | <0.001** | Adequate relief of IBS |
| | | Abdominal | <0.001** | <0.001** | symptoms, abdominal |
| | | pain/discomfort | | | pain/discomfort, and |
| | | Abnormal bowel habits | <0.001** | <0.001** | abnormal bowel habits |
| S241.3.009 | 806/629 | IBS symptoms | <0.001** | <0.001** | Adequate relief of IBS |
| | | Abdominal | <0.001** | <0.001** | symptoms, abdominal |
| | | pain/discomfort | | | pain/discomfort, and |
| | | Abnormal bowel habits | <0.001** | <0.001** | abnormal bowel habits |
| S241.3.011 | 770/591^{c} | IBS symptoms | <0.001** | <0.001** | Adequate relief of IBS |
| | | Abdominal | <0.001** | <0.001** | symptoms, abdominal |
| | | pain/discomfort | | | pain/discomfort, and |
| | | Abnormal bowel habits | <0.001** | <0.001** | abnormal bowel habits |

Supporting Efficacy Studies

| S241.2.112 | 490/348 | IBS symptoms | | | |
|---|---|---|---|---|---|
| | | cilansetron 1 mg TID | NA | 0.008** | |
| | | cilansetron 2 mg TID | NA | 0.31 | |
| | | cilansetron 8 mg TID | NA | 0.15 | |
| | | cilansetron 16 mg TID | NA | 0.014* | Relief of IBS symptoms |
| S241.2.113 | 485/368 | IBS symptoms | | | |
| | | cilansetron 1 mg TID | NA | 0.479 | |
| | | cilansetron 2 mg TID | NA | 0.013* | |
| | | cilansetron 4 mg TID | NA | 0.980 | |
| | | cilansetron 16 mg TID | NA | 0.061 | Relief of IBS symptoms |
| *Significant at the 0.050 level for treatment effect; | | | | | |
| **significant at the 0.010 level for treatment effect. | | | | | |
| ^{a}P-values for Studies S241.3.006, S241.3.009, and S241.3011 are for comparisons of cilansetron 2 mg TID versus placebo. Comparisons for Studies S241.2.112 and S241.2.113 are for cilansetron versus placebo. | | | | | |
| ^{b}In Studies S241.2.112 and S241.2.113, the p-value was adjusted for multiple comparisons using the Bonferroni correction. | | | | | |
| ^{c}Subjects who completed the three-month treatment period. | | | | | |
| Note: | | | | | |
| Due to carly study termination by the Sponsor only 20 subjects in Study S241.3.001 and no subject in Study S241.3.002 completed the study, therefore, efficacy results were not evaluated. | | | | | |
| Note: | | | | | |
| Primary endpoint for Studies S241.3.006 and S241.3.011 was the number of months a subject was a responder for IBS Symptoms. Primary endpoint for Study S241.3.009 was overall responder rate for IBS Symptoms. Other endpoints shown for these studies are considered secondary endpoints. | | | | | |
| Data source: Study S241.3.006: CSR Table 10.1.2.1.1 Left. Vol. 9 Righ, CSR Table 10.1.2.2.1 Left. Vol. 9 Righ; Study S241.3.011: CSR Table 10.1.2.1.1 Left. Vol. 9 Righ, CSR Table 10.1.2.2.1 Left. Vol. 9 Righ; Study S241.3.009: CSR Table 10.1.2.1.16 Left. Vol. 9 Righ, CSR Table 10.1.2.2.9 Left. Vol. 9 Righ; | | | | | |
| # Study S241.2.112: Table 16 Left. Vol. 9 Righ; Study S241.2.113: Table 16 Left. Vol. 9 Righ | | | | | |

Narrative Descriptions of Pivotal and Supportive Efficacy Studies

Clinical studies S241.2.112, S241.2.113, S241.3.006, S241.3.009, and S241.3.011 were performed to assess the efficacy of cilansetron in treating IBS-D in men and women. Some of the findings of the studies are summarized here, but a more complete discussion of each study is presented later in the application.

Study S241.2.112 was a Phase II, double-blind, placebo-controlled, randomized, multicenter parallel-group, dose-ranging study to investigate the efficacy and safety of cilansetron in non-constipated subjects with irritable bowel syndrome. The study was conducted in 51 study centers in the U.S. A total of at least 88 subjects were planned per treatment group evaluable for efficacy, in total 440 to complete the study; 975 subjects were screened; 490 were randomized and 481 analyzed for safety.

Diagnosis and main criteria for inclusion were IBS subjects satisfying the Rome criteria (1992) and minimum abdominal pain/discomfort, stool frequency and consistency determined during the run-in period. Subjects were treated with cilansetron 1 mg, 2 mg, 8 mg, and 16 mg tablets given orally TID or matching placebo tablets given orally TID for 12 weeks.

Efficacy was evaluated by weekly global questions concerning adequate relief of IBS symptoms (abdominal pain/discomfort, abnormal bowel habits). A responder is a subject who responded “Yes” at least 50% of the time after randomization, with a minimum treatment duration of four weeks.

Safety was evaluated by adverse events, concomitant medications, laboratory evaluations, vital signs, physical examination and electrocardiogram (ECG).

Among other findings (discussed more fully below), it was determined in this particular study that: (i) the primary endpoint, adequate relief of IBS symptoms (abdominal pain/discomfort, abnormal bowel habits), as assessed by subject response was statistically significantly achieved for 1 mg and 16 mg TID (each 22% better than placebo). Dosages of 2 mg and 8 mg TID resulted in responder rates 13% and 15% higher than found with placebo, although statistical significance was not achieved at these doses; (ii) adequate relief responder rates for both male and female subjects were generally comparable except for the 1 mg cilansetron group, which showed a higher rate (26% better than placebo) in females. The effect size in males was larger due to a lower placebo response. Based on these results, but taking into account the lower sample size of the male subjects, there is no apparent gender difference with adequate relief response rates with cilansetron; (iii) the 2 mg and 16 mg cilansetron doses showed additional efficacy on the daily assessment of abdominal pain. The 16 mg dose effect was more pronounced at each point in time; the 2 mg dose effect was less pronounced and did not occur throughout the study; (iv) cilansetron increased stool firmness and decreased stool frequency in a dose-dependent fashion up to the 8 mg dose. Further reduction in these parameters was not observed with the 16 mg dose; (v) although the 1 mg dose provided adequate relief of IBS symptoms, it did not exhibit significant effects on the secondary parameters, with the possible exception of stool consistency. The 2 mg cilansetron dose group showed a slightly stronger efficacy evidence with regard to some of the secondary parameters compared to the 1 mg cilansetron group. The 2 mg group showed a larger increase in stool firmness over time towards harder stools than the 1 mg and the number of stools decreasted more with 2 mg cilansetron treatment than with 1 mg cilansetron; (vi) all treatments showed trends towards improvement in the quality of life factors ‘physical functioning’, ‘role physical’, ‘bodily pain’, ‘general health’, ‘vitality’, ‘social functioning’, ‘role-emotional’ and ‘mental health’, although the general quality of life (QOL) scale used may have lacked sensitivity to assess QOL in IBS subjects.

Safety

In total, there were 399 subjects (83%) with treatment-emergent adverse events (TEAEs); 71% with placebo and 89%, 86%, 84% and 82% with 1 mg, 2 mg, 8 mg and 16 mg cilansetron treatment, respectively. Constipation, nausea, dyspepsia, abdominal distension and headache were the most frequently reported AEs. Only the rate of constipation was observed to be clearly increased with cilansetron treatment compared to placebo treatment. This effect was dose-dependent up to the 8 mg dose of cilansetron. No serious adverse events (SAE) of constipation was observed. None of the subjects died during the course of the study. Eight subjects reported 17 SAEs. Five SAEs in four subjects were judged to be related (possibly) to the trial medication: aggravated tachycardia with 2 mg cilansetron treatment, hemorrhoids and confusion with 8 mg cilansetron treatment and diverticulitis and moderate abdominal pain with 16 mg cilansetron treatment. No cases of ischemic colitis were reported during the study. A mild dose-dependent within normal range and not clinically relevant increase in creatinine level was observed with cilansetron treatment (from 2% with 1 mg to 8% with 16 mg cilansetron treatment). Two subjects showed clinically significant elevated creatinine levels during study treatment, however both values were judged to be unrelated and unlikely related to the cilansetron treatment that was given. No clinically significant effects on other laboratory values including liver parameters, lipid metabolism, hematology, or urinary parameters were observed. Electrocardiogram data showed no clinically relevant changes versus Baseline, no dose related trends and no clinically relevant differences between the treatment groups.

Study S241.2.113 was a Phase II, double-blind, placebo-controlled, randomized, multi-center, parallel-group, dose-ranging study to investigate the efficacy and safety of cilansetron in nonconstipated subjects with irritable bowel syndrome. The study was conducted in 55 study centers in Canada, France, Austria, Germany, United Kingdom, Italy, Belgium, and The Netherlands. A total of at least 88 subjects per treatment group evaluable for efficacy were planned, in total 440 to complete the study; 776 subjects were screened; 485 subjects were randomized (479 in the safety sample). Diagnosis and main criteria for inclusion were IBS subjects satisfying the Rome criteria (1992) and minimum abdominal pain/discomfort, stool frequency and consistency determined during the run-in period.

Subjects were treated with cilansetron 1 mg, 2 mg, 4 mg and 16 mg tablets given orally TID or matching placebo tablets given orally TID for 12 weeks.

The primary efficacy parameter was the weekly global question concerning adequate relief of IBS symptoms (abdominal pain/discomfort, abnormal bowel habits). A responder is a subject who responded “Yes” at least 50% of the time after randomization, with a minimum treatment duration of four weeks.

Safety was evaluated by AEs, concomitant medications, laboratory evaluations, vital signs, physical examination and ECG.

Among other findings (discussed more fully below), it was determined in this particular study that: (i) the primary endpoint, adequate relief of IBS symptoms (abdominal pain/discomfort, abnormal bowel habits), as assessed by subject response, was statistically significant for 2 mg TID (22% better than placebo). Dosages of 1 mg, 4 mg and 16 mg TID resulted in responder rates of 11%, 8% and 18% higher than found with placebo, but statistical significance was not achieved at these doses; (ii) the 2 mg cilansetron dose showed the largest improvement on the daily assessment of abdominal pain. Cilansetron increased stool firmness, especially with the 4 mg and 16 mg dosages, and decreased stool frequency with no apparent effect difference between the dosages; (iii) cilansetron treatment showed most improvement compared to placebo treatment in all QOL factors of the SF-36 scale. A difference of more than 10 points relative to placebo treatment were observed for the 1 mg, 2 mg and 4 mg cilansetron groups for the factors Role-physical and Role-emotional. For all treatments, trends towards improvement were mainly observed in the quality of life factors ‘physical functioning’, ‘role physical’, ‘bodily pain’, ‘general health’, ‘vitality’, ‘social functioning’, ‘role-emotional’ and ‘mental health’, however the general QOL scale used may have lacked sensitivity to assess QOL in IBS subjects; (iii) adequate relief responder rates for both male and female subjects were generally comparable. In the 1 mg and 2 mg cilansetron groups, the males showed a slightly higher responder rate, while in the 4 mg cilansetron group the females showed a higher rate. The highest effect was seen in the 2 mg group, overall and separately for the gender subgroups. Based on these results, but taking into account the lower sample size of the male subjects, there is no apparent gender difference with adequate relief response rates with cilansetron.

Safety:

In total, there were 359 subjects (75%) with TEAEs; 71% with placebo and 74%, 74%, 76% and 80% with 1 mg, 2 mg, 4 mg and 16 mg cilansetron treatment. Constipation, dyspepsia, nausea, headache and abdominal distension were the most frequently reported AEs. Only the rate of constipation was observed to be clearly increased with cilansetron treatment compared to placebo treatment. This effect was dose-dependent up to the 4 mg dose of cilansetron. No SAE of constipation was observed. Abdominal pain was observed more frequently with cilansetron treatment than with placebo, however not in a dose-dependent fashion. None of the subjects died during the course of the study. Six subjects reported six SAEs: severe hemorrhagic duodenal ulcer (placebo), renal colic (placebo), unintentional pregnancy (1 mg), severe angina attack (4 mg), severe blood clotting (thrombosis) (16 mg) and moderate phlebitis (16 mg). All of these SAEs were judged by the investigator to be unrelated to the study medication. No cases of ischemic colitis or complications of severe constipation were reported during the study. ECG data showed no clinically relevant changes versus Baseline, no dose related trends and no clinically relevant changes between the treatment groups. A slight dose dependent increase in mean change of creatinine values from Baseline to each study visit was observed. No other relevant changes in laboratory parameters, vital signs, or physical examination findings were observed.

Study S241.3.006 was a Phase III, double-blind, placebo-controlled, randomized, multicenter study to investigate the safety and efficacy of 2 mg TID of cilansetron over 12 weeks in diarrhea-predominant irritable bowel syndrome subjects. The study was conducted in 129 study centers in the United States. At total of 2080 subjects consented to participate, and 7 1 1 subjects were randomized. A total of 692 subjects were analyzed for efficacy and 701 subjects were analyzed for safety. Diagnosis and main criteria for inclusion were IBS subjects satisfying the Rome I criteria (1 992); the diarrhea predominant sub-population of the Rome II criteria; and a minimum abdominal pain/discomfort, stool frequency and consistency determined during the run-in period. Subjects were treated with cilansetron 2 mg tablets given orally TID or matching placebo tablets given orally TID for 12 weeks double-blind treatment.

The primary efficacy parameter was the number of months that a subject was a responder for IBS symptoms (abdominal pain, abdominal discomfort, abnormal bowel habits) over the 12-week treatment period. A responder for a month (monthly responder) was defined as a subject who answered the weekly question with “yes” at least twice for that month.

Safety was evaluated by physical examination, ECG, vital signs, clinical laboratory evaluations, concomitant medications, and AEs. An independent Gastrointestinal Events Review Board (GERB) was established to advise on gastrointestinal (GI) AEs during the course of the study and other safety events as considered necessary.

Among other findings (discussed more fully below), it was determined in this particular study that: (i) strong evidence for the superiority of cilansetron 2 mg TID over placebo was provided in this study for both male and female subjects over three months of treatment. Treatment differences were seen within the first week of treatment and the effect remained stable throughout the three months of the study; (ii) the evidence for the superiority of cilansetron 2 mg TID over placebo was established by the primary efficacy analysis, number of months a subject was a responder for the weekly diary question on adequate relief of IBS symptoms, as reflected by the proportion of subjects who were responders for zero, one, two, or three months during the three-month treatment period (37%, 13%, 15%, and 35% in the cilansetron 2 mg TID group versus 56%, 12%, 14%, and 18% in the placebo group, respectively, p<O.OO1); (iii) statistically significant differences were demonstrated in favor of cilansetron compared with placebo by the main secondary efficacy analyses during the three-month treatment period, as reflected by the proportion of subjects who were responders for zero, one, two, or three months for the weekly diary questions on adequate relief of abdominal pain/discomfort (34%, 12%, 14%, and 40% in the cilansetron 2 mg TID group versus 49%, 12%, 16%, and 23% in the placebo group, respectively, p<0.001) and abnormal bowel habits (36%, 12%, 15%, and 38% in the cilansetron 2 mg TID group versus 58%, 13%, 12%, and 17% in the placebo group, respectively, p<0.001); (iv) the superiority of cilansetron 2 mg TID versus placebo was also demonstrated by the statistically significant treatment differences in the overall responder rate for the weekly diary questions on adequate relief of symptoms, i.e., significantly more subjects were responders in the cilansetron 2 mg TID group compared with the placebo group during the three-month treatment period (observed case analysis on the intent-to-treat (ITT) population: IBS symptoms, 49% versus 28%, p<0.001; abdominal pain discomfort, 52% versus 37%; p<0.001 ; and abnormal bowel habits, 51% versus 26%, p<0.001); (v) as would be predicted from the strong evidence for the efficacy of cilansetron 2 mg TID according to the primary and main secondary efficacy analyses (number of months that a subject was a responder) and the overall responder rate, all other analyses of the responses to the weekly diary questions on adequate relief, i.e., responder rate at each month, adequate relief rate, and number of subjects with adequate relief by week, showed similar superiority of cilansetron treatment over placebo. The proportion of subjects with adequate relief was greater for cilansetron 2 mg TID versus placebo already at Week 1 and increased until Week. The proportion of subjects with adequate relief for cilansetron 2 mg TID remained relative stable from Week 5 to the end of the treatment period. Statistically significant treatment group difference in favor of cilansetron was already seen in responder rate for the first month of treatment and the favorable effect of cilansetron was sustained for the three month treatment period; (vi) analysis of EBS symptoms recorded in the daily diary showed a statistically significant beneficial effect of cilansetron 2 mg TID on the symptoms of abdominal pain/discomfort, urgency, feeling of incomplete evacuation, and bloating or feeling of abdominal distension. Cilansetron treatment was also associated with statistically significant increased stool firmness and decreased stool frequency with increased straining; (vii) the cilansetron 2 mg TID group demonstrated a statistically significant greater improvement compared with the placebo group in the IBS-QOL total score and each subscore interference with activity, body image, health worry, food avoidance, social reaction, sexual, relationships, and dysphoria). Analysis of responses to the interruption of activities assessment demonstrated a clinically meaningful greater improvement in the cilansetron 2 mg TID group compared with the placebo group in the subjects' ability to effectively perform their everyday activities; (viii) all subgroups of gender, age, race, body mass index (BMI), IBS severity, and IBS duration showed similar responses to cilansetron treatment compared to the overall ITT population. Treatment differences across the pooled centers showed cilansetron 2 mg TID consistently with a more favorable response over placebo.

Safety

Cilansetron 2 mg TID was safe and well tolerated during this trial. A total of 64% versus 55% of subjects in cilansetron 2 mg TID and placebo groups experienced at least one TEAE in the study. The most frequently reported TEAEs with a higher incidence in the cilansetron 2 mg TID group compared with the placebo group were GI disorders including constipation (19% versus 4%), abdominal pain NOS (6% versus 1%), and fecal occult blood positive (6% versus 3%). Other common events possibly associated with cilansetron treatment were upper respiratory tract infections, headache, and blood creatinine phosphokinase increased. Female subjects had a higher incidence of constipation in both the cilansetron 2 mg TID and placebo groups (23% versus 5%) compared with male subjects (8% versus <1%). Less common events of special interest with a possible association with cilansetron treatment were flatulence (10 subjects versus three subjects) and abdominal distension (seven subjects versus three subjects). No cases of ischemic colitis were reported by the Investigator. One subject in the cilansetron 2 mg TID group had an SAE of constipation with additional findings of acute colonic ulcers and acute colitis. No subject in either treatment group experienced epilepsy or seizures. One subject in each treatment group experienced the SAE of deep venous thrombosis NOS. Laboratory data showed a possible association of cilansetron treatment with decreased leukocytes, increased lymphocytes, decreased neutrophils, increased triglycerides, and increased uric acid. No other treatment group differences were noted for any laboratory parameter. Analyses of vital sign, ECG, and physical findings did not reveal any clinically relevant effect of cilansetron.

Study S241.3.009 was a Phase III, a double-blind, placebo-controlled, randomized, multinational study to investigate the safety and efficacy of 2 mg TID of cilansetron over 26 weeks in diarrhea-predominant IBS subjects. The study was conducted in 146 multinational study centers. A total of 1307 subjects consented to participate, and 806 subjects were randomized. A total of 792 subjects were analyzed for efficacy and 799 subjects were analyzed for safety. Diagnosis and main criteria for inclusion were IBS subjects satisfying the Rome I criteria (1992); the diarrhea-predominant sub-population of the Rome II criteria; and a minimum abdominal pain/discomfort, stool frequency and consistency determined during the run-in period. Subjects were treated with cilansetron 2 mg tablets given orally TID or matching placebo tablets given orally TID for 26 weeks.

The primary efficacy parameter was the overall response rate for IBS symptoms, based on the weekly question concerning adequate relief of IBS symptoms (abdominal pain/discomfort, abnormal bowel habits) over the 26 weeks period. A responder was a subject who responded “yes” at least 50% of the time during his/her treatment period, with minimum treatment duration of four weeks. The two main secondary efficacy parameters were overall responder rate based on the weekly question on adequate relief of abdominal pain/discomfort and abnormal bowel habits during the 26-week treatment period. An additional key secondary Efficacy parameter was the number of months (one month equals four weeks) up to six months (24-weeks) that a subject was a responder for IBS symptoms, where a responder was defined as a subject who answered the weekly question with “yes” at least twice for that month. The responder rate for the weekly diary questions on adequate relief at each month and all months, the adequate relief rate, the number of subjects with adequate relief by week, daily diary questions, IBS-QOL scores, and the interruption of activities were also assessed. Safety was evaluated by physical examination, ECG, vital signs, laboratory evaluations, concomitant medications, and AEs.

Among other findings (discussed more fully below), it was determined in this particular study that: (i) strong evidence for the superiority of cilansetron 2 mg TID over placebo was provided in this study for both male and female subjects over 26 weeks of treatment. The onset of the effect was within the first month of treatment and the effect remained stable throughout the six months; (ii) the evidence for the superiority of cilansetron 2 mg TID over placebo was established by the primary Efficacy analysis, overall responder rate for the weekly diary question on adequate relief of IBS symptoms, i.e., there were significantly more responders in the cilansetron 2 mg TID group compared with the placebo group during the 26-week treatment period (observed case analysis—ITT population: 59% versus 45%, respectively, p<0.001); (iii) statistically significant differences were demonstrated in favor of cilansetron compared with placebo by the main secondary efficacy analyses during the 26-week treatment period, overall responder rate for the weekly diary questions on adequate relief of abdominal pain/discomfort (61% versus 46%, p<0.001) and abnormal bowel habits (64% versus 46%, p<0.001); (iv) the superiority of cilansetron 2 mg TID versus placebo was also demonstrated by the statistically significant treatment differences in the number of months a subject was a responder for the weekly diary questions on adequate relief, i.e., subjects in the cilansetron 2 mg TID group were responders for more months during the study compared to subjects in the placebo group for each of the three weekly diary questions during the six-month treatment period; (v) as would be predicted from the strong evidence for the Efficacy of cilansetron 2 mg TID according to the primary, main secondary, and additional key secondary analyses, all other analyses of the responses to the weekly diary questions on adequate relief, i.e., responder rate at each month, adequate relief rate, and number of subjects with adequate relief by week, showed similar superiority of cilansetron treatment over placebo. The proportion of subjects with adequate relief was clinically higher for cilansetron 2 mg TID versus placebo already at Week 1 and increased further until Week 5. The proportion of subjects with adequate relief for cilansetron 2 mg TID continued to increase to a plateau at approximately Week 9, with a slight increase for the remainder of the treatment period. Statistically significant treatment group difference in favor of cilansetron was already seen for the first month of treatment and the favorable effect of cilansetron was sustained for up to six months of treatment; (vi) analysis of IBS symptoms recorded in the daily diary showed a statistically significant beneficial effect of cilansetron 2 mg TID on the symptoms of abdominal pain/discomfort, urgency, and bloating or feeling of abdominal distension. Cilansetron treatment was also associated with statistically significant increased stool firmness, decreased stool frequency, and increased straining. Cilansetron had no effect on the feeling of incomplete evacuation; (vii) the cilansetron 2 mg TID group demonstrated a statistically significant greater improvement compared with the placebo group in the IBS-QOL total score and each subscore interference with activity, body image, health worry, food avoidance, social reaction, relationships, and dysphoria) with the exception of the sexual subscore, which, however, also showed a trend in favor of cilansetron. Analysis of responses to the interruption of activities assessment demonstrated a clinically meaningful greater improvement in the cilansetron 2 mg TID group compared with the placebo group in the subjects' ability to effectively perform their everyday activities; (viii) all subgroups showed similar treatment responses to cilansetron treatment with the exception of subjects with BMI≧30 kg/m². All subgroups of age, race, and IBS severity showed similar response compared to the overall ITT population. There appeared to be geographical differences in the primary, main secondary, and key secondary efficacy variables, mostly due to differences in placebo response among the pooled country groups; (ix) for EBS symptoms, there was a statistically significant treatment group difference in favor of cilansetron 2 mg TID versus placebo in female subjects and a trend towards statistical significance in male subjects. For the other two weekly diary questions of abdominal pain/discomfort and abnormal bowel habits, the treatment differences were statistically significant in both males and females. Female subjects demonstrated slightly more separation of cilansetron response from placebo response compared with male subjects and there was a significant treatment by gender interaction for the primary efficacy variable.

Safety

Cilansetron 2 mg TID was safe and well tolerated during this 26-week trial. A total of 56% versus 50% of subjects in cilansetron 2 mg TID and placebo groups experienced at least one TEAE in the study. The most frequently reported TEAEs with a higher incidence in the cilansetron 2 mg TID group compared with the placebo group were GI disorders including constipation (12% versus 3%), abdominal pain (9% versus 6%), and abdominal distension (3% versus 1%). Other events possibly associated with cilansetron treatment were headache and various infections including rhinoviral infections, upper respiratory tract infections, and nasopharyngitis. Female subjects had a higher incidence of constipation in both the cilansetron 2 mg TID and placebo groups (17% versus 5%) compared with male subjects (7% versus 4%). Less common events of special interest with a possible association with cilansetron treatment were suspected ischemic colitis (three subjects), rectal hemorrhage (three subjects), and abdominal pain aggravated (five subjects). One non-serious TEAE of epilepsy NOS (identified as special interest AEs by the GERB) was reported in a subject with previous history of epileptic seizures. One SAE of deep venous thrombosis was reported in a subject with history of thrombophlebitis. The safety profile of the two subjects who took fluvoxamine as a concomitant medication was unremarkable. No complications of constipation or ischemic colitis were observed. Laboratory data showed an association of cilansetron treatment with elevated gammaglutamyltransferase (GGT) and triglycerides. In male subjects, there was a higher incidence of bilirubin 20.6 mg/dL increase in the cilansetron 2 mg TID group (8%) compared with the placebo group (4%). No other clinically meaningful treatment group differences were noted for any laboratory parameter. Analysis of ECG data showed a slight treatment group difference in the mean change from Baseline at Endpoint for QTc(B) and QTc(F) that was mostly due to decreases within the placebo group. There was a slightly higher frequency of prolonged QT interval and markedly long QTc(F) in the cilansetron 2 mg TID group compared with the placebo group, however, QTc(B) did not reveal any meaningful differences. For most subjects in the cilansetron 2 mg TIE) group, the QT abnormalities occurred during the first three months of treatment and were detected only once during the study. Further analyses of the QT interval adjusting for markedly abnormal Baseline values did not confirm an association of QT prolongation with cilansetron treatment. Two subjects in the cilansetron 2 mg TID group experienced a trial fibrillation and one additional subject had a SAE of complete atrioventricular block and syncope unrelated to study medication. Electrocardiogram data are not frequent enough to draw any conclusions. The ECG data will be investigated more comprehensively with other clinical studies evaluating safety in the integrated data. Analyses of vital sign and physical findings did not reveal any clinically relevant effect of cilansetron.

Study S241.3.011 was a Phase III, double-blind, placebo-controlled, randomized, multinational study to investigate the safety and efficacy of 2 mg TID of cilansetron for a period of 12 weeks followed by a rerandomized, placebo-controlled four-week treatment period, in diarrhea-predominant IBS subjects. The study was conducted in six countries including the US. A total of 2115 subjects consented to participate, and 770 subjects were randomized. A total of 746 subjects were analyzed for efficacy and 755 subjects were analyzed for safety. Diagnosis and main criteria for inclusion were IBS subjects satisfying the Rome I criteria (1992); the diarrhea predominant sub-population of the Rome II criteria; and a minimum abdominal pain/discomfort, stool frequency and consistency determined during the run-in period. Subjects were treated with cilansetron 2 mg tablets given orally TID or matching placebo tablets given orally TID for 12 weeks followed by a rerandomized, placebo-controlled four-week treatment period.

The primary efficacy parameter was the number of months that a subject was a responder for IBS symptoms (abdominal pain, abdominal discomfort, abnormal bowel habits) over the 12-week treatment period. A responder for a month (monthly responder) was defined as a subject who answered the weekly question with “yes” at least twice for that month. The two main secondary efficacy parameters were the monthly responder rates based on the “adequate relief” questions regarding abdominal pain/discomfort and abnormal bowel habits during the 12-week treatment period. The overall response rate and responder rate for the weekly diary questions on adequate relief at each month and all months, the adequate relief rate, the number of subjects with adequate relief by week, daily diary questions, IBS-QOL scores, and the interruption of activities were also assessed. Safety was evaluated by physical examination, ECG, vital signs, laboratory evaluations, concomitant medications, and AEs.

Among other findings (discussed more fully below), it was determined in this particular study that: (i) strong evidence for the superiority of cilansetron 2 mg TID over placebo was provided in this study for both male and female subjects over three months of treatment. Treatment in favor of cilansetron 2 mg TID over placebo were seen within the first week of treatment and the effect remained stable throughout the three-month treatment period. There was an immediate (within a week) loss of the treatment effect following blinded withdrawal of cilansetron 2 mg TID with no evidence of rebound within four weeks. Withdrawal of cilansetron treatment also led to an unfavorable effect on the subjects' quality of life; (ii) the evidence for the superiority of cilansetron 2 mg TID over placebo was established by the primary Efficacy analysis, number of months a subject was a responder for the weekly diary question on adequate relief of EBS symptoms, as reflected by the proportion of subjects who were responders for zero, one, two, or three months during the three-month treatment period (28%, 14%, 19%, and 39% in the cilansetron 2 mg TID group versus 54%, 13%, 15%, and 18% in the placebo group, respectively, p<0.001); (iii) statistically significant differences were demonstrated in favor of cilansetron compared with placebo by the main secondary Efficacy analyses during the three-month treatment period, as reflected by the propotion of subjects who were responders for zero, one, two, or three months for the weekly diary questions on adequate relief of abdominal pain/discomfort (27%, 12%, 19%, and 42% in the cilansetron 2 mg TID group versus 44%, 15%, 16%, and 25% in the placebo group, respectively, p<0.001) and abnormal bowel habits (30%, 13%, 18%, and 40% in the cilansetron 2 mg TID group versus 55%, 12%, 17%, and 17% in the placebo group, respectively, p<0.001); (iv) the superiority of cilansetron 2 mg TID versus placebo was also demonstrated by the statistically significant treatment differences in the overall responder rate for the weekly diary questions on adequate relief of symptoms, i.e., significantly more subjects were responders in the cilansetron 2 mg TID group compared with the placebo group during the three-month treatment period (observed case analysis on the ITT population: IBS symptoms, 55%. versus 29%, p<0.001; abdominal pain/discomfort, 59% versus 37%, p<0.001 ; and abnormal bowel habits, 54% versus 28%, p<0.001); (v) as would be predicted from the strong evidence for the efficacy of cilansetron 2 mg TID according to the primary and main secondary efficacy analyses (number of months that a subject was a responder) and the overall responder rate, all other analyses of the responses to the weekly diary questions on adequate relief, i.e., responder rate at each month, adequate relief rate, and number of subjects with adequate relief by week, showed similar superiority of cilansetron treatment over placebo. The proportion of subjects with adequate relief was greater for cilansetron 2 mg TID versus placebo already at Week 1, increased until Week 3, and remained relatively stable to the end of the three-month treatment period. Statistically significant treatment group difference in favor of cilansetron was already seen in responder rate for the first month of treatment and the favorable effect of cilansetron was sustained for the three-month treatment period; (vi) analysis of IBS symptoms recorded in the daily diary showed a statistically significant beneficial-effect of cilansetron 2 mg TID on the symptoms of abdominal pain/discomfort, urgency, and bloating or feeling of abdominal distension. Cilansetron treatment was also associated with statistically significant increased stool firmness, decreased stool frequency, and increased straining. Cilansetron had no effect on the feeling of incomplete evacuatio; (vii) the cilansetron 2 mg TID group demonstrated a statistically significant greater improvement compared with the placebo group in the IBS-QOL total score and each subscore (interference with activity, body image, health worry, food avoidance, social reaction, sexual, relationships, and dysphoria). Analysis of responses to the interruption of activities assessment demonstrated a clinically meaningful greater improvement in the cilansetron 2 mg TID group compared with the placebo group in the subjects' ability to effectively perform their everyday activities; (ix) all subgroups of age category, race, BMI category, severity of IBS, duration of IBS, and tobacco use showed similar responses to cilansetron treatment compared to the overall ITF population. A statistically significant treatment by gender interaction was detected for the primary and main secondary Efficacy variables and for the overall responder rate. Although the treatment differences were larger in male subjects compared with female subjects for these variables, they reached statistical significance in both male and female subjects; (x) data collected during the rerandomization period provided evidence that the beneficial effects of cilansetron 2 mg TID was maintained during the fourth month of treatment as reflected by the number of months a subjects was a responder and the overall responder rate for the weekly diary questions in the C/C group compared with the P/P group. There was an observed improvement with respect to the percentage of subjects with adequate relief of IBS symptoms for placebo subjects who transitioned to cilansetron versus placebo subjects who continued on placebo. The effect appeared within one week after rerandomization, and was sustained throughout the four-week period. Following blinded withdrawal of cilansetron 2 mg TID, almost half of the responders at Month 3 shifted to non-responders by Month 4. There was an immediate (within a week) loss of the treatment effect following blinded withdrawal of cilansetron 2 mg TID with no evidence of rebound within four weeks as reflected by the proportion of subjects with adequate relief for the weekly diary questions and confirmed by the daily diary assessments. Withdrawal of cilansetron treatment also led to an unfavorable effect on the subjects' quality of life and their ability to effectively perform their everyday activities.

Safety

Cilansetron 2 mg TID was safe and well tolerated during-this 16-week trial. A total of 263 of 380 subjects (69%) versus 243 of 375 subjects (65%) in the cilansetron 2 mg TID and placebo groups, respectively, experienced at least one TEAE during the three-month treatment period. During the four-week rerandomization period, the incidence of rerandomization-emergent AEs was higher in the P/C treatment group (44%) compared with the C/C (35%), C/P (34%), or P/P (36%) treatment groups. The incidence of at least one TEAE during the entire four-month treatment period was 78% versus 75% in the C/C and P/P treatment groups , respectively. The most frequently reported TEAEs during the three-month treatment period with a higher incidence in the cilansetron 2 mg TID group compared with the placebo group were GI disorders including constipation (20% versus 7%) and abdominal distension (5% versus 3%). Other common events possibly associated with cilansetron treatment were back pain, upper respiratory tract infections, nausea and vomiting symptoms, and headache.

Female subjects had a higher incidence of constipation in both the cilansetron 2 mg TID and placebo groups (26% versus 8%) compared with male subjects (8% versus 4%) during the three-month treatment period. Back pain and abdominal pain NOS appeared to be associated with cilansetron treatment only in f d e subjects but not in male subjects. The treatment group difference for constipation increased with increasing age and trends for similar type of interaction (i.e., larger treatment group differences with older age) were seen for back pain, upper respiratory tract infections, and diarrhea. The opposite trend was seen for flatulence. Other TEAEs in the GI system organ class with a small, not clinically relevant treatment group difference included abdominal pain NOS (6% versus 5%), flatulence (4% versus 3%), and abdominal pain upper (2% versus 1%) during the three-month treatment period. Twenty-five subjects (7%) in the cilansetron 2 mg TID group versus 24 subjects (6%) in the placebo group experienced fecal occult blood positive and/or blood in stool. One subject in the cilansetron 2 mg TID group with a history of mild colitis experienced the TEAE of colitis NOS. Clinic notes and a forrnal letter of confirmation from the principle investigator confirm that this event was not colitis, but an exacerbation of IBS symptoms. No cases of fecal impaction, obstruction, perforation, mega rectum, or colectomy were reported. No subject in either treatment group reported epilepsy or seizures/convulsions during the four months of this study. During the three-month treatment period, two subjects in the cilansetron 2 mg TID group and one subject in the placebo group experienced syncope and one additional subject in the cilansetron 2 mg TID group experienced an SAE of syncope aggravated. During the four-week rerandomization period, one subject in the C/C group reported syncope and one subject in the P/P group reported loss of consciousness. One subject in the cilansetron 2 mg TID group and no subjects in the placebo group experienced thrombosis (superficial thrombosis of right forearm). No cases of deep vein thrombosis were reported in this study. One subject in the cilansetron 2 mg TID group died in this study as a result of a road traffic accident during the three-month treatment period. Serious AEs were reported for 2% (eight of 380 subjects, cilansetron 2 mg TID) versus 4% (three of 375 subjects, placebo) during the three-month treatment period. Eleven percent versus 9% of subjects experienced TEAEs that led to permanent discontinuation of study medication in the cilansetron 2 mg TID and placebo groups, respectively. The most common TEAE that led to discontinuation was constipation, which had a higher incidence in the cilansetron 2 mg TID group compared with the placebo group. Analyses of laboratory data, vital signs, ECG, and physical findings did not reveal any clinically relevant effect of cilansetron.

Comparison and Analyses of Results Across Studies

This section summarizes all efficacy data collected in the Phase III pivotal Efficacy Studies S241.3.006, S24 1.3.009, and S241.3.011. Efficacy data from the Phase II dose response Studies S241.2.112 and S241.2.113 are presented below in support of the dosing recommendation.

Studies S241.3.006, S241.3.009, and S241.3.011 were very similar in design. All three pivotal efficacy studies were double-blind, placebo-controlled, randomized, multicenter, multinational (except S241.3.006), parallel-group studies to investigate the safety and efficacy of cilansetron 2 mg TID in diarrhea-predominant IBS subjects over 12 weeks (S241.3.006 and S241.3.011) or 26 weeks (S24 1.3.009) followed by a four-week rerandomized treatment period (S24 1.3.011). These three studies had essentially identical inclusion/exclusion criteria, procedures, and assessments. The important differences between the three studies included the following:
Study S241.3.006 was conducted at 129 study centers in the United States. Study S241.3.009 was conducted at 146 study centers in 19 countries outside the United States with the following 18 countries that randomized subjects: Australia, Belgium, Bulgaria, Canada, Denmark, France, Germany, India, Ireland, Israel, Netherlands, New Zealand, Poland, Romania, Russia, South Africa, Spain, and Ukraine. Study S241.3.011 was conducted at 202 study sites in six countries (Argentina, Brazil, Chile, Columbia, Peru, and the United States) with most subjects randomized in the United States (83%, 640 of 770 randomized subjects).
The duration of treatment was 12 weeks in Study S241.3.006, 26 weeks in Study S241.3.009, and a total of 16 weeks in Study S241.3.011 (12 weeks treatment followed by a four-week rerandomized treatment period).
The primary efficacy parameter in Studies S241.3.006 and S241.3.011 was changed prior to study database lock and unblinding by a protocol amendment (based on discussions with the FDA). The new primary efficacy parameter in Studies S241.3.006 and S241.3.011 corresponds to a key secondary efficacy parameter in Study S241.3.009 (for details, see Section 2.7.3.3.2).

Efficacy data from the three pivotal efficacy studies were not integrated. Integration was not performed because the results were consistent across the three pivotal efficacy studies. All presentations herein are based on analyses for the individual studies and supported by the summary tables, figures, and listings.

All analyses presented in this section have been conducted according to the Statistical Analysis Plans written for the individual protocols. For Study S241.3.009, the Statistical Analysis Plan included additional (secondary) efficacy analyses using only the first 12 of 26 weeks of treatment to allow comparison of the S241.3.009 data with the S241.3.006 and S241.3.011 data.

All analyses presented in this section were carried out using the ITT population. The ITT population consisted of all subjects who were randomized, took at least one dose of study medication, and answered the weekly question about IBS symptoms at least once after randomization. Additional analyses were performed for the weekly diary questions using the per-protocol population (ITT subjects with no major protocol deviations). Any differences in the results between the per-protocol population and the ITT population were noted in the text.

Study Populations

Summary of Study Populations Across Studies

Of all subjects who signed an informed consent, 34% (711 of 2080, Study S241.3.006), 36% (770 of 2115, Study S241 AO11), and 62% (806 of 1307, Study S241.3.009) subjects were randomized in the three pivotal efficacy studies to either cilansetron 2 mg TE) or placebo. Study S24 1.3-006 was conducted at 129 study centers in the United States. Study S241.3.011 was conducted at 202 study sites in six countries (Argentina, Brazil, Chile, Columbia, Peru, and the United States) with most subjects randomized in the United States (83%, 640 of 770 randomized subjects). Study S241.3 .009 was conducted at 146 study centers in 19 countries outside the United States with the following 18 countries that randomized subjects: Australia, Belgium, Bulgaria, Canada, Denmark, France, Germany, India, Ireland, Israel, Netherlands, New Zealand, Poland, Romania, Russia, South Africa, Spain, and Ukraine.

The withdrawal rates in the cilansetron 2 mg TED and placebo groups were 20% versus 15% (Study S241.3.006, period of three months), 23% versus 24% (Study S24 1.3.011, period of three months), and 21% versus 23% (Study S241.3.009, period of six months), respectively. The most common reasons for withdrawal were AEs and lack of Efficacy with the cilansetron 2 mg TID group typically having slightly higher incidences for AEs and slightly lower or almost equal incidences.for lack of efficacy compared with the placebo group. In Study S24 1.3.0 1.1, 576 of 770 randomized subjects were rerandomized to either cilansetron 2 mg TID or, placebo at the end of the three-month treatment period and 93% to 99% of rerandomized subjects completed the additional four weeks of rerandomized treatment.

Of all randomized subjects, 97% to 98% across the studies were included in the ITT population (randomized subjects who took at least one dose of study medication and answered the weekly question about IBS symptoms at least once after randomization) and 64% to 75% were included in the per-protocol population (ITT subjects with no major protocol deviations). Analysis populations are summarized across studies in Table 2.7.3:3.

TABLE 2.7.3: 3
Analysis Populations Across Studies (All Subjects Randomized)

| | | Study S241.3.006 | | Study S241.3.011 | | Study S241.3.009 | |
|---|---|---|---|---|---|---|---|
| | | Months 1 to 3 | | Months 1 to 3 | | Months 1 to 6 | |
| | | Cilansetron | | Cilansetron | | Cilansetron | |
| Parameter | Statistic | 2 mg TID | Placebo | 2 mg TID | Placebo | 2 mg TID | Placebo |
| Number of subjects randomized | N | 352 | 359 | 388 | 382 | 401 | 405 |
| Number of subjects in the safety population | n (%) | 349 (>99) | 352 (98) | 380 (98) | 375 (98) | 397 (>99) | 402 (>99) |
| Reasons excluded from the safety population | | | | | | | |
| Did not take study medication | n (%) | 1 (<1) | 5 (1) | 5 (1) | 4 (1) | 3 (<1) | 3 (<1) |
| No post-Baseline safety evaluation | n (%) | 2 (<1) | 4 (1) | 7 (2) | 6 (2) | 4 (<1) | 2 (<1) |
| Number of subjects in the ITT population | n (%) | 344 (98) | 348 (97) | 377 (97) | 369 (97) | 395 (99) | 397 (98) |
| Reasons excluded from the ITT population | | | | | | | |
| Did not take study medication | n (%) | 1 (<1) | 5 (1) | 5 (1) | 4 (1) | 3 (<1) | 3 (<1) |
| No post-Baseline response on weekly question | n (%) | 8 (2) | 11 (3) | 11 (3) | 12 (3) | 5 (1) | 8 (2) |
| on adequate relief of IBS symptoms | | | | | | | |
| Number of subjects in the per-protocol population | n (%) | 227 (64) | 259 (72) | 253 (65) | 236 (62) | 300 (75) | 303 (75) |
| Reasons excluded from the | | | | | | | |
| per-protocol population | | | | | | | |
| Not in the ITT population | n (%) | 8 (2) | 11 (3) | 11 (3) | 13 (3) | 6 (1) | 8 (2) |
| Major protocol deviations | n (%) | 122 (35) | 97 (27) | 130 (34) | 144 (38) | 98 (24) | 100 (25) |

Note:
Percentages are based on the number of subjects randomized.
Note:
Subjects may be counted more than once across reasons for exclusion from each population.
Note:
Subject 3011-1115601 was randomized to cilansetron 2 mg TID for the three-month treatment period but received placebo. Subject 3009-583016 was randomized to cilansetron 2 mg TID but received placebo. These two subjects are summarized under the placebo column.
Data source: Study S241.3.006: CSR Table 10.1.1.3.1 Left. Vol. 9 Righ; Study S241.3.011: CSR Table 10.1.1.3.1 Left. Vol. 9 Righ; Study S241.3.009: CSR Table 10.1.1.3.1 Left. Vol. 9 Righ

The treatment groups were relatively well balanced for the demographic and Baseline characteristics in each study except for the slightly higher proportion of male subjects in the cilansetron 2 mg group (47%) compared with the placebo group (43%) in Study S241.3.009. There were some differences in demographic and other characteristics across the three pivotal efficacy studies:
There was a lower proportion of male subjects in Study S241.3.006 (29% versus 30% in the cilansetron 2 mg TID and placebo groups, respectively) and Study S241.3.011 (31% versus 33%, respectively) compared with Study S241.3.009 (47% versus 43%, respectively).
A slightly higher proportion of subjects were 65 years or older in Study S241.3.006 (13% in both groups) and Study S241.3.0 1 1 (10% versus 13%) compared with Study S241.3.009 6% versus 8%). Correspondingly, the mean age was slightly higher in Study S241.3.006 (48.1 years versus 49.3 years) and Study S241.3.011 (48.6 years versus 48.3 years) compared with Study S24 1.3. 009 (44.6 years versus 44.5 years).
The proportion of Caucasian subjects was 94% in Study 5241.3.006, 86% in Study S241.3.011 (with 11% of subjects in the “Other” category), and 90% in Study S241.3.009 (with 10% in the Asian category).
The proportion of subjects with current use of tobacco products was 21% in Study S241.3 .006, 20% in Study S241.3.011, and 24% in Study S2413.009.
A higher proportion of subjects had a BMI of 30 kg/m2 or greater in Study S241.3.006 (33% versus 36%) and Study S24 1.3.0 1 1 (29% versus 35%) compared with Study S241.3 .009 (16% versus 15%). Correspondingly, the mean BMI was slightly higher in Study S241.3.006 (27.79 kg/m2 versus 27.65 kg/m2) and Study S24 1 .3.0 1 1 (27.71 kg/m2 versus 28.09 kg/m2) compared with Study S241 (25.36 kg/m2 versus 24.90 kg/m2).
Based on the interruption of activities assessment, a higher proportion of subjects had severe IBS in Study S241.3.006 (57% versus 56%) compared with Study S241.3.011 (41% versus 42%) or Study S241.3.009 (3 8% versus 39%).
The longest duration of IBS was observed among subjects enrolled in Study S241.3.006 (15 1.7 months versus 152.7 months) followed by Study S241.3.011 (119.7 months versus 121.8 months) and Study S241.3.009 (88 months versus 86 months).
Based on the Rome II criteria, both the diarrhea-related and constipation-related symptoms during the six months prior to enrollment were more commonly reported in general in Studies S241.3 .006 and S24 1.3.0 1 1 compared with Study S241.3.009.
More subjects took concomitant medications in Study S241.006 (96% versus 93%, period of three months) and Study S241.3.011 (86% versus 88%, period of three months) compared with Study S241.3.009 (65% versus 61%, period of six months).

The mean overall compliance was slightly lower in Study S241.3.006 (92.64% versus 93.79%) and Study S241.3.011 (91.64% versus 92.26%) compared with Study S241.3.009 (98.71% versus 97.66%).

Disposition of Subiects

Study S241.3.006

Subject disposition-overall is displayed in Table 2.7.3:4.

TABLE 2.7.3:4
Subject Disposition
Study S241.3.006 (All Subjects Enrolled)

| | | Cilansetron | | |
|---|---|---|---|---|
| Parameter | Statistic | 2 mg TID | Placebo | Overall |
| Number of subjects enrolled | n | | | 2080 |
| Number of subjects who entered diary run-in period^{a} | n (%) | | | 1367 (66) |
| Number of subjects in diary run-in period not randomized^{a} | n (%) | | | 656 (32) |
| Number of subjects randomized^{a} | n (%) | 352 | 359 | 711 (34) |
| Number of subjects who completed the study | n (%) | 283 (80) | 304 (85) | 587 (83) |
| Number of subjects who withdrew from the study | n (%) | 69 (20) | 55 (15) | 124 (17) |
| Primary reason for withdrawal: | | | | |
| Adverse event and lack of efficacy | n (%) | 30 (9) | 19 (5) | 49 (7) |
| Baseline complaint/adverse event | n (%) | 12 (3) | 4 (1) | 16 (2) |
| Lack of efficacy | n (%) | 5 (1) | 13 (4) | 18 (3) |
| Protocol violation | n (%) | 9 (3) | 12 (3) | 21 (3) |
| Administrative reason | n (%) | 2 (1) | 0 | 2 (<1) |
| Other | n (%) | 11 (3) | 7 (2) | 18 (3) |
| Number of subjects who died | n (%) | 0 | 0 | 0 |

^{a}Percentages are based on the number of subjects enrolled.
Note:
All subjects who signed an informed consent are considered as enrolled.
Note:
Percentages are based on the number of subjects randomized unless otherwise specified.
Note:
Two subjects in the placebo group (3006-609008 and 3006-691003) withdrew from the study due to AEs that were not treatment-emergent; therefore, these subjects and events are not included in the TEAE table summarizing discontinuation due to AE.
Data source: CSR Table 10.1.1.1.1 Left. Vol. 9 Righ

Of the 2080 subjects who were enrolled (i.e., signed an informed consent), 1367 subjects entered the diary run-in period and 711 subjects were randomized: 352 subjects to cilansetron 2 mg TID and 359 subjects to placebo. Of the 711 subjects randomized, a higher percentage of subjects in the cilansetron 2 mg TID group (20%) did not complete the study compared to the placebo group (15%). The most common primary reasons for withdrawal were AEs (combined categories of “AE and lack of Efficacy” and “Baseline complaint/AE, 12% versus 6%) and lack of efficacy (combined categories of “lack of efficacy” and “AE and lack of efficacy”, 10% versus 9% in the cilansetron 2 mg TIED and placebo groups, respectively) followed by the categories of “other” (3% versus 2%), and protocol violation (3% in both groups). Most subjects in the category “other” withdrew consent. Except for the higher percent of subjects in the cilansetron group (12%) compared with the placebo group (6%) with AEs as primary reason for withdrawal, no important treatment group differences were observed for reasons for withdrawal.

Study S241.3.011

Three-Month Treatment Period

Subject disposition overall is displayed in Table 2.7.35

TABLE 2.7.3:5
Subject Disposition for the Three-Month Treatment Period
Study S241.3.011 (All Subjects Enrolled)

| | | Cilansetron | | |
|---|---|---|---|---|
| Parameter | Statistic | 2 mg TID | Placebo | Overall |
| Number of subjects enrolled | n | | | 2115 |
| Number of subjects who entered diary run-in period^{a} | n (%) | | | 1408 (67) |
| Number of subjects in diary run-in period not randomized^{a} | n (%) | | | 638 (30) |
| Number of subjects randomized^{a} | n (%) | 388 | 382 | 770 (36) |
| Number of subjects who completed the three-month treatment | n (%) | 299 (77) | 292 (76) | 591 (77) |
| period | | | | |
| Number of subjects who withdrew from the study during the | n (%) | 89 (23) | 90 (24) | 179 (23) |
| three-month treatment period | | | | |
| Primary reason for withdrawal: | | | | |
| Adverse event and lack of efficacy | n (%) | 25 (6) | 25 (7) | 50 (6) |
| Baseline complaint/adverse event | n (%) | 16 (4) | 7 (2) | 23 (3) |
| Lack of efficacy | n (%) | 9 (2) | 26 (7) | 35 (5) |
| Protocol violation | n (%) | 14 (4) | 12 (3) | 26 (3) |
| Administrative reason | n (%) | 1 (<1) | 0 | 1 (<1) |
| Other | n (%) | 24 (6) | 20 (5) | 44 (6) |
| Number of subjects who died during the three-month treatment | n (%) | 1 (<1) | 0 | 1 (<1) |
| period | | | | |
| Number of subjects rerandomized | n (%) | 290 (75) | 286 (75) | 576 (75) |
| ^{a}Percentages are based on the number of subjects enrolled. All subjects who signed an informed consent are considered as enrolled. | | | | |
| Note: | | | | |
| Percentages are based on the number of subjects randomized unless otherwise specified. | | | | |
| Note: Subject 3011-1108210 in the placebo group and Subject 3011-1108217 in the cilansetron group withdrew due to AEs that started on the date the subjects started rerandomized study medication. As a result, these subjects are summarized in the TEAE tables under the Discontinuation Due to AE row for both the three-month and four-week rerandomization period tables. However, these subjects are summarized in the Completed row in this table because they were rerandomized | | | | |
| # and took rerandomized study medication. | | | | |

Note:
Subject 3011-1121205 is summarized in the Completed row in the cilansetron group because the subject was on study medication for at least 85 days. However, this subject is summarized in the TEAE table as having discontinued due to an AE during the three-month treatment period since the subject was not rerandomized and discontinued due to an AE.
Note:
Subject 3011-1102216 in the placebo group was randomized and discontinued the study due to a Baseline complaint. This subject did not take study medication and, therefore, is not included in the TEAE table summarizing discontinuation due to AE.
Note:
Subject 3011-1115601 was randomized to cilansetron for the three-month treatment period but received placebo. This subject is summarized in the placebo treatment group in all summaries for this study.
Data source: CSR Table 10.1.1.1.1 Left. Vol. 9 Righ

Of the 2115 subjects who were enrolled (i.e., signed an informed consent), 1408 subjects entered the diary run-in period and 770 subjects were randomized: 388 subjects to cilansetron 2 mg TID and 382 subjects to placebo. Of the 770 subjects randomized, a similar percentage of subjects in the cilansetron 2 mg TID group (23%) and the placebo group (24%) did not complete the three-month treatment period. The most common primary reasons for withdrawal were lack of efficacy (combined categories of “lack of efficacy” and “AE and lack of efficacy”, 9% versus 13% in the cilansetron 2 mg TID and placebo groups, respectively) and AEs (combined categories of “AE and lack of efficacy” and “Baseline complaint/AE”, 11% versus 8%) followed by the categories of “other” (6% versus 5%), protocol violation (4% versus 3%), and administrative (<1% versus 0%, respectively). Most subjects in the category “other” withdrew consent. Except for withdrawal due to lack of Efficacy (9% versus 13%) and AEs (1 1%-versus 8%), no important treatment group differences were observed for reasons for withdrawal. One subject in the cilansetron 2 mg TID group died (road traffic accident) during the three-month treatment period.

Of the 770 subjects randomized, 75% in both treatment groups (290 subjects in the cilansetron 2 mg TID group and 286 subjects to placebo) were rerandomized.

Four-Week Rerandomization Period

Subjects who were initially randomized to the cilansetron 2 mg TID group and rerandomized to placebo, will be referred to as the C/P treatment group; subjects initially randomized to the placebo group and rerandomized to placebo will be referred to as the P/P treatment group; the P/C and C/C treatment group nomenclature follow the same principle. Of the 591 subjects who completed the three-month treatment period, 576 subjects were rerandomized and thus divided into the following treatment groups: 150 subjects in the C/C treatment group, 140 subjects in the C/P treatment group, 136 subjects in the P/C treatment group, and 150 subjects in the P/P treatment group. Of the 576 subjects rerandomized, a higher percentage of subjects in the C/C treatment group (99%) completed the study compared with the C/P (95%), P/C (93%), and the P/P (95%) treatment groups. The most common primary reasons for withdrawal were lack of efficacy (combined categories of “lack of Efficacy” and “AE and lack of efficacy”, (C/C treatment group: 1%; C/P treatment group: 3%; P/C treatment group: 5%; P/P treatment group: 1%) and AEs (combined categories of “AE and lack of efficacy” and “Baseline complaint/AE”, C/C treatment group: 0%; C/P treatment group: 2%; P/C treatment group: 4%; P/P treatment group: 1%) followed by the categories of “other”, protocol violation, and administrative. Except for the P/C treatment group having a higher percentage of withdrawal compared to the other treatment groups due to lack of efficacy and AEs, no treatment group differences were observed for reasons for withdrawal.

Study S241.3.009

Subject disposition overall is displayed in Table 2.7.3:6.

TABLE 2.7.3:6
Subject Disposition
Study S241.3.009 (All Subjects Enrolled)

| | | Cilansetron | | |
|---|---|---|---|---|
| Parameter | Statistic | 2 mg TID | Placebo | Overall |
| Number of subjects enrolled | n | | | 1307 |
| Number of subjects who entered diary run-in period^{a} | n (%) | | | 1117 (85) |
| Number of subjects in diary run-in period not randomized^{a} | n (%) | | | 311 (24) |
| Number of subjects randomized^{a} | n (%) | 401 | 405 | 806 (62) |
| Number of subjects who completed the study | n (%) | 316 (79) | 313 (77) | 629 (78) |
| Number of subjects who withdrew from the study | n (%) | 85 (21) | 92 (23) | 177 (22) |
| Primary reason for withdrawal: | | | | |
| Adverse event and lack of efficacy | n (%) | 19 (5) | 22 (5) | 41 (5) |
| Baseline complaint/adverse event | n (%) | 16 (4) | 7 (2) | 23 (3) |
| Lack of efficacy | n (%) | 22 (5) | 33 (8) | 55 (7) |
| Protocol violation | n (%) | 8 (2) | 11 (3) | 19 (2) |
| Administrative reason | n (%) | 1 (<1) | 0 | 1 (<1) |
| Other | n (%) | 19 (5) | 19 (5) | 38 (5) |
| Number of subjects who died | n (%) | 0 | 0 | 0 |

^{a}Percentages are based on the number of subjects enrolled.
Note:
All subjects who signed an informed consent are considered as enrolled.
Note:
Percentages are based on the number of subjects randomized unless otherwise specified.
Note:
Subject 3009-583016 was randomized to cilansetron 2 mg TID, but received placebo; therefore, this subject is summarized under the placebo column.
Data source: CSR Table 10.1.1.1.1 Left. Vol. 9 Righ

Of the 1307 subjects who were enrolled (i.e., signed an informed consent), 1117 subjects entered the diary run-in period and 806 subjects were randomized: 401 subjects to cilansetron 2 mg TID and 405 subjects to placebo. Of the 806 subjects randomized, a similar percentage of subjects in the cilansetron 2 mg TID group (21%) and the placebo group (23%) did not complete the study.

The most common primary reasons for withdrawal were lack of Efficacy (combined categories of “lack of efficacy” and “AE and lack of efficacy”, 10% versus 13% in the cilansetron 2 mg TID and placebo groups, respectively) and AEs (combined categories of “AE and lack of efficacy” and “Baseline complaint/AE”, 9% versus 7%) followed by the categories of “other” (5% in both groups), protocol violation (2% versus 3%), and administrative reason (<1% versus 0%, respectively). Most subjects in the category “other” withdrew consent. No important treatment group difference was observed for any of the reasons for withdrawal.

The overall withdrawal rate was relatively stable when examined by one-month intervals during the first three months of the study (6%, 6%, and 5%) and decreased from 16% during the first three-month interval to 6% during the second three-month interval. No meaningful treatment group differences were observed in the reasons for withdrawal during any of the time intervals. A total of 18 countries enrolled subjects in this study with seven countries enrolling at least 5% of the sub jects: Ukraine (18%), Russia (1-5%), Germany (13%), Canada (10%), India (9%), Romania (7%), and Bulgaria (6%). None of the individual study sites enrolled more than 3% of the subjects in either treatment group.

Data Sets Analyzed

Study S241.3.006

Analysis populations are summarized for all randomized subjects in Table 2.7.3:7.

TABLE 2.7.3:7
Analysis Populations
Study S241.3.006 (All Subjects Randomized)

| Parameter | Statistic | Cilansetron 2 mg TID | Placebo | Overall |
|---|---|---|---|---|
| Number of subjects randomized | N | 352 | 359 | 711 |
| Number of subjects in the safety population | n (%) | 349 (>99) | 352 (98) | 701 (99) |
| Reasons excluded from the safety population | | | | |
| Did not take study medication | n (%) | 1 (<1) | 5 (1) | 6 (<1) |
| No post-Baseline safety evaluation | n (%) | 2 (<1) | 4 (1) | 6 (<1) |
| Number of subjects in the ITT population | n (%) | 344 (98) | 348 (97) | 692 (97) |
| Reasons excluded from the ITT population | | | | |
| Did not take study medication | n (%) | 1 (<1) | 5 (1) | 6 (<1) |
| No post-Baseline response on weekly question on | n (%) | 8 (2) | 11 (3) | 19 (3) |
| adequate relief of IBS symptoms | | | | |
| Number of subjects in the per-protocol population | n (%) | 227 (64) | 259 (72) | 486 (68) |
| Reasons excluded from the per-protocol population | | | | |
| Not in the ITT population | n (%) | 8 (2) | 11 (3) | 19 (3) |
| Major protocol deviations | n (%) | 122 (35) | 97 (27) | 219 (31) |

Note:
Percentages are based on the number of subjects randomized.
Note:
Subjects may be counted more than once across reasons for exclusion from each population.
Data source: CSR Table 10.1.1.3.1 Left. Vol. 9 Righ

Of the 711 subjects randomized, 10 subjects (three subjects in the cilansetron 2 mg TID group versus seven subjects in the placebo group) were excluded from the safety population for not taking any study medication and/or lack of any post-Baseline safety data. Of the 711 subjects randomized, 19 subjects (eight subjects in the cilansetron 2 mg TID group versus 11 subjects in the placebo group) were excluded from the ITT population for not taking any study medication and/or lack of any post-Baseline data regarding the weekly question for adequate relief of IBS symptoms. An additional 206 subjects (117 subjects in the cilansetron 2 mg TID group versus 89 subjects in the placebo group) were excluded from the per-protocol population due to major protocol deviations.

Study S241.3.011

Three-Month Treatment Period

Analysis populations are summarized for all randomized subjects during the three-month treatment period in Table 2.7.3:8.

TABLE 2.7.3:8
Analysis Populations for the Three-Month Treatment Period
Study S241.3.011 (All Subjects Randomized)

| Parameter | Statistic | Cilansetron 2 mg TID | Placebo | Overall |
|---|---|---|---|---|
| Number of subjects randomized | N | 388 | 382 | 770 |
| Number of subjects in the safety population | n (%) | 380 (98) | 375 (98) | 755 (98) |
| Reasons excluded from the safety population | | | | |
| Did not take study medication | n (%) | 5 (1) | 4 (1) | 9 (1) |
| No post-Baseline safety evaluation | n (%) | 7 (2) | 6 (2) | 13 (2) |
| Number of subjects in the ITT population | n (%) | 377 (97) | 369 (97) | 746 (97) |
| Reasons excluded from the ITT population | | | | |
| Did not take study medication | n (%) | 5 (1) | 4 (1) | 9 (1) |
| No post-Baseline response on weekly question on | n (%) | 11 (3) | 12 (3) | 23 (3) |
| adequate relief of IBS symptoms | | | | |
| Number of subjects in the per-protocol population | n (%) | 253 (65) | 236 (62) | 489 (64) |
| Reasons excluded from the per-protocol population | | | | |
| Not in the ITT population | n (%) | 11 (3) | 13 (3) | 24 (3) |
| Major protocol deviation | n (%) | 130 (34) | 144 (38) | 274 (36) |

Note:
Percentages are based on the number of subjects randomized.
Note:
Subjects may be counted more than once across reasons for exclusion from each population.
Note:
Subject 3011-1115601_was randomized to cilansetron 2 mg TID for the three-month treatment period but received placebo. This subject is summarized in the placebo treatment group in all summaries for this study.
Data source: CSR Table 10.1.1.3.1 Left. Vol. 9 Righ

Of the 770 subjects randomized, 15 subjects (eight subjects, 2% in the cilansetron 2 mg TIED group versus seven subjects, 2% in the placebo group) were excluded from the safety population for not taking any study medication and/or lack of any post-Baseline safety data. Of the 770 subjects randomized, 24 subjects (11 subjects, 3% in the cilansetron 2 mg TID group versus 13 subjects, 3% in the placebo group) were excluded from the ITT population for not taking any study medication and/or lack of any post-Baseline data regarding the weekly question for adequate relief of IBS symptoms. An additional 257 subjects (124 subjects, 32% in the cilansetron 2 mg TID group versus 133 subjects, 35% in the placebo group) were excluded from the per-protocol population due to major protocol deviations.

Four-Week Rerandomization Period

Analysis populations are summarized for all rerandomized subjects in Table 2.7.3:9.

TABLE 2.7.3: 9
Analysis Populations for the Four-Week Rerandomization Period
Study S241.3.011 (All Subjects Rerandomized)

| Parameter | Statistic | C/C | C/P | P/C | P/P | Overall |
|---|---|---|---|---|---|---|
| Number of subjects rerandomized | N | 150 | 140 | 136 | 150 | 576 |
| Number of subjects in the rerandomized safety population | n (%) | 150 (100) | 138 (99) | 135 (>99) | 149 (>99) | 572 (>99) |
| Reasons excluded from the rerandomized safety population | | | | | | |
| Did not take study medication | n (%) | 0 | 0 | 1 (<1) | 1 (<1) | 2 (<1) |
| No post- rerandomization safety evaluation | n (%) | 0 | 2 (1) | 1 (<1) | 0 | 3 (<1) |
| Number of subjects in the rerandomized efficacy population | n (%) | 149 (>99) | 139 (>99) | 135 (>99) | 146 (97) | 569 (99) |
| Reasons excluded from the rerandomized efficacy population | | | | | | |
| Did not take study medication | n (%) | 0 | 0 | 1 (<1) | 1 (<1) | 2 (<1) |
| No post-rerandomization response on weekly question n (%) | 1 (<1) | 0 | 1 (<1) | 4 (3) | 6 (1) | |

Note:
C = cilansetron 2 mg TID; P = placebo.
Note:
Percentages are based on the number of subjects rerandomized.
Note:
Subject 3011-1116601 was rerandomized to placebo, but received cilansetron during the four-week rerandomization period. This subject is summarized under the P/C column.
Note:
Subject 3011-1103139 was dispensed rerandomized study medication but was not rerandomized via the Interactive Voice Response System (IVRS). This subject is included in the Rerandomized Safety Population but is excluded from the Rerandomized Efficacy Population because the subject was not rerandomized.
Note:
Subjects may be counted more than once across reasons for exclusion form each population.
Data source: CSR Table 10.1.1.11.2 Left. Vol. 9 Righ

Of the 576 subjects rerandomized, four subjects (two subjects in the C/P treatment group, one subject in the P/C treatment group, and one subject in the P/P treatment group) were excluded from the safety population for not taking any study medication and/or lack of any post-rerandomization safety data. Of the 576 subjects rerandomized, seven subjects. (one subject each in the C/C, C/P, and the P/C treatment groups, and four subjects in the P/P treatment group) were excluded from the rerandomized efficiacy population for not taking any study medication and/or lack of any post-rerandomization data regarding the weekly question for adequate relief of IBS symptoms.

Study S24 1.3.009

Analysis populations are summarized for all randomized subjects in Table 2.7.3:10.

TABLE 2.7.3:10
Analysis Populations
Study S241.3.009 (All Subjects Randomized)

| | | Cilansetron | | |
|---|---|---|---|---|
| Parameter | Statistic | 2 mg TID | Placebo | Overall |
| Number of subjects randomized | N | 401 | 405 | 806 |
| Number of subjects in the safety population | n (%) | 397 (>99) | 402 (>99) | 799 (>99) |
| Reasons excluded from the safety population | | | | |
| Did not take study medication | n (%) | 3 (<1) | 3 (<1) | 6 (<1) |
| No post-Baseline safety evaluation | n (%) | 4 (<1) | 2 (<1) | 6 (<1) |
| Number of subjects in the ITT population | n (%) | 395 (99) | 397 (98) | 792 (98) |
| Reasons excluded from the ITT population | | | | |
| Did not take study medication | n (%) | 3 (<1) | 3 (<1) | 6 (<1) |
| No post-Baseline response on weekly question on | n (%) | 5 (1) | 8 (2) | 13 (2) |
| adequate relief | | | | |
| Number of subjects in the per-protocol population | n (%) | 300 (75) | 303 (75) | 603 (75) |
| Reasons excluded from the per-protocol population | | | | |
| Not in the ITT population | n (%) | 6 (1) | 8 (2) | 14 (2) |
| Major protocol deviation | n (%) | 98 (24) | 100 (25) | 198 (25) |

Note:
Percentages are based on the number of subjects randomized.
Note:
Subject 3009-583016 was randomized to cilansetron 2 mg TID, but received placebo; therefore, this subject is summarized under the placebo column.
Note:
Subjects may be counted more than once across reasons for exclusion from each population.
Data source: CSR Table 10.1.1.3.1 Left. Vol. 9 Righ

Of the 806 subjects randomized, seven subjects (four subjects in the cilansetron 2 mg TID group versus three subjects in the placebo group) were excluded from the safety population for not taking any study medication and/or lack of any post-Baseline safety data. Of the 806 subjects randomized, 14 subjects (six subjects in the cilansetron 2 mg TID group versus eight subjects in the placebo group) were excluded from the ITT population for not taking any study medication and/or lack of any post-Baseline data regarding the weekly question for adequate relief of IBS symptoms. An additional 198 subjects (98 subjects [24%] in the cilansetron 2 mg TID group versus 100 subjects [25%] in the placebo group) were excluded from the per-protocol population due to major protocol deviations.

Demographics

Study S241.3.006

An overall summary of demographic data for the ITT population is displayed in Table 2.7.3:11.

TABLE 2.7.3:11
Subject Demographics
Study S241.3.006 (ITT Population)

| | | Cilansetron 2 mg TID | Placebo | Overall |
|---|---|---|---|---|
| Parameter | Statistic | (N = 344) | (N = 348) | (N = 692) |
| Age (years) | n | 344 | 348 | 692 |
| | Mean (S.E) | 48.1 (0.7) | 49.3 (0.7) | 48.7 (0.5) |
| | Median | 48 | 50 | 49 |
| | Min-Max | 18-86 | 18-79 | 18-86 |
| Age category (years) | n | 344 | 348 | 692 |
| 18-40 | n (%) | 106 (31) | 92 (26) | 198 (29) |
| 41-64 | n (%) | 194 (56) | 212 (61) | 406 (59) |
| ≧65 | n (%) | 44 (13) | 44 (13) | 88 (13) |
| Gender | n | 344 | 348 | 692 |
| Male | n (%) | 101 (29) | 104 (30) | 205 (30) |
| Female | n (%) | 243 (71) | 244 (70) | 487 (70) |
| Race | n | 344 | 348 | 692 |
| Caucasian | n (%) | 323 (94) | 330 (95) | 653 (94) |
| Black | n (%) | 5 (1) | 11 (3) | 16 (2) |
| Asian | n (%) | 5 (1) | 0 | 5 (<1) |
| Other | n (%) | 11 (3) | 7 (2) | 18 (3) |
| Current use of tobacco products | n | 344 | 348 | 692 |
| Yes | n (%) | 72 (21) | 76 (22) | 148 (21) |
| No | n (%) | 272 (79) | 272 (78) | 544 (79) |
| Prior laxative use | n | 344 | 348 | 692 |
| Yes | n (%) | 4 (1) | 6 (2) | 10 (1) |
| No | n (%) | 340 (99) | 342 (98) | 682 (99) |
| Laxative use | n | 344 | 348 | 692 |
| Never | n (%) | 340 (99) | 342 (98) | 682 (99) |
| Rarely (1 to 3 times) | n (%) | 4 (1) | 4 (1) | 8 (1) |
| Occasionally (4 to 6 times) | n (%) | 0 | 2 (<1) | 2 (<1) |
| Frequently (7 to 12 times) | n (%) | 0 | 0 | 0 |
| Regularly (>12 times) | n (%) | 0 | 0 | 0 |
| BMI (kg/m²) | n | 344 | 348 | 692 |
| | Mean (S.E) | 27.79 (0.27) | 27.65 (0.28) | 27.72 (0.19) |
| | Median | 27.4 | 27.1 | 27.3 |
| | Min-Max | 17.8-39.5 | 17.7-39.7 | 17.7-39.7 |
| BMI Category | n | 344 | 348 | 692 |
| <30 kg/m² | n (%) | 230 (67) | 224 (64) | 454 (66) |
| ≧30 kg/m² | n (%) | 114 (33) | 124 (36) | 238 (34) |

Note:
Percentages are based on the number of subjects in the ITT Population with a response for each assessment.
Data source: CSR Table 10.1.1.6.1 Left. Vol. 9 Righ and CSR Table 10.1.1.7.1 Left. Vol. 9 Righ

The age of the subjects ranged from 18 years to 86 years in the ITT population and the mean age was similar between the groups (48.1 years in the cilansetron 2 mg TID group and 49.3 years in the placebo group). Most subjects in the cilansetron 2 mg TID and placebo groups were in the 41 to 64 years age category (56% versus 61%) followed by the 18 to 40 years category (31% versus 26%), while 13% of subjects in both treatment groups were 65 years or older. There was a total of 30% male subjects (29% in cilansetron 2 mg TID group versus 30% in the placebo group). The racial composition was similar between the groups with 94% versus 95% of subjects in the Caucasian category and 3% or less of the subjects in the other categories. Twenty-one percent of the subjects were using tobacco products at Baseline and only 1% of subjects had a history of prior laxative use. Analysis of demographic data for the per-protocol population showed results consistent with the nIT analyses.

Analysis of demographic data by gender revealed younger overall age among male subjects (47.2 years) compared with female subjects (49.3 years). The treatment groups were well balanced within both subpups for the demographic variables except for small treatment group differences for age in both female and male subjects. Female subjects in the cilansetron 2 mg TID group were younger (48.1 years) compared with female subjects in the placebo group (50.5 years) and male subjects in the cilansetron 2 mg TID group were older (48.1 years) compared with male subjects in the placebo group (46.4 years). The treatment group differences for age in male and female subjects were not considered meaningful.

Study S241 1.3.01

Three-Month Treatment Period

An overall summary of demographic data for the ITT population is displayed in Table 2.7.3:12.

TABLE 2.7.3:12
Subject Demographics for the Three-Month Treatment Period
Study S241.3.011 (ITT Population)

| | | Cilansetron 2 mg TID | Placebo | Overall |
|---|---|---|---|---|
| Parameter | Statistic | (N = 377) | (N = 369) | (N = 746) |
| Age (years) | n | 377 | 369 | 746 |
| | Mean (S.E.) | 48.6 (0.6) | 48.3 (0.7) | 48.5 (0.5) |
| | Median | 49 | 48 | 49 |
| | Min-Max | 18-81 | 19-82 | 18-82 |
| Age category (years) | n | 377 | 369 | 746 |
| 18-40 | n (%) | 98 (26) | 109 (30) | 207 (28) |
| 41-64 | n (%) | 241 (64) | 213 (58) | 454 (61) |
| ≧65 | n (%) | 38 (10) | 47 (13) | 85 (11) |
| Gender | n | 377 | 369 | 746 |
| Male | n (%) | 115 (31) | 123 (33) | 238 (32) |
| Female | n (%) | 262 (69) | 246 (67) | 508 (68) |
| Race | n | 377 | 369 | 746 |
| Caucasian | n (%) | 321 (85) | 318 (86) | 639 (86) |
| Black | n (%) | 12 (3) | 8 (2) | 20 (3) |
| Asian | n (%) | 2 (<1) | 1 (<1) | 3 (<1) |
| Other | n (%) | 42 (11) | 42 (11) | 84 (11) |
| Current use of tobacco products | n | 377 | 368 | 745 |
| Yes | n (%) | 76 (20) | 76 (21) | 152 (20) |
| No | n (%) | 301 (80) | 292 (79) | 593 (80) |
| Prior laxative use | n | 377 | 369 | 746 |
| Yes | n (%) | 13 (3) | 10 (3) | 23 (3) |
| No | n (%) | 364 (97) | 359 (97) | 723 (97) |
| Laxative use | n | 377 | 369 | 746 |
| Never | n (%) | 364 (97) | 359 (97) | 723 (97) |
| Rarely (1 to 3 times) | n (%) | 7 (2) | 10 (3) | 17 (2) |
| Occasionally (4 to 6 times) | n (%) | 6 (2) | 0 | 6 (<1) |
| Frequently (7 to 12 times) | n (%) | 0 | 0 | 0 |
| Regularly (>12 times) | n (%) | 0 | 0 | 0 |
| BMI (kg/m²) | n | 377 | 368 | 745 |
| | Mean (S.E.) | 27.71 (0.26) | 28.09 (0.27) | 27.90 (0.19) |
| | Median | 27.3 | 27.6 | 27.3 |
| | Min-Max | 16.1-44.4 | 17.6-41.3 | 16.1-44.4 |
| BMI Category | n | 377 | 368 | 745 |
| <30 kg/m² | n (%) | 266 (71) | 241 (65) | 507 (68) |
| ≧30 kg/m² | n (%) | 111 (29) | 127 (35) | 238 (32) |

Note:
Percentages are based on the number of subjects in the ITT Population with a response for each assessment.
Data source: CSR Table 10.1.1.6.1 Left. Vol. 9 Righ and CSR Table 10.1.1.7.1 Left. Vol. 9 Righ

The age of the subjects ranged from 18 years to 82 years in the ITT population and the mean age was similar between the groups (48.6 years in the cilansetron 2 mg TID group and 48.3 years in the placebo group). Most subjects in the cilansetron 2 mg TID and placebo groups were in the 41 to 64 years age category (64% -versus 58%) followed by the 18 to 40 years category (26% versus 30%), while 10% versus 13% of subjects were 65 years or older. Approximately one third of subjects were male in both the cilansetron 2 mg TIED group (31%) and the placebo group (33%). The racial composition was similar between the groups with 85% versus 86% of subjects in the Caucasian category and 11% of the subjects in both groups in the category of other. Approximately one-fifth of the subjects were using tobacco products at Baseline and only 3% of subjects had a history of prior laxative use. Analysis of demographic data for the per-protocol population showed results consistent with the IllT analyses.

Analysis of demographic data by gender revealed younger overall age for male subjects (46.2 years) compared with female subjects (49.5 years). In addition, male subjects in the cilansetron 2 mg TID group were slightly older than male subjects in the placebo group (47.7 years versus 44.9 years). For all other demographic variables, the treatment groups were well balanced within both subgroups.

Four-Week Rerandomization Period

Analysis of demographic data for the rerandomized safety population showed results consistent with the ITT analyses. The age of the subjects ranged from 18 years to 82 years in the rerandomized safety population and the mean age was similar across the treatment groups (49.2 years in the C/C treatment group, 48.6 years in the C/P treatment group, 47.4 years in the P/C treatment group, and 48.9 years in the P/P treatment group). Most subjects were in the 41 to 64 years age category (55% to 70% across the groups) followed by the 18 to 40 years category (24% to 32%), while 7% to 13% of subjects were 65 years or older. Approximately one third of subjects (33% to 35% across the groups) were male. The racial composition was similar across the groups with most subjects (83% to 86%) in the Caucasian category followed by the category of other (9% to 15%). Approximately one-fifth of the subjects (17% to 22%) were using tobacco products at Baseline.

Study S241.3.009

An overall summary of demographic data for the ITT population is displayed in Table 2.7.3:13.

TABLE 2.7.3:13
Subject Demographics
Study S241.3.009 (ITT Population)

| | | Cilansetron 2 mg TID | Placebo | Overall |
|---|---|---|---|---|
| Parameter | Statistic | (N = 395) | (N = 397) | (N = 792) |
| Age (years) | n | 395 | 397 | 792 |
| | Mean (S.E) | 44.6 (0.7) | 44.5 (0.7) | 44.6 (0.5) |
| | Median | 45 | 45 | 45 |
| | Min-Max | 18-84 | 18-79 | 18-84 |
| Age category (years) | n | 395 | 397 | 792 |
| 18-40 | n (%) | 154 (39) | 158 (40) | 312 (39) |
| 41-64 | n (%) | 217 (55) | 206 (52) | 423 (53) |
| ≧65 | n (%) | 24 (6) | 33 (8) | 57 (7) |
| Gender | n | 395 | 397 | 792 |
| Male | n (%) | 186 (47) | 172 (43) | 358 (45) |
| Female | n (%) | 209 (53) | 225 (57) | 434 (55) |
| Race | n | 395 | 397 | 792 |
| Caucasian | n (%) | 358 (91) | 355 (89) | 713 (90) |
| Black | n (%) | 0 | 0 | 0 |
| Asian | n (%) | 36 (9) | 40 (10) | 76 (10) |
| Other | n (%) | 1 (<1) | 2 (<1) | 3 (<1) |
| Current use of tobacco products | n | 395 | 397 | 792 |
| Yes | n (%) | 98 (25) | 89 (22) | 187 (24) |
| No | n (%) | 297 (75) | 308 (78) | 605 (76) |
| Prior laxative use | n | 395 | 397 | 792 |
| Yes | n (%) | 5 (1) | 8 (2) | 13 (2) |
| No | n (%) | 390 (99) | 389 (98) | 779 (98) |
| Laxative use | n | 395 | 397 | 792 |
| Never | n (%) | 390 (99) | 389 (98) | 779 (98) |
| Rarely (1 to 3 times) | n (%) | 5 (1) | 6 (2) | 11 (1) |
| Occasionally (4 to 6 times) | n (%) | 0 | 2 (<1) | 2 (<1) |
| Frequently (7 to 12 times) | n (%) | 0 | 0 | 0 |
| Regularly (>12 times) | n (%) | 0 | 0 | 0 |
| BMI (kg/m²) | n | 395 | 396 | 791 |
| | Mean (S.E) | 25.36 (0.21) | 24.90 (0.22) | 25.13 (0.15) |
| | Median | 24.6 | 24.4 | 24.5 |
| | Min-Max | 18.0-38.7 | 17.0-39.3 | 17.0-39.3 |
| BMI Category | n | 395 | 396 | 791 |
| <30 kg/m² | n (%) | 330 (84) | 338 (85) | 668 (84) |
| ≧30 kg/m² | n (%) | 65 (16) | 58 (15) | 123 (16) |

Note:
Percentages are based on the number of subjects in the ITT Population with a response for each assessment.
Data source: CSR Table 10.1.1.6.1 Left. Vol. 9 Righ and CSR Table 10.1.1.7.1 Left. Vol. 9 Righ

The age of the subjects ranged from 18 years to 84 years in the ITT population and the mean age was similar between the groups (44.6 years in the cilansetron 2 mg TID group and 44.5 years in the placebo group). Most subjects in the cilansetron 2 mg TID and placebo groups were in the 41 to 64 years age category (55% versus 52%) followed by the 18 to 40 years category (39% versus 40%), while only 6% versus 8% of subjects were 65 years or older. The proportion of male subjects was slightly higher in the cilansetron 2 mg TIED group (47%) compared with the placebo group (43%). The racial composition was similar between the groups with 91% versus 89% of subjects in the Caucasian category and 9% versus 10% of the subjects in the Asian category. Approximately one-fourth of the subjects were using tobacco products at Baseline and only 2% of subjects had a history of prior laxative use. Analysis of demographic data for the Per-protocol population showed results consistent with the ITT analyses.

Analysis of demographic data by gender revealed younger overall age, lower proportion of Caucasian, and higher proportion of Asian subjects among male subjects compared with female subjects (age, 42.6 years versus 46.2 years; Caucasians, 82% versus 96%; and Asians, 17% versus 3%). The treatment groups were well balanced within both subgroups for the demographic variables.

Analysis of demographic data by country revealed some differences among the countries. For example, of the countries that enrolled at least 5% of the total subject population, the mean age was higher in Canada (47.7 years) and Germany (47.9 years) versus India (37.7 years); dominance of Asian race in India (100%) versus over 90% Caucasian in other countries; male dominance in Bulgaria (52%), India (82%) versus small proportion of males in Canada (20%). In addition, treatment group imbalances were observed within some of these countries for some of the demographic variables, for example, Romania, current use of tobacco products 29% (cilansetron 2 mg TID) versus 7% (placebo); Russia, male subjects 49% (cilansetron 2 mg TID) versus 29% (placebo); South Africa, male subjects 50% (cilansetron 2 mg TID) versus 29% (placebo). The clinical consequences to efficacy and how efficacy is modified due to the differences among the countries and the treatment group imbalances within the countries are difficult to assess.

Other Baseline Characteristics

Study S241.1006

Baseline characteristics of height, weight, BMI, and vital signs were assessed for the treatment group. A summary for BMI is displayed in Table 2.7.3:11. The mean height was 1.68 m in both groups, the mean weight was 78.9 kg (cilansetron 2 mg TID) versus 78.3 kg (placebo), and the mean BMI was 27.79 kg/m² (cilansetron 2 mg TID) versus 27.65 kg/m² (placebo) with 33% (cilansetron 2 mg TID) versus 36% (placebo) of subjects in the ≧30 kg/m² BMI category. No clinically relevant differences were detected between the groups for Baseline mean systolic (122.8 mmHg versus 122.1 mmHg) or diastolic (76.1 mmHg versus 75.8 mmHg) blood pressure, pulse rate (72.7 bpm versus 72.6 bpm), or body temperature (36.62° C. in both treatment groups). Per-protocol analyses showed results consistent with the ITT analyses.

Baseline assessment of organic or parasitic diseases were assessed for the treatment group. Approximately one-fifth of the subjects had the optional tests of stool parasites, ova, and culture performed at Baseline. No abnormal or positive results were obtained in either group for any of the tests. Of the 692 subjects in the ITT population, 690 subjects had a Baseline assessment of organic and parasitic diseases. Minor abnormalities, which did not exclude the subjects from participation in the study, were revealed by colonoscopy in 29% of subjects in both treatment groups prior to randomization and were hemorrhoids, diverticulosis, or polyps in most cases. Per-protocol analyses showed results consistent with the ITT analyses.

Baseline assessment of interruption of activities were assessed for the treatment group. Most subjects in the cilansetron 2 mg TID and placebo groups thought their IBS symptoms often (more than one quarter of the time, 25% versus 27%), very often (more than one half of the time, 27% versus 32%), or almost always (30% versus 24%) significantly interfered with their ability to effectively perform their activities. Based on the responses to the interruption of activities assessment, most subjects had IEBS of either moderate severity (responses of occasionally or often, 40% in both treatment groups) or severe (responses of very often or almost always, 57% versus 56%). Per-protocol analyses showed results consistent with the ITT analyses. Analyses by gender showed a slightly lower proportion of subjects with severe IBS at Baseline among male subjects (52%) compared with female subjects (59%). This difference was not considered meaningful.

IBS history and disease definition (Rome I criteria) were assessed for the treatment group. No clinically relevant differences were observed between the treatment groups for any of the Rome I criteria. The mean duration of IBS was 15 1.7 months and 152.7 months in the cilansetron 2 mg TID and placebo groups, respectively. Per-protocol analyses showed results consistent with the ITT analyses. No relevant gender-related overall differences were observed, however, among male subjects a longer duration of IBS was observed in the cilansetron 2 mg TID group (160.6 months) compared with the placebo group (146.4 months).

Diarrhea-predominant IBS subject eligibility (Rome I1 criteria) were assessed for the treatment group. No clinically relevant differences were observed between the treatment groups for any of the criteria. Most subjects in both treatment groups had the following diarrhea-related symptoms almost always or very often (more than half of the time) during the six months prior to enrollment: more than three bowel movements a day (70% versus 68%), loose, mushy, or watery bowel movements (87% versus 89%), and urgency (81% versus 79%). Some subjects also experienced the following constipation-related symptoms at least occasionally during the six months prior to enrollment: fewer than three bowel movements per week (3% in both groups), lumpy or hard bowel movements (21% versus 20%), and need to strain (15% versus 18%). Per-protocol analyses showed results consistent with the FIT analyses. No clinically relevant gender-related differences were observed except for urgency. Female subjects experienced urgency more often than male subjects did during the six months prior to enrollment (category of almost always, female subjects, 49% versus male subjects, 40%).

Study S241.3.011

Three-Month Treatment Period

Baseline characteristics of height, weight, BMI, and vital signs were assessed for the treatment group. A summary for BMI is displayed in Table 2.7.3:11. The mean height was 1.67 m in both groups, the mean weight was 77.6 kg (cilansetron 2 mg TID) versus 79.0 kg (placebo), and the mean BMI was 27.71 kg/m2 (cilansetron 2 mg TID) versus 28.09 kg/m2 (placebo) with 29% (cilansetron 2 mg TID) versus 35% (placebo) of subjects in the ≧30 kg/m2 BMI category. No clinically relevant differences were detected between the groups for Baseline mean systolic (122.8 mmHg versus 124.3 mmHg) or diastolic (75.9 mmHg versus 76.3 mmHg) blood pressure, pulse rate (73.6 bpm versus 73.9 bpm), or body temperature (36.56° C. versus 36.60° C.). Per-protocol analyses showed results consistent with the FIT analyses.

Baseline assessment of organic or parasitic diseases were assessed for the treatment group. Approximately one-fourth of the subjects had the optional tests of stool parasites, ova, and culture done at Baseline. Abnormal or positive results were obtained in 4% of subjects in either group for any of the tests. Of the 746 subjects in the ITT population, 723 subjects had colonoscopy done. Minor abnormalities were revealed in 18% versus 19% of subjects in the groups and were hemorrhoids, diverticulosis, or polyps in most cases. Per-protocol analyses showed results consistent with the nT analyses.

Baseline assessment of interruption of activities were assessed for the treatment group. Most subjects in the cilansetron,2 mg TIED and placebo groups thought their IBS symptoms often (more than one-quarter of the time, 34% versus 33%), very often (more than one half of the time, 24% versus 22%), or almost always (18% versus 19%) significantly interfered with their ability to effectively perform their activities. Based on the responses to the interruption of activities assessment, most subjects had IBS of either moderate severity (responses of occasionally or often, 51% versus 53%) or severe (responses of very often or almost always, 41% versus 42%). Per-protocol analyses showed results consistent with the YFF analyses. No gender-related differences were observed in the interruption of activities assessment. Analysis of Baseline severity of IBS by gender revealed an overall lower proportion of subjects with severe IBS among male subjects (33%) compared with female subjects (45%). No meaningfbl treatment group differences were noted within the subgroups.

IBS history and disease definition (Rome I criteria) were assessed for the treatment group. No clinically relevant differences were observed between the treatment groups for any of the Rome I criteria. The mean duration of IBS was 119.7 months versus 121.8 months in the cilansetron 2 mg TID and placebo groups, respectively. Per-protocol analyses showed results consistent with the nT analyses. Analysis of demographic data by gender showed shorter duration of IBS overall, among male subjects (106.2 months) compared with female subjects (127.6 months).

Diarrhea-predominant IBS subject eligibility (Rome II criteria) were assessed for the treatment group. No clinically relevant differences were observed between the treatment groups for any of the criteria. Most subjects in both treatment groups had the following diarrhea-related symptoms almost always or very often (more than half of the time) during the six months prior to enrollment: more than three bowel movements a day (67% versus 64%), loose, mushy, or watery bowel movements (84% versus 85%), and urgency (73% versus 76%). Some subjects also experienced the following constipation-related symptoms at least occasionally during the six months prior to enrollment: fewer than three bowel movements per week (3% versus 5%), lumpy or hard bowel movements (28% versus 27%), and need to strain (26% versus 24%). Per-protocol analyses showed results consistent with the ITT analyses. No clinically relevant gender-related differences were observed except for urgency. Female subjects experienced urgency more often than male subjects did during the six months prior to enrollment (category of almost always, female subjects, 43% versus male subjects, 25%).

Four-Week Rerandomization Period

Analysis of Baseline characteristics (height, weight, BMI, systolic and diastolic blood pressure, pulse rate, and body temperature) for a rerandomized safety population showed results consistent with the ITm analyses. The mean height was 1.7 m in all four treatment groups and the mean weight ranged from 77.4 kg to 79.5 kg across the treatment groups. The mean BMI varied from 27.3 kg/m² to 28.3 kg/m² with 25% (C/P) to 38% (P/C) of subjects in the ≧30 kg/m² BMI category. No clinically relevant differences were detected across the groups for Baseline mean systolic (ranging from 120.4 mmHg to 124.9 mmHg) or diastolic (ranging from 74.6 mmHg to 76.7 mmHg) blood pressure, pulse rate (ranging from 72.6 bpm to 73.5 bpm), or body temperature (ranging from 36.5° C. to 36.6° C.).

Analysis of Baseline characteristics for the rerandomized safety population showed that subjects in the rerandomized safety population had less severe IBS across all four treatment groups compared with subjects in the ITT population. Most subjects in the rerandomized safety population thought their IBS symptoms not at all or rarely (30% to 52% across the treatment groups) or occasionally (more than one-tenth of the time, 29% to 36%) significantly interfered with their ability to effectively perform their activities. Based on the responses to the interruption of activities assessment, most subjects had IBS of either mild or moderate severity in each treatment group. The proportion of subjects with severe JIBS was smaller among subjects who were on cilansetron 2 mg TID during the three-month treatment period (C/C, 7%; C/P, 6%) compared with subjects who were on placebo during the three-month treatment period (P/C, 18%; P/P, 18%). Correspondingly, the proportion of subjects with mild IBS was larger among subjects who were on cilansetron 2 mg TID during the three-month treatment period (C/C, 52%; C/P, 50%) compared with subjects who were on placebo during the three-month treatment period (P/C, 40%; P/P, 30%).

Study S241.3.009

Baseline characteristics of height, weight, BMI, and vital signs were assessed for the treatment group. A summary for BMI is displayed in Table 2.7.3:11. The mean height was 1.69 m in both groups, the mean weight was 72.7 kg (cilansetron 2 mg TID) versus 71.0 kg (placebo), and the mean BMI was 25.36 kg/m² (cilansetron 2 mg TID) versus 24.90 kg/m² (placebo) with 16% (cilansetron 2 mg TID) versus 15% (placebo) of subjects in the ≧30 kg/M² BMI category. No clinically relevant differences were detected between the groups for Baseline mean systolic (124.3 mmHg versus 123.8 mmHg) or diastolic (77.0 mmHg versus 76.8 mmHg) blood pressure, pulse rate (73.4 bpm versus 73.2 bpm), or body temperature (36.62° C. versus 36.63° C.). Per-protocol analyses showed results consistent with the ITT analyses except that the mean weight was slightly higher in the cilansetron 2 mg TID group (72.8 kg) compared with the placebo group (70.4 kg). Analysis by country revealed some differences among the countries. For example, of the countries that enrolled at least 5% of the total subject population, the mean BMI was higher in Canada (27.00 kg/m²) and lower in India (22.89 kg/r²) than in the overall population. In addition, treatment group imbalances were observed within some of these countries, for example, in Bulgaria, the mean BMI was higher in the cilansetron 2 mg TID group (26.18 kg/m²) compared with the placebo group (23.57 kg/m²). The clinical consequences to efficacy and how efficacy is modified due to the differences among the countries and the treatment group imbalances within the countries are difficult to assess.

Baseline assessment of organic or parasitic diseases was performed. Approximately half of the subjects had the optional tests of stool parasites, ova, and culture done at Baseline. Abnormal or positive results were obtained in <1% of subjects in either group for any of the tests. Of the 792 subjects in the ITT population, 702 subjects had colonoscopy done at Baseline. Minor abnormalities, which did not exclude the subjects from participation in the study, were revealed in 12% versus 11% of subjects in the groups and were hemorrhoids, diverticulosis, or polyps in most cases.

Baseline assessment of interruption of activities was performed for the treatment group. Most subjects in the cilansetron 2 mg TID and placebo groups thought their IBS symptoms often (more than one-quarter of the time, 40% versus 39%) or very often (more than one half of the time, 26% versus 26%) significantly interfered with their ability to effectively perform their activities. Based on the responses to the interruption of activities assessment, most subjects had IBS of either moderate severity (responses of occasionally or often, 57% versus 56%) or severe (responses of very often or almost always, 38% versus 39%). Per-protocol analyses showed results consistent with the ITT analyses. No gender-related differences were observed in the interruption of activities assessment or the Baseline severity of IBS. Analyses by country revealed some differences among the countries. For example, among the seven countries that enrolled at least 5% of the total population, severe IBS was more common overall in Germany (52%) than in Romania (21%). In addition, treatment group imbalances were observed within some of the countries for IBS severity, for example, the proportion of subjects with severe IBS was 38% (cilansetron 2 mg TED) versus 23% (placebo) in Bulgaria, 38% (cilansetron 2 mg TID) versus 54% (placebo) in Canada, 13% (cilansetron 2 mg TID) versus 28% (placebo) in Romania, and 32% (cilansetron 2 mg TED) versus 43% (placebo) in Russia. The clinical consequences to efficacy and how efficacy is modified due to the differences among the countries and the treatment group imbalances within the countries are difficult to assess.

IBS history and disease definition (Rome I criteria) was assessed for the treatment group. No clinically relevant differences were observed between the treatment groups for any of the Rome I criteria. The mean duration of EBS was 88 months and 86 months in the cilansetron 2 mg TID and placebo groups, respectively. Per-protocol analyses showed results consistent with the ITT analyses. No clinically relevant gender-related differences were observed. Analyses by country revealed some difference among the countries. For example, among the seven countries that enrolled at least 5% of the total population, longer overall duration of IBS was observed in Canada (131.9 months) and Germany (105.2 months) versus shorter duration of EBS in Bulgaria (67.3 months), Romania (57.1 months), Russia (69.5 months), and Ukraine (50.1 months). In addition, treatment group imbalances were observed within some of the countries for the duration of IBS, for example, in Romania (38.5 months versus 72.4 months in the cilansetron 2 mg TE) group compared with the placebo group) and Russia (81.0 months versus 58.2 months). The clinical consequences to efficacy and how efficacy is modified due to the differences among the countries and the treatment group imbalances within the countries are difficult to assess.

Diarrhea-predominant EBS subject eligibility (Rome II criteria) was assessed for the treatment group. No clinically relevant differences were observed between the treatment groups for any of the criteria. Most subjects in both treatment groups had the following diarrhea-related symptoms almost always or very often (more than half of the time) during the six months prior to enrollment: more than three bowel movements a day (55% versus 57%), loose, mushy, or watery bowel movements (68% versus 69%), and urgency (45% versus 42%). Some subjects also experienced the following constipation-related symptoms at least occasionally during the six months prior to enrollment: fewer than three bowel movements per week (3% in both groups), lumpy or hard bowel movements (11% versus 9%), and need to strain (18% in both groups). Per-protocol analyses showed results consistent with the ITT analyses. No clinically relevant gender-related differences were observed. In some of the countries (e.g., Canada, Germany), subjects appeared to be more diarrhea-predominant (i.e., more frequently experienced the diarrhea-related symptoms of more than three bowel movements per day, loose/mushy/watery stools, and urgency) compared with other countries. In addition, treatment group imbalances were also observed within some of these countries. The clinical consequences to efficacy and how efficacy is modified due to the differences among the countries and the treatment group imbalances within the countries are difficult to assess.

Previous and Concomitant Medication

Study S241.3.006

Most subjects in the cilansetron 2 mg TID and placebo groups (96% versus 93%) took at least one concomitant medication. Categories of the most frequently used (≧10% in either treatment group) concomitant medications included other analgesics and antipyretics (35% versus 32%), antiinflammatory/antirheumatic products (32% versus 27%), antidepressants (26% versus 25%), multivitamins (combinations) (24% versus 26%), drugs for treatment of peptic ulcer (24% versus 22%), topical products for joint and muscular pain (22% versus 20%), estrogens (19% versus 20%), hormones and related agents (19% in both groups), antihistamines for systemic use (19% versus 17%), other gynecologicals (19% versus 17%), calcium (14% versus 19%), stomatological preparations (15% in both groups), antithrombotic agents (13% versus 14%), cholesterol and triglyceride reducers (13% in both groups), hormonal contraceptives for systemic use (15% versus 11%), other plain vitamin preparations (11% versus 14%), laxatives (14% versus 9%), beta blocking agents (10% versus 11%), decongestants and other nasal preparations for topical use (11% in both groups), thyroid preparations (8% versus 11%), and anxiolytics (10% versus 7%). No clinically relevant treatment group difference was observed for any category.

The WHO generic terms with >10% of subjects in either group included paracetamol (18% versus 14% in the cilansetron 2 mg T]ID and placebo groups, respectively), acetylsalicylic acid (11% versus 13%), ibuprofen (13% in both groups), multivitamins (19% versus 20%), conjugated estrogen (11% in both groups), calcium (10% versus 9%), and tocopherol (10% versus 13%). There were no relevant treatment group differences or trends observed for any WHO generic term. No impact on efficacy was noted with these medications.

Study S241.3.011

Three-Month Treatment Period

Most subjects in the cilansetron 2 mg TID and placebo groups (86% versus 88%) took at least one concomitant medication. Categories of the most frequently used (≧10% in either treatment group) concomitant medications included other analgesics and antipyretics (29% versus 30%), antiinflammatory/antirheumatic products, non-steroids (27% versus 22%), drugs for treatment of peptic ulcer (21% versus 25%), antidepressants (23% in both groups), antihistamines for systemic use (19% versus 18%), topical products for joint and muscular pain (18% versus 15%), multivitamins, combinations (15% versus 17%), estrogens (12% versus 17%), hormones and related agents (12% versus 17%), other gynecologicals (15% versus 13%), cholesterol and triglyceride reducers (14% versus 12%), beta blocking agents (12% versus 11%), stomatological preparations (11% versus 12%), calcium (11% in both groups), corticosteroids, plain (11% in both groups), laxatives (13% versus 7%), anxiolytics (6% versus 12%), hormonal contraceptives for systemic use (9% versus 10%), decongestants and other nasal preparations for topical use (8% versus 11%), and other antiasthmatics, inhalants (7% versus 10%). No relevant treatment group differences were observed for any category except for laxatives and anxiolytics.

The WHO generic terms with >10% of subjects in either group included paracetamol (15% versus 17% in the cilansetron 2 mg TID and placebo groups, respectively), ibuprofen (11% versus 8%), multivitamins (12% versus 13%), and estrogens conjugated (7% versus 10%). There were no relevant treatment group differences or trends observed for any WHO generic term. No impact on efficacy was noted with these medications.

Four-Week Rerandomization Period

Most subjects (82% to 86%) in the treatment groups took least one concomitant medication and 51% to 62% of subjects took at least one key concomitant medication. Categories of the most frequently used (≧10% in either treatment group) key concomitant medications across the treatment groups included other analgesics and antipyretics (23% to 28%), antidepressants (19% to 23%), and antihistamines for systemic use (2% to 10%). No clinically relevant treatment group differences were observed for any category. The WHO generic terms with ≧10% of subjects in either group included paracetamol (11% to 14% across the treatment groups). There were no relevant treatment group differences or trends observed for any WHO generic term. No impact on safety or efficacy was noted with these medications.

Study S241.3.009

Most subjects in the cilansetron 2 mg TID and placebo groups (65% versus 61%) took at least one concomitant medication. Categories of the most frequently used (≧5% in either group) concomitant medications included other analgesics and antipyretics (21% versus 20%), beta blocking agents (10% versus 9%), drugs for treatment of peptic ulcer (10% versus 9%), hormonal contraceptives for systemic use (9% versus 8%), non-steroidal antuinflammatory/antirheumatic products (8% versus 8%), ACE-inhibitors (7% versus 9%), antidepressants (7% versus 6%), hormones and related agents (6% versus 7%), decongestants and other nasal preparations for topical use (6% versus 7%), estrogens (5% versus 8%), topical products forjoint and muscular pain (6% versus 5%), stomatological preparations (5% versus 6%), antithrombotic agents (5% versus 6%), all other therapeutic products (5% versus 5%), laxatives (5% versus 4%), anxiolytics (5% versus 4%), cholesterol and triglyceride reducers (5% versus 4%), corticosteroids (4% versus 5%), other gynecologicals (5% versus 4%), thyroid preparations (5% versus 3%), and beta-lactam antibacterials and penicillins (5% versus 2%). No clinically relevant treatment group difference was observed for any category.

The WHO generic terms with ≧5% of subjects in either group included paracetamol (15% in both treatment groups) and acetylsalicylic acid (4% versus 5% in the cilansetron 2 mg TIID and placebo groups, respectively) with no relevant treatment group differences or trends observed for any WHO generic term. No impact on efficacy is expected with these medications.

Measurements of Treatment Compliance

Study S241.3.006

The mean overall compliance was 92.64% versus 93.79% in thee. cilansetron 2 mg TIED and placebo groups, respectively, with 90% versus 93% of subjects in the 800/% to 120% compliance rate category. Three subjects (3006-525002, 3006-608007, and 3006-613025) in the cilansetron 2 mg TID group had >120% compliance.

Study S241.3.011

The mean overall compliance was 91.64% versus 92.26% in the cilansetron 2 mg TID and placebo groups, respectively, with 87% versus 89% of subjects in the 80% to 120%.compliance rate category. Three subjects in the cilansetron 2 mg TIID group had >120% compliance.

Study S241.3.009

The mean overall compliance was 98.71% versus 97.66% in the cilansetron 2 mg TID and placebo groups, respectively, with 95% versus 93% of subjects in the 80% to 120% compliance rate category. Six subjects in the cilansetron 2 mg TID group had >120% compliance.

Comparison of Efficacy Results of All Studies

Efficacy Measurements

Studies S24 1.3 .006, S24 1.3.0 1 1, and S24 1.3.009 used the following efficacy measurements:
The subjects answered the following three questions with “yes” or “no” using a phone diary at the end of each week:
   a) During the last week, did you experience adequate relief of your IBS symptoms, such as abdominal pain, abdominal discomfort, abnormal bowel habits?
   b) During the last week, did you experience adequate relief of your abdominal pain and/or discomfort?
   c) During the last week, did you experience adequate relief of your abnormal bowel habits?
The subjects rated the following symptoms using the phone diary at the end of each day:
   abdominal pain/discomfort (five-point scale);
   stool consistency (seven-point Bristol Stool Scale);
   stool frequency (number of bowel movements each day);
   urgency (five-point scale);
   straining (yes or no);
   feeling of incomplete defecation (yes or no);
   bloating or feeling of abdominal distension (five-point scale).
In Studies S241 A006 and S241.3.009, subjects completed the IBS-QOL Survey at Baseline (Day 1) and at the end of treatment. In Study S24 1.3.0 1 1, subjects completed the IBS-QOL Survey at Baseline (Day 1), at the end of the three-month treatment period, and at the end of rerandomized treatment. The evaluation of these data followed the common evaluation recommended by the authors of this questionnaire.
The subjects answered the following question at the pre-treatment visit, Month 1, Month 2 (S241.3.006 and S241.3.011 only), Month 3, Month 4 (S241.3.011 only), and Month 6 (S241.3.009 only) or at the early termination visit using a five-point scale:
   Over the past four weeks, how have your IBS symptoms significantly interfered with your ability to effectively perform your activities (e.g. work, school, recreation, household, social or travel activities)?
      Efficacy Parameters

In all three pivotal efficacy studies, the primary and main secondary efficacy parameters were based on the weekly diary-questions on adequate relief and included the following:
1. Number of months (one month equals four weeks) that a subject was a responder for the weekly diary questions on adequate relief. A responder for a month (monthly responder) was defined as a subject who answered the weekly question with “yes” at least twice for that month. For Studies S241.3.006 and S241.3.011, the primary efficacy parameter was the number of months that a subject was a responder for IBS symptoms during the 12-week treatment period. The two main secondary efficacy parameters were the number of months that a subject was a responder for abdominal pain/discomfort and abnormal bowel habits during the 12-week treatment period. For Study S241.3.009, the number of months that a subject was a responder for the weekly diary questions on adequate relief during Months 1 to 3 (the first 12 weeks) and Months 1 to 6 (24-week) was an additional key secondary efficacy parameter.
2. Overall responder rate for the weekly diary questions on adequate relief. A responder was defined as a subject who answered the weekly question with “yes” for at least half of the answers available with a minimum of four weeks treatment duration. If a subject had less than four weeks treatment duration, the subject was considered a non-responder. The overall responder rate was the percentage of responders within a treatment group. For Study S241.3.009, the primary efficacy parameter was the overall responder rate for IBS symptoms during the 26-week treatment period.

The two main secondary efficacy parameters were the overall responder rate for abdominal pain/discomfort and abnormal bowel habits during the 26-week treatment period. For Studies S241.3.006 and S241.3.011, the overall responder rate during the 12-week treatment period was a secondary efficacy parameter.

Other secondary efficacy parameters for all three studies included:
Responder rate for the weekly diary questions on adequate relief at each month, where a responder for a month (monthly responder) was defined as a subject who answered the weekly question with “yes” at least twice for that month. The responder rate was the percentage of responders within a treatment group. The all-month responder rate for the weekly diary questions on adequate relief was also evaluated, where an all-month responder was defined as a subject who answered the weekly question with “yes” at least twice for each month. The all-month responder rate was the percentage of all-month responders within a treatment group;
Adequate relief rate for the weekly diary questions was calculated for each subject as the number of weekly assessments with adequate relief (“yes”) divided by the total number of weekly assessments;
Percentage of subjects with adequate relief for the weekly diary questions at each week;
Daily diary questions regarding abdominal pain/discomfort (five-point scale), stool consistency (seven-point scale), stool frequency (number of bowel movements each day), straining (yes or no), urgency (five-point scale), feeling of incomplete evacuation (yes or no), and bloating or feeling of abdominal distention (five-point scale);
QOL data assessed by the IBS-QOL questionnaire; and
Question to determine the interruption of activities.
   Imputation Methods for Missing; Efficacy Data

Efficacy analyses were carried out using observed data and imputed data. The following three imputation approaches were used for the weekly diary questions, with the specific methodology depending upon the type of analysis:
1.Last observation carried forward (LOCF) approach for overall responder rate, adequate relief rate, and responder rate by week (weekly LOCF)
   If a subject had a missing response for a weekly diary question (i.e., EBS symptoms, abdominal pain/discomfort, or abnormal bowel habits), the last non-missing weekly response was carried forward for the missing response. If a subject had a missing response for one or more weeks at the beginning of the treatment period, the answer of “no adequate relief” was imputed for those weeks.
2. LOCF approach for monthly responder (monthly LOCF)
   A subject was defined as a responder for a month (monthly responder) if he/she answered the weekly question with “yes” at least twice for that month regardless of the number of missing responses for the weekly diary question, where a month was defined as a four-week period. Subjects who had at least one weekly question response but did not have at least two answers of “yes” for that month were considered a non-responder for that month. If a subject had missing responses for the entire month, the subject had the last non-missing monthly responder determination (responder or non-responder) carried forward for the missing response. If a subject had missing responses for the entire month of Month 1, the subject was considered a non-responder.
3. Conservative approach for all efficacy analyses
   If a subject had a missing response for a weekly diary question (i.e., IBS symptoms, abdominal pain/discomfort, or abnormal bowel habits), the answer of “no adequate relief” was imputed for the missing response.

Imputation was not carried out for other efficacy parameters (i.e., daily diary questions, IBS-QOL questionnaire, or interruption of activities).

Imputation of missing values did not appear to have an impact on the efficacy analysis. Subject compliance with the weekly diary questions was very good. Analyses for missing data for the weekly diary question on adequate relief of EBS symptoms are presented below.

Number of Months a Subject Was a Responder for the Weekly Diary Questions on Adequate Relief

Summary Across Studies

Strong evidence for the superiority of cilansetron 2 mg TID over placebo was established in all three pivotal efficacy studies as reflected by the number of months a subject was a responder for the weekly diary question on adequate relief of EBS symptoms (primary efficacy parameter in Studies S241.3.006 and S241.3.011 and key secondary efficacy parameter in Study S241.3.009) during the three-month treatment period (p<0.001 in all three studies) and during the six-month treatment period (p<0.001, Study S241.3.009). Similarly, statistically significant differences were demonstrated in favor of cilansetron 2 mg TID compared with placebo in all three pivotal efficacy studies by the number of months a subject was a responder for the weekly diary questions on adequate relief of abdominal pain/discomfort and abnormal bowel habits (main secondary efficacy parameters in Studies S241.3.006 and S241.3.011 and key secondary efficacy parameters in Study S241.3.009) during the three-month treatment period (p<0.001 for both diary questions in all three studies) and during the six-month treatment period (p<0.001 for both diary questions in Study S241.3.009).

In Study S241.3.011, there was a statistically significant treatment by gender interaction for all three diary questions. In Study S241.3.009, there was a statistically significant treatment by pooled country interaction for all three diary questions at both three and six months of treatment. The nature of these interactions will be further explored below.

The number of months a subject was a responder for the weekly diary questions on adequate relief during the entire four months of Study S241.3.011 in subjects who were rerandomized to the same treatment as they were taking during the first three months of the study showed results consistent with the beneficial effects of cilansetron 2 mg TID that was maintained for up to six months as described above.

Study S241.3.606

Results for the ITT population using monthly LOCF imputation are displayed for each of the three weekly diary questions on adequate relief in Table 2.7.3:14.

TABLE 2.7.3:14
Primary and Main Secondary Efficacy Parameters: Number of
Months a Subject Was a Responder for the Weekly Diary Questions
on Adequate Relief During the Three-Month Treatment Period
(Monthly LOCF Imputation)
Study S241.3.006 (ITT Population)

| Number of months a subject was a responder | | Cilansetron 2 mg TID | Placebo | |
|---|---|---|---|---|
| during the three-month treatment period | Statistic | (N = 344) | (N = 348) | P-Value^{a} |
| Primary efficacy parameter: | | | | |
| Adequate relief of IBS symptoms | | | | |
| Zero months | n (%) | 127 (37) | 195 (56) | |
| One month | n (%) | 45 (13) | 43 (12) | |
| Two months | n (%) | 53 (15) | 47 (14) | |
| Three months | n (%) | 119 (35) | 63 (18) | |
| Treatment effect | | | | <0.001** |
| Treatment by gender interaction | | | | 0.685 |
| Treatment by pooled center interaction | | | | 0.965 |
| Main secondary efficacy parameter: | | | | |
| Adequate relief of abdominal pain/discomfort | | | | |
| Zero months | n (%) | 116 (34) | 172 (49) | |
| One month | n (%) | 42 (12) | 43 (12) | |
| Two months | n (%) | 47 (14) | 54 (16) | |
| Three months | n (%) | 139 (40) | 79 (23) | |
| Treatment effect | | | | <0.001** |
| Treatment by gender interaction | | | | 0.514 |
| Treatment by pooled center interaction | | | | 0.600 |
| Main secondary efficacy parameter: | | | | |
| Adequate relief of abnormal bowel habits | | | | |
| Zero months | n (%) | 123 (36) | 203 (58) | |
| One month | n (%) | 40 (12) | 45 (13) | |
| Two months | n (%) | 52 (15) | 41 (12) | |
| Three months | n (%) | 129 (38) | 59 (17) | |
| Treatment effect | | | | <0.001** |
| Treatment by gender interaction | | | | 0.275 |
| Treatment by pooled center interaction | | | | 0.765 |

*Significant at the 0.050 level for treatment effect;
**Significant at the 0.010 level for treatment effect;
⁺Significant at the 0.100 level for interaction.
^{a}P-values for treatment effect are based on a non-parametric ANCOVA model with an effect for treatment and Baseline IBS severity as a covariate, stratified by pooled center and gender. P-values for treatment by stratification variable interactions (i.e., pooled center and gender) are based on an ANCOVA analysis with terms for treatment, pooled center, gender, Baseline IBS severity, and treatment by stratification variable interaction in the model.
Note:
Percentages are based on the number of subjects in the ITT Population.
Note:
A subject is defined as a responder for a month if he/she reports adequate relief (‘yes’) for at least two of the four weeks during that month.
Note:
Weekly diary observations made on or after the date of randomization and up through two days after the date of last dose of study medication are included in this summary.
Data source: CSR Table 10.1.2.1.1 Left. Vol. 9 Righ

Subjects in the cilansetron 2 mg TID group were responders for more months in general compared to subjects in the placebo group for the diav question on adequate relief of EBS symptoms as reflected by the statistically significant (p<0.001) treatment group difference in the distribution of subjects among the number of months categories (monthly LOCF imputation approach, ITT population). The treatment by gender and pooled center interactions were not significant for the diary question on adequate relief of IBS symptoms. Analysis using the conservative imputation approach showed similar results to the analysis using the monthly LOCF imputation. Per-protocol analyses showed results consistent with the ITT analyses. Subjects in the cilansetron 2 mg TID group were responders for more months in general compared to subjects in the placebo group for the diary question on adequate relief of abdominal pain/discomfort symptoms as reflected by the statistically significant (p<0.001) treatment group difference in the distribution of subjects among the number of months categories (monthly LOCF imputation approach, ITT population). The treatment by gender and pooled center interactions were not significant for the diary question on adequate relief of abdominal pain/discomfort symptoms. Analysis using the conservative imputation approach showed similar results to the analysis using the monthly LOCF imputation. Per-protocol analyses showed results consistent with the ITT analyses.

Subjects in the cilansetron 2 mg TID were responders for more months in general compared to subjects in the placebo group for the diary question on adequate relief of abnormal bowel habits as reflected by the statistically significant (p<0.001) treatment group difference in the distribution of subjects among the number of months categories (monthly LOCF imputation approach, ITT population). The treatment by gender and pooled center interactions were not significant for the diary question on adequate relief of abnormal bowel habits. Analysis using the conservative imputation approach showed similar results to the analysis using the monthly LOCF imputation. Per-protocol analyses showed results consistent with the ITT analyses.

Additional analyses of the number of months a subject was a responder during the three-month treatment period by gender, age category, race, BMI, severity of IBS, duration of IBS, tobacco use, and pooled center are presented below.

Study S241.3.011

Three-Month Treatment Period

Results for the ITT population using monthly LOCF analysis are displayed for each of the three weekly diary questions on adequate relief in Table 2.7.3:15.

TABLE 2.7.3:15
Primary and Main Secondary Efficacy Parameters: Number of
Months a Subject Was a Responder for the Weekly Diary Questions
on Adequate Relief During the Three-Month Treatment Period
(Monthly LOCF Imputation)
Study S241.3.011 (ITT Population)

| Number of months a subject was a responder | | Cilansetron 2 mg TID | Placebo | |
|---|---|---|---|---|
| during the three-month treatment period | Statistic | (N = 377) | (N = 369) | P-Value^{a} |
| Primary efficacy parameter: | | | | |
| Adequate relief of IBS symptoms | | | | |
| Zero months | n (%) | 105 (28) | 198 (54) | |
| One month | n (%) | 54 (14) | 47 (13) | |
| Two months | n (%) | 70 (19) | 56 (15) | |
| Three months | n (%) | 148 (39) | 68 (18) | |
| Treatment effect | | | | <0.001** |
| Treatment by gender interaction | | | | 0.001⁺ |
| Treatment by pooled center interaction | | | | 0.250 |
| Main secondary efficacy parameter: | | | | |
| Adequate relief of abdominal pain/discomfort | | | | |
| Zero months | n (%) | 101 (27) | 162 (44) | |
| One month | n (%) | 45 (12) | 55 (15) | |
| Two months | n (%) | 72 (19) | 59 (16) | |
| Three months | n (%) | 159 (42) | 93 (25) | |
| Treatment effect | | | | <0.001** |
| Treatment by gender interaction | | | | 0.001⁺ |
| Treatment by pooled center interaction | | | | 0.423 |
| Main secondary efficacy parameter: | | | | |
| Adequate relief of abnormal bowel habits | | | | |
| Zero months | n (%) | 112 (30) | 204 (55) | |
| One month | n (%) | 50 (13) | 43 (12) | |
| Two months | n (%) | 66 (18) | 61 (17) | |
| Three months | n (%) | 149 (40) | 61 (17) | |
| Treatment effect | | | | <0.001** |
| Treatment by gender interaction | | | | 0.018⁺ |
| Treatment by pooled center interaction | | | | 0.470 |

*Significant at the 0.050 level for treatment effect;
**Significant at the 0.010 level for treatment effect;
⁺Significant at the 0.100 level for interaction.
^{a}P-values for treatment effect are based on a non-parametric ANCOVA model with an effect for treatment and Baseline IBS severity as a covariate, stratified by pooled center and gender. P-values for treatment by stratification variable interactions (i.e., pooled center and gender) are based on an ANCOVA analysis with terms for treatment, pooled center, gender, Baseline IBS severity, and treatment by stratification variable interaction in the model.
Note:
Percentages are based on the number of subjects in the ITT Population.
Note:
A subject is defined as a responder for a month if he/she reports adequate relief (‘yes’) for at least two of the four weeks during that month.
Note:
Weekly diary observations made on or after the date of randomization and up through two days after the date of last dose of study medication during the 12-week treatment period (or the date of first dose of study medication during the rerandomization period if earlier) are included in this summary.
Data source: CSR Table 10.1.2.1.1 Left. Vol. 9 Righ

Subjects in the cilansetron 2 mg TID group were responders for more months in general compared to subjects in the placebo group for the diary question on adequate relief of IBS symptoms as reflected by the statistically significant (p<0.001) treatment group difference in the distribution of subjects across the number of months categories (monthly LOC F imputation approach, ITT population). There was a significant treatment by gender interaction, however, there was no significant treatment by pooled center interaction for the diary question on adequate relief of IBS symptoms. Analysis using the conservative imputation approach showed similar results to the analysis using the monthly LOCF imputation. Per-protocol analyses showed results consistent with the ITT analyses except that there was a significant treatment by pooled center interaction using the conservative imputation approach.

Subjects in the cilansetron 2 mg TID group were responders for more months in general compared to subjects in the placebo group for the diary question on adequate relief of abdominal pain/discomfort symptoms as reflected by the statistically significant (p<0.001) treatment group difference in the distribution of subjects across the number of months categories (monthly LOCF imputation approach, ITT population). There was a significant treatment by gender interaction, however, there was no significant treatment by pooled center interaction for the diary question on adequate relief of abdominal pain/discomfort symptoms. Analysis using the conservative imputation approach showed similar results to the analysis using the monthly LOCF imputation. Per-protocol analyses showed results consistent with the ITT analyses except that there was a significant treatment by pooled center interaction.

Subjects in the cilansetron 2 mg TID group were responders for more months in general compared to subjects in the placebo group for the diary question on adequate relief of abnormal bowel habits as reflected by the statistically significant (p<0.00l) treatment group difference in the distribution of subjects across the number of months categories (monthly LOCF imputation approach, ITT population). There was a significant treatment by gender interaction, however, there was no significant treatment by pooled center interaction for the diary question on adequate relief of abnormal bowel habits. Analysis using the conservative imputation approach showed similar results to the analysis using the monthly LOCF imputation. Per-protocol analyses showed results consistent with the ITF analyses.

Additional analyses of the number of months a subject was a responder during the three-month treatment period by gender, age category, race, BMI, severity of IBS, duration of IBS, tobacco use, and pooled center are presented below.

Entire Four-Month Treatment Period

Subjects in the C/C treatment group were responders for more months in general compared with subjects in the P/P treatment group for each of the three weekly diary questions on adequate relief (monthly LOCF imputation approach). Analysis using the conservative imputation approach showed similar results to the analysis using the monthly LOCF imputation.

Study S241.3.009

Months 1 to 3

The number of months a subject was a responder for the weekly diary questions on adequate relief during Months 1 to 3 is presented for the IT population in Table 2.7.3:16.

TABLE 2.7.3:16
Number of Months a Subject Was a Responder for the Weekly Diary
Questions on Adequate Relief During Months 1 to 3 (Monthly LOCF
Imputation)
Study S241.3.009 (ITT Population)

| Number of months a subject was a responder | | Cilansetron 2 mg TID | Placebo | |
|---|---|---|---|---|
| during Months 1 to 3 | Statistic | (N = 395) | (N = 397) | P-Value^{a} |
| IBS symptoms | | | | |
| Zero months | n (%) | 110 (28) | 159 (40) | |
| One month | n (%) | 55 (14) | 82 (21) | |
| Two months | n (%) | 80 (20) | 71 (18) | |
| Three months | n (%) | 150 (38) | 85 (21) | |
| Treatment effect | | | | <0.001** |
| Treatment by gender interaction | | | | 0.282 |
| Treatment by pooled country interaction | | | | 0.003⁺ |
| Abdominal pain/discomfort | | | | |
| Zero months | n (%) | 105 (27) | 159 (40) | |
| One month | n (%) | 61 (15) | 72 (18) | |
| Two months | n (%) | 70 (18) | 76 (19) | |
| Three months | n (%) | 159 (40) | 90 (23) | |
| Treatment effect | | | | <0.001** |
| Treatment by gender interaction | | | | 0.277 |
| Treatment by pooled country interaction | | | | 0.006⁺ |
| Abnormal bowel habits | | | | |
| Zero months | n (%) | 98 (25) | 151 (38) | |
| One month | n (%) | 49 (12) | 82 (21) | |
| Two months | n (%) | 83 (21) | 77 (19) | |
| Three months | n (%) | 165 (42) | 87 (22) | |
| Treatment effect | | | | <0.001** |
| Treatment by gender interaction | | | | 0.633 |
| Treatment by pooled country interaction | | | | 0.033⁺ |

*Significant at the 0.050 level for treatment effect;
**Significant at the 0.010 level for treatment effect;
⁺Significant at the 0.100 level for interaction.
^{a}P-values for treatment effect are based on a non-parametric ANCOVA model with an effect for treatment and Baseline IBS severity as a covariate, stratified by pooled country and gender. P-values for treatment by stratification variable interactions (i.e., pooled country and gender) are based on an ANCOVA analysis with terms for treatment, stratification variable, Baseline IBS severity, and treatment by stratification variable interaction in the model.
Note:
Percentages are based on the number of subjects in the ITT Population.
Note:
A subject is defined as a responder for a month if he/she reports adequate relief (‘yes’) for at least two of the four weeks during that month.
Note:
Weekly diary observations made on or after the date of randomization and up through two days after the date of last dose of study medication are included in this summary.
Data source: CSR Table 10.1.2.1.1 Left. Vol. 9 Righ

During Months 1 to 3, subjects in the cilansetron 2 mg TID group were responders for more months in general compared to subjects in the placebo group for each of the three weekly diary questions on adequate relief as reflected by the statistically significant (p<0.001) treatment group difference in the distribution of subjects among the number of months categories (monthly LOCF imputation approach, ITT population). There was a significant treatment by pooled country interaction (p=0.003 to 0.033) for each diary question. The treatment by gender interaction was not significant for any of the three diary questions. Analysis using the conservative imputation approach showed similar results to the analysis using the monthly LOCF imputation. Per-protocol analyses showed results consistent with the ITT analyses except that the treatment by gender interaction was significant for abdominal pain/discomfort when using the monthly LOCF imputation approach.

Months 1 to 6

The number-of months a subject was a responder for the weekly diary questions on adequate relief during the six-month treatment period is presented for the ITT population in Table 2.7.3:17.

TABLE 2.7.3:17
Additional Key Secondary Efficacy Parameter: Number of Months a
Subject Was a Responder for the Weekly Diary Questions on
Adequate Relief During Months 1 to 6 (Monthly LOCF Imputation)
Study S241.3.009 (ITT Population)

| Number of months a subject was a responder | | Cilansetron 2 mg TID | Placebo | |
|---|---|---|---|---|
| during Months 1 to 6 | Statistic | (N = 395) | (N = 397) | P-Value^{a} |
| IBS symptoms | | | | |
| Zero months | n (%) | 90 (23) | 121 (30) | |
| One month | n (%) | 25 (6) | 50 (13) | |
| Two months | n (%) | 24 (6) | 31 (8) | |
| Three months | n (%) | 25 (6) | 31 (8) | |
| Four months | n (%) | 52 (13) | 43 (11) | |
| Five months | n (%) | 58 (15) | 55 (14) | |
| Six months | n (%) | 121 (31) | 66 (17) | |
| Treatment effect | | | | 0.001** |
| Treatment by gender interaction | | | | 0.186 |
| Treatment by pooled country interaction | | | | <0.001⁺ |
| Abdominal pain/discomfort | | | | |
| Zero months | n (%) | 85 (22) | 117 (29) | |
| One month | n (%) | 26 (7) | 50 (13) | |
| Two months | n (%) | 26 (7) | 28 (7) | |
| Three months | n (%) | 31 (8) | 38 (10) | |
| Four months | n (%) | 41 (10) | 36 (9) | |
| Five months | n (%) | 53 (13) | 52 (13) | |
| Six months | n (%) | 133 (34) | 76 (19) | |
| Treatment effect | | | | 0.001** |
| Treatment by gender interaction | | | | 0.228 |
| Treatment by pooled country interaction | | | | 0.007⁺ |
| Abnormal bowel habits | | | | |
| Zero months | n (%) | 85 (22) | 116 (29) | |
| One month | n (%) | 17 (4) | 55 (14) | |
| Two months | n (%) | 22 (6) | 33 (8) | |
| Three months | n (%) | 33 (8) | 29 (7) | |
| Four months | n (%) | 41 (10) | 36 (9) | |
| Five months | n (%) | 61 (15) | 66 (17) | |
| Six months | n (%) | 136 (34) | 62 (16) | |
| Treatment effect | | | | 0.001** |
| Treatment by gender interaction | | | | 0.379 |
| Treatment by pooled country interaction | | | | 0.008⁺ |

*Significant at the 0.050 level for treatment effect;
**Significant at the 0.010 level for treatment effect;
⁺Significant at the 0.100 level for interaction.
^{a}P-values for treatment effect are based on a non-parametric ANCOVA model with an effect for treatment and Baseline IBS severity as a covariate, stratified by pooled country and gender. P-values for treatment by stratification variable interactions (i.e., pooled country and gender) are based on an ANCOVA analysis with terms for treatment, stratification variable, Baseline IBS severity, and treatment by stratification variable interaction in the model.
Note:
Percentages are based on the number of subjects in the ITT Population.
Note:
A subject is defined as a responder for a month if he/she reports adequate relief (‘yes’) for at least two of the four weeks during that month.
Note:
Weekly diary observations made on or after the date of randomization and up through two days after the date of last dose of study medication are included in this summary.
Data source: CSR Table 10.1.2.1.16 Left. Vol. 9 Righ

Subjects in the cilansetron 2 mg TID group were responders for more months in general compared to subjects in the placebo group for each of the three weekly diary questions on adequate relief as reflected by the statistically significant (p<0.001) treatment group difference in the distribution of subjects among the number of months categories (monthly LOCF imputation approach, ITT population). There was a significant treatment by pooled country interaction (p<0.001 to 0.008) for each diary question. The treatment by gender interaction was not significant for any of the three diary questions. Analysis using the conservative imputation approach showed similar results to the analysis using the monthly LOCF imputation. Perprotocol analyses showed results consistent with the ITT analyses except that the treatment by pooled country interaction was not significant for abdominal pain/discomfort when using the conservative imputation approach.

Overall Responder Rate for the Weekly Diary Questions on Adequate Relief

Summary Across Studies

Strong evidence for the superiority of cilansetron 2 mg TID over placebo was established in all three pivotal efficacy studies as reflected by the overall responder rate for the weekly diary question on adequate relief of IBS symptoms (primary efficacy parameter in Study S241.3.009 and secondary efficacy parameter in Studies S241.3.006 and S241.3.011) during the three-month treatment period (p<0.001 in all three studies) and during the six-month treatment period (p<0.001, Study S241.3.009). Similarly, statistically significant differences were demonstrated in favor of cilansetron 2 mg TID compared with placebo in all three pivotal efficacy studies by the overall responder rate for the weekly diary questions on adequate relief of abdominal pain/discomfort and abnormal bowel habits (main secondary efficacy parameters in Study S241.3.009 and secondary efficacy parameters in Studies S241.3.006 and S241.3.011) during the three-month treatment period (p<0.001 for both diary questions in all three studies) and during the six-month treatment period (p<0.001 for both diary questions in Study S241.3.009). Of note, the treatment group differences were slightly smaller in Study S241.3.009 in general at both three months and six months, mostly due to the slightly higher placebo responder rates in this study compared with the other two studies.

There was a statistically significant treatment by gender interaction for all three diary questions in Study S241.3.011 and for adequate relief of IBS symptoms in Study S241.3.009 at six months of treatment. There was also a statistically significant treatment by pooled country interaction for all three diary questions in Study S241.3.009 at both three and six months of treatment. The nature of these interactions will be further explored below.

The overall responder rate during the entire four months of Study S241.3.011 in subjects who were rerandomized to the same treatment as they were taking during the first three months of the study showed results consistent with the beneficial effects of cilansetron 2 mg TID that was maintained for up to six months as described above.

Study S241.3.006

The secondary efficacy parameter, overall responder rate for the weekly diary questions during the three-month treatment period is presented for the IT population in Table 2.7.3:18.

TABLE 2.7.3:18
Overall Responder Rate for the Weekly Diary Questions on Adequate
Relief During the Three-Month Treatment Period (Observed Case)
Study S241.3.006 (ITT Population)

| Number of subjects who were responders | | Cilansetron 2 mg TID | Placebo | |
|---|---|---|---|---|
| during the three-month treatment period | Statistic | (N = 344) | (N = 348) | P-Value^{a} |
| Adequate relief of IBS symptoms | n (%) | 168 (49) | 99 (28) | |
| Treatment effect | | | | <0.001** |
| Treatment by gender interaction | | | | 0.694 |
| Treatment by pooled center interaction | | | | 0.843 |
| Adequate relief of abdominal pain/discomfort | n (%) | 179 (52) | 129 (37) | |
| Treatment effect | | | | <0.001** |
| Treatment by gender interaction | | | | 0.246 |
| Treatment by pooled center interaction | | | | 0.803 |
| Adequate relief of abnormal bowel habits | n (%) | 174 (51) | 89 (26) | |
| Treatment effect | | | | <0.001** |
| Treatment by gender interaction | | | | 0.512 |
| Treatment by pooled center interaction | | | | 0.339 |

*Significant at the 0.050 level for treatment effect;
**Significant at the 0.010 level for treatment effect;
⁺Significant at the 0.100 level for interaction.
^{a}P-values for treatment effect are based on a non-parametric analysis of covariance (ANCOVA) model with an effect for treatment and Baseline IBS severity as a covariate, stratified by pooled center and gender. P-values for treatment by stratification variable interactions (i.e., pooled center and gender) are based on an ANCOVA analysis with terms for treatment, pooled center, gender, Baseline IBS severity, and treatment by stratification variable interaction in the model.
Note:
Percentages are based on the number of subjects in the ITT Population.
Note:
A responder for the three-month treatment period is defined as a subject who answers the weekly question with “yes” for at least half of the answers available with a minimum of four-week treatment duration during the 12-week treatment period. If a subject had less than four-week treatment duration, the subject was considered a non-responder.
Note:
Weekly diary observations made on or after the date of randomization and up through two days after the date of last dose of study medication are included in this summary.
Data source: CSR Table 10.1.2.2.1 Left. Vol. 9 Righ

The overall responder rate for each of the three weekly diary questions on adequate relief during the three-month treatment period was significantly higher in the cilansetron 2 mg TID group compared with the placebo group (observed case analysis on the ITT population: IBS symptoms, 49% versus 28%, p<0.001; abdominal pain/discomfort, 52% versus 37%; p<0.001 ; and abnormal bowel habits, 51% versus 26%, p<0.001). The treatment by gender and pooled center interactions were not significant for any of the three diary questions. Analysis using the weekly LOCF approach showed similar results to the observed case analysis. Per-protocol analyses showed results consistent with the ITT analyses.

Study S241.3.011

Three-Month Treatment Period

The secondary efficacy parameter, overall responder rate for the weekly diary questions during the three-month treatment period is presented for the ITT population in Table 2.7.3:19.

TABLE 2.7.3:19
Overall Responder Rate for the Weekly Diary Questions on Adequate
Relief During the Three-Month Treatment Period (Observed Case)
Study S241.3.011 (ITT Population)

| Number of subjects who were responders | | Cilansetron 2 mg TID | Placebo | |
|---|---|---|---|---|
| during the three-month treatment period | Statistic | (N = 377) | (N = 369) | P-Value^{a} |
| Adequate relief of IBS symptoms | n (%) | 206 (55) | 108 (29) | |
| Treatment effect | | | | <0.001** |
| Treatment by gender interaction | | | | 0.009⁺ |
| Treatment by pooled center interaction | | | | 0.274 |
| Adequate relief of abdominal pain/discomfort | n (%) | 221 (59) | 136 (37) | |
| Treatment effect | | | | <0.001** |
| Treatment by gender interaction | | | | 0.014⁺ |
| Treatment by pooled center interaction | | | | 0.118 |
| Adequate relief of abnormal bowel habits | n (%) | 203 (54) | 105 (28) | |
| Treatment effect | | | | <0.001** |
| Treatment by gender interaction | | | | 0.051⁺ |
| Treatment by pooled center interaction | | | | 0.618 |

*Significant at the 0.050 level for treatment effect;
**Significant at the 0.010 level for treatment effect;
⁺Significant at the 0.100 level for interaction.
^{a}P-values for treatment effect are based on a non-parametric ANCOVA model with an effect for treatment and Baseline IBS severity as a covariate, stratified by pooled center and gender. P-values for treatment by stratification variable interactions (i.e., pooled center and gender) are based on an ANCOVA analysis with terms for treatment, pooled center, gender, Baseline IBS severity, and treatment by stratification variable interaction in the model.
Note:
Percentages are based on the number of subjects in the ITT Population.
Note:
A responder for the three-month treatment period is defined as a subject who answers the weekly question with “yes” for at least half of the answers available with a minimum of four-week treatment duration during the 12-week treatment period. If a subject had less than a four-week treatment duration, the subject was considered a non-responder.
Note:
Weekly diary observations made on or after the date of randomization and up through two days after the date of last dose of study medication during the 12-week treatment period (or the date of first dose of study medication during the rerandomization period if earlier) are included in this summary.
Data source: CSR Table 10.1.2.2.1 Left. Vol. 9 Righ

The overall responder rate for each of the three weekly diary questions on adequate relief during the three-month treatment period was significantly higher in the cilansetron 2 mg TID group compared with the placebo group (observed case analysis on the ITT population: IBS symptoms, 55% versus 29%, p<0.001 ; abdominal pain/discomfort, 59% versus 37%; p<0.001; and abnormal bowel habits, 54% versus 28%, p<0.001). There was a significant treatment by gender interaction, however, there was no significant treatment by pooled center interaction for the three diary questions. Analysis using the weekly LOCF approach showed similar results to the observed case analysis. Per-protocol analyses showed results consistent with the nll analyses except that there was a significant treatment by pooled center interaction for abdominal pain/discomfort.

Entire Four-Month Treatment Period

The overall responder rate for each of the three weekly diary questions on adequate relief during the entire four-month treatment period was higher in the C/C treatment group compared with the P/P treatment group (observed case analysis: IBS symptoms, 66% versus 28%; abdominal pain/discomfort, 70% versus 36%; and abnormal bowel habits, 69% versus 28%, respectively). Analysis using the weekly LOCF approach showed similar results to the observed case analysis. Of note, the treatment group differences in the overall responder rate were 12% to 15% larger across the diary questions for the entire four-month treatment period compared with the three-months treatment period. This was due to the higher overall responder rate for the entire four-month treatment period compared with the three-months treatment period for subjects taking cilansetron 2 mg TID, while no substantial differences were seen for subjects taking placebo.

Study S241.3.009

Months 1 to 3

The overall responder rate for the weekly diary questions on adequate relief during Months 1 to 3 is presented for the ITT population in Table 2.7.3:20.

TABLE 2.7.3:20
Overall Responder Rate for the Weekly Diary Questions on Adequate
Relief During Months 1 to 3 (Observed Case)
Study S241.3.009 (ITT Population)

| Number of subjects who were responders | | Cilansetron 2 mg TID | Placebo | |
|---|---|---|---|---|
| during Months 1 to 3 | Statistic | (N = 395) | (N = 397) | P-Value^{a} |
| Adequate relief of IBS symptoms | n (%) | 219 (55) | 153 (39) | |
| Treatment effect | | | | <0.001** |
| Treatment by gender interaction | | | | 0.310 |
| Treatment by pooled country interaction | | | | 0.001⁺ |
| Adequate relief of abdominal pain/discomfort | n (%) | 221 (56) | 166 (42) | |
| Treatment effect | | | | <0.001** |
| Treatment by gender interaction | | | | 0.548 |
| Treatment by pooled country interaction | | | | 0.012⁺ |
| Adequate relief of abnormal bowel habits | n (%) | 243 (62) | 164 (41) | |
| Treatment effect | | | | <0.001** |
| Treatment by gender interaction | | | | 0.474 |
| Treatment by pooled country interaction | | | | 0.006⁺ |

*Significant at the 0.050 level for treatment effect;
**Significant at the 0.010 level for treatment effect;
⁺Significant at the 0.100 level for interaction.
^{a}P-values for treatment effect are based on a non-parametric analysis of covariance (ANCOVA) model with an effect for treatment and Baseline IBS severity as a covariate, stratified by pooled country and gender. P-values for treatment by stratification variable interactions (i.e., pooled country and gender) are based on an ANCOVA analysis with terms for treatment, pooled country, gender, Baseline IBS severity, and treatment by stratification variable interaction in the model.
Note:
Percentages are based on the number of subjects in the ITT Population.
Note:
A responder for Months 1-3 treatment period is defined as a subject who answers the weekly question with “yes” for at least half of the answers available with a minimum of four-week treatment duration during the first 12-week treatment period. If a subject had less than four-week treatment duration for the first 12-week treatment period, the subject was considered a non-responder for Months 1-3 treatment period.
Note:
Weekly diary observations made on or after the date of randomization and up through two days after the date of last dose of study medication are included in this summary.
Data source: CSR Table 10.1.2.2.1 Left. Vol. 9 Righ

The overall responder rate during Months 1 to 3 was significantly higher in the cilansetron 2 mg TID group compared with the placebo group for each of the three weekly diary questions (observed case analysis on the ITT population: 55% versus 39% for adequate relief of IBS symptoms, 56% versus 42% for adequate relief of abdominal pain/discomfort, and 62% versus 41% for adequate relief of abnormal bowel habits, p<0.001 for each diary question). There was no significant treatment by gender interaction, however, there was a significant treatment by pooled country interaction for each diary question as similarly seen in other analyses. Analysis using the weekly LOCF approach showed similar results to the observed case analysis. Per-protocol analyses showed results consistent with the ITT analyses except that there was a significant treatment by gender interaction for IBS symptoms.

Months 1 to 6

The primary and main secondary efficacy parameters, overall responder rate for the weekly diary questions on adequate relief during the 26-week treatment period is presented for the ITT population in Table 2.7.3:21.

TABLE 2.7.3:21
Primary and Main Secondary Efficacy Parameter: Overall Responder
Rate for the Weekly Diary Questions on Adequate Relief During the
26-Week Treatment Period (Observed Case)
Study S241.3.009 (ITT Population)

| Number of subjects who were responders | | Cilansetron 2 mg TID | Placebo | |
|---|---|---|---|---|
| during the 26-week treatment period | Statistic | (N = 395) | (N = 397) | P-Value^{a} |
| Primary efficacy parameter: | | | | |
| Adequate relief of IBS symptoms | n (%) | 234 (59) | 179 (45) | |
| Treatment effect | | | | <0.001** |
| Treatment by gender interaction | | | | 0.089⁺ |
| Treatment by pooled country interaction | | | | 0.002⁺ |
| Main secondary efficacy parameter: | | | | |
| Adequate relief of abdominal pain/discomfort | n (%) | 239 (61) | 181 (46) | |
| Treatment effect | | | | <0.001** |
| Treatment by gender interaction | | | | 0.299 |
| Treatment by pooled country interaction | | | | 0.001⁺ |
| Main secondary efficacy parameter: | | | | |
| Adequate relief of abnormal bowel habits | n (%) | 253 (64) | 182 (46) | |
| Treatment effect | | | | <0.001** |
| Treatment by gender interaction | | | | 0.358 |
| Treatment by pooled country interaction | | | | 0.006⁺ |

*Significant at the 0.050 level for treatment effect;
**Significant at the 0.010 level for treatment effect;
⁺Significant at the 0.100 level for interaction.
^{a}P-values for treatment effect are based on a conditional logistic regression model with an effect for treatment, stratified by pooled country and gender. P-values for treatment by stratification variable interactions (i.e., pooled country and gender) are based on a logistic regression with terms for treatment, pooled country, gender, and treatment by stratification variable interaction in the model.
Note:
Percentages are based on the number of subjects in the ITT Population.
Note:
A responder for the six-month treatment period is defined as a subject who answers the weekly question with “yes” for at least half of the answers available with a minimum of four-week treatment duration during the 26-week treatment period. If a subject had less than four-week treatment duration, the subject was considered a non-responder.
Note:
Weekly diary observations made on or after the date of randomization and up through two days after the date of last dose of study medication are included in this summary.
Data source: CSR Table 10.1.2.2.9 Left. Vol. 9 Righ

The overall responder rate for the weekly diary question on adequate relief of IBS symptoms during the 26-week treatment period was significantly higher in the cilansetron 2 mg TID group compared with the placebo group (observed case analysis on the ITT population: 59% versus 45%, p<0.001). However, there was a significant treatment by gender (p=0.089) and treatment by pooled country (p=0.002) interaction. Analysis using the weekly LOCF approach showed similar results (60% versus 45%, p<0.001) to the observed case analysis except that the treatment by gender interaction was not significant. Per-protocol analyses showed results consistent with the ITT analyses.

The overall responder rate for the weekly diary question on adequate relief of abdominal pain/discomfort during the 26-week treatment period was significantly higher in the cilansetron 2 mg TID group compared with the placebo group (observed case analysis on the ITT population: 61% versus 46%, p<0.001). There was no significant treatment by gender interaction (p=0.299), however, there was a significant treatment by pooled country interaction (p=0.001). Analysis using the weekly LOCF approach showed similar results (61% versus 46%, p<0.001) to the observed case analysis. Per-protocol analyses showed results consistent with the ITT analyses.

The overall responder rate for the weekly diary question on adequate relief of abnormal bowel habits during the 26-week/six-month treatment period was significantly higher in the cilansetron 2 mg TID group compared with the placebo group (observed case analysis on the ITT population: 64% versus 46%, p<0.001). There was no significant treatment by gender interaction (p=0.358), however, there was a significant treatment by pooled country interaction (p=0.006). Analysis using the weekly LOCF approach showed similar results (63% versus 46%, p<0.001) to the observed case analysis. Per-protocol analyses showed results consistent with the ITT analyses.

Additional analyses of the overall responder rate by gender, age category, race, BMI, severity of IBS, duration of IBS, tobacco use, and pooled country are presented below.

Responder Rate for the Weekly Diary Questions on Adequate Relief at Each Month and All Months

Summary Across Studies

The responder rate for the weekly diary questions on adequate relief was significantly higher (p≦0.002) in the cilansetron 2 mg TID group compared with the placebo group for each of the three weekly diary questions on adequate relief at each month during the three months of treatment in Studies S241.3.006 and S241.3.011, and at each month during the six months of treatment in Study S241.3.009. The percentage of subjects who were responders for each month during the three-month treatment period was 35% to 40% (cilansetron 2 mg TID) versus 17% to 23% (placebo) across the three diary questions in Study S241.3.006 (p<0.001), 39% to 42% (cilansetron 2 mg TID) versus 17% to 25% (placebo) in Study S241.3.0011 (p<0.001), and 38% to 42% (cilansetron 2 mg TID) versus 21% to 23% (placebo) in Study S241.3.009 (p<0.001). The percentage of subjects who were responders for each month during the six-month treatment penrod in Study S241.3.009 was 31% to 34% (cilansetron 2 mg TID) versus 16% to 19% (placebo) across the three diary questions demonstrating that the favorable effect of cilansetron was sustained for up to six months. Following blinded withdrawal of cilansetron 2 mg TID in Study S241.3.011, almost half of the responders at Month 3 shifted to non-responders by Month 4. There was an observed improvement with respect to the percentage of subjects with adequate relief of IBS symptoms for placebo subjects who transitioned to cilansetron versus placebo subjects who continued on placebo. The effect appeared within one week after rerandomization, and was sustained throughout the four-week period.

Study S241.3.006

The responder rate for the weekly diary questions on adequate relief at each month and all months is presented for the ITT population for the three-month treatment period in Table 2.7.3:22.

TABLE 2.7.3:22
Responder Rate for the Weekly Diary Questions on Adequate Relief
at Each Month and All Months During the Three-Month Treatment
Period (Monthly LOCF Imputation)
Study S241.3.006 (ITT Population)

| | | Cilansetron | | |
|---|---|---|---|---|
| | | 2 mg TID | Placebo | |
| Responder rate | Statistic | (N = 344) | (N = 348) | P-Value^{a} |
| Adequate relief of IBS | | | | |
| symptoms | | | | |
| Month 1 | n (%) | 176 (51) | 104 (30) | <0.001** |
| Month 2 | n (%) | 163 (47) | 114 (33) | <0.001** |
| Month 3 | n (%) | 169 (49) | 108 (31) | <0.001** |
| Each month of Months | n (%) | 119 (35) | 63 (18) | <0.001** |
| 1 to 3 | | | | |
| Adequate relief of | | | | |
| abdominal pain/ | | | | |
| discomfort | | | | |
| Month 1 | n (%) | 192 (56) | 127 (36) | <0.001** |
| Month 2 | n (%) | 178 (52) | 134 (39) | <0.001** |
| Month 3 | n (%) | 183 (53) | 127 (36) | <0.001** |
| Each month of Months | n (%) | 139 (40) | 79 (23) | <0.001** |
| 1 to 3 | | | | |
| Adequate relief of | | | | |
| abnormal bowel | | | | |
| habits | | | | |
| Month 1 | n (%) | 180 (52) | 97 (28) | <0.001** |
| Month 2 | n (%) | 178 (52) | 104 (30) | <0.001** |
| Month 3 | n (%) | 173 (50) | 103 (30) | <0.001** |
| Each month of Months | n (%) | 129 (38) | 59 (17) | <0.001** |
| 1 to 3 | | | | |

*Significant at the 0.050 level for treatment effect;
**Significant at the 0.010 level for treatment effect.
^{a}P-values for treatment effect are based on a non-parametric analysis of covariance (ANCOVA) model with an effect for treatment and Baseline IBS severity as a covariate, stratified by pooled center and gender.
Note:
Percentages are based on the number of subjects in the ITT Population.
Note:
A subject is defined as a responder for a month if he/she reports adequate relief (‘yes’) for at least two of the four weeks during that month.
Note:
Weekly diary observations made on or after the date of randomization and up through two days after the date of last dose of study medication are included in this summary.
Data source: CSR Table 10.1.2.3.1 Left, Vol. 9 Righ

Based on ITT analyses using the monthly LOCF imputation method, the responder rate for the weekly diary questions on adequate relief was significantly higher (p<0.001) in the cilansetron 2 mg TID group compared with the placebo group for each of the three weekly diary questions on adequate relief at each month during the three months of treatment. The responder rate was 51% to 56% (cilansetron 2 mg TID) versus 28% to 36% (placebo) across the three diary questions for Month 1 and remained relatively stable for the rest of the study with a responder rate of 49% to 53% (cilansetron 2 mg TID) versus 30% to 36% (placebo) at Month 3 of treatment. The percentage of subjects who were responders for each month during the three-month treatment period was 35% to 40% (cilansetron 2 mg TIED) versus 17% to 23% (placebo) across the three diary questions. Analyses using the conservative imputation approach showed similar results to the analysis using the monthly LOCF imputation. Per-protocol analyses showed results consistent with the ITT analyses.

Study S241.3.011

Three-Month Treatment Period

The responder rate for the weekly diary questions on adequate relief at each month and all months during the three-month treatment period is presented in Table 2.7.3:23.

TABLE 2.7.3:23
Responder Rate for the Weekly Diary Questions on Adequate Relief
at Each Month and All Months During the Three-Month Treatment
Period (Monthly LOCF Imputation)
Study S241.3.011 (ITT Population)

| | | Cilansetron | | |
|---|---|---|---|---|
| | | 2 mg TID | Placebo | |
| Responder rate | Statistic | (N = 377) | (N = 369) | P-Value^{a} |
| Adequate relief of IBS | | | | |
| symptoms | | | | |
| Month 1 | n (%) | 216 (57) | 113 (31) | <0.001** |
| Month 2 | n (%) | 214 (57) | 123 (33) | <0.001** |
| Month 3 | n (%) | 208 (55) | 127 (34) | <0.001** |
| Each month of Months | n (%) | 148 (39) | 68 (18) | <0.001** |
| 1 to 3 | | | | |
| Adequate relief of | | | | |
| abdominal pain/ | | | | |
| discomfort | | | | |
| Month 1 | n (%) | 217 (58) | 146 (40) | <0.001** |
| Month 2 | n (%) | 229 (61) | 153 (41) | <0.001** |
| Month 3 | n (%) | 220 (58) | 153 (41) | <0.001** |
| Each month of Months | n (%) | 159 (42) | 93 (25) | <0.001** |
| 1 to 3 | | | | |
| Adequate relief of | | | | |
| abnormal bowel | | | | |
| habits | | | | |
| Month 1 | n (%) | 214 (57) | 103 (28) | <0.001** |
| Month 2 | n (%) | 209 (55) | 119 (32) | <0.001** |
| Month 3 | n (%) | 206 (55) | 126 (34) | <0.001** |
| Each month of Months | n (%) | 149 (40) | 61 (17) | <0.001** |
| 1 to 3 | | | | |

*Significant at the 0.050 level for treatment effect;
**Significant at the 0.010 level for treatment effect.
^{a}P-values for treatment effect are based on a non-parametric ANCOVA model with an effect for treatment and Baseline IBS severity as a covariate, stratified by pooled center and gender.
Note:
Percentages are based on the number of subjects in the ITT Population.
Note:
A subject is defined as a responder for a month if he/she reports adequate relief (‘yes’) for at least two of the four weeks during that month.
Note:
Weekly diary observations made on or after the date of randomization and up through two days after the date of last dose of study medication during the 12-week treatment period (or the date of first dose of study medication during the rerandomization period if earlier) are included in this summary.
Data source: CSR Table 10.1.2.3.1 Left, Vol. 9 Righ

Based on ITT analyses using the monthly LOCF imputation method, the responder rate for the weekly diary questions on adequate relief was significantly higher (p<0.001) in the cilansetron 2 mg TID group compared with the placebo group for each of the three weekly diary questions on adequate relief at each month during the three months of treatment. The responder rate was 57% to 58% (cilansetron 2 mg TID) versus 28% to 40% (placebo) across the three diary questions for Month 1 and remained relatively stable for the rest of the three-month treatment period with a responder rate of 55% to 58% (cilansetron 2 mg TID) versus 34% to 41% (placebo) at Month 3 of treatment. The percentage of subjects who were responders for each month during the three-month treatment period was 39% to 42% (cilansetron 2 mg TID) versus 17% to 25% (placebo) across the three diary questions. Analyses using the conservative imputation approach showed similar results to the analysis using the monthly LOCF imputation. Per-protocol analyses showed results consistent with the ITT analyses.

Four-Week Rerandomization Period

Based on rerandomized efficacy population using the conservative imputation method, the responder rate for the weekly diary questions on adequate relief was higher in general in subjects who were rerandomized to cilansetron 2 mg TID (C/C, 63% to 66% and P/C, 48% to 52% across the diary questions) compared with subjects who were rerandomized to placebo (C/P, 35% to 45% and P/P, 38% to 49%). In addition, within subjects who were rerandomized to cilansetron 2 mg TID, the responder rate was higher in subjects who were taking cilansetron 2 mg TID during the first three months of treatment (C/C, 63% to 66%) compared with subjects who were taking placebo during the first three months of treatment (P/C, 48% to 52%). Although comparisons between the first three months and the fourth month are of limited value due to the difference in the analysis populations used, it should be noted that responder rates were slightly higher for Month 4 compared-with Month 3 among both cilansetron-treated and placebo-treated subjects.

Shifts in the Number of Responders from Month 3 to Month 4

Shifts from responder at Month 3 to non-responder at Month 4 were consistently more common in the C/P treatment group compared with the other three treatment groups for each of the three diary questions regardless of the imputation approach (conservative or weekly LOCF) used for the analyses. Correspondingly, shifts from non-responder at Month 3 to responder at Month 4 were consistently less common in the C/P treatment group compared with the other three groups. Within the C/P treatment group, almost half of the responders at Month 3 shifted to non-responders by Month 4 (41%,to 49% across the diary questions using conservative imputation), while only a small portion of non-responders at Month 3 shifted to responders by Month 4 (9% to 17% across the diary questions). In comparison, within the C/C treatment group, shifts from non-responder to responder (27% to 33% across the diary questions using conservative imputation) were consistently more common compared with shifts from responder to non-responder (18% to 19%) for each of the three diary questions regardless of the imputation approach used for the analyses. Differences within the other two treatment groups (P/C and P/P) were less consistent across the diary questions.

Study S241.3.009

The responder rate for the weekly diary questions on adequate relief at each month and all months is presented for the ITT population during Months 1 to 3 and Months 4 to 6 in Table 2.7.3:24.

TABLE 2.7.3:24
Responder Rate for the Weekly Diary Questions on Adequate Relief
at Each Month and All Months (Monthly LOCF Imputation)
Study S241.3.009 (ITT Population)

| | | Cilansetron | | |
|---|---|---|---|---|
| | | 2 mg TID | Placebo | |
| Responder rate | Statistic | (N = 395) | (N = 397) | P-Value^{a} |
| Adequate relief of IBS | | | | |
| symptoms | | | | |
| Month 1 | n (%) | 193 (49) | 130 (33) | <0.001** |
| Month 2 | n (%) | 233 (59) | 163 (41) | <0.001** |
| Month 3 | n (%) | 239 (61) | 186 (47) | <0.001** |
| Month 4 | n (%) | 237 (60) | 197 (50) | 0.002** |
| Month 5 | n (%) | 237 (60) | 184 (46) | <0.001** |
| Month 6 | n (%) | 233 (59) | 188 (47) | <0.001** |
| Each month of Months | n (%) | 150 (38) | 85 (21) | <0.001** |
| 1 to 3 | | | | |
| Each month of Months | n (%) | 193 (49) | 147 (37) | <0.001** |
| 4 to 6 | | | | |
| Each month of Months | n (%) | 121 (31) | 66 (17) | — |
| 1 to 6 | | | | |
| Adequate relief of | | | | |
| abdominal pain/ | | | | |
| discomfort | | | | |
| Month 1 | n (%) | 199 (50) | 136 (34) | <0.001** |
| Month 2 | n (%) | 237 (60) | 174 (44) | <0.001** |
| Month 3 | n (%) | 242 (61) | 184 (46) | <0.001** |
| Month 4 | n (%) | 241 (61) | 198 (50) | 0.001** |
| Month 5 | n (%) | 242 (61) | 191 (48) | <0.001** |
| Month 6 | n (%) | 237 (60) | 197 (50) | 0.002** |
| Each month of Months | n (%) | 159 (40) | 90 (23) | <0.001** |
| 1 to 3 | | | | |
| Each month of Months | n (%) | 202 (51) | 154 (39) | <0.001** |
| 4 to 6 | | | | |
| Each month of Months | n (%) | 133 (34) | 76 (19) | — |
| 1 to 6 | | | | |
| Adequate relief of | | | | |
| abnormal bowel | | | | |
| habits | | | | |
| Month 1 | n (%) | 214 (54) | 134 (34) | <0.001** |
| Month 2 | n (%) | 245 (62) | 172 (43) | <0.001** |
| Month 3 | n (%) | 251 (64) | 191 (48) | <0.001** |
| Month 4 | n (%) | 245 (62) | 194 (49) | <0.001** |
| Month 5 | n (%) | 247 (63) | 177 (45) | <0.001** |
| Month 6 | n (%) | 243 (62) | 186 (47) | <0.001** |
| Each month of Months | n (%) | 165 (42) | 87 (22) | <0.001** |
| 1 to 3 | | | | |
| Each month of Months | n (%) | 204 (52) | 143 (36) | <0.001** |
| 4 to 6 | | | | |
| Each month of Months | n (%) | 136 (34) | 62 (16) | — |
| 1 to 6 | | | | |

*Significant at the 0.050 level for treatment effect;
**Significant at the 0.010 level for treatment effect.
^{a}P-values for treatment effect are based on a non-parametric analysis of covariance (ANCOVA) model with an effect for treatment and Baseline IBS severity as a covariate, stratified by pooled country and gender.,
Note:
Percentages are based on the number of subjects in the ITT Population.
Note:
A subject is defined as a responder for a month if he/she reports adequate relief (‘yes’) for at least two of the four weeks during that month.
Note:
Weekly diary observations made on or after the date of randomization and up through two days after the date of last dose of study medication are included in this summary.
Data source: CSR Table 10.1.2.3.1 Left, Vol. 9 Righ, CSR Table 10.1.2.3.5 Left, Vol. 9 Righ, CSR Table 10.1.2.1.12 Left, Vol. 9 Righ and CSR Table 10.1.2.1.16 Left, Vol. 9 Righ

Based on ITT analyses using the monthly LOCF imputation method, the responder rate for the weekly diary questions on adequate relief was significantly higher (p<0.00l to p=0.002) in the cilansetron 2 mg TIE) group compared with the placebo group for each of the three weekly diary questions on adequate relief at each month during the six months of treatment. The responder rate was 49% to 54% (cilansetron 2 mg TID) versus 33% to 34% (placebo) across the three diary questions for Month 1 and increased to 61% to 64% (cilansetron 2 mg TID) versus 46% to 48% (placebo) by Month 3 of treatment. At Month 6, the responder rate was 59% to 62% (cilansetron 2 mg TID) versus 47% to 50% (placebo) across the three diary questions demonstrating that the favorable effect of cilansetron was sustained for up to six months. The percentage of subjects who were responders for each month during Months 1 to 3 was 38% to 42% (cilansetron 2 mg TIED) versus 21% to 23% (placebo) and for each month during Months 4 to 6 the responder rate was 49% to 52% (cilansetron 2 mg TID) versus 36% to 39% (placebo) across the three diary questions. The percentage of subjects who were responders for each month during Months 1 to 6 was 31% to 34% (cilansetron 2 mg TID) versus 16% to 19% (placebo) across the three diary questions. Analyses using the conservative imputation approach showed similar results to the analysis using the monthly LOCF imputation. Per-protocol analyses showed results consistent with the ITT analyses.

Adequate Relief Rate for Weekly Diary Ouestions

Summary Across Studies

In all three studies, the mean adequate relief rate (the individual proportion of “yes”) during the three-month treatment period was significantly higher (p<0.001) in the cilansetron 2 mg TID group compared with the placebo group for each of the three weekly diary questions. The median adequate relief rates during the three-month treatment period were 0.50 to 0.58 (cilansetron 2 mg TID) versus 0.08 to 0.17 (placebo) across the three diary questions in Study S241.3.006, 0.58 for all three questions (cilansetron 2 mg TID) versus 0.17 to 0.28 (placebo) in Study S241.3.011, and 0.58 to 0.67 (cilansetron 2 mg TID) versus 0.33 for all three diary questions (placebo) in Study S241.3.009. The median adequate relief rates during the six-month treatment period in Study S241.3.009 were 0.63 to 0.70 (cilansetron 2 mg TID) versus 0.40 to 0.42 (placebo) across the three diary questions.

Study S241.3.006

Based on ITT analyses using the observed case approach, the mean adequate relief rate (the individual proportion of “yes”) for the weekly diary questions during the three-month treatment period was significantly higher (p<0.001) in the cilansetron 2 rug TID group compared with the placebo group for each of the three weekly diary questions on adequate relief. The median adequate relief rates were 0.50 versus 0.08 for IBS symptoms, 0.58 versus 0.17 for abdominal pain/discomfort, and 0.50 versus 0.08 for abnormal bowel habits in the cilansetron 2 mg TID and placebo groups, respectively. Analyses using the weekly LOCF imputation approach showed similar results to the observed case analysis. Per-protocol analyses showed results consistent with the ITT analyses.

Study S241.3.011

Based on ITT analyses using the observed case approach, the mean adequate relief rate (the individual proportion of “yes”) for the weekly diary questions during the three-month treatment period was significantly higher (p<0.001) in the cilansetron 2 mg TID group compared with the placebo group for each of the three weekly diary questions on adequate relief. The median adequate relief rates were 0.58 versus 0.17 for IBS symptoms, 0.58 versus 0.28 for abdominal pain/discomfort, and 0.58 versus 0.17 for abnormal bowel habits in the cilansetron 2 mg TID and placebo groups, respectively. Analyses using the weekly LOCF imputation approach showed similar results to the observed case analysis. Per-protocol analyses showed results consistent with the ITT analyses.

Study S241.3.009

Months 1 to 3

Based on ITT analyses using the observed case approach, the mean adequate relief rate (the proportion of “yes”) for the weekly diary questions during Months 1 to 3 was significantly higher (p<0.001) in the cilansetron 2 mg TID group compared with the placebo group for each of the three weekly diary questions on adequate relief. The median adequate relief rates were 0.58 versus 0.33 for IBS symptoms, 0.58 versus 0.33 for abdominal pain/discomfort, and 0.67 versus 0.33 for abnormal bowel habits in the cilansetron 2 mg TID and placebo groups, respectively.

Analyses using the weekly LOCF imputation approach showed similar results to the observed case analysis. Per-protocol analyses showed results consistent with the ITT analyses.

Months 1 to 6

Based on ITT analyses using the observed case approach, the mean adequate relief rate (the proportion of “yes”) for the weekly diary questions during the six months of treatment was significantly higher (p<0.001) in the cilansetron 2 mg TID group compared with the placebo-group for each of the three weekly diary questions on adequate relief. The median adequate relief rates were 0.64 versus 0.40 for IBS symptoms, 0.63 versus 0.42 for abdominal pain/discomfort, and 0.70 versus 0.40 for abnormal bowel habits in the cilansetron 2 mg TID and placebo groups, respectively. Analyses using the weekly LOCF imputation approach showed similar results to the observed case analysis. Per-protocol analyses showed results consistent with the ITT analyses.

Number of Subjects with Adequate Relief for Weekly Diary Questions by Week

Summary Across Studies

The proportion of subjects with adequate relief for the weekly diary questions was higher in the cilansetron 2 mg Ti) group compared with the placebo group for each question at each week during the three months (Studies S241.3.006 and S241.3.011) and six months (Study S241.3.009) of treatment. In Study S241.3.006, the proportion of subjects with adequate relief was 41% to 43% (cilansetron 2 mg TID) versus 19% to 26% (placebo) across the three diary questions at Week 1 and increased to 51% to 55% (cilansetron 2 mg TID) versus 30% to 38% (placebo) at Week 12. In Study S241.3.011, the proportion of subjects with adequate relief was 35% to 45% (cilansetron 2 mg TID) versus 21% to 29% (placebo) at Week 1 and increased to 56% to 62% (cilansetron 2 mg TID) versus 36% to 40% (placebo) at Week 12. In Study S241.3.009, the proportion of subjects with adequate relief was 32% to 37% (cilansetron 2 mg TID) versus 24% to 25% (placebo) at Week 1 and increased to 70% to 71% (cilansetron 2 mg TID) versus 53% to 57% (placebo) at Week 26 demonstrating that the favorable effect of cilansetron was sustained for up to six months. Generalized Estimating Equation analysis with factors for treatment group, pooled country, gender, Baseline IBS severity, and time (week) resulted in a significant treatment effect (p<0.001) and time effect (p<0.001) for each of the three weekly diary questions in each of the three pivotal efficacy studies. In Study S241.3.011, there was an immediate (within a week) loss of the treatment effect following blinded withdrawal of cilansetron 2 mg TID with no evidence of rebound within four weeks.

Study S241.3.006

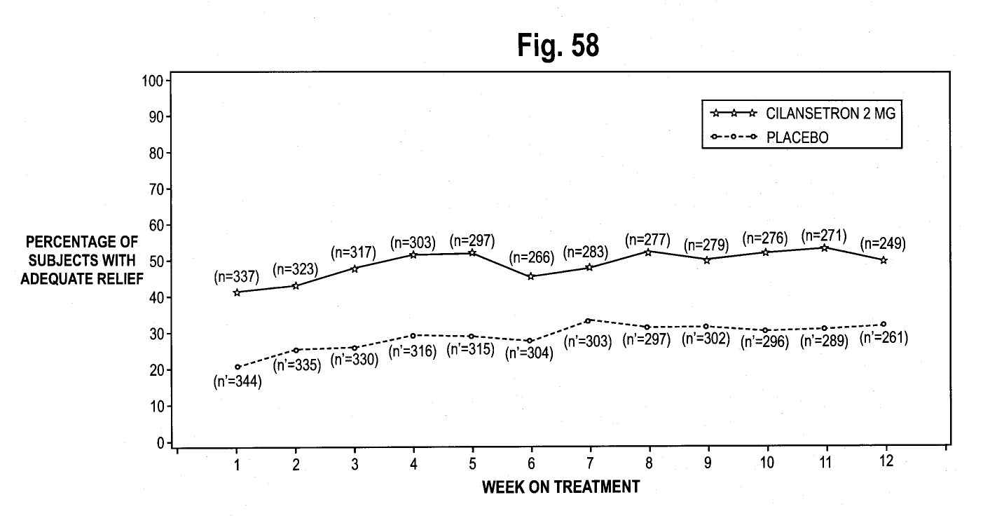

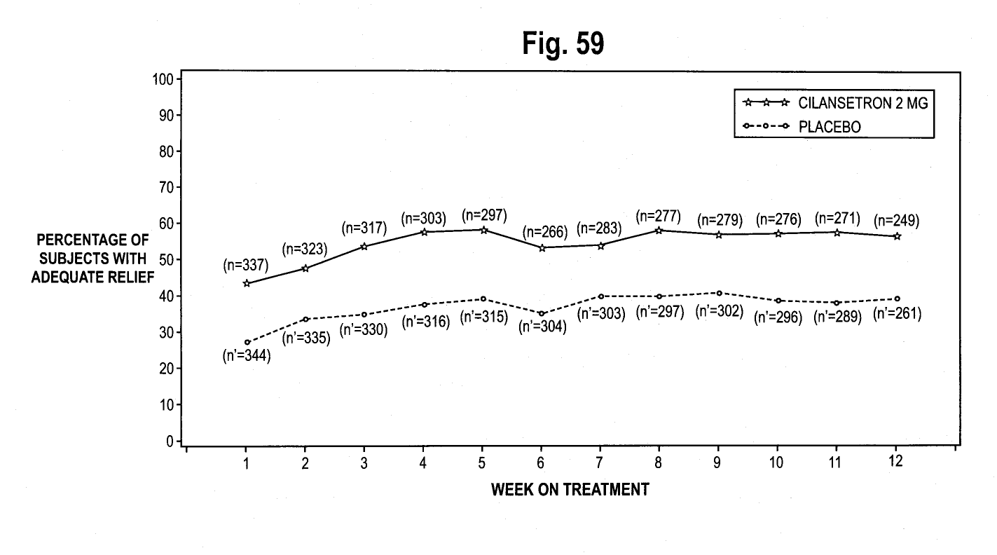

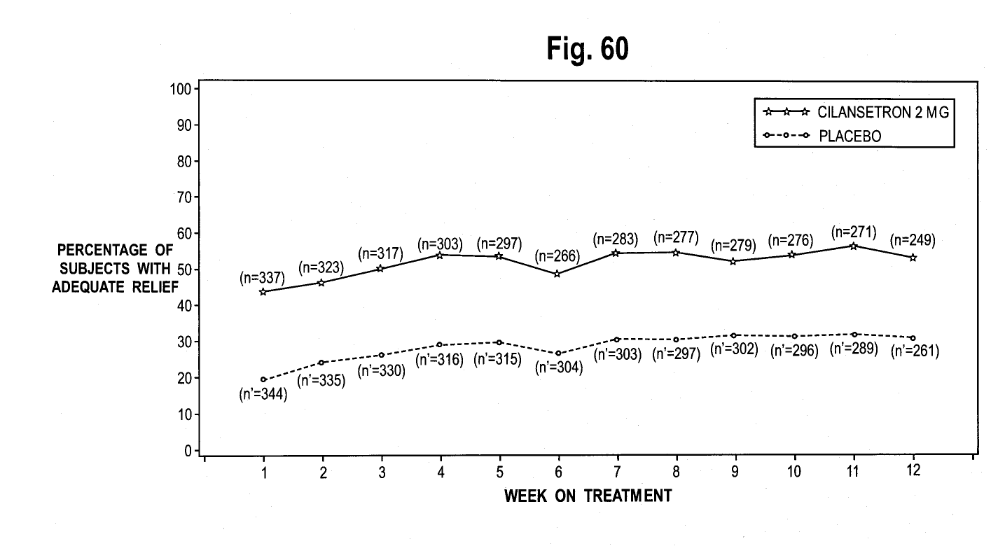

Summaries (observed case) of adequate relief for the weekly diary questions by week are displayed in FIG. 58 for IBS symptoms, FIG. 59 for abdominal pain/discomfort, and FIG. 60 for abnormal bowel habits.

Based on ITT analyses using the observed case approach, the proportion of subjects with adequate relief for the weekly diary questions was higher in the cilansetron 2 mg TID group compared with the placebo group for each question at each week during the three months of treatment. The proportion of subjects with adequate relief was 41% to 43% (cilansetron 2 Xmg TID) versus 19% to 26% (placebo) across the three diary questions at Week 1 and increased to 48% to 53% (cilansetron 2 mg TID) versus 25% to 34% (placebo) by Week 3 of treatment. The proportion of subjects with adequate relief continued to increase until Week 5 (53% to 57% versus 29% to 38%) and remained relatively stable to the end of the treatment period (Week 12). At Week 12, the proportion of subjects with adequate relief was 51% to 55% (cilansetron 2 mg TID) versus 30% to 38% (placebo) across the three diary questions demonstrating that the favorable effect of cilansetron was sustained for up to 12 weeks. Generalized Estimating Equation analysis with factors for treatment group, pooled center, gender, Baseline IBS severity, and time (week) resulted in a significant treatment effect @<0.001) and time effect @<0.001) for each of the three weekly diary questions. Analyses using the weekly LOCF imputation method showed similar results to the observed case analysis. Per-protocol analyses showed results consistent with the FIT analyses.

Study S241.3.011

Three-Month Treatment Period

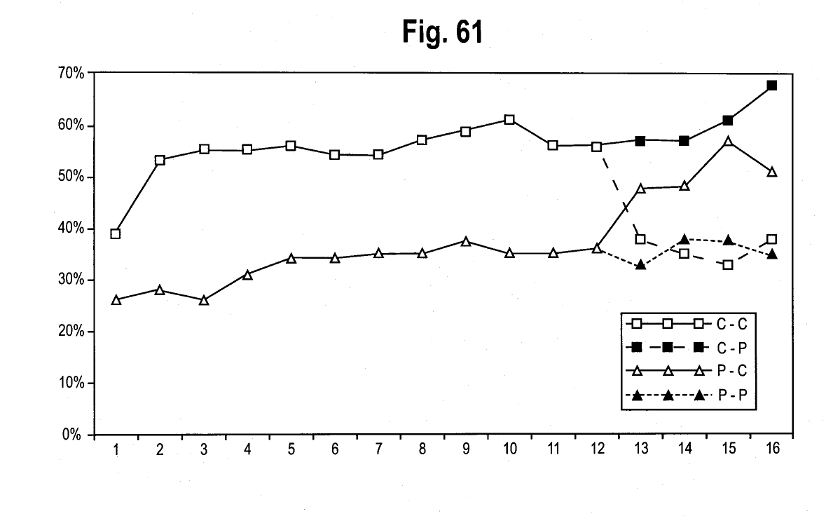

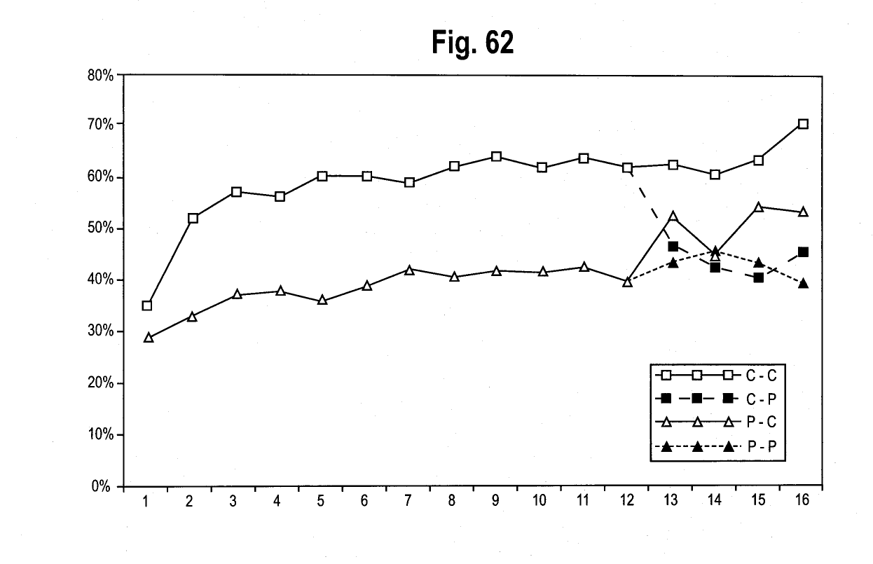

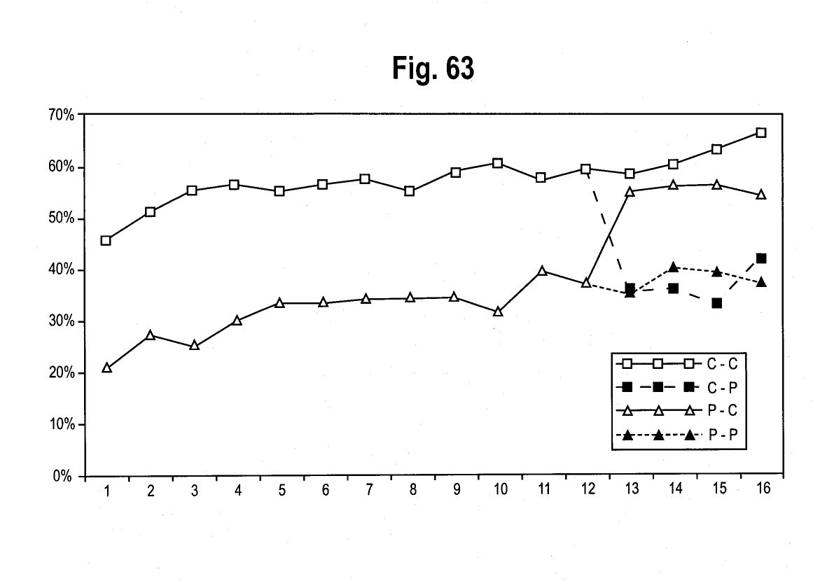

The percentage of subjects (observed case) with adequate relief during the entire four-month treatment period by week is displayed in FIG. 61 for IBS symptoms, FIG. 62 for abdominal pain/discomfort, and FIG. 63 for abnormal bowel habits.

Based on ITT analyses using the observed case approach, the proportion of subjects with adequate relief for the weekly diary questions was higher in the cilansetron 2 mg TID group compared with the placebo group for each question at each week during the three months of treatment. At Week 1, the proportion of subjects with adequate relief was 35% to 45% (cilansetron 2 mg TID) versus 21% to 29% (placebo) across the three diary questions, increased to 55% to 57% (cilansetron 2mg TID) versus 25% to 37% (placebo) by Week 3 of treatment, and remained relatively stable to the end of the treatment period (Week 12). At Week 12, the proportion of subjects with adequate relief was 56% to 62% (cilansetron 2 mg TID) versus 36% to 40% (placebo) across the three diary questions demonstrating that the favorable effect of cilansetron 2 mg TID was sustained for up to 12 weeks. Generalized Estimating Equation analysis with factors for treatment group, pooled center, gender, Baseline IBS severity, and time (week) resulted in a significant treatment effect (p<0.001) and time effect (p<0.001) for each of the three weekly diary questions. Analyses using the weekly LOCF imputation method showed similar results to the observed case analysis. Per-protocol analyses showed results consistent with the FIT analyses.

Four-Week Rerandomization Period

The percentage of subjects (observed case) with adequate relief during the entire four-month treatment period by week is displayed in FIG. 61 for IBS symptoms, FIG. 62 for abdominal pain/discomfort, and FIG. 63 for abnormal bowel habits.

Using the observed case approach, the proportion of subjects with adequate relief was higher in subjects rerandomized to cilansetron treatment (C/C, 57% to 71% and P/C, 45% to 57%) compared with subjects rerandomized to placebo (C/P, 33% to 47% and P/P, 33% to 46%) for each question at each week during the four-week rerandomization period. In addition, within subjects who were rerandomized to cilansetron 2 mg TID, the proportion of subjects with adequate relief was higher in subjects who were taking cilansetron 2 mg TED during the first three months of treatment (C/C, 57% to 7 1%) compared with subjects who were taking placebo during the first three months of treatment (P/C, 45% to 57%) for each question at each week during the four weeks of the rerandomization period. It should be noted that there appeared to be an immediate (within a week) loss of the treatment effect in cilansetron-treated subjects who were rerandomized to placebo as, starting at Week 13, adequate relief rates in the C/P group were in the range of rates in the P/P group and substantially below rates seen in the C/C treatment group for each of the three diary questions. Analyses using the weekly LOCF imputation method showed similar results to the observed case analysis.

Study S241 .3.009

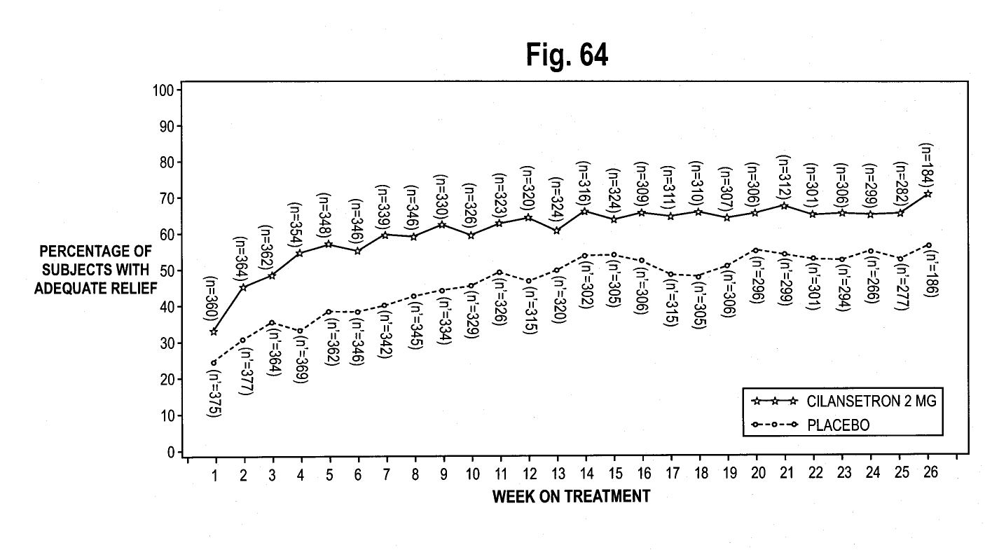

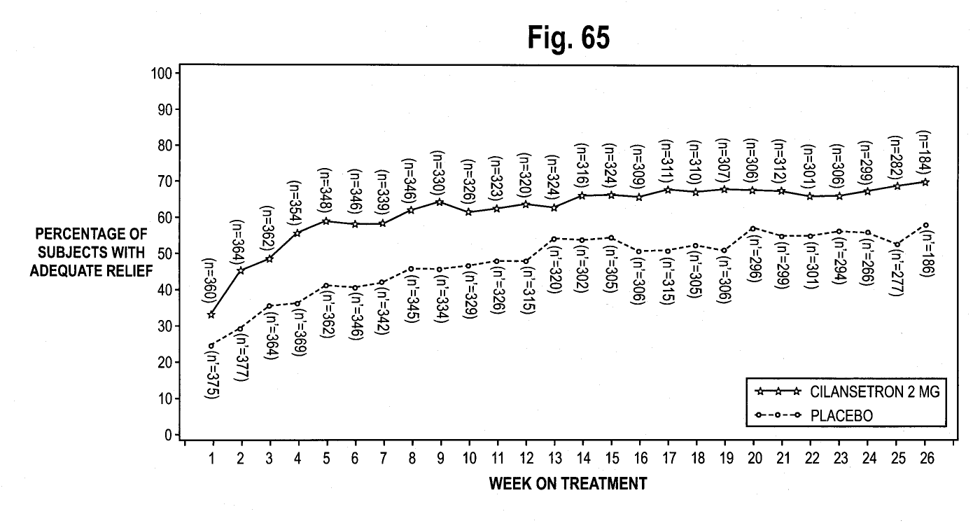

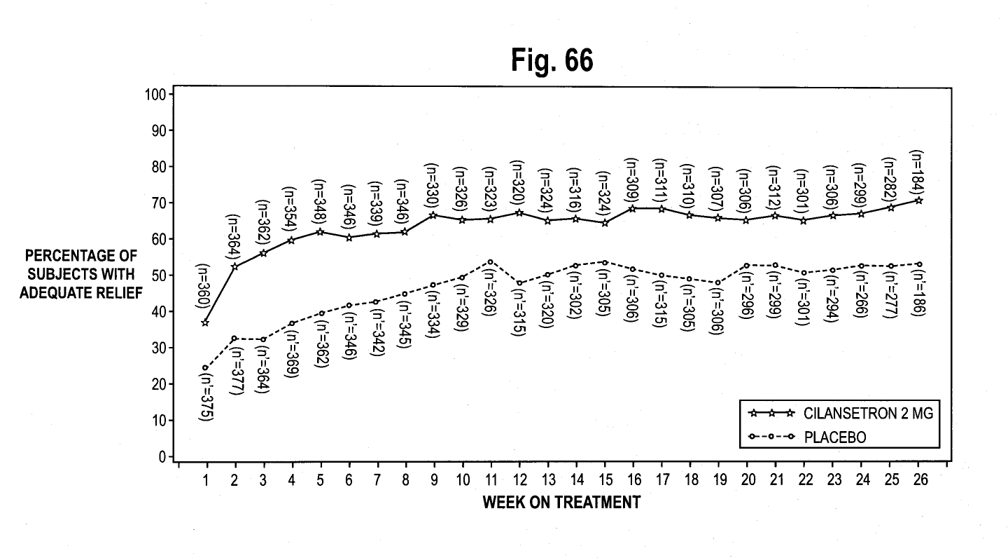

Summaries (observed case) of adequate relief for the weekly diary questions by week are displayed in FIG. 64 for IBS symptoms, FIG. 65 for abdominal pain/discomfort, and FIG. 66 for abnormal bowel habits.

Based on ITF analyses using the observed case approach, the proportion of subjects with adequate relief for the weekly diary questions was significantly higher in the cilansetron 2 mg TID group compared with the placebo group for each question at each week during the six months of treatment. The proportion of subjects with adequate relief was 32% to 37% (cilansetron 2 mg TID) versus 24% to 25% (placebo) across the three diary questions at Week 1 and increased to 57% to 61% (cilansetron 2 mg TID) versus 38% to 41% (placebo) by Week 5 of treatment. The proportion of subjects with adequate relief continued to increase and reached a plateau at approximately Week 9 (62% to 66%, cilansetron 2 mg TIED versus 44% to 47%, placebo) and continued to increase slightly for the rest of the treatment period. At Week 26, the proportion of subjects with adequate relief was 70% to 71% (cilansetron 2 mg TID) versus 53% to 57% (placebo) across the three diary questions demonstrating that the favorable effect of cilansetron was sustained for up to six months. Generalized Estimating Equation analysis with factors for treatment group, pooled country, gender, Baseline IBS severity, and time (week) resulted in a significant treatment effect (p<0.001) and time effect (p<0.001) for each of the three weekly diary questions. Analyses using the weekly LOCF imputation method showed similar results to the observed case analysis. Per-protocol analyses showed results consistent with the ITT analyses.

Summary of Daily Diary Assessments Across Studies

Analysis of ]]BS symptoms recorded in the daily diary showed a statistically significant and clinically meaningful beneficial effect of cilansetron 2 mg TID on the symptoms of abdominal pain/discomfort, urgency, and bloating or feeling of abdominal distension at the end of treatment in all three pivotal efficacy studies. The rate of pain-free days and the rate of urgency-free days during the treatment period overall and at the end of treatment was generally consistent with the beneficial effect of cilansetron 2 mg TIED described above, except Study S241.3.009, which showed clinically unremarkable median urgency-free days in both treatment groups. In Study S241.3.006, a statistically significant beneficial effect of cilansetron 2 mg TIE) at the end of treatment was also seen on the feeling of incomplete evacuation. Cilansetron 2 mg TID treatment was also associated with statistically significant increased stool firmness, decreased stool frequency, and increased straining at the end of treatment in all three studies. The mean scores for daily stool consistency suggesting increased stool firmness in the three studies did not reach the other end of the spectrum of hard and lumpy stools (scores of 1 and 2 in the Bristol stool form scale). The mean stool frequency decreased in the cilansetron 2 mg group to 2.45 (Study S241.3.006), 2.41 (Study S241.3.011), and 1.96 (Study S241.3.009) stools per day at the end of treatment. In Study S241.3.011, there was an immediate (within a week) loss of the treatment effect following blinded withdrawal of cilansetron 2 mg TID with no evidence of rebound within four weeks.

Abdominal Pain/Discomfort

Study S241.3.006

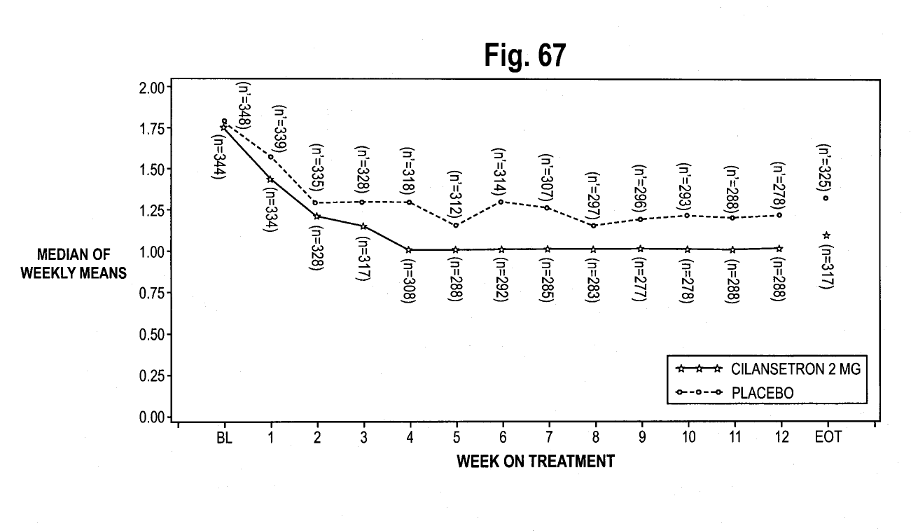

A summary of weekly means for daily abdominal pain/discomfort is presented for the ITT population in FIG. 67.

At Baseline, the mean scores for daily abdominal pain/discomfort (0=none, 1=mild, 2=moderate, 3=severe, 4=intolerable) were similar in the treatment groups (1.77 versus 1.75). Weekly mean scores for daily abdominal pain/discomfort gradually decreased in both treatment groups during the study with the cilansetron 2 mg TID group having a lower mean score at every week compared with the placebo group. At the end of treatment, a significantly larger decrease from Baseline was observed in the cilansetron 2 mg TID group compared with the placebo group (average change of weekly means: −0.69 versus −0.48, p<0.001).

The rate of pain-free days was also calculated from treatment start to treatment stop and presented for the ITT population in Table 2.7.3.6.3.1. During the three-month treatment period, the rate of pain-free days was almost twice as high in the cilansetron 2 mg TID group compared with the placebo group (median rate of 0.19 versus 0.10 for the three-month treatment period).

Study S241.3.011

Three-Month Treatment Period

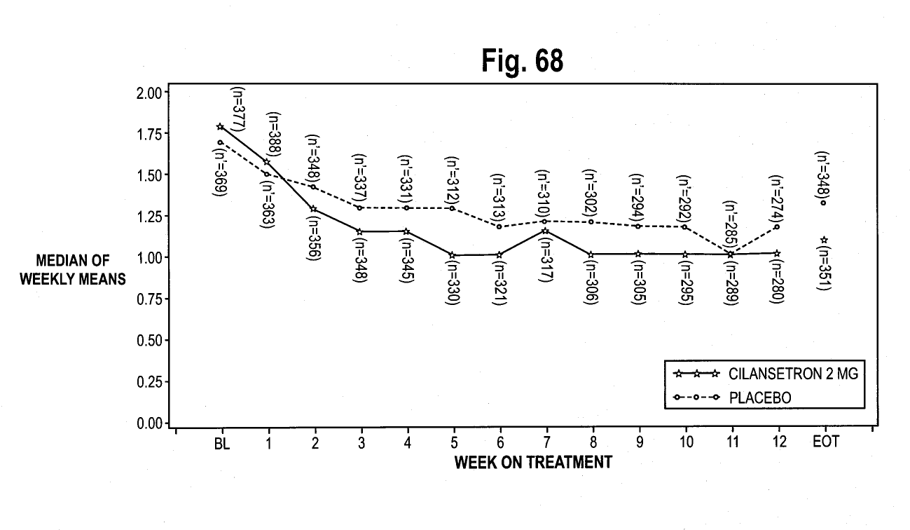

A summary of weekly means for daily abdominal pain/discomfort is presented for the ITT population in FIG. 68.

At Baseline, the mean scores for daily abdominal pain/discomfort (0=none, 1=mild, 2=moderate, 3=severe, 4=intolerable) were similar in the treatment groups (1.79 versus 1.75). Weekly mean scores for daily abdominal pain/discomfort gradually decreased in both treatment groups during the study with the cilansetron 2 mg TID group having a lower mean score at every week compared with the placebo group. At the end of treatment, a significantly larger decrease from Baseline was observed in the cilansetron 2 mg TID group compared with the placebo group (mean change of −0.69 versus −0.47, p=0.010).

At Baseline, the median number of pain-free days during the run-in period was zero in both treatment groups. At the end of treatment (during the last two weeks of study medication), a larger increase from Baseline was observed in the cilansetron 2 mg TID group compared with the placebo group (median rate of 0.15 versus 0.08 at the end of treatment).

During the three-month treatment period, the rate of pain-free days was approximately twice as high in the cilansetron 2 mg TID group compared with the placebo group (median rate of 0.22 versus 0.11 for the three-month treatment period).

Four-Week Rerandomization Period

At rerandomization Baseline, the means scores for daily abdominal pain/discomfort were lower in subjects who were treated with cilansetron 2 mg TID during the three-month treatment period (C/C, 1.05 and C/P, 1.11) compared with subjects who were treated with placebo (P/C, 1.20 and P/P, 1.29). Weekly mean scores for daily abdominal pain/discomfort decreased during the four-week rerandomization period in subjects who were rerandomized to cilansetron treatment (mean change from rerandomization Baseline at the end of treatment, C/C, −0.09 and P/C, −0.08) and remained relatively stable in the P/P treatment group (mean change of −0.04). In contrast, weekly mean scores for daily abdominal pain/discomfort increased during the four-week rerandomization period in the C/P treatment group (mean change of 0.16).

Study S241.3.009

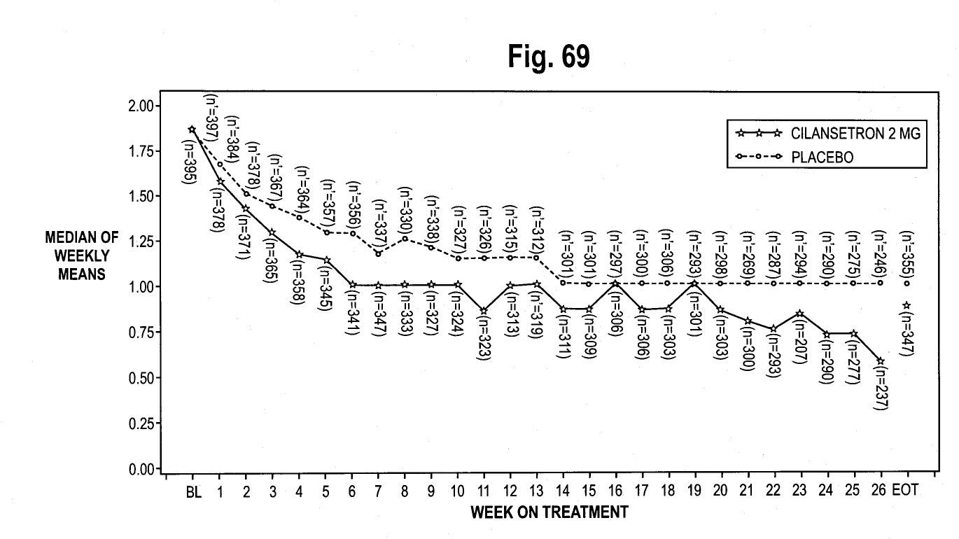

A summary of weekly means for daily abdominal pain/discomfort is presented for the ITT population in FIG. 69.

At Baseline, the mean scores for daily abdominal pain/discomfort (0=none, 1=mild, 2=moderate, 3=severe, 4=intolerable) were similar in the treatment groups (1.83 versus 1.82). Weekly mean scores for daily abdominal pain/discomfort gradually decreased in both treatment groups during the study with the cilansetron 2 mg TID group having a lower mean score at every week compared with the placebo group. At the end of treatment, a significantly larger decrease from Baseline was observed in the cilansetron 2 mg TID group compared with the placebo group (−0.92 versus −0.71, p<0.001).

At Baseline, the median number of pain-free days during the run-in period was zero in both treatment groups. At the end of treatment, a larger increase from Baseline was observed in the cilansetron 2 mg TID group compared with the placebo group (0.29 versus 0.08).

The rate of pain-free days was also calculated from treatment start to treatment stop and presented for the ITT population in Table 2.7.3.6.3.2 (overall) and Table 2.7.3.6.3.3 (Month 1 to 3 and Month 4 to 6). During the six-month treatment period, the rate of pain-free days was more than twice as high in the cilansetron 2 mg TID group compared with the placebo group (median rate of 0.19 versus 0.08 for the six-month treatment period). Similarly, the rate of pain-free days was more than twice as high in the cilansetron 2 mg TID group compared with the placebo group during Month 1 to Month 3 (median rate of 0.13 versus 0.05 for the three-month treatment period) and during Month 4 to Month 6 (median rate of 0.31 versus 0.12 for the three-month treatment period).

Stool Consistency

Study S241.3.006

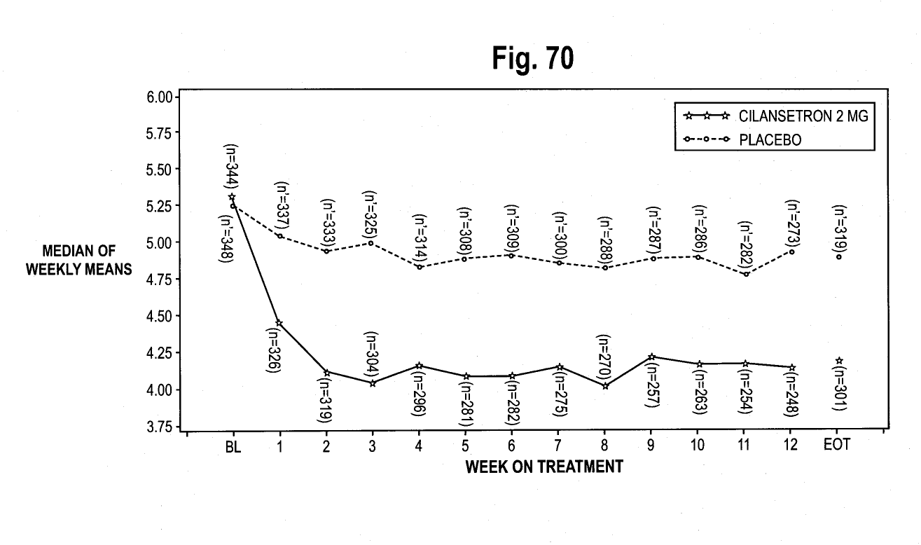

A summary of weekly means for daily stool consistency is presented for the ITT population in FIG. 70.

At Baseline, the mean scores for daily stool consistency (score range from 1 to 7, 1=separate hard lumps like nuts; 7=watery, no solid pieces) were similar in the treatment groups (5.31 versus 5.29). Weekly mean scores for daily stool consistency gradually decreased in both treatment groups during the study with the cilansetron 2 mg TID group having a lower mean score at every week compared with the placebo group. At the end of treatment, a significantly larger decrease from Baseline was observed in the cilansetron 2 mg TID group compared with the placebo group (−1.22 versus −0.48, p<0.001) with mean scores of 4.10 in the cilansetron 2 mg TID group versus 4.81 in the placebo group.

Study S241.3.011

Three-Month Treatment Period

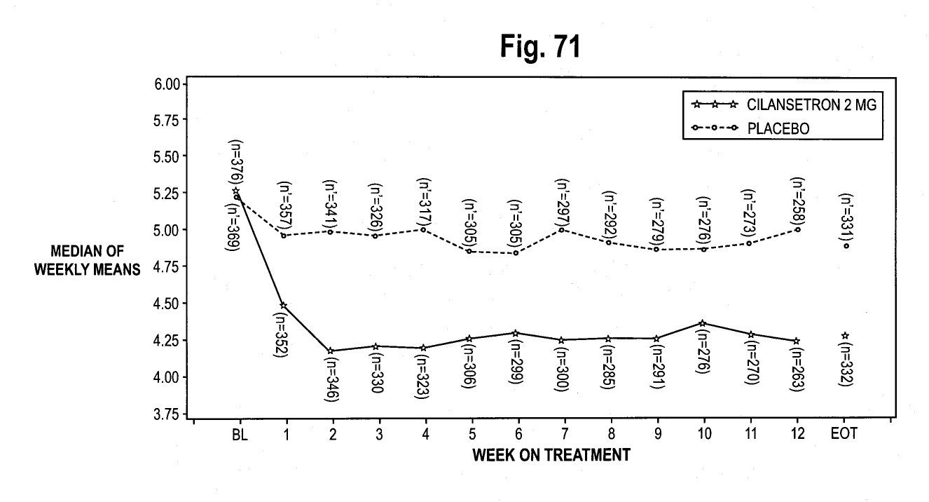

A summary of weekly means for daily stool consistency is presented for the ITT population in FIG. 71.

At Baseline, the mean scores for daily stool consistency (score range from 1 to 7, 1=separate hard lumps like nuts; 7=watery, no solid pieces) were similar in the treatment groups (5.29 versus 5.23). Weekly mean scores for daily stool consistency decreased in both treatment groups during the three-month treatment period with the cilansetron 2 mg TID group having a lower mean score at every week compared with the placebo group. At the end of treatment, a significantly larger decrease from Baseline was observed in the cilansetron 2 mg TID group compared with the placebo group (mean change of −1.02 versus −0.37, p<0.001) with mean scores of 4.28 in the cilansetron 2 mg TID group versus 4.87 in the placebo group.

Four-Week Rerandomization Period

At rerandomization Baseline, the mean scores for daily stool consistency were lower in subjects who were treated with cilansetron 2 mg TID during the three-month treatment period (C/C, 4.18 and C/P, 4.33) compared with subjects who were treated with placebo (P/C, 4.78 and P/P, 4.96). Weekly mean scores for daily stool consistency decreased during the four-week rerandomization period in the P/C treatment group (mean change from rerandomization Baseline at end of treatment, −0.84) and slightly decreased in the C/C (mean change of −0.09) and P/P (mean change of −0.13) treatment groups. In contrast, weekly mean scores for daily stool consistency increased during the four-week rerandomization period in the C/P treatment group (mean change of 0.55).

Study S241.3.009

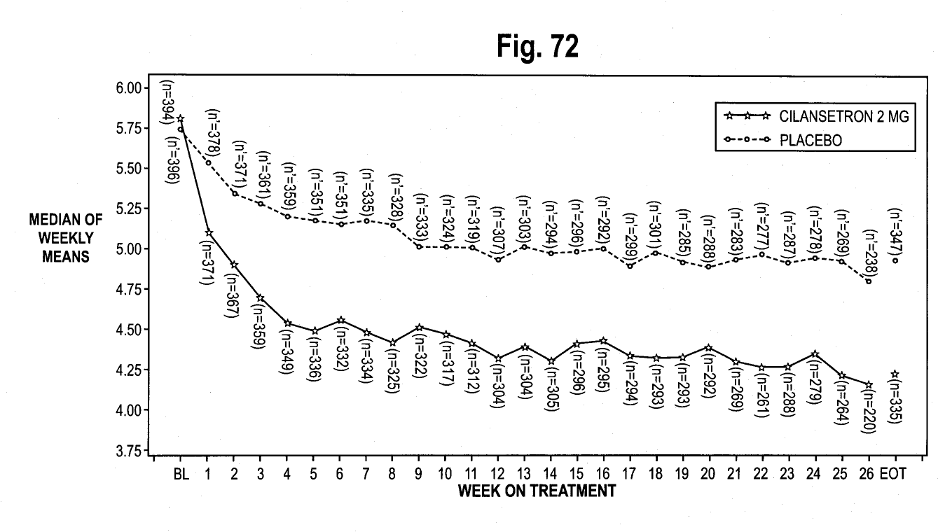

A summary of weekly means for daily stool consistency is presented for the ITT population in FIG. 72.

At Baseline, the mean scores for daily stool consistency (score range from 1 to 7, 1=separate hard lumps like nuts; 7=watery, no solid pieces) were similar in the treatment groups (5.62 versus 5.59). Weekly mean scores for daily stool consistency gradually decreased in both treatment groups during the study with the cilansetron 2 mg TID group having a lower mean score at every week compared with the placebo group. At the end of treatment, a significantly larger decrease from Baseline was observed in the cilansetron 2 mg TID group compared with the placebo group (−1.40 versus −0.73, p<0.001) with mean scores of 4.27 in the cilansetron 2 mg TIED group versus 4.87 in the placebo group.

Stool Frequency

Study S241.3.006

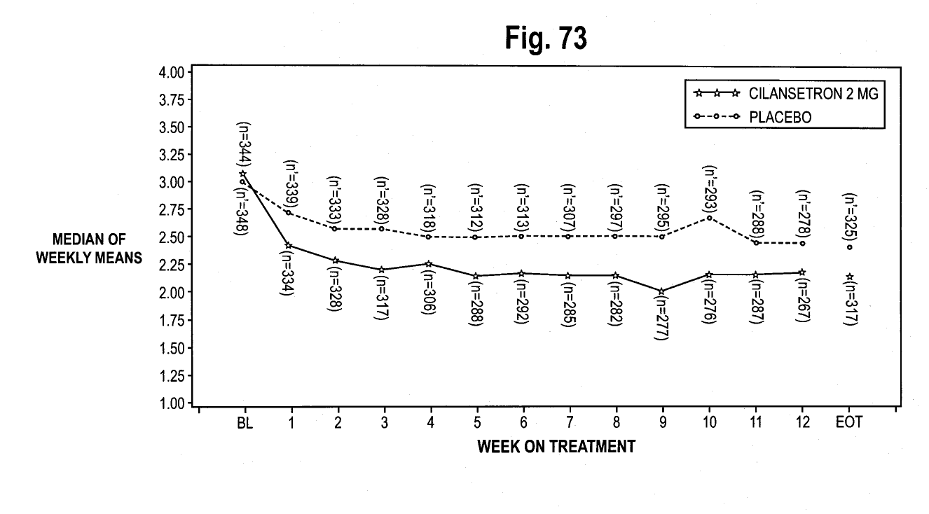

A summary of weekly means for daily stool frequency is presented for the IT population in FIG. 73.

At Baseline, the mean number of stools per day was similar in the treatment groups (3.40 versus 3.34). Weekly mean number of stools per day gradually decreased in both treatment groups during the study with the cilansetron 2 mg TID group having a lower mean at every week compared with the placebo group. At the end of treatment, a significantly larger decrease from Baseline was observed in the cilansetron 2 mg TID group compared with the placebo group (−0.98 versus −0.51, p<0.001) with mean number of stools per day of 2.45 in the cilansetron 2 mg TID group versus 2.83 in the placebo group.

Study S241.3.011

Three-Month Treatment Period

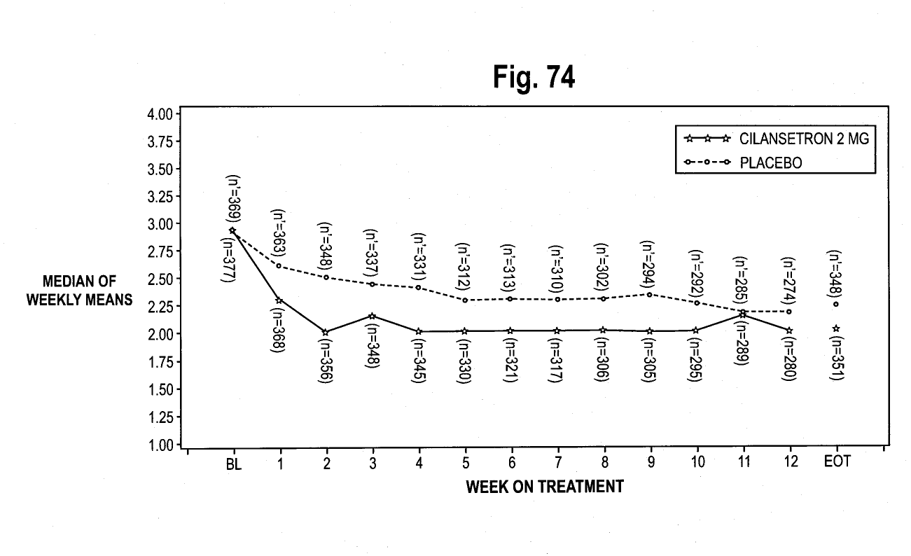

A summary of weekly means for daily stool frequency is presented for the ITT population in FIG. 74.

At Baseline, the mean number of stools per day was similar in the treatment groups (3.30 versus 3.13). Weekly mean number of stools per day decreased in both treatment groups during the three-month treatment period with the cilansetron 2 mg TID group having a lower mean at every week compared with the placebo group. At the end of treatment, a significantly larger decrease from Baseline was observed in the cilansetron 2 mg TID group compared with the placebo group (mean change of −0.91 versus −0.61, p<0.001) with mean number of stools per day of 2.41 in the cilansetron 2 mg TID group versus 2.52 in the placebo group.

Four-Week Rerandomization Period

At rerandomization Baseline, the mean number of stools per day was lower in the C/P (2.24) treatment group compared with the other three treatment groups (2.40 to 2.49). Weekly mean number of stools per day decreased during the four-week rerandomization period in the P/C treatment group (mean change from rerandomization Baseline at end of treatment, −0.31) and remained relatively stable in the C/C (mean change of −0.02) and P/P (mean change of −0.05) treatment groups. In contrast, weekly mean number of stools per day increased during the four week rerandomization period in the C/P treatment group (mean change of 0.22).

Study S241.3.009

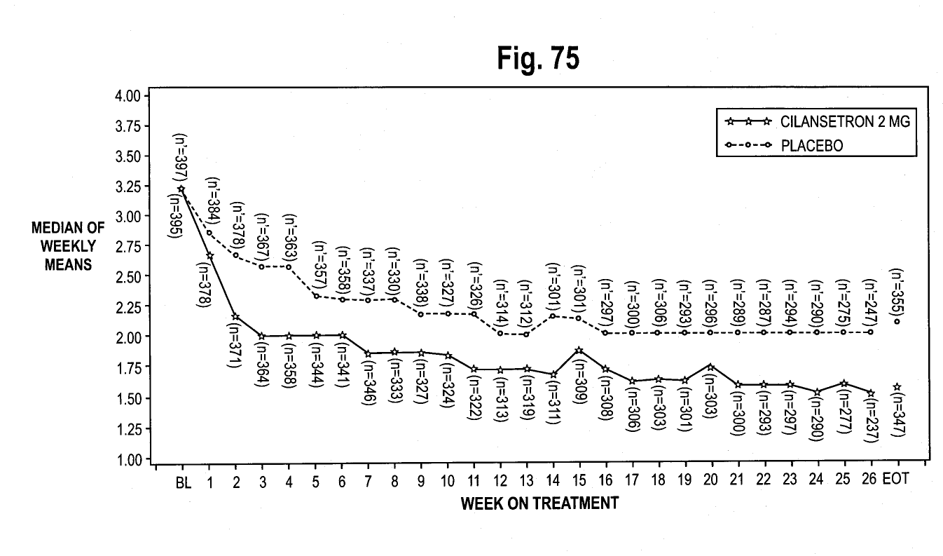

A summary of weekly means for daily stool frequency is presented for the ITT population in FIG. 75.

At Baseline, the mean number of stools per day was similar in the treatment groups (3.51 versus 3.57). Weekly mean number of stools per day gradually decreased in both treatment groups during the study with the cilansetron 2 mg TID group having a lower mean at every week compared with the placebo group. At the end of treatment, a significantly larger decrease from Baseline was observed in the cilansetron 2 mg TID group compared with the placebo group (−1.60 versus −1.18, p<0.001) with mean number of stools per day of 1.96 in the cilansetron 2 mg TID group versus 2.42 in the placebo group.

Straining

Study S241.3.006

At Baseline, mean straining (“yes”=1, “no”=0) was equal in the treatment groups (0.20). Weekly mean straining was increased relative to Baseline in the cilansetron 2 mg TID group during the first six weeks of treatment then started to decrease at Week 7. In contrast, no clinically relevant change from Baseline was observed in the placebo group. At the end of treatment, a significantly larger increase from Baseline was observed in the cilansetron 2 mg TID group compared with the placebo group (0.09 versus 0.01, p<0.001).

Study S241.3.011

Three-Month Treatment Period

At Baseline, mean straining(“yes”=1, “no”=0) was similar in the treatment groups (0.24 versus 0.23). Weekly mean straining increased relative to Baseline in the cilansetron 2 mg TID group during Week 1 of treatment, then slightly decreased and remained relatively stable after Week 4 for the rest of the study. In contrast, no clinically relevant change from Baseline was observed in the placebo group. The cilansetron 2 mg TID group had a higher mean at every week compared with the placebo group. At the end of treatment, a significantly larger increase from Baseline was observed in the cilansetron 2 mg TID group compared with the placebo group (mean change of 0.05 versus −0.03, p<0.001).

Four-Week Rerandomization Period

At rerandomization Baseline, mean straining was higher in the C/C (0.27) and C/P (0.25) treatment groups compared with the P/C (0.20) and P/P (0.19) treatment groups. Weekly mean straining increased during the four-week rerandomization period in the P/C treatment group (mean change from rerandomization Baseline at end of treatment, 0.10) and remained relatively stable in the C/C (mean change of 0.02) and P/P (mean change of 0.01) treatment groups. In contrast, weekly mean straining decreased during the four-week rerandomization period in the C/P treatment group (mean change of −0.05).

Study S241.3.009

At Baseline, mean straining (“yes”=1, “no”=0) was equal in the treatment groups (0.21). Weekly mean straining slightly increased in the cilansetron 2 mg TID group during the first seven weeks of treatment then started to decrease at Week 8. In contrast, no clinically relevant change from Baseline was observed in the placebo group. At the end of treatment, a significantly larger increase from Baseline was observed in the cilansetron 2 mg TID group compared with the placebo group (0.06 versus 0.00, p<0.001).

Urgency

Study S241.3.006

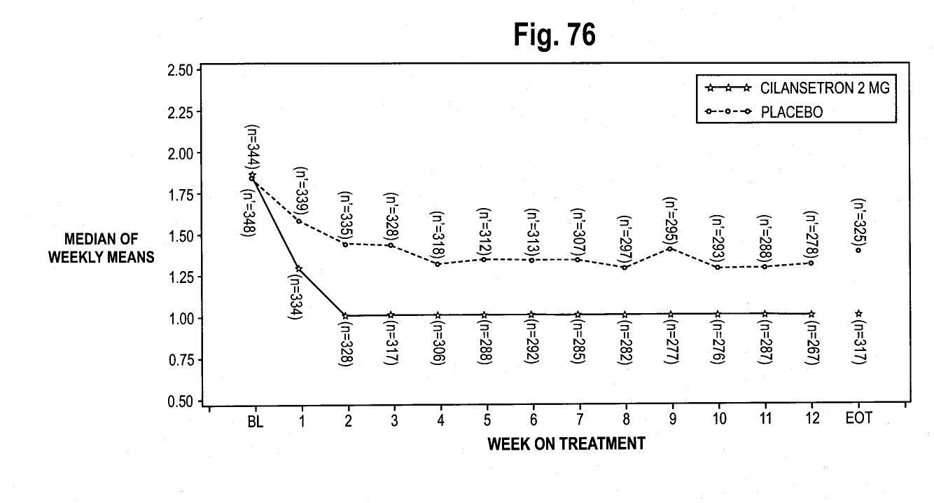

A summary of weekly means for daily urgency is presented for the ITT population in FIG. 76.

At Baseline, the mean scores for daily urgency (0=none, 1=mild, 2=moderate, 3=severe, 4=intolerable) were similar in the treatment groups (1.88 versus 1.85). Weekly mean scores for daily urgency gradually decreased in both treatment groups during the study with the cilansetron 2 mg TID group having a lower mean score at every week compared with the placebo group. At the end of treatment, a significantly larger decrease from Baseline was observed in the cilansetron 2 mg TID group compared with the placebo group (−0.87 versus −0.50, p<0.001).

At Baseline, the median number of urgency-free days during the run-in period was zero days in both treatment groups. At the end of treatment, a larger median increase from Baseline was observed in the cilansetron 2 mg TID group compared with the placebo group (median rate at the end of treatment; 0.27 versus 0.08).

The rate of urgency-free days was calculated from treatment start to treatment stop and presented for the ITT population in Table 2.7.3.6.4.1. During the three-month treatment period, the rate of urgency-free days was higher in the cilansetron 2 mg TID group compared with the placebo group (median rate of 0.32 versus 0.12 for the three-month treatment period).

Study S241.3.011

Three-Month Treatment Period

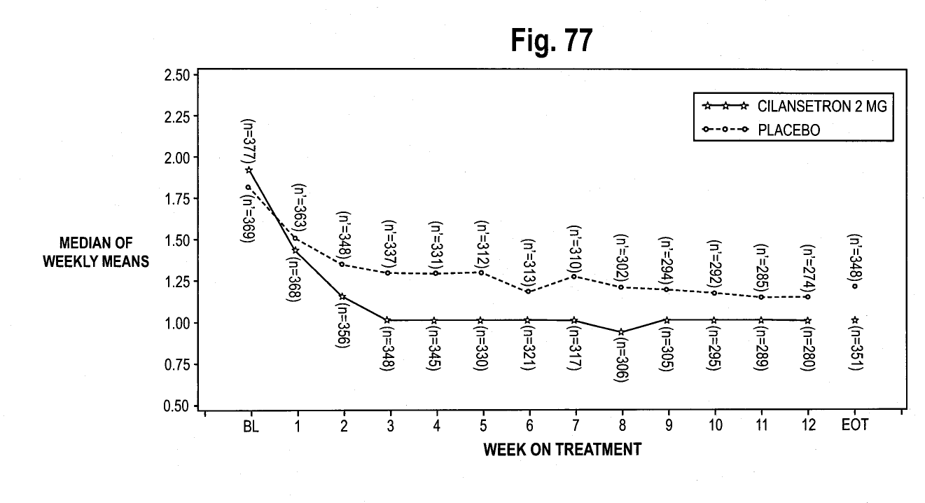

A summary of weekly means for daily urgency is presented for the ITT population in FIG. 77.

At Baseline, the mean scores for daily urgency (0=none, 1=mild, 2=moderate, 3=severe, 4=intolerable) were similar in the treatment groups (1.89 versus 1.79). Weekly mean scores for daily urgency gradually decreased in both treatment groups during the three-month treatment period with the cilansetron 2 mg TID group having a lower mean score at every week compared with the placebo group. At the end of treatment, a significantly larger decrease from Baseline was observed in the cilansetron 2 mg TID group compared with the placebo group (mean change of −0.78 versus −0.47, p<0.001).

At Baseline, the median rate of urgency-free days during the run-in period was zero days in both treatment groups. At the end of treatment (during the last two weeks of study medication), a larger increase from Baseline was observed in the cilansetron 2 mg TID group compared with the placebo group (median rate of 0.25 versus 0.14 at the end of treatment).

During the three-month treatment period, the rate of urgency-free days was higher in the cilansetron 2 mg TID group compared with the placebo group (median rate of 0.31 versus 0.16 for the three-month treatment period).

Four-Week Rerandomization Period

At Rerandomization Baseline, the mean scores for daily urgency were lower in the C/C (1.04) and C/P (1.05) treatment groups compared with the P/C (1.24) and PIP (1.34) treatment groups. Weekly mean scores for daily urgency decreased during the four-week rerandomization period in the P/C treatment group (mean change from rerandomization Baseline at end of treatment, −0.27) and remained relatively stable in the C/C (mean change of −0.05) and P/P (mean change of −0.09) treatment groups. In contrast, weekly mean scores for daily urgency increased during the four-week rerandomization period in the C/P treatment group (mean change of 0.25).

Study S241.3.009

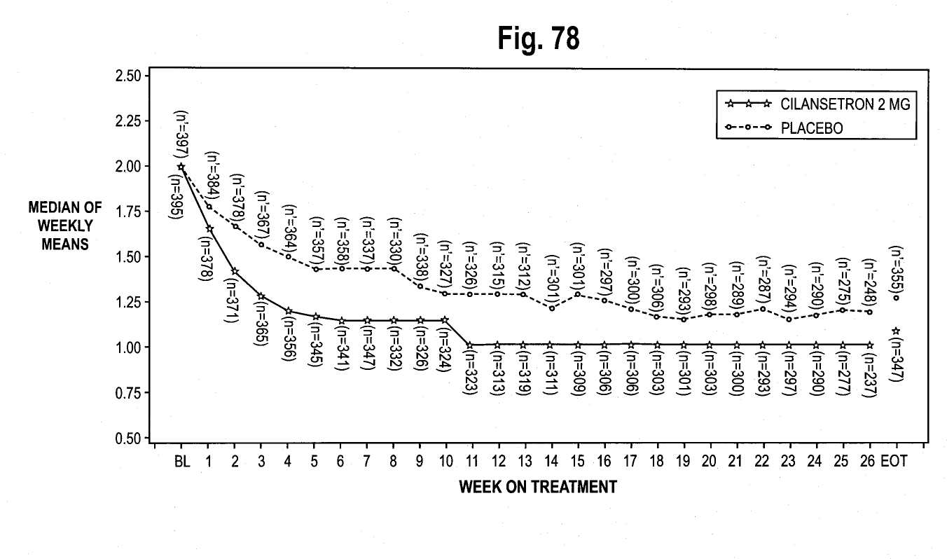

A summary of weekly means for daily urgency is presented for the In population in FIG. 78.

At Baseline, the mean scores for daily urgency (0=none, 1=mild, 2=moderate, 3=severe, 4=intolerable) were similar in the treatment groups (1.90 versus 1.91). Weekly mean scores for daily urgency gradually decreased in both treatment groups during the study with the cilansetron 2 mg TID group having a lower mean score at every week compared with the placebo group. At the end of treatment, a significantly larger decrease from Baseline was observed in the cilansetron 2 mg TID group compared with the placebo group (−0.80 versus −0.62, p<0.001).

At Baseline, the median number of urgency-free days during the run-in period was zero days in both treatment groups. At the end of treatment, a larger increase from Baseline was observed in the cilansetron 2 mg TID group compared with the placebo group (0.08 versus 0.00).

The rate of urgency-free days was also calculated from treatment start to treatment stop and presented for the ITT population in Table 2.7.3.6.4 2 (overall) and Table 2.7.3.6.4.3 (Month 1 to 3 and Month 4 to 6). During the six-month treatment period, the rate of urgency-free days was higher in the cilansetron 2 mg TID group compared with the placebo group (median rate of 0.07 versus 0.03 for the six-month treatment period). Similarly, the rate of urgency-free days was higher in the cilansetron 2 mg TID group compared with the placebo group during Month 1 to Month 3 (median rate of 0.05 versus 0.02 for the three-month treatment period) and during Month 4 to Month 6 (median rate of 0.10 versus 0.03 for the three-month treatment period).

Feeling of Incomplete Evacuation

Study S241.3.006

At Baseline, the means for daily feeling of incomplete evacuation (“yes”=1, “no”=0) assessed were similar in the treatment groups (0.55 versus 0.54). Weekly mean scores for daily feeling of incomplete evacuation gradually decreased in both treatment groups during the study with the cilansetron 2 mg TID group having a lower mean score at every week compared with the placebo group. At the end of treatment, a significantly larger decrease from Baseline was observed in the cilansetron 2 mg TID group compared with the placebo group (−0.18 versus −0.12, p=0.020).

Study S241.3.011

Three-Month Treatment Period

At Baseline, the means for daily feeling of incomplete evacuation (“yes”=1, “no”=0) assessed were similar in the treatment groups (0.60 versus 0.55). Weekly means for daily feeling of incomplete evacuation slightly decreased in both treatment groups during the three-month treatment period. At the end of treatment, mean change from Baseline was similar in the cilansetron 2 mg TID and placebo groups (−0.20 versus −0.14, respectively, p=0.136).

Four-Week Rerandomization Period

At Rerandomization Baseline, the means for daily feeling of incomplete evacuation were similar in the treatment groups (0.36 to 0.40). Weekly means for daily feeling of incomplete evacuation remained relatively unchanged during the four-week rerandomization period in all four treatment groups with no substantial change from Rerandomization Baseline at the end of treatment (mean change, C/C, 0.01; C/P, 0.05; P/C, −0.02; and PIP, −0.02).

Study S241.3.009

At Baseline, the means for daily feeling of incomplete evacuation (“yes”=1, “no”=0) were similar in the treatment groups (0.56 versus 0.58). A trend towards a decrease was observed in the weekly means for daily feeling of incomplete evacuation in both treatment groups during the study with the cilansetron 2 mg TID group having a lower mean score at every week compared with the placebo group. At the end of treatment, the decrease from Baseline was not significantly different in the treatment groups (−0.25 versus −0.21. in the cilansetron 2 mg TID group compared with the placebo group, respectively, p=0.118).

Bloating or Feeling of Abdominal Distention

Study 5241.3.006

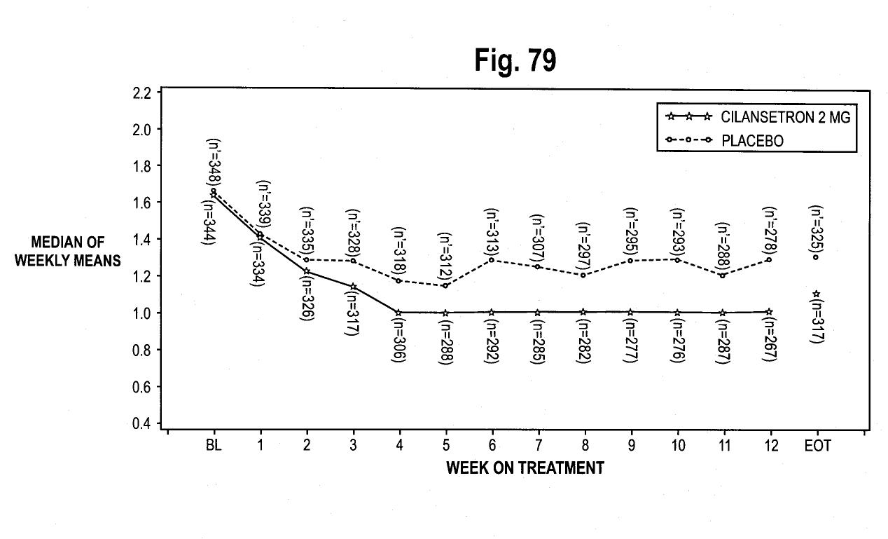

A summary of weekly means for daily bloating or feeling of abdominal distention is presented for the ITT population in FIG. 79.

At Baseline, the mean scores for daily bloating or feeling of abdominal distention (0=none, 1=mild, 2=moderate, 3=severe, 4=intolerable) were similar in the treatment groups (1.62 versus 1.60). Weekly mean scores for daily bloating or feeling of abdominal distention gradually decreased in both treatment groups during the study with the cilansetron 2 mg TID group having a lower mean score at every week compared with the placebo group. At the end of treatment, a significantly larger decrease from Baseline was observed in the cilansetron 2 mg TID group compared with the placebo group (−0.48 versus −0.31, p=0.002).

Study S241.3.011

Three-Month Treatment Period

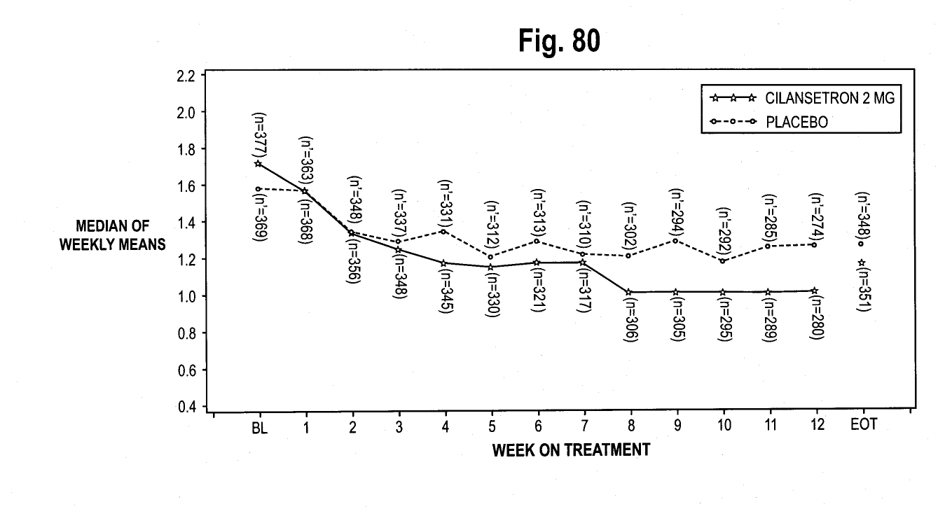

A summary of weekly means for daily bloating or feeling of abdominal distention is presented for the ITT population in FIG. 80.

At Baseline, the mean scores for daily bloating or feeling of abdominal distention (0=none, 1=mild, 2=moderate, 3=severe, 4=intolerable) were similar in the treatment groups (1.68 versus 1.61). Weekly mean scores for daily bloating or feeling of abdominal distention gradually decreased in both treatment groups during the three-month treatment period with the cilansetron 2 mg TID group having a lower mean score at every week after Week 1 compared with the placebo group. At the end of treatment, a significantly larger decrease from Baseline was observed in the cilansetron 2 mg TID group compared with the placebo group (mean change of −0.43 versus −0.30, p=0.03 1).

Four-Week Rerandomization Period

At rerandomization Baseline, the mean score for daily bloating or feeling of abdominal distention was lower in the C/C (1.11) and C/P (1.21) treatment groups compared with the P/C (1.25) and P/P (1.37) treatment groups. Weekly mean scores for daily bloating or feeling of abdominal distention slightly decreased during the four-week rerandomization period in the C/C treatment group (mean change from rerandomization Baseline at end of treatment, −0.05) and remained relatively stable in the P/C (mean change of −0.03) and P/P (mean change of 0.01) treatment groups. In contrast, weekly mean scores for daily bloating or feeling of abdominal distention increased during the four-week rerandomization period in the C/P treatment group (mean change of 0.16).

Study S241.3.009

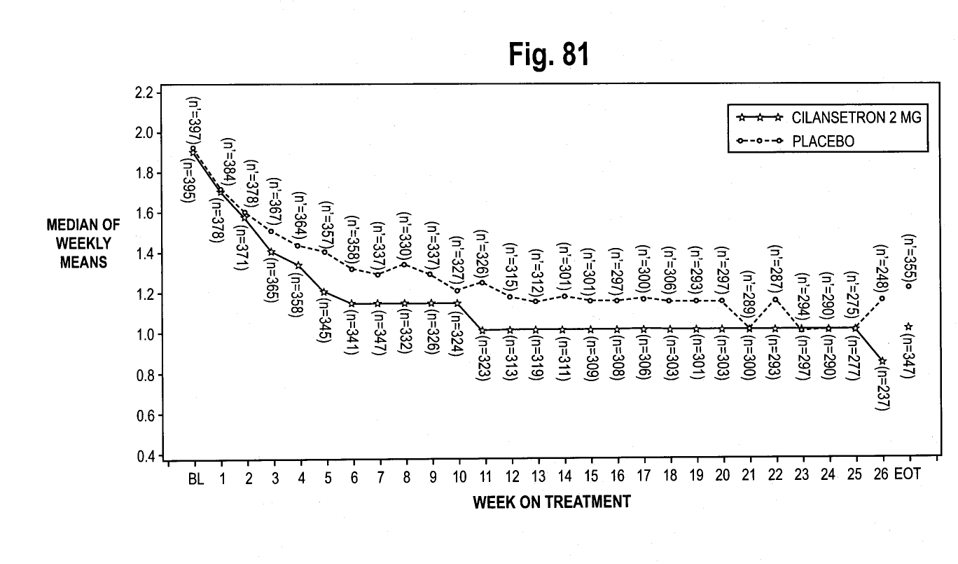

A summary of weekly means for daily bloating or feeling of abdominal distention is presented for the ITT population in FIG. 81.

At Baseline, the mean scores for daily bloating or feeling of abdominal distention (0=none, 1=mild, 2=moderate, 3=severe, 4=intolerable) were similar in the treatment groups (1.80 versus 1.81). Weekly mean scores for daily bloating or feeling of abdominal distention gradually decreased in both treatment groups during the study with the cilansetron 2 mg TID group having a lower mean score at every week compared with the placebo group. At the end of treatment, a significantly larger decrease from Baseline was observed in the cilansetron 2 mg TID group compared with the placebo group (−0.75 versus −0.57, p=0.001).

Irritable Bowel Syndrome—Quality of Life Assessment

Summary Across Studies

Subjects with diarrhea-predominant IBS have a constellation of symptoms that significantly affect their well-being and their ability to function normally. Limits in daily activities and food avoidance are just some examples in how they may have to adapt because of their illness. Because of this, subjects with diarrhea-predominant IBS may feel a sense of helplessness and a loss of control of their lives. Therefore, it is important to evaluate the changes in quality of life for these subjects in order to understand the impact of their IBS symptoms under treatment.

The subjects' overall quality of life was moderately affected by the disease at Baseline based on the mean overall EBS-QOL score (50.6 to 55.5 in the treatment groups across the studies on a scale of 0 to 100). Subscales of the IBS-QOL questionnaire such as food avoidance (mean scores of 36.3 to 46.6), interference with activity (38.6 to 45.2), and dysphoria (45.8 to 49.3) showed the most impairment in the subjects' quality of life at Baseline in all three studies. The remaining subscales showed modestly higher levels of quality of life at Baseline reflecting a slight to moderate impact of the disease on social reaction (56.4 to 62.1), body image (59.6 to 67.3), relationships (60.8 to 65.3), health worry (63.1 to 67.2), and sex (66.4 to 76.3).

In all three pivotal efficacy studies, the cilansetron 2 mg TID group demonstrated a statistically significant greater improvement from Baseline to Endpoint compared with the placebo group in the overall IBS-QOL score (p<0.001) and each subscore (interference with activity, body image, health worry, food avoidance, social reaction, sexual, relationships, and dysphoria, p<0.001 to p=0.014) with the exception of the sexual subscore in Study S241.3.009 (p=0.169). In Study S241.3.011, blinded withdrawal of cilansetron treatment led to an unfavorable effect on the subjects' quality of life.

Clinical improvement was demonstrated in the overall IBS-QOL with treatment differences of 7.37 to 7.90 across the studies in the mean change from Baseline score. In general, the subscales (e.g., interference with activity, dysphoria, and food avoidance) that showed the greatest clinical improvement in cilansetron 2 mg TID were those that were most important to the subjects (i.e., the lowest levels of quality life and a moderate to quite a bit of IBS symptoms impact at Baseline). The treatment differences across the studies in the mean change from Baseline score were 9.73 to 10.85 for interference with activity, 7.34 to 8.78 for dysphoria, and 8.92 to 10.09 for food avoidance.

Study S241.3.006

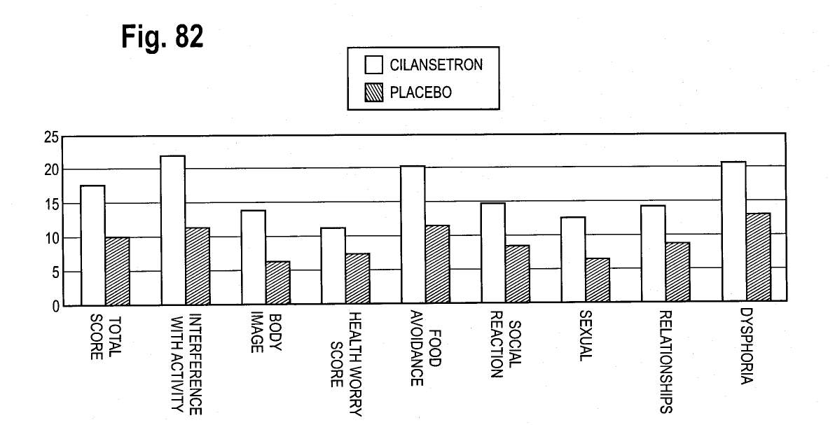

The IBS-QOL is presented for the ITT population in FIG. 82 (change from Baseline).

The Baseline values for the overall score and each individual score were comparable between the cilansetron 2 mg TID and placebo groups. The mean overall IBS-QOL score was 50.6 in the cilansetron 2 mg TID group versus 52.8 in the placebo group at Baseline. The subscales that showed the most impairment in the subjects' quality of life at Baseline included interference with activity (38.6 versus 41.6), food avoidance (36.3 versus 37.2) and dysphoria (45.8 versus 49.3). The remaining subscales had modestly higher scores at Baseline, including body image (59.6 versus 60.7), health worry (65.1 versus 67.2), social reaction (56.4 versus 58.8), sexual (66.4 versus 68.6), and relationships 960.8 versus 62.2).

The cilansetron 2 mg TID group demonstrated a statistically significant greater improvement from Baseline to Endpoint compared with the placebo group in the overall IBS-QOL score (p<0.001) and each subscore (interference with activity, body image, health worry, food avoidance, social reaction, sexual, relationships, and dysphoria, p<0.001 to p=0.008). For the overall score, the treatment difference in the mean change from Baseline at Endpoint was 7.37. Subscales with the greatest treatment difference included interference with activity (9.73), body image (7.43), food avoidance (8.92), and dysphoria (7.34). Smaller treatment differences were observed for health worry (3.21), social reaction (6.19), sexual (5.64), and relationships (5.19).

Study S241.3.011

Three-Month Treatment Period

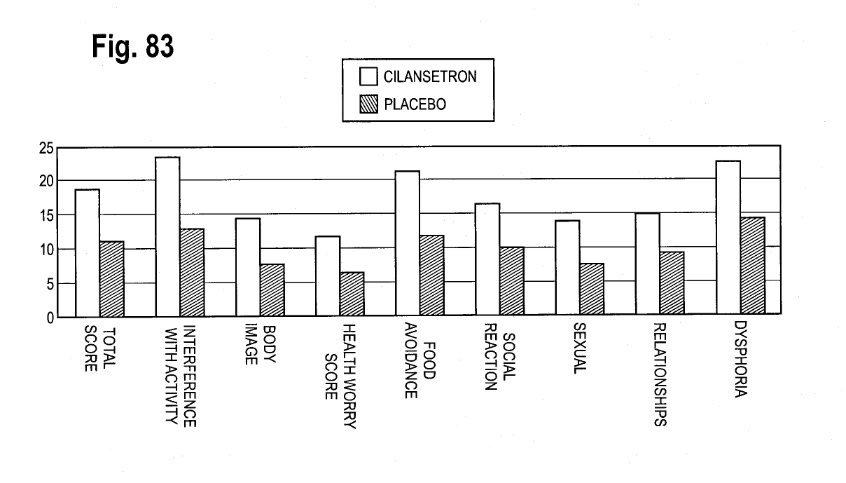

The IBS-QOL is presented for the ITT population in FIG. 83 (change from Baseline).

The Baseline values for the overall score and each individual subscore were comparable between the cilansetron 2 mg TID and placebo groups. The mean overall IBS-QOL score was 52.4 in the cilansetron 2 mg TID group versus 53.4 in the placebo group at Baseline. The subscales that showed the most impairment in the subjects' quality of life at Baseline included interference with activity (41.3 versus 42.0), food avoidance (37.4 versus 37.7), and dysphoria (48.3 versus 49.1). The remaining subscales had modestly higher scores at Baseline, including body image (61.0 versus 61.4), health worry (63.6 versus 65.6), social reaction (58.9 versus 60.4), sexual (69.4 versus 70.9), and relationships (63.6 versus 65.3).

The cilansetron 2 mg TID group demonstrated a statistically significant greater improvement from Baseline to Endpoint compared with the placebo group in the overall IBS-QOL score (p<0.001) and each subscore (interference with activity, body image, health worry, food avoidance, social reaction, sexual, relationships, and dysphoria, p<0.001 for each subscore). For the overall score, the treatment difference in the mean change from-Baseline at endpoint was 7.53. Subscales with the greatest treatment difference included interference with activity (10.57), food avoidance (9.64), and dysphoria (8.16). Smaller treatment differences were observed for body image (6.60), health worry (4.05), social reaction (5.53), sexual (5.93), and relationships (4.72).

Four-Week Rerandomization Period

The Baseline values of the overall score for IBS-QOL during a four-week rerandomization period were higher in the C/C and C/P treatment groups compared with the P/C and P/P treatment groups.

During the four-week rerandomization period, the overall IBS-QOL score increased (improved) in the P/C treatment group (mean change from rerandomization Baseline at endpoint, 5.1), slightly increased in the P/P treatment group (mean change of 3.3) and remained relatively stable in the C/C treatment group (mean change of 0.9). In contrast, the overall IBS-QOL score decreased during the four-week rerandomization period in the C/P treatment group (mean change of −4.9).

Similar trends within the individual treatment groups were observed as described for the overall IBS-QOL score for the subscores of interference with activity, body image, food avoidance, and dysphoria. For the subscores of social reaction, sexual, and relationships, the mean score decreased in the C/P treatment group, while the other treatment groups showed an increase or no substantial change from rerandomization Baseline. For the subscore of health worry, no substantial change was seen in any of the treatment groups except for a small increase in the P/P treatment group.

Study S241.3.009

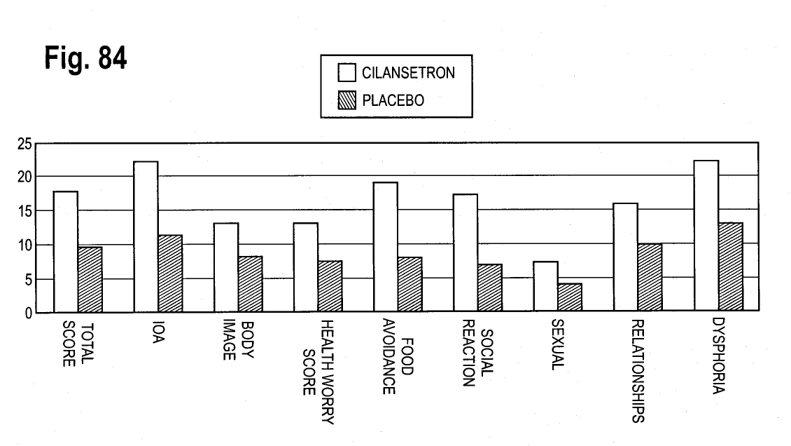

The IBS-QOL is presented for the ITT population in FIG. 84 (change from Baseline). The IBS-QOL questionnaire was completed only in countries for which there was a validated translation available. Subjects in the following countries did not complete the IBS-QOL questionnaire: Bulgaria, Israel, Poland, Romania, Russia, and Ukraine.

The Baseline values for the overall score and each individual subscore were comparable between the cilansetron 2 mg TED and placebo groups. The mean overall IBS-QOL score was 55.0 in the cilansetron 2 mg TID group versus 55.5 in the placebo group at Baseline. The subscales that showed the most impairment in the subjects' quality of life at Baseline included interference with activity (45.2 in both groups), food avoidance (42.9 versus 46.6), and dysphoria (48.8 versus 48.9). The remaining subscales had modestly higher scores at Baseline, including body image (67.3 versus 66.4), health worry (63.1 in both groups), social reaction (58.6 versus 62.1), sexual (76.3 versus 74.8), and relationships (64.9 versus 65.1).

The cilansetron 2 mg TID group demonstrated a statistically significant greater improvement from Baseline to Endpoint compared with the placebo group in the overall IBS-QOL score (p<0.001) and each subscore (interference with activity, body image, health worry, food avoidance, social reaction, relationships, and dysphoria, p<0.001 to p=O.0¹⁴) with the exception of the sexual subscore (p=0. 169). For the overall score, the treatment difference in the mean change from Baseline at Endpoint was 7.90. Subscales with the greatest treatment difference included interference with activity (10.85), food avoidance (10.09), and dysphoria (8.78). Smaller treatment differences were observed for body image (5.04), health worry (5.41), social reaction (8.70), sexual (2.93), and relationships (5.66).

Interruption of Activities Assessment

Summary Across Studies

Analysis of responses to the interruption of activities assessment demonstrated a clinically meaningful greater improvement in the cilansetron 2 mg TID group compared with the placebo group in the subjects' ability to effectively perform their everyday activities in all three pivotal efficacy studies. The largest treatment group differences were observed for the “not at all or rarely” category at each post-Baseline visit and at Endpoint. Across the three studies, the proportion of subjects who were “not at all or rarely” bothered by their IBS symptoms increased from 3%, 8%, and 6% at Baseline in Studies S241.3.006, S241.3.011, and S241.3.009, respectively, to 42%, 46%, and 54% at Endpoint in the cilansetron 2 mg TID group versus 3%, 6%, and 5% at Baseline to 25%, 32%, and 35% at Endpoint in the placebo group, respectively. In Study S241.3.011, blinded withdrawal of cilansetron treatment led to an unfavorable effect on the subjects' ability to effectively perform their everyday activities.

Study S241.3.006

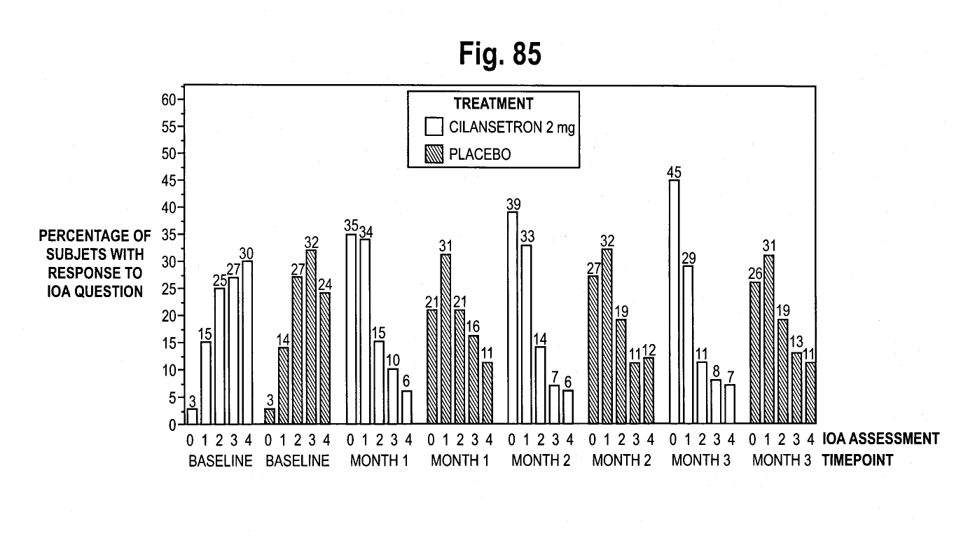

Responses to the interruption of activities assessment are summarized for the ITT population in FIG. 85.

Analysis of responses to the interruption of activities assessment demonstrated a clinically meaningful greater improvement in the cilansetron 2 mg TID group compared with the placebo group in the subjects' ability to effectively perform their everyday activities. The largest treatment group differences were observed for the “not at all or rarely” category at each post-Baseline visit and at Endpoint. The proportion of subjects who were “not at all or rarely” bothered by their IBS symptoms increased from 3% at Baseline to 42% at Endpoint in the cilansetron 2 mg TID group versus 3% at Baseline to 25% at Endpoint in the placebo group.

Study S241.3.011

Three-Month Treatment Period

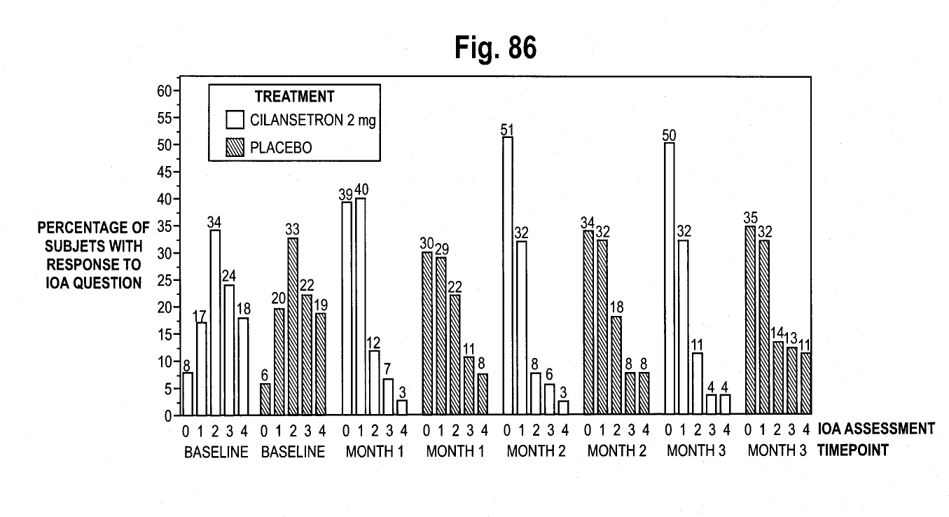

Responses to the interruption of activities assessment during the three-month treatment period are summarized for the ITT population in FIG. 86.

Analysis of responses to the interruption of activities assessment demonstrated a clinically meaningful greater improvement in the cilansetron 2 mg TID group compared with the placebo group in the subjects' ability to effectively perform their everyday activities. The largest treatment group differences were observed for the “not at all or rarely” category at each post-Baseline visit and at Endpoint. The proportion of subjects who were “not at all or rarely” bothered by their IBS symptoms increased from 8% at Baseline to 46% at Endpoint in the cilansetron 2 mg TID group versus 6% at Baseline to 32% at Endpoint in the placebo group.

Four-Week Rerandomization Period

Responses to the interruption of activities assessment during a four-week rerandomization period indicated a higher proportion of subjects in the C/C (52%) and C/P (50%) treatment groups compared with the P/C (40%) and P/P (30%) responded to the interruption of activities assessment that they were “not at all of rarely” bothered by their IBS symptoms. For the proportion of subjects who were “not at all or rarely” bothered by their IBS symptoms, no meaningful change from rerandomization Baseline to Endpoint was seen in the C/C treatment group (from 52% to 54%) while an increase was seen in the P/C (from 40% to 50%) and the P/P (from 30% to 37%) treatment groups. In contrast, the proportion of subjects who were “not at all or rarely” bothered by their IBS symptoms decreased in the C/P treatment group form 50% at rerandomization Baseline to 37% at Endpoint.

Study S241.3.009

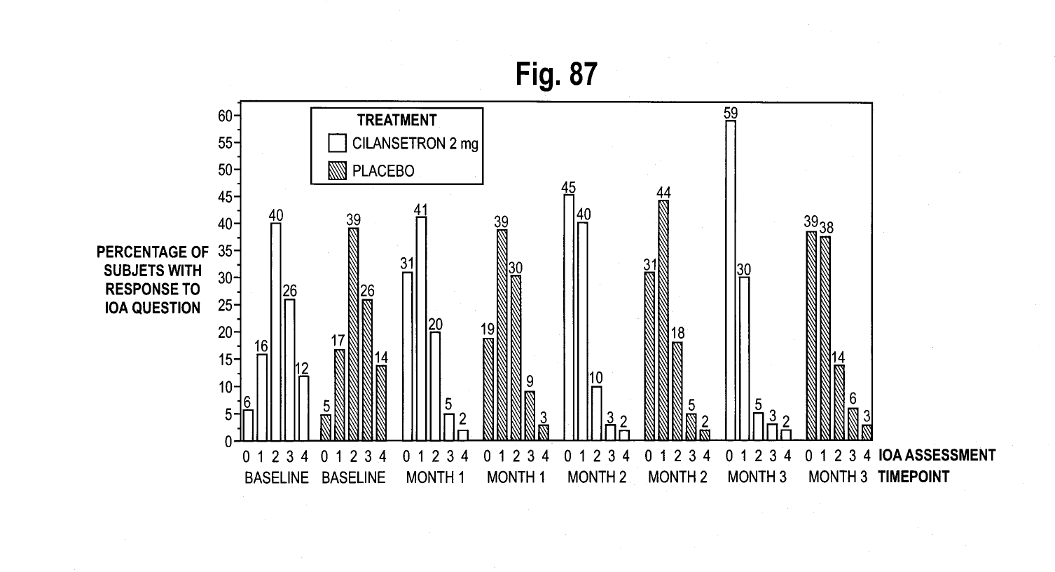

Responses to the interruption of activities assessment are summarized for the lIT population in FIG. 87.

Analysis of responses to the interruption of activities assessment demonstrated a clinically meaningful greater improvement in the cilansetron 2 mg TID group compared with the placebo group in the subjects' ability to effectively perform their everyday activities. The largest treatment group differences were observed for the “not at all or rarely” category at each post-Baseline visit and at Endpoint. The proportion of subjects who were “not at all or rarely” bothered by their IBS symptoms increased from 6% at Baseline to 54% at Endpoint in the cilansetron 2 mg TID group versus 5% at Baseline to 35% at Endpoint in the placebo group.

Comparison of Results in Subpopulations

The following subgroup analyses were performed for the efficacy parameters of the number of months that a subject was a responder and overall responder rate for the weekly diary questions on adequate relief in the pivotal efficacy studies (S241.3.006, S241.3.009, and S241.3.011):
Gender
Age category (18-40 years; 41-64 years; or ?65 years) Race (Caucasians or non-Caucasians)
BMI category (<30 kg/m2 or □3-0 kg/m2) Severity of IBS (severe or non-severe)
Duration of IBS (524 months; >24 months to 548 months; >48 months to :5120 months; or >120 months)
Tobacco use
Pooled center (Studies S241.3.006 and S241.3.011) and pooled country (Study S241.3.009)
   Summary of Results in Subpopulations Across Studies

It is concluded that gender should not have an impact on the efficacy of cilansetron 2 mg TID. Analyses by gender of the number of months a subject was a responder and the overall responder rate showed that both male and female subjects had statistically significant treatment responses according to both parameters for each of the three weekly diary questions in all three pivotal efficacy studies, with the exception of overall responder rate for IBS symptoms for male subjects in Study S241.3.009. Although not statistically significant, there was a higher overall responder rate for IBS symptoms in the cilansetron 2 mg TID group (55%) compared with the placebo group (45%) within male subjects in Study S241.3.009. There was a statistically significant gender interaction for the primary efficacy parameter in Study S241.3.009 and for the primary and main secondary efficacy parameters in Study S241.3.011. However, the gender differences in these two studies were opposite in nature, i.e., in Study S241.3.011, the treatment group differences were larger in male subjects compared with female subjects, and, in Study S241.3.009, the treatment group differences were smaller in male subjects compared with female subjects.

Analyses by age category (18-40 years, 41-64 years, >65 years) of the number of months a subject was a responder and the overall responder rate did not reveal any consistent trends.

Analyses by race (Caucasian, non-Caucasian) of the number of months a subject was a responder and the overall responder rate did not reveal consistent trends. Of note, the small size of the non-Caucasian subgroup limits the conclusions that can be made from these analyses.

Analyses by BMI category (<30 kg/m², ≧30 kg/m²) of the number of months a subject was a responder and the overall responder rate showed that subjects in both BMI categories had statistically significant treatment responses according to both parameters for each of the three weekly diary questions in all three pivotal Efficacy studies with the exception of the ≧30 kg/m² BMI category in Study S241.3.009. For subjects in this BMI category in Study S241.3.009, the overall responder rate was 1% to 10% lower in the cilansetron 2 mg TID group compared with the placebo group across the diary questions. It should be noted that in Study S241.3.009, only 16% (cilansetron 2 mg TID) versus 15% (placebo) of subjects were in the ≧30 kg/m² category compared with 33% versus 36% in Study 5241.3.006 or 29% versus 35% in Study S241.3.01 1. Given the relatively small sample size and the singularity of this finding, it is concluded that BMI should not have an impact on the efficacy of cilansetron 2 mg TID.

Analyses by disease severity (severe, non-severe) of the number of months a subject was a responder and the overall responder rate showed that subjects in both severity categories had statistically significant treatment responses according to both parameters for each of the three weekly diary questions in all three pivotal efficacy studies.

Analyses by duration of IBS (≦24 months, >24 months to ≦48 months, >48 months to ≦120 months, >120 months) of the number of months a subject was a responder and the overall responder rate did not reveal any consistent trends.

Analyses by tobacco use of the number of months a subject was a responder and the overall responder rate did not reveal any consistent trends.

Analyses by pooled center and by pooled country of the number of months a subject was a responder and the overall responder rate did not reveal any meaningful trends.

Analyses by Gender

Study S241.3.006

The number of months a subject was a responder for the weekly diary questions on adequate relief during the three-month treatment period (monthly LOCF imputation) is presented for the ITT population by gender in Table 2.7.3:26.

TABLE 2.7.3:26
Number of Months a Subject Was a Responder for the Weekly Diary
Questions on Adequate Relief During the Three-Month Treatment
Period by Gender (Monthly LOCF Imputation)
Study S241.3.006 (ITT Population)

| Number of months a | | | | |
|---|---|---|---|---|
| subject was a responder | | Cilansetron | | |
| during the three-month | | 2 mg TID | Placebo | |
| treatment period | Statistic | (N = 344) | (N = 348) | P-Value^{a} |
| Male subjects | N | 101 | 104 | |
| IBS symptoms | | | | 0.005** |
| Zero months | n (%) | 52 (51) | 72 (69) | |
| One month | n (%) | 9 (9) | 10 (10) | |
| Two months | n (%) | 15 (15) | 8 (8) | |
| Three months | n (%) | 25 (25) | 14 (13) | |
| Abdominal | | | | 0.001** |
| pain/discomfort | | | | |
| Zero months | n (%) | 46 (46) | 67 (64) | |
| One month | n (%) | 10 (10) | 12 (12) | |
| Two months | n (%) | 9 (9) | 8 (8) | |
| Three months | n (%) | 36 (36) | 17 (16) | |
| Abnormal bowel habits | | | | 0.003** |
| Zero months | n (%) | 48 (48) | 71 (68) | |
| One month | n (%) | 13 (13) | 11 (11) | |
| Two months | n (%) | 15 (15) | 8 (8) | |
| Three months | n (%) | 25 (25) | 14 (13) | |
| Female subjects | N | 243 | 244 | |
| IBS symptoms | | | | <0.001** |
| Zero months | n (%) | 75 (31) | 123 (50) | |
| One month | n (%) | 36 (15) | 33 (14) | |
| Two months | n (%) | 38 (16) | 39 (16) | |
| Three months | n (%) | 94 (39) | 49 (20) | |
| Abdominal | | | | <0.001** |
| pain/discomfort | | | | |
| Zero months | n (%) | 70 (29) | 105 (43) | |
| One month | n (%) | 32 (13) | 31 (13) | |
| Two months | n (%) | 38 (16) | 46 (19) | |
| Three months | n (%) | 103 (42) | 62 (25) | |
| Abnormal bowel habits | | | | <0.001** |
| Zero months | n (%) | 75 (31) | 132 (54) | |
| One month | n (%) | 27 (11) | 34 (14) | |
| Two months | n (%) | 37 (15) | 33 (14) | |
| Three months | n (%) | 104 (43) | 45 (18) | |

*Significant at the 0.050 level for treatment effect;
**Significant at the 0.010 level for treatment effect.
^{a}P-values for treatment effect are based on a non-parametric ANOVA model with an effect for treatment.
Note:
Percentages are based on the number of subjects in the ITT Population.
Note:
A subject is defined as a responder for a month if he/she reports adequate relief (‘yes’) for at least two of the four weeks during that month.
Note:
Weekly diary observations made on or after the date of randomization and up through two days after the date of last dose of study medication are included in this summary.
Data source: CSR Table 10.1.2.1.5 Left, Vol. 9 Righ

Analyses by gender of the number of months a subject was a responder for the weekly diary questions on adequate relief showed statistically significant treatment group differences in favor of cilansetron compared with placebo for each of the three weekly diary questions in -both male and female subjects.

The overall responder rate for the weekly diary questions on adequate relief during the three-month treatment period (observed case) is presented for the ITT population by gender in Table 2.7.3:27.

TABLE 2.7.3:27
Overall Responder Rate for the Weekly Diary Questions on Adequate
Relief During the Three-Month Treatment Period by Gender
(Observed Case)
Study S241.3.006 (ITT Population)

| Number of subjects who | | | | |
|---|---|---|---|---|
| were responders | | Cilansetron | | |
| during the three-month | | 2 mg TID | Placebo | |
| treatment period | Statistic | (N = 344) | (N = 348) | P-Value^{a} |
| Male subjects | N | 101 | 104 | |
| Adequate relief of IBS | n (%) | 41 (41) | 19 (18) | <0.001** |
| symptoms | | | | |
| Adequate relief of | n (%) | 45 (45) | 24 (23) | 0.001** |
| abdominal pain/ | | | | |
| discomfort | | | | |
| Adequate relief of | n (%) | 39 (39) | 18 (17) | <0.001** |
| abnormal bowel | | | | |
| habits | | | | |
| Female subjects | N | 243 | 244 | |
| Adequate relief of IBS | n (%) | 127 (52) | 80 (33) | <0.001** |
| symptoms | | | | |
| Adequate relief of | n (%) | 134 (55) | 105 (43) | 0.008** |
| abdominal pain/ | | | | |
| discomfort | | | | |
| Adequate relief of | n (%) | 135 (56) | 71 (29) | <0.001** |
| abnormal bowel habits | | | | |

*Significant at the 0.050 level for treatment effect;
**Significant at the 0.010 level for treatment effect.
^{a}P-values for treatment effect are based on a non-parametric analysis of Variance (ANOVA) model with an effect for treatment.
Note:
Percentages are based on the number of subjects in the ITT Population within the appropriate subgroup.
Note:
Weekly diary observations made on or after the date of randomization and up through two days after the date of last dose of study medication are included in this summary.
Data source: CSR Table 10.1.2.2.5 Left, Vol. 9 Righ

Analyses by gender showed that the overall responder rate during the three-month treatment period was higher in the cilansetron 2 mg TID group compared with the placebo group for each of the three weekly diary questions in both males (IBS symptoms, 41% versus 18%, p<0.001; abdominal pain/discomfort, 45% versus 23%, p=0.001; and abnormal bowel habits, 39% versus 17%, p<0.001) and females (IBS symptoms, 52% versus 33%, p<0.001; abdominal pain/discomfort, 55% versus 43%, p=0.008; and abnormal bowel habits, 56% versus 29%, p<0.001). The treatment differences were statistically significant for each of the three weekly diary questions on adequate relief in both males and females.

Study S241.3.011

The number of months a subject was a responder for the weekly diary questions on adequate relief during the three-month treatment period (monthly LOCF Imputation) is presented for the nTT population by gender in Table 2.7.3:28.

TABLE 2.7.3:28
Number of Months a Subject Was a Responder for the Weekly Diary
Questions on Adequate Relief During the Three-Month Treatment
Period by Gender (Monthly LOCF Imputation)
Study S241.3.011 (ITT Population)

| Number of months a | | | | |
|---|---|---|---|---|
| subject was a responder | | Cilansetron | | |
| during the three-month | | 2 mg TID | Placebo | |
| treatment period | Statistic | (N = 377) | (N = 369) | P-Value^{a} |
| Male subjects | N | 115 | 123 | |
| IBS symptoms | | | | <0.001** |
| Zero months | n (%) | 26 (23) | 76 (62) | |
| One month | n (%) | 19 (17) | 20 (16) | |
| Two months | n (%) | 18 (16) | 11 (9) | |
| Three months | n (%) | 52 (45) | 16 (13) | |
| Abdominal pain/ | | | | <0.001** |
| discomfort | | | | |
| Zero months | n (%) | 27 (23) | 65 (53) | |
| One month | n (%) | 15 (13) | 24 (20) | |
| Two months | n (%) | 15 (13) | 11 (9) | |
| Three months | n (%) | 58 (50) | 23 (19) | |
| Abnormal bowel habits | | | | <0.001** |
| Zero months | n (%) | 33 (29) | 80 (65) | |
| One month | n (%) | 16 (14) | 16 (13) | |
| Two months | n (%) | 18 (16) | 12 (10) | |
| Three months | n (%) | 48 (42) | 15 (12) | |
| Female subjects | N | 262 | 246 | |
| IBS symptoms | | | | <0.001** |
| Zero months | n (%) | 79 (30) | 122 (50) | |
| One month | n (%) | 35 (13) | 27 (11) | |
| Two months | n (%) | 52 (20) | 45 (18) | |
| Three months | n (%) | 96 (37) | 52 (21) | |
| Abdominal pain/ | | | | 0.003** |
| discomfort | | | | |
| Zero months | n (%) | 74 (28) | 97 (39) | |
| One month | n (%) | 30 (11) | 31 (13) | |
| Two months | n (%) | 57 (22) | 48 (20) | |
| Three months | n (%) | 101 (39) | 70 (28) | |
| Abnormal bowel habits | | | | <0.001** |
| Zero months | n (%) | 79 (30) | 124 (50) | |
| One month | n (%) | 34 (13) | 27 (11) | |
| Two months | n (%) | 48 (18) | 49 (20) | |
| Three months | n (%) | 101 (39) | 46 (19) | |

*Significant at the 0.050 level for treatment effect;
**Significant at the 0.010 level for treatment effect.
^{a}P-values for treatment effect are based on a non-parametric ANOVA model with an effect for treatment.
Note:
Percentages are based on the number of subjects in the ITT Population.
Note:
A subject is defined as a responder for a month if he/she reports adequate relief (‘yes’) for at least two of the four weeks during that month.
Note:
Weekly diary observations made on or after the date of randomization and up through two days after the date of last dose of study medication during the 12-week treatment period (or the date of first dose of study medication during the rerandomization period if earlier) are included in this summary.
Data source: CSR Table 10.1.2.1.5 Left, Vol. 9 Righ

Analyses by gender of the number of months a subject was a responder for the weekly diary questions on adequate relief showed statistically significant treatment group differences in favor of cilansetron 2 mg TID compared with placebo for each of the three weekly diary questions in both male and female subjects. However, there was a trend for larger treatment effects for each diary question in male subjects compared with female subjects.

The overall responder rate for the weekly diary questions on adequate relief during the three-month treatment period (observed case) is presented for the ITT population by gender in Table 2.7.3:29.

TABLE 2.7.3:29
Overall Responder Rate for the Weekly Diary Questions on Adequate
Relief During the Three-Month Treatment Period by Gender
(Observed Case)
Study S241.3.011 (ITT Population)

| Number of subjects who | | | | |
|---|---|---|---|---|
| were responders during | | Cilansetron | | |
| the three-month | | 2 mg TID | Placebo | |
| treatment period | Statistic | (N = 377) | (N = 369) | P-Value^{a} |
| Male subjects | N | 115 | 123 | |
| Adequate relief of IBS | n (%) | 68 (59) | 26 (21) | <0.001** |
| symptoms | | | | |
| Adequate relief of | n (%) | 70 (61) | 33 (27) | <0.001** |
| abdominal pain/ | | | | |
| discomfort | | | | |
| Adequate relief of | n (%) | 63 (55) | 25 (20) | <0.001** |
| abnormal bowel habits | | | | |
| Female subjects | N | 262 | 246 | |
| Adequate relief of IBS | n (%) | 138 (53) | 82 (33) | <0.001** |
| symptoms | | | | |
| Adequate relief of | n (%) | 151 (58) | 103 (42) | <0.001** |
| abdominal pain/ | | | | |
| discomfort | | | | |
| Adequate relief of | n (%) | 140 (53) | 80 (33) | <0.001** |
| abnormal bowel habits | | | | |

*Significant at the 0.050 level for treatment effect;
**Significant at the 0.010 level for treatment effect.
^{a}P-values for treatment effect are based on a non-parametric ANOVA model with an effect for treatment.
Note:
Percentages are based on the number of subjects in the ITT Population within the appropriate subgroup.
Note:
A responder for the three-month treatment period is defined as a subject who answers the weekly question with “yes” for at least half of the answers available with a minimum of four-week treatment duration during the 12-week treatment period. If a subject has less than a four-week treatment duration, the subject will be considered a non-responder.
Note:
Weekly diary observations made on or after the date of randomization and up through two days after the date of last dose of study medication during the 12-week treatment period (or the date of first dose of study medication during the rerandomization period if earlier) are included in this summary.
Data source: CSR Table 10.1.2.2.5 Left, Vol. 9 Righ

Analyses by gender showed that the overall responder rate during the three-month treatment period was significantly higher in the cilansetron 2 mg TID group compared with the placebo group for each of the three weekly diary questions in both males (IBS symptoms, 59% versus 21%, p<0.001; abdominal pain/discomfort, 61% versus 27%, p<0.001; and abnormal bowel habits, 55% versus 20%, p<0.001) and females (IBS symptoms, 53% versus 33%, p<0.001; abdominal pain/discomfort, 58% versus 42%, p<0.001; and abnormal bowel habits, 53% versus 33%, p<0.001). The treatment group differences were larger in male subjects (34% to 38% across the diary questions) compared with female subjects (16% to 20%). This quantitative difference led to the gender interaction observed in the primary and main secondary efficacy analysis.

Study S241.3.009

The number of months a subject was a responder for the weekly diary questions on adequate relief during Months 1 to 3 (monthly LOCF Imputation) is presented for the IT population by gender in Table 2.7.3:30.

TABLE 2.7.3:30
Number of Months a Subject Was a Responder for the Weekly Diary
Questions on Adequate Relief During Months 1 to 3 by Gender
(Monthly LOCF Imputation)
Study S241.3.009 (ITT Population)

| Number of months a | | | | |
|---|---|---|---|---|
| subject was a responder | | Cilansetron | | |
| during Months | | 2 mg TID | Placebo | |
| 1 to 3 | Statistic | (N = 395) | (N = 397) | P-Value^{a} |
| Male subjects | N | 186 | 172 | |
| IBS symptoms | | | | 0.001** |
| Zero months | n (%) | 58 (31) | 71 (41) | |
| One month | n (%) | 27 (15) | 33 (19) | |
| Two months | n (%) | 33 (18) | 34 (20) | |
| Three months | n (%) | 68 (37) | 34 (20) | |
| Abdominal pain/ | | | | 0.001** |
| discomfort | | | | |
| Zero months | n (%) | 55 (30) | 71 (41) | |
| One month | n (%) | 31 (17) | 34 (20) | |
| Two months | n (%) | 34 (18) | 32 (19) | |
| Three months | n (%) | 66 (35) | 35 (20) | |
| Abnormal bowel habits | | | | <0.001** |
| Zero months | n (%) | 54 (29) | 70 (41) | |
| One month | n (%) | 24 (13) | 39 (23) | |
| Two months | n (%) | 34 (18) | 34 (20) | |
| Three months | n (%) | 74 (40) | 29 (17) | |
| Female subjects | N | 209 | 225 | |
| IBS symptoms | | | | <0.001** |
| Zero months | n (%) | 52 (25) | 88 (39) | |
| One month | n (%) | 28 (13) | 49 (22) | |
| Two months | n (%) | 47 (22) | 37 (16) | |
| Three months | n (%) | 82 (39) | 51 (23) | |
| Abdominal pain/ | | | | <0.001** |
| discomfort | | | | |
| Zero months | n (%) | 50 (24) | 88 (39) | |
| One month | n (%) | 30 (14) | 38 (17) | |
| Two months | n (%) | 36 (17) | 44 (20) | |
| Three months | n (%) | 93 (44) | 55 (24) | |
| Abnormal bowel habits | | | | <0.001** |
| Zero months | n (%) | 44 (21) | 81 (36) | |
| One month | n (%) | 25 (12) | 43 (19) | |
| Two months | n (%) | 49 (23) | 43 (19) | |
| Three months | n (%) | 91 (44) | 58 (26) | |

*Significant at the 0.050 level for treatment effect;
**Significant at the 0.010 level for treatment effect.
^{a}P-values for treatment effect are based on a non-parametric ANOVA model with an effect for treatment.
Note:
Percentages are based on the number of subjects in the ITT Population.
Note:
A subject is defined as a responder for a month if he/she reports adequate relief (‘yes’) for at least two of the four weeks during that month.
Note:
Weekly diary observations made on or after the date of randomization and up through two days after the date of last dose of study medication are included in this summary.
Data source: CSR Table 10.1.2.1.5 Left, Vol. 9 Righ

Analyses by gender of the number of months a subject was a responder for the weekly diary questions on adequate relief showed statistically significant treatment group differences in favor of cilansetron compared with placebo for each of the three weekly diary questions independent of gender.

The overall responder rate for the weekly diary questions on adequate relief during the six-month treatment period (observed case) is presented for the ITT population by gender in Table 2.7.3:31.

TABLE 2.7.3:31
Overall Responder Rate for the Weekly Diary Questions on Adequate
Relief During the 26-Week Treatment Period by Gender (Observed
Case)
Study S241.3.009 (ITT Population)

| Number of subjects who | | | | |
|---|---|---|---|---|
| were responders during | | Cilansetron | | |
| the 26-week | | 2 mg TID | Placebo | |
| treatment period | Statistic | (N = 395) | (N = 397) | P-Value^{a} |
| Male subjects | N | 186 | 172 | |
| Adequate relief of IBS | n (%) | 102 (55) | 78 (45) | 0.073 |
| symptoms | | | | |
| Adequate relief of | n (%) | 106 (57) | 77 (45) | 0.021* |
| abdominal pain/ | | | | |
| discomfort | | | | |
| Adequate relief of | n (%) | 112 (60) | 75 (44) | 0.002** |
| abnormal bowel habits | | | | |
| Female subjects | N | 209 | 225 | |
| Adequate relief of IBS | n (%) | 132 (63) | 101 (45) | <0.001** |
| symptoms | | | | |
| Adequate relief of | n (%) | 133 (64) | 104 (46) | <0.001** |
| abdominal pain/ | | | | |
| discomfort | | | | |
| Adequate relief of | n (%) | 141 (67) | 107 (48) | <0.001** |
| abnormal bowel habits | | | | |

*Significant at the 0.050 level for treatment effect;
**Significant at the 0.010 level for treatment effect.
^{a}P-values for treatment effect are based on a non-parametric analysis of Variance (ANOVA) model with an effect for treatment.
Note:
Percentages are based on the number of subjects in the ITT Population within the appropriate subgroup.
Note:
Weekly diary observations made on or after the date of randomization and up through two days after the date of last dose of study medication are included in this summary.
Data source: CSR Table 10.1.2.2.13 Left, Vol. 9 Righ

Analyses by gender showed that the overall responder rate for the weekly diary question on adequate relief of IBS symptoms during the 26-week treatment period was higher in the cilansetron 2 mg TID group compared with the placebo group for both males (55% versus 45%, p=0.073) and females (63% versus 45%, p<0.001), however, the treatment difference was smaller for males than females and was statistically significant only for females. This quantitative difference led to the gender interaction observed in the primary efficacy analysis. The cilansetron 2 mg TID group showed similar higher responder rates for both males and females as compared to placebo for the other two weekly diary questions on abdominal pain/discomfort (males: 57% versus 45%, p=0.021; females: 64% versus 46%, p<0.001) and abnormal bowel habits (males: 60% versus 44%, p=0.002; females: 67% versus 48%, p<0.001). The treatment differences for abdominal pain/discomfort and abnormal bowel habits were statistically significant in both males and females.

Analyses by Age Category

Study S241.3.006

The number of months a subject was a responder for the weekly diary questions on adequate relief during the three-month treatment period (monthly LOU imputation) was assessed. Analyses by age category of the number of months a subject was a responder for the weekly diary questions on adequate relief showed statistically significant treatment group differences in favor of cilansetron compared with placebo for each of the three weekly diary questions in all three age categories.

The overall responder rate for the weekly diary questions on adequate relief during the three-month treatment period (observed case) showed that the overall responder rate during the three-month treatment period was higher in the cilansetron 2 mg TID group compared with the placebo group for all three weekly diary questions in each of the three age categories. For abdominal pain/discomfort, the treatment group difference was not significant in the 18-40 years and ≧65 years age categories. No consistent trends in treatment group differences were noted by age category.

Study S241.3.011

The number of months a-subject was a responder for the weekly diary questions on adequate relief during the three-month treatment period (monthly LOCF Imputation) was assessed. Analyses by age category of the number of months a subject was a responder for the weekly diary questions on adequate relief showed statistically significant treatment group differences in favor of cilansetron 2 mg TID compared with placebo for each of the three weekly diary questions in the 18-40 years and 41-64 years age categories. Treatment group differences in favor of cilansetron 2 mg TID were also seen in the ≧65 years age category; however, they were not statistically significant possibly due to the small sample size of this age category.

The overall responder rate for the weekly diary questions on adequate relief during the three-month treatment period (observed case) showed that the overall responder rate during the three-month treatment period was higher in the cilansetron 2 mg TID group compared with the placebo group for all three weekly diary questions in each of the three age categories. The treatment group differences were not statistically significant in the ≧65 years age category probably due to the small size of this subgroup. No consistent trends in treatment group differences were noted by age.

Study S241.3.009

The number of months a subject was a responder for the weekly diary questions on adequate relief during Months 1 to 3 (monthly LOCF Imputation) was assessed. Analyses by age category of the number of months a subject was a responder for the weekly diary questions on adequate relief showed statistically significant treatment group differences in favor of cilansetron compared with placebo for each of the three weekly diary questions independent of the age category.

The overall responder rate for the weekly diary questions on adequate relief during the six-month treatment period (observed case) showed that the overall responder rate during the 26-week treatment period was higher in the cilansetron 2 mg TID group compared with the placebo group for all three weekly diary questions in each of the three age categories. For each diary question, the treatment difference was comparable among the age categories, except for the ≧65 years age category where for IBS symptoms and abnormal bowel habits the treatment difference was approximately twice as large compared with the other two age categories (18-40 years, N=312, 11% to 18% difference across the diary questions; 41-64 years, N=423, 14% to 18% difference across the diary questions; and ≧65 years, N=57, 15% to 34% difference across the diary questions). Of note, the ≧65 years age category included only 7% of the ITT population.

Analyses by Race

Study S241.3.006

The number of months a subject was a responder for the weekly diary questions on adequate relief during the three-month treatment period (monthly LOCF imputation) was assessed. Analyses by race of the number of months a subject was a responder for the weekly diary questions on adequate relief showed treatment group differences in favor of cilansetron compared with placebo for each of the three weekly diary questions independent of the race (Caucasian versus non-Caucasian). The treatment group differences were not significant for the non-Caucasian category, probably due to the small size of this subgroup (6%).

The overall responder rate for the weekly diary questions on adequate relief during the three-month treatment period (observed case) showed that the overall responder rate during the three-month treatment period was higher in the cilansetron 2 mg TID group compared with the placebo group for the weekly diary questions on IBS symptoms and abnormal bowel habits in both Caucasians (N=653, 21% and 25% treatment group difference across the diary questions) and non-Caucasians (N=39, 13% and 24% treatment group difference across the diary questions). For the weekly diary question on abdominal pain/discomfort, the overall responder rate during the three-month treatment period was higher in the cilansetron 2 mg TID group compared with the placebo group for Caucasians (52% versus 36%), but not for non-Caucasians (48% versus 50%). Of note, the non-Caucasian category included only 6% of the total subject population, which limits the conclusions that can be made from the analyses by race.

Study S241.3.011

The number of months a subject was a responder for the weekly diary questions on adequate relief during the three-month treatment period (monthly LOCF Imputation) was assessed. Analyses by race of the number of months a subject was a responder for the weekly diary questions on adequate relief of IBS symptoms and abnormal bowel habits showed statistically significant treatment group differences in favor of cilansetron 2 mg TID compared with placebo for both Caucasians and non-Caucasians. For the weekly diary question of abdominal pain/discomfort, the treatment group difference was significant for the Caucasian category, however, there was no significant treatment group difference for non-Caucasians, possibly due to the small sample size (14%).

The overall responder rate for the weekly diary questions on adequate relief during the three-month treatment period (observed case) showed that the overall responder rate during the three-month treatment period was higher in the cilansetron 2 mg TID group compared with the placebo group for each of the three weekly diary questions in both Caucasians (IBS symptoms, 53% versus 27%, p<0.001; abdominal pain/discomfort, 57% versus 35%, p<0.001; and abnormal bowel habits, 51% versus 26%, p<0.001) and non-Caucasians (IBS symptoms, 66% versus 41%, p=0.010; abdominal pain/discomfort, 66% versus 51%, p=0.1 15; and abnormal bowel habits, 70% versus 43%, p=0.006). The treatment differences were statistically significant for the weekly diary questions on IBS symptoms and abnormal bowel habits in both Caucasians and non-Caucasians. For the weekly diary question on abdominal pain/discomfort, there was a statistically significant treatment difference for Caucasians, but not non-Caucasians, possibly due to the small sample size in this subgroup.

Study S241.3.009

The number of months a subject was a responder for the weekly diary questions on adequate relief during Months 1 to 3 (monthly LOCF Imputation) was assessed. Analyses by race of the number of months a subject was a responder for the weekly diary questions on adequate relief showed treatment group differences in favor of cilansetron compared with placebo for each of the three weekly diary questions independent of the race (Caucasian versus non-Caucasian). The treatment group differences were not significant for the non-Caucasian category, probably due to the small size (10%) of this subgroup.

The overall responder rate for the weekly diary questions on adequate relief during the six-month treatment period (observed case) showed that the overall responder rate during the 26-week treatment period was higher in the cilansetron 2 mg TID group compared with the placebo group for each of the three weekly diary questions in both Caucasians (N=713, 15% to 18% treatment group difference across the diary questions) and non-Caucasians (N=79, 8% to 14% difference across the diary questions).

Analyses by Bodv Mass Index Category

Study S241.3.006

The number of months a subject was a responder for the weekly diary questions on adequate relief during the three-month treatment period (monthly LOCF imputation) is presented for the ITT population by BMI category in Table 2.7.3:32.

TABLE 2.7.3:32
Number of Months a Subject Was a Responder for the Weekly Diary
Questions on Adequate Relief During the Three-Month Treatment
Period by Baseline BMI Category (Monthly LOCF Imputation)
Study S241.3.006 (ITT Population)

| Number of months a | | | | |
|---|---|---|---|---|
| subject was a responder | | Cilansetron | | |
| during the three-month | | 2 mg TID | Placebo | |
| treatment period | Statistic | (N = 344) | (N = 348) | P-Value^{a} |
| Baseline BMI: | N | 230 | 224 | |
| <30 kg/m² | | | | |
| IBS symptoms | | | | <0.001** |
| Zero months | n (%) | 86 (37) | 130 (58) | |
| One month | n (%) | 29 (13) | 26 (12) | |
| Two months | n (%) | 35 (15) | 28 (13) | |
| Three months | n (%) | 80 (35) | 40 (18) | |
| Abdominal pain/ | | | | <0.001** |
| discomfort | | | | |
| Zero months | n (%) | 79 (34) | 116 (52) | |
| One month | n (%) | 26 (11) | 22 (10) | |
| Two months | n (%) | 29 (13) | 37 (17) | |
| Three months | n (%) | 96 (42) | 49 (22) | |
| Abnormal bowel habits | | | | <0.001** |
| Zero months | n (%) | 83 (36) | 135 (60) | |
| One month | n (%) | 27 (12) | 25 (11) | |
| Two months | n (%) | 37 (16) | 28 (13) | |
| Three months | n (%) | 83 (36) | 36 (16) | |
| Baseline BMI: | N | 114 | 124 | |
| ≧30 kg/m² | | | | |
| IBS symptoms | | | | 0.003** |
| Zero months | n (%) | 41 (36) | 65 (52) | |
| One month | n (%) | 16 (14) | 17 (14) | |
| Two months | n (%) | 18 (16) | 19 (15) | |
| Three months | n (%) | 39 (34) | 23 (19) | |
| Abdominal | | | | 0.012* |
| pain/discomfort | | | | |
| Zero months | n (%) | 37 (32) | 56 (45) | |
| One month | n (%) | 16 (14) | 21 (17) | |
| Two months | n (%) | 18 (16) | 17 (14) | |
| Three months | n (%) | 43 (38) | 30 (24) | |
| Abnormal bowel habits | | | | <0.001** |
| Zero months | n (%) | 40 (35) | 68 (55) | |
| One month | n (%) | 13 (11) | 20 (16) | |
| Two months | n (%) | 15 (13) | 13 (10) | |
| Three months | n (%) | 46 (40) | 23 (19) | |

*Significant at the 0.050 level for treatment effect;
**Significant at the 0.010 level for treatment effect.
^{a}P-values for treatment effect are based on a non-parametric ANOVA model with an effect for treatment.
Note:
Percentages are based on the number of subjects in the ITT Population.
Note:
A subject is defined as a responder for a month if he/she reports adequate relief (‘yes’) for at least two of the four weeks during that month.
Note:
Weekly diary observations made on or after the date of randomization and up through two days after the date of last dose of study medication are included in this summary.
Data source: CSR Table 10.1.2.1.10 Left, Vol. 9 Righ

Analyses by BMI category of the number of months a subject was a responder for the weekly diary questions on adequate relief showed statistically significant treatment group differences in favor of cilansetron compared with placebo for each of the three weekly diary questions in both BMI categories (BMI <30 kg/m² and BMI ≧30 kg/m²).

The overall responder rate for the weekly diary questions on adequate relief during the three-month treatment period (observed case) is presented for the ITT population by BMI category in Table 2.7.3:33.

Analyses by BMI category showed that the overall responder rate during the three-month treatment period was higher in the cilansetron 2 mg TID group compared with the placebo group for each of the three weekly diary questions for subjects with BMI <30 kg/m² (N=454, 15% to 25% treatment group difference across the diary questions) and BMI ≧30 kg/m² (N=238, 14% to 26% difference across the diary questions). The treatment differences were statistically significant for each of the three weekly diary questions on adequate relief in both BMI categories.

Study S241.3.011

The number of months a subject was a responder for the weekly diary questions on adequate relief during the three-month treatment period. (monthly LOU imputation) is presented for the ITT population by BMI category in Table 2.7.3:34.

TABLE 2.7.3:34
Number of Months a Subject Was a Responder for the Weekly Diary
Questions on Adequate Relief During the Three-Month Treatment
Period by BMI Category (Monthly LOCF Imputation)
Study S241.3.011 (ITT Population)

| Number of months a | | | | |
|---|---|---|---|---|
| subject was a responder | | Cilansetron | | |
| during the three-month | | 2 mg TID | Placebo | |
| treatment period | Statistic | (N = 377) | (N = 369) | P-Value^{a} |
| Baseline BMI: | N | 266 | 241 | |
| <30 kg/m² | | | | |
| IBS symptoms | | | | <0.001** |
| Zero months | n (%) | 73 (27) | 130 (54) | |
| One month | n (%) | 38 (14) | 27 (11) | |
| Two months | n (%) | 48 (18) | 40 (17) | |
| Three months | n (%) | 107 (40) | 44 (18) | |
| Abdominal | | | | <0.001** |
| pain/discomfort | | | | |
| Zero months | n (%) | 73 (27) | 107 (44) | |
| One month | n (%) | 30 (11) | 34 (14) | |
| Two months | n (%) | 50 (19) | 35 (15) | |
| Three months | n (%) | 113 (42) | 65 (27) | |
| Abnormal bowel habits | | | | <0.001** |
| Zero months | n (%) | 78 (29) | 134 (56) | |
| One month | n (%) | 33 (12) | 31 (13) | |
| Two months | n (%) | 51 (19) | 37 (15) | |
| Three months | n (%) | 104 (39) | 39 (16) | |
| Baseline BMI: | N | 111 | 127 | |
| ≧30 kg/m² | | | | |
| IBS symptoms | | | | <0.001** |
| Zero months | n (%) | 32 (29) | 68 (54) | |
| One month | n (%) | 16 (14) | 20 (16) | |
| Two months | n (%) | 22 (20) | 15 (12) | |
| Three months | n (%) | 41 (37) | 24 (19) | |
| Abdominal | | | | <0.001** |
| pain/discomfort | | | | |
| Zero months | n (%) | 28 (25) | 55 (43) | |
| One month | n (%) | 15 (14) | 21 (17) | |
| Two months | n (%) | 22 (20) | 23 (18) | |
| Three months | n (%) | 46 (41) | 28 (22) | |
| Abnormal bowel habits | | | | <0.001** |
| Zero months | n (%) | 34 (31) | 70 (55) | |
| One month | n (%) | 17 (15) | 12 (9) | |
| Two months | n (%) | 15 (14) | 23 (18) | |
| Three months | n (%) | 45 (41) | 22 (17) | |

*Significant at the 0.050 level for treatment effect;
**Significant at the 0.010 level for treatment effect.
^{a}P-values for treatment effect are based on a non-parametric ANOVA model with an effect for treatment.
Note:
Percentages are based on the number of subjects in the ITT Population.
Note:
A subject is defined as a responder for a month if he/she reports adequate relief (‘yes’) for at least two of the four weeks during that month.
Note:
Weekly diary observations made on or after the date of randomization and up through two days after the date of last dose of study medication during the 12-week treatment period (or the date of first dose of study medication during the rerandomization period if earlier) are included in this summary.
Data source: CSR Table 10.1.2.1.10 Left, Vol. 9 Righ

Analyses by BMI category of the number of months a subject was a responder for the weekly diary questions on adequate relief showed statistically significant treatment group differences in favor of cilansetron 2 mg TID compared with placebo for each of the three weekly diary questions in both BMI categories (BMI <30 kg/m² and BMI ≧30 kg/M²).

The overall responder rate for the weekly diary questions on adequate relief during the three-month treatment period (observed case) is presented for the ITT population by BMI category in Table 2.7.3:35.

TABLE 2.7.3:35
Overall Responder Rate for the Weekly Diary Questions on Adequate
Relief During the Three-Month Treatment Period by BMI Category
(Observed Case)
Study S241.3.011 (ITT Population)

| Number of subjects who | | | | |
|---|---|---|---|---|
| were responders during | | Cilansetron | | |
| the three-month | | 2 mg TID | Placebo | |
| treatment period | Statistic | (N = 377) | (N = 369) | P-Value^{a} |
| Baseline BMI: | N | 266 | 241 | |
| <30 kg/m² | | | | |
| Adequate relief of IBS | n (%) | 148 (56) | 73 (30) | <0.001** |
| symptoms | | | | |
| Adequate relief of | n (%) | 158 (59) | 94 (39) | <0.001** |
| abdominal pain/ | | | | |
| discomfort | | | | |
| Adequate relief of | n (%) | 150 (56) | 67 (28) | <0.001** |
| abnormal bowel habits | | | | |
| Baseline BMI: | N | 111 | 127 | |
| ≧30 kg/m² | | | | |
| Adequate relief of IBS | n (%) | 58 (52) | 34 (27) | <0.001** |
| symptoms | | | | |
| Adequate relief of | n (%) | 63 (57) | 41 (32) | <0.001** |
| abdominal pain/ | | | | |
| discomfort | | | | |
| Adequate relief of | n (%) | 53 (48) | 37 (29) | 0.003** |
| abnormal bowel habits | | | | |

*Significant at the 0.050 level for treatment effect;
**Significant at the 0.010 level for treatment effect.
^{a}P-values for treatment effect are based on a non-parametric ANOVA model with an effect for treatment.
Note:
Percentages are based on the number of subjects in the ITT Population within the appropriate subgroup.
Note:
A responder for the three-month treatment period is defined as a subject who answers the weekly question with “yes” for at least half of the answers available with a minimum of four-week treatment duration during the 12-week treatment period. If a subject has less than a four-week treatment duration, the subject will be considered a non-responder.
Note:
Weekly diary observations made on or after the date of randomization and up through two days after the date of last dose of study medication during the 12-week treatment period (or the date of first dose of study medication during the rerandomization period if earlier) are included in this summary.
Data source: CSR Table 10.1.2.2.10 Left, Vol. 9 Righ

Analyses by BMI category showed that the overall responder rate during the three-month treatment period was higher in the cilansetron 2 mg TID group compared with the placebo group for each of the three weekly diary questions for subjects with BMI <30 kg/m² (N=507, 20% to 28% treatment group difference across the diary questions) and BMI ≧30 kg/M² (N=238, 19% to 25% difference across the diary questions). The treatment differences were statistically significant for each of the three weekly diary questions on adequate relief in both BMI categories.

Study S241.3.009

The number of months a subject was a responder for the weekly diary questions on adequate relief during Months 1 to 3 (monthly LOCF Imputation) is presented for the ITT population by BMI category in Table 2.7.3:36.

TABLE 2.7.3:36
Number of Months a Subject Was a Responder for the Weekly Diary
Questions on Adequate Relief During Months 1 to 3 by BMI Category
(Monthly LOCF Imputation)
Study S241.3.009 (ITT Population)

| Number of months a | | | | |
|---|---|---|---|---|
| subject was a responder | | Cilansetron | | |
| during Months | | 2 mg TID | Placebo | |
| 1 to 3 | Statistic | (N = 395) | (N = 397) | P-Value^{a} |
| Baseline BMI: | N | 330 | 338 | |
| <30 kg/m² | | | | |
| IBS symptoms | | | | <0.001** |
| Zero months | n (%) | 86 (26) | 142 (42) | |
| One month | n (%) | 49 (15) | 66 (20) | |
| Two months | n (%) | 64 (19) | 58 (17) | |
| Three months | n (%) | 131 (40) | 72 (21) | |
| Abdominal | | | | <0.001** |
| pain/discomfort | | | | |
| Zero months | n (%) | 82 (25) | 142 (42) | |
| One month | n (%) | 51 (15) | 62 (18) | |
| Two months | n (%) | 58 (18) | 61 (18) | |
| Three months | n (%) | 139 (42) | 73 (22) | |
| Abnormal bowel habits | | | | <0.001** |
| Zero months | n (%) | 78 (24) | 134 (40) | |
| One month | n (%) | 39 (12) | 71 (21) | |
| Two months | n (%) | 69 (21) | 63 (19) | |
| Three months | n (%) | 144 (44) | 70 (21) | |
| Baseline BMI: | N | 65 | 58 | |
| ≧30 kg/m² | | | | |
| IBS symptoms | | | | 0.763 |
| Zero months | n (%) | 24 (37) | 16 (28) | |
| One month | n (%) | 6 (9) | 16 (28) | |
| Two months | n (%) | 16 (25) | 13 (22) | |
| Three months | n (%) | 19 (29) | 13 (22) | |
| Abdominal | | | | 0.579 |
| pain/discomfort | | | | |
| Zero months | n (%) | 23 (35) | 16 (28) | |
| One month | n (%) | 10 (15) | 10 (17) | |
| Two months | n (%) | 12 (18) | 15 (26) | |
| Three months | n (%) | 20 (31) | 17 (29) | |
| Abnormal bowel habits | | | | 0.868 |
| Zero months | n (%) | 20 (31) | 17 (29) | |
| One month | n (%) | 10 (15) | 11 (19) | |
| Two months | n (%) | 14 (22) | 13 (22) | |
| Three months | n (%) | 21 (32) | 17 (29) | |

*Significant at the 0.050 level for treatment effect;
**Significant at the 0.010 level for treatment effect.
^{a}P-values for treatment effect are based on a non-parametric ANOVA model with an effect for treatment.
Note:
Percentages are based on the number of subjects in the ITT Population.
Note:
A subject is defined as a responder for a month if he/she reports adequate relief (‘yes’) for at least two of the four weeks during that month.
Note:
Weekly diary observations made on or after the date of randomization and up through two days after the date of last dose of study medication are included in this summary.
Data source: CSR Table 10.1.2.1.10 Left, Vol. 9 Righ

Analyses by BMI category of the number of months a subject was a responder for the weekly diary questions on adequate relief showed less separation of cilansetron from placebo (and a lack of statistically significant difference) in subjects with BMI ≧30 kg/M² compared to subjects with BMI <30 kg/m² for each of the three weekly diary questions.

The overall responder rate for the weekly diary questions on adequate relief during the six-month treatment period (observed case) is presented for the [TM population by BMI category in Table 2.7.3:37.

TABLE 2.7.3:37
Overall Responder Rate for the Weekly Diary Questions on Adequate
Relief During the 26-Week Treatment Period by BMI Category
(Observed Case)
Study S241.3.009 (ITT Population)

| Number of subjects who | | | | |
|---|---|---|---|---|
| were responders | | Cilansetron | | |
| during the 26-week | | 2 mg TID | Placebo | |
| treatment period | Statistic | (N = 395) | (N = 397) | P-Value^{a} |
| Baseline BMI: | N | 330 | 338 | |
| <30 kg/m² | | | | |
| Adequate relief of IBS | n (%) | 207 (63) | 149 (44) | <0.001** |
| symptoms | | | | |
| Adequate relief of | n (%) | 207 (63) | 152 (45) | <0.001** |
| abdominal pain/ | | | | |
| discomfort | | | | |
| Adequate relief of | n (%) | 220 (67) | 151 (45) | <0.001** |
| abnormal bowel habits | | | | |
| Baseline BMI: | N | 65 | 58 | |
| ≧30 kg/m² | | | | |
| Adequate relief of IBS | n (%) | 27 (42) | 30 (52) | 0.260 |
| symptoms | | | | |
| Adequate relief of | n (%) | 32 (49) | 29 (50) | 0.932 |
| abdominal pain/ | | | | |
| discomfort | | | | |
| Adequate relief of | n (%) | 33 (51) | 30 (52) | 0.916 |
| abnormal bowel habits | | | | |

*Significant at the 0.050 level for treatment effect;
**Significant at the 0.010 level for treatment effect.
^{a}P-values for treatment effect are based on a non-parametric analysis of Variance (ANOVA) model with an effect for treatment.
Note:
Percentages are based on the number of subjects in the ITT Population within the appropriate subgroup.
Note:
Weekly diary observations made on or after the date of randomization and up through two days after the date of last dose of study medication are included in this summary.
Data source: CSR Table 10.1.2.2.18 Left, Vol. 9 Righ

Analyses by BMI category showed that the overall responder rate during the 26-week treatment period was higher in the cilansetron 2 mg TID group compared with the placebo group for each of the three weekly diary questions for subjects with BMI <30 kg/m2 (N=668, 18% to 22% treatment group difference across the diary questions). In contrast, for subjects with BMI ≧30 kg/M², the overall responder rate was lower in the cilansetron 2 mg TID group compared with the placebo group for each diary question (N=123, −10% to −1% difference across the diary questions).

Analyses by Disease Severity

Study S241.3.006

The number of months a subject was a responder for the weekly diary questions on adequate relief during the three-month treatment period (monthly LOCF imputation) is presented for the ITT population by severity of IBS in Table 2.7.3:38.

TABLE 2.7.3:38
Number of Months a Subject Was a Responder for the Weekly Diary
Questions on Adequate Relief During the Three-Month Treatment
Period by Baseline Severity of IBS (Monthly LOCF Imputation)
Study S241.3.006 (ITT Population)

| Number of months a | | | | |
|---|---|---|---|---|
| subject was a responder | | Cilansetron | | |
| during the three-month | | 2 mg TID | Placebo | |
| treatment period | Statistic | (N = 344) | (N = 348) | P-Value^{a} |
| Baseline severity of | N | 196 | 196 | |
| IBS: severe | | | | |
| IBS symptoms | | | | <0.001** |
| Zero months | n (%) | 81 (41) | 115 (59) | |
| One month | n (%) | 27 (14) | 20 (10) | |
| Two months | n (%) | 29 (15) | 27 (14) | |
| Three months | n (%) | 59 (30) | 34 (17) | |
| Abdominal | | | | 0.017* |
| pain/discomfort | | | | |
| Zero months | n (%) | 74 (38) | 100 (51) | |
| One month | n (%) | 33 (17) | 22 (11) | |
| Two months | n (%) | 22 (11) | 27 (14) | |
| Three months | n (%) | 67 (34) | 47 (24) | |
| Abnormal bowel habits | | | | <0.001** |
| Zero months | n (%) | 81 (41) | 116 (59) | |
| One month | n (%) | 24 (12) | 20 (10) | |
| Two months | n (%) | 30 (15) | 28 (14) | |
| Three months | n (%) | 61 (31) | 32 (16) | |
| Baseline severity of | N | 148 | 152 | |
| IBS: non-severe | | | | |
| IBS symptoms | | | | <0.001** |
| Zero months | n (%) | 46 (31) | 80 (53) | |
| One month | n (%) | 18 (12) | 23 (15) | |
| Two months | n (%) | 24 (16) | 20 (13) | |
| Three months | n (%) | 60 (41) | 29 (19) | |
| Abdominal | | | | <0.001** |
| pain/discomfort | | | | |
| Zero months | n (%) | 42 (28) | 72 (47) | |
| One month | n (%) | 9 (6) | 21 (14) | |
| Two months | n (%) | 25 (17) | 27 (18) | |
| Three months | n (%) | 72 (49) | 32 (21) | |
| Abnormal bowel habits | | | | <0.001** |
| Zero months | n (%) | 42 (28) | 87 (57) | |
| One month | n (%) | 16 (11) | 25 (16) | |
| Two months | n (%) | 22 (15) | 13 (9) | |
| Three months | n (%) | 68 (46) | 27 (18) | |

*Significant at the 0.050 level for treatment effect;
**Significant at the 0.010 level for treatment effect.
^{a}P-values for treatment effect are based on a non-parametric ANOVA model with an effect for treatment.
Note:
Percentages are based on the number of subjects in the ITT Population.
Note:
A subject is defined as a responder for a month if he/she reports adequate relief (‘yes’) for at least two of the four weeks during that month.
Note:
Weekly diary observations made on or after the date of randomization and up through two days after the date of last dose of study medication are included in this summary.
Data source: CSR Table 10.1.2.1.9 Left, Vol. 9 Righ

Analyses by IBS severity of the number of months a subject was a responder for the weekly diary questions on adequate relief showed statistically significant treatment group differences in favor of cilansetron compared with placebo for each of the three weekly diary questions in both subgroups of IBS severity (severe versus non-severe). However, there was a trend for a larger treatment effect in the subgroup of subjects with non-severe IBS.

The overall responder rate for the weekly diary questions on adequate relief during the threemonth treatment period (observed case) is presented for the ITT population by severity of IBS in Table 2.7.3:39.

TABLE 2.7.3:39
Overall Responder Rate for the Weekly Diary Questions on Adequate
Relief During the Three-Month Treatment Period by Severity of IBS
(Observed Case)
Study S241.3.006 (ITT Population)

| Number of subjects who | | | | |
|---|---|---|---|---|
| were responders during | | Cilansetron | | |
| the three-month | | 2 mg TID | Placebo | |
| treatment period | Statistic | (N = 344) | (N = 348) | P-Value^{a} |
| Baseline severity of | N | 196 | 196 | |
| IBS: severe | | | | |
| Adequate relief of | n (%) | 87 (44) | 55 (28) | <0.001** |
| IBS symptoms | | | | |
| Adequate relief of | n (%) | 90 (46) | 70 (36) | 0.040* |
| abdominal pain/ | | | | |
| discomfort | | | | |
| Adequate relief of | n (%) | 90 (46) | 52 (27) | <0.001** |
| abnormal bowel habits | | | | |
| Baseline severity of | N | 148 | 152 | |
| IBS: non-severe | | | | |
| Adequate relief of IBS | n (%) | 81 (55) | 44 (29) | <0.001** |
| symptoms | | | | |
| Adequate relief of | n (%) | 89 (60) | 59 (39) | <0.001** |
| abdominal pain/ | | | | |
| discomfort | | | | |
| Adequate relief of | n (%) | 84 (57) | 37 (24) | <0.001** |
| abnormal bowel habits | | | | |

*Significant at the 0.050 level for treatment effect;
**Significant at the 0.010 level for treatment effect.
^{a}P-values for treatment effect are based on a non-parametric analysis of Variance (ANOVA) model with an effect for treatment.
Note:
Percentages are based on the number of subjects in the ITT Population within the appropriate subgroup.
Note:
Weekly diary observations made on or after the date of randomization and up through two days after the date of last dose of study medication are included in this summary.
Data source: CSR Table 10.1.2.2.9 Left, Vol. 9 Righ

Analyses by IBS severity showed that the overall responder rate during the three-month treatment period was higher in the cilansetron 2 mg TID group compared with the placebo group for each of the three weekly diary questions for both severe IBS (N=392, 10% to 19% treatment group difference across the diary questions) and non-severe IBS (N=300, 21% to 33% treatment group difference across the diary questions). The treatment differences were statistically significant for each of the three weekly diary questions on adequate relief in subjects with both severe and non-severe IBS.

Study S241.3.011

The number of months a subject was a responder for the weekly diary questions on adequate relief during the three-month treatment period (monthly LOCF Imputation) is presented for the ITT population by severity of IBS in Table 2.7.3:40.

TABLE 2.7.3:40
Number of Months a Subject Was a Responder for the Weekly Diary
Questions on Adequate Relief During the Three-Month Treatment
Period by Severity of IBS (Monthly LOCF Imputation)
Study S241.3.011 (ITT Population)

| Number of months a | | | | |
|---|---|---|---|---|
| subject was a responder | | Cilansetron | | |
| during the three-month | | 2 mg TID | Placebo | |
| treatment period | Statistic | (N = 377) | (N = 369) | P-Value^{a} |
| Baseline severity of | N | 155 | 153 | |
| IBS: severe | | | | |
| IBS symptoms | | | | <0.001** |
| Zero months | n (%) | 43 (28) | 81 (53) | |
| One month | n (%) | 25 (16) | 18 (12) | |
| Two months | n (%) | 31 (20) | 23 (15) | |
| Three months | n (%) | 56 (36) | 31 (20) | |
| Abdominal | | | | <0.001** |
| pain/discomfort | | | | |
| Zero months | n (%) | 41 (26) | 67 (44) | |
| One month | n (%) | 20 (13) | 26 (17) | |
| Two months | n (%) | 32 (21) | 22 (14) | |
| Three months | n (%) | 62 (40) | 38 (25) | |
| Abnormal bowel habits | | | | <0.001** |
| Zero months | n (%) | 44 (28) | 85 (56) | |
| One month | n (%) | 24 (15) | 18 (12) | |
| Two months | n (%) | 26 (17) | 23 (15) | |
| Three months | n (%) | 61 (39) | 27 (18) | |
| Baseline severity of | N | 222 | 215 | |
| IBS: non-severe | | | | |
| IBS symptoms | | | | <0.001** |
| Zero months | n (%) | 62 (28) | 117 (54) | |
| One month | n (%) | 29 (13) | 29 (13) | |
| Two months | n (%) | 39 (18) | 32 (15) | |
| Three months | n (%) | 92 (41) | 37 (17) | |
| Abdominal | | | | <0.001* |
| pain/discomfort | | | | |
| Zero months | n (%) | 60 (27) | 95 (44) | |
| One month | n (%) | 25 (11) | 29 (13) | |
| Two months | n (%) | 40 (18) | 36 (17) | |
| Three months | n (%) | 97 (44) | 55 (26) | |
| Abnormal bowel habits | | | | <0.001** |
| Zero months | n (%) | 68 (31) | 119 (55) | |
| One month | n (%) | 26 (12) | 25 (12) | |
| Two months | n (%) | 40 (18) | 37 (17) | |
| Three months | n (%) | 88 (40) | 34 (16) | |

*Significant at the 0.050 level for treatment effect;
**Significant at the 0.010 level for treatment effect.
^{a}P-values for treatment effect are based on a non-parametric ANOVA model with an effect for treatment.
Note:
Percentages are based on the number of subjects in the ITT Population.
Note:
A subject is defined as a responder for a month if he/she reports adequate relief (‘yes’) for at least two of the four weeks during that month.
Note:
Weekly diary observations made on or after the date of randomization and up through two days after the date of last dose of study medication during the 12-week treatment period (or the date of first dose of study medication during the rerandomization period if earlier) are included in this summary.
Data source: CSR Table 10.1.2.1.9 Left, Vol. 9 Righ

Analyses by IBS severity of the number of months a subject was a responder for the weekly diary questions on adequate relief showed statistically significant treatment group differences in favor of cilansetron 2 mg TID compared with placebo for each of the three weekly diary questions in both subgroups of IBS severity (severe versus non-severe).

The overall responder rate for the weekly diary questions on adequate relief during the three-month treatment period (observed case) is presented for the ITT population by severity of IBS in Table 2.7.3:41.

TABLE 2.7.3:41
Overall Responder Rate for the Weekly Diary Questions on Adequate
Relief During the Three-Month Treatment Period by Severity of IBS
(Observed Case)
Study S241.3.011 (ITT Population)

| Number of subjects who | | | | |
|---|---|---|---|---|
| were responders during | | Cilansetron | | |
| the three-month | | 2 mg TID | Placebo | |
| treatment period | Statistic | (N = 377) | (N = 369) | P-Value^{a} |
| Baseline severity of | N | 155 | 153 | |
| IBS: severe | | | | |
| Adequate relief of | n (%) | 82 (53) | 49 (32) | <0.001** |
| IBS symptoms | | | | |
| Adequate relief of | n (%) | 90 (58) | 55 (36) | <0.001** |
| abdominal pain/ | | | | |
| discomfort | | | | |
| Adequate relief of | n (%) | 82 (53) | 43 (28) | <0.001** |
| abnormal bowel habits | | | | |
| Baseline severity of | N | 222 | 215 | |
| IBS: non-severe | | | | |
| Adequate relief of IBS | n (%) | 124 (56) | 58 (27) | <0.001** |
| symptoms | | | | |
| Adequate relief of | n (%) | 131 (59) | 80 (37) | <0.001** |
| abdominal pain/ | | | | |
| discomfort | | | | |
| Adequate relief of | n (%) | 121 (55) | 61 (28) | <0.001** |
| abnormal bowel habits | | | | |

*Significant at the 0.050 level for treatment effect;
**Significant at the 0.010 level for treatment effect.
^{a}P-values for treatment effect are based on a non-parametric ANOVA model with an effect for treatment.
Note:
Percentages are based on the number of subjects in the ITT Population within the appropriate subgroup.
Note:
A responder for the three-month treatment period is defined as a subject who answers the weekly question with “yes” for at least half of the answers available with a minimum of four-week treatment duration during the 12-week treatment period. If a subject has less than a four-week treatment duration, the subject will be considered a non-responder.
Note:
Weekly diary observations made on or after the date of randomization and up through two days after the date of last dose of study medication during the 12-week treatment period (or the date of first dose of study medication during the rerandomization period if earlier) are included in this summary.
Data source: CSR Table 10.1.2.2.9 Left, Vol. 9 Righ

Analyses by IBS severity showed that the overall responder rate during the three-month treatment period was higher in the cilansetron 2 mg TID group compared with the placebo group for each of the three weekly diary questions for both severe IBS (N=308, 21% to 25% treatment group difference across the diary questions) and non-severe IBS (N=437, 22% to 29% treatment group difference across the diary questions). The treatment differences were statistically significant for each of the three weekly diary questions on adequate relief in subjects with both severe and non-severe IBS.

Study 5241.3.009

The number of months a subject was a responder for the weekly diary questions on adequate relief during Months 1 to 3 (monthly LOCF Imputation) is presented for the ITT population by severity of IBS in Table 2.7.3:42.

TABLE 2.7.3:42
Number of Months a Subject Was a Responder for the Weekly Diary
Questions on Adequate Relief During Months 1 to 3 by Severity of
IBS (Monthly LOCF Imputation)
Study S241.3.009 (ITT Population)

| Number of months a | | | | |
|---|---|---|---|---|
| subject was a responder | | Cilansetron | | |
| during Months | | 2 mg TID | Placebo | |
| 1 to 3 | Statistic | (N = 395) | (N = 397) | P-Value^{a} |
| Baseline severity of | N | 148 | 156 | |
| IBS: severe | | | | |
| IBS symptoms | | | | 0.001** |
| Zero months | n (%) | 47 (32) | 66 (42) | |
| One month | n (%) | 18 (12) | 31 (20) | |
| Two months | n (%) | 29 (20) | 29 (19) | |
| Three months | n (%) | 54 (36) | 30 (19) | |
| Abdominal | | | | 0.002** |
| pain/discomfort | | | | |
| Zero months | n (%) | 44 (30) | 62 (40) | |
| One month | n (%) | 19 (13) | 30 (19) | |
| Two months | n (%) | 29 (20) | 33 (21) | |
| Three months | n (%) | 56 (38) | 31 (20) | |
| Abnormal bowel habits | | | | <0.001** |
| Zero months | n (%) | 41 (28) | 62 (40) | |
| One month | n (%) | 18 (12) | 30 (19) | |
| Two months | n (%) | 29 (20) | 30 (19) | |
| Three months | n (%) | 60 (41) | 34 (22) | |
| Baseline severity of | N | 246 | 241 | |
| IBS: non-severe | | | | |
| IBS symptoms | | | | <0.001** |
| Zero months | n (%) | 63 (26) | 93 (39) | |
| One month | n (%) | 37 (15) | 51 (21) | |
| Two months | n (%) | 51 (21) | 42 (17) | |
| Three months | n (%) | 95 (39) | 55 (23) | |
| Abdominal | | | | <0.001** |
| pain/discomfort | | | | |
| Zero months | n (%) | 61 (25) | 97 (40) | |
| One month | n (%) | 42 (17) | 42 (17) | |
| Two months | n (%) | 41 (17) | 43 (18) | |
| Three months | n (%) | 102 (41) | 59 (24) | |
| Abnormal bowel habits | | | | <0.001** |
| Zero months | n (%) | 57 (23) | 89 (37) | |
| One month | n (%) | 31 (13) | 52 (22) | |
| Two months | n (%) | 54 (22) | 47 (20) | |
| Three months | n (%) | 104 (42) | 53 (22) | |

*Significant at the 0.050 level for treatment effect;
**Significant at the 0.010 level for treatment effect.
^{a}P-values for treatment effect are based on a non-parametric ANOVA model with an effect for treatment.
Note:
Percentages are based on the number of subjects in the ITT Population.
Note:
A subject is defined as a responder for a month if he/she reports adequate relief (‘yes’) for at least two of the four weeks during that month.
Note:
Weekly diary observations made on or after the date of randomization and up through two days after the date of last dose of study medication are included in this summary.
Data source: CSR Table 10.1.2.1.9 Left, Vol. 9 Righ

Analyses by IBS severity of the number of months a subject was a responder for the weekly diary questions on adequate relief showed statistically significant treatment group differences in favor of cilansetron compared with placebo for each of the three weekly diary questions independent of IBS severity (severe versus non-severe).

The overall responder rate for the weekly diary questions on adequate relief during the six-month treatment period (observed case) is presented for the ITT population by severity of IBS in Table 2.7.3:43.

TABLE 2.7.3:43
Overall Responder Rate for the Weekly Diary Questions on Adequate
Relief During the 26-Week Treatment Period by Severity of IBS
(Observed Case)
Study S241.3.009 (ITT Population)

| Number of subjects who | | | | |
|---|---|---|---|---|
| were responders during | | Cilansetron | | |
| the 26-week | | 2 mg TID | Placebo | |
| treatment period | Statistic | (N = 395) | (N = 397) | P-Value^{a} |
| Baseline severity of | N | 148 | 156 | |
| IBS: severe | | | | |
| Adequate relief of | n (%) | 84 (57) | 61 (39) | 0.002** |
| IBS symptoms | | | | |
| Adequate relief of | n (%) | 88 (59) | 63 (40) | <0.001** |
| abdominal pain/ | | | | |
| discomfort | | | | |
| Adequate relief of | n (%) | 91 (61) | 63 (40) | <0.001** |
| abnormal bowel habits | | | | |
| Baseline severity of | N | 246 | 241 | |
| IBS: non-severe | | | | |
| Adequate relief of | n (%) | 149 (61) | 118 (49) | 0.010* |
| IBS symptoms | | | | |
| Adequate relief of | n (%) | 150 (61) | 118 (49) | 0.008** |
| abdominal pain/ | | | | |
| discomfort | | | | |
| Adequate relief of | n (%) | 161 (65) | 119 (49) | <0.001** |
| abnormal bowel habits | | | | |

*Significant at the 0.050 level for treatment effect;
**Significant at the 0.010 level for treatment effect.
^{a}P-values for treatment effect are based on a non-parametric analysis of Variance (ANOVA) model with an effect for treatment.
Note:
Percentages are based on the number of subjects in the ITT Population within the appropriate subgroup.
Note:
Weekly diary observations made on or after the date of randomization and up through two days after the date of last dose of study medication are included in this summary.
Data source: CSR Table 10.1.2.2.17 Left, Vol. 9 Righ

Analyses by IBS severity showed that the overall responder rate during the 26-week treatment period was higher in the cilansetron 2 mg TID group compared with the placebo group for each of the three weekly diary questions for both severe IBS (N=304, 18% to 21% treatment group difference across the diary questions) and non-severe IBS (N=487, 12% to 16% difference across the diary questions).

Analyses by Duration of Disease

Study S241.3.006

The number of months a subject was a responder for the weekly diary questions on adequate relief during the three-month treatment period (monthly LOU imputation) is presented for the ITT population by duration of IBS in Table 2.7.3:44.

TABLE 2.7.3:44
Number of Months is Subject Was a Responder for the Weekly Diary
Questions on Adequate Relief During the Three-Month Treatment
Period by Duration of IBS (Monthly LOCF Imputation)
Study S241.3.006 (ITT Population)

| Number of months a subject | | | | |
|---|---|---|---|---|
| was a responder during the | | Cilansetron 2 mg TID | Placebo | |
| three-month treatment period | Statistic | (N = 344) | (N = 348) | P-Value* |
| Duration of IBS: ≦ 24 months | N | 51 | 47 | |
| IBS symptoms | | | | 0.037* |
| Zero months | n (%) | 19 (37) | 26 (55) | |
| One month | n (%) | 5 (10) | 5 (11) | |
| Two months | n (%) | 8 (16) | 7 (15) | |
| Three months | n (%) | 19 (37) | 9 (19) | |
| Abdominal pain/discomfort | | | | 0.007** |
| Zero months | n (%) | 5 (29) | 23 (49) | |
| One month | n (%) | 7 (14) | 8 (17) | |
| Two months | n (%) | 4 (8) | 7 (15) | |
| Three months | n (%) | 25 (49) | 9 (19) | |
| Abnormal bowel habits | | | | 0.024* |
| zero months | n (%) | 18 (35) | 25 (53) | |
| One month | n (%) | 4 (8) | 4 (9) | |
| Two months | n (%) | 7 (14) | 9 (19) | |
| Three months | n (%) | 22 (43) | 9 (19) | |
| Duration of IBS: > 24 months to ≦ 48 months | N | 43 | 52 | |
| IBS symptoms | | | | 0.114 |
| Zero months | n (%) | 13 (30) | 26 (50) | |
| One month | n (%) | 12 (28) | 9 (17) | |
| Two months | n (%) | 4 (9) | 6 (12) | |
| Three months | n (%) | 14 (33) | 11 (21) | |
| Abdominal pain/discomfort | | | | 0.339 |
| Zero months | n (%) | 14 (33) | 19 (37) | |
| One month | n (%) | 8 (19) | 12 (23) | |
| Two months | n (%) | 6 (14) | 9 (17) | |
| Three months | n (%) | 15 (35) | 12 (23) | |
| Abnormal bowel habits | | | | 0.043* |
| Zero months | n (%) | 13 (30) | 27 (52) | |
| One month | n (%) | 10 (23) | 8 (15) | |
| Two months | n (%) | 7 (16) | 9 (17) | |
| Three months | n (%) | 13 (30) | 8 (15) | |
| Duration of IBS: > 48 months to ≦120 months | N | 104 | 103 | |
| IBS symptoms | | | | 0.003** |
| Zero months | n (%) | 35 (34) | 54 (52) | |
| One month | n (%) | 15 (14) | 13 (13) | |
| Two months | n (%) | 19 (18) | 18 (17) | |
| Three months | n (%) | 35 (34) | 18 (17) | |
| Abdominal pain/discomfort | | | | 0.026* |
| Zero months | n (%) | 34 (33) | 49 (48) | |
| One month | n (%) | 12 (12) | 8 (8) | |
| Two months | n (%) | 18 (17) | 21 (20) | |
| Three months | n (%) | 40 (38) | 25 (24) | |
| Abnormal bowel habits | | | | <0.001** |
| Zero months | n (%) | 36 (35) | 58 (56) | |
| One month | n (%) | 12 (12) | 17 (17) | |
| Two months | n (%) | 19 (18) | 10 (10) | |
| Three months | n (%) | 37 (36) | 18 (17) | |
| Duration of IBS: > 120 months | N | 146 | 146 | |
| IBS symptoms | | | | <0.001** |
| Zero months | n (%) | 60 (41) | 89 (61) | |
| One month | n (%) | 13 (9) | 16 (11) | |
| Two months | n (%) | 22 (15) | 16 (11) | |
| Three months | n (%) | 51 (35) | 25 (17) | |
| Abdominal pain/discomfort | | | | <0.001** |
| Zero months | n (%) | 53 (36) | 81 (55) | |
| One month | n (%) | 15 (10) | 15 (10) | |
| Two months | n (%) | 19 (13) | 17 (12) | |
| Three months | n (%) | 59 (40) | 33 (23) | |
| Abnormal bowel habits | | | | <0.001** |
| Zero months | n (%) | 56 (38) | 93 (64) | |
| One month | n (%) | 14 (10) | 16 (11) | |
| Two months | n (%) | 19 (13) | 13 (9) | |
| Three months | n (%) | 57 (39) | 24 (16) | |

*Significant at the 0.050 level for treatment effect;
**Significant at the 0.010 level for treatment effect.
*P-values for treatment effect are based on a non-parametric ANOVA model with an effect for treatment.
Note:
Percentages are based on the number of subjects in the ITT Population.
Note:
A subject is defined as a responder for a month if he/she reports adequate relief (‘yes’) for at least two of the four weeks during that month.
Note:
Weekly diary observations made on or after the date of randomization and up through two days after the date of last dose of study medication are included in this summary.
Data source: CSR Table 10.1.2.1.11 Left, Vol. 9 Righ

Analyses by duration of IBS of the number of months a subject was a responder for the weekly diary questions on adequate relief showed statistically significant treatment group differences in favor of cilansetron compared with placebo for each of the three weekly diary questions, except that the treatment group difference was not significant for IBS symptoms and abdominal pain/discomfort in the EBS duration >24 months to ≦48 months category probably due to the small sample sizes. No consistent trends by duration of EBS were noted for any of the three weekly diary questions.

The overall responder rate for the weekly diary questions on adequate relief during the three-month treatment period (observed case) is presented for the ITT population by duration of IBS in Table 2.7.3:45.

TABLE 2.7.3:45
Overall Responder Rate for the Weekly Diary Questions on Adequate
Relief During the Three-Month Treatment Period by Duration of IBS
(Observed Case)
Study S241.3.006 (ITT Population)

| Number of subjects who were responders during the | | Cilansetron 2 mg TID | Placebo | |
|---|---|---|---|---|
| three-month treatment period | Statistic | (N = 344) | (N = 348) | P-Value^{a} |
| Duration of IBS: ≦24 months | N | 51 | 47 | |
| Adequate relief of IBS symptoms | n (%) | 25 (49) | 17 (36) | 0.201 |
| Adequate relief of abdominal pain/discomfort | n (%) | 27 (53) | 16 (34) | 0.061 |
| Adequate relief of abnormal bowel habits | n (%) | 27 (53) | 16 (34) | 0.061 |
| Duration of IBS: >24 months to ≦48 months | N | 43 | 52 | |
| Adequate relief of IBS symptoms | n (%) | 20 (47) | 18 (35) | 0.241 |
| Adequate relief of abdominal pain/discomfort | n (%) | 22 (51) | 21 (40) | 0.296 |
| Adequate relief of abnormal bowel habits | n (%) | 20 (47) | 15 (29) | 0.077 |
| Duration of IBS: >48 months to ≦120 months | N | 104 | 103 | |
| Adequate relief of IBS symptoms | n (%) | 54 (52) | 29 (28) | <0.001** |
| Adequate relief of abdominal pain/discomfort | n (%) | 58 (56) | 46 (45) | 0.111 |
| Adequate relief of abnormal bowel habits | n (%) | 54 (52) | 25 (24) | <0.001** |
| Duration of IBS: >120 months | N | 146 | 146 | |
| Adequate relief of IBS symptoms | n (%) | 69 (47) | 35 (24) | <0.001** |
| Adequate relief of abdominal pain/discomfort | n (%) | 72 (49) | 46 (32) | 0.002** |
| Adequate relief of abnormal bowel habits | n (%) | 73 (50) | 33 (23) | <0.001** |

*Significant at the 0.050 level for treatment effect;
**Significant at the 0.010 level for treatment effect.
^{a}P-values for treatment effect are based on a non-parametric analysis of Variance (ANOVA) model with an effect for treatment.
Note:
Percentages are based on the number of subjects in the ITT Population within the appropriate subgroup.
Note:
Weekly diary observations made on or after the date of randomization and up through two days after the date of last dose of study medication are included in this summary.
Data source: CSR Table 10.1.2.2.11 Left. Vol. 9 Righ

Analyses by duration of IBS showed that the overall responder rate during the three-month treatment period was higher in the cilansetron 2 mg TID group compared with the placebo group for each of the three weekly diary questions in each of the four IBS duration categories. No consistent trends in treatment group differences were noted by IBS duration category. The treatment group difference was not statistically significant in the ≦24 months and 24 months to ≦48 months subgroups, which was probably due to the smaller sample size in these subgroups compared with the other two subgroups.

Study S241.3.011

The number of months a subject was a responder for the weekly diary questions on adequate relief during the three-month treatment period (monthly LOCF Imputation) is presented for the ITT population by duration of IBS in Table 2.7.3:46.

TABLE 2.7.3:46
Number of Months a Subject Was a Responder for the Weekly Diary
Questions on Adequate Relief During the Three-Month Treatment
Period by Duration of IBS (Monthly LOCF Imputation)
Study S241.3.011 (ITT Population)

| Number of months a subject | | | | |
|---|---|---|---|---|
| was a responder during the | | Cilansetron 2 mg TID | Placebo | |
| three-month treatment period | Statistic | (N = 377) | (N = 369) | P-Value^{a} |
| Duration of IBS: > 24 months | N | 70 | 66 | |
| IBS symptoms | | | | <0.001** |
| Zero months | n (%) | 15 (21) | 33 (50) | |
| One month | n (%) | 11 (16) | 11 (17) | |
| Two months | n (%) | 16 (23) | 8 (12) | |
| Three months | n (%) | 18 (40) | 14 (2)) | |
| Abdominal pain/discomfort | | | | 0.025* |
| Zero months | n (%) | 20 (29) | 28 (42) | |
| One month | n (%) | 7 (10) | 12 (18) | |
| Two months | n (%) | 16 (23) | 9 (14) | |
| Three months | n (%) | 27 (39) | 17 (26) | |
| Abnormal bowel habits | | | | <0.001** |
| Zero months | n (%) | 17 (24) | 33 (50) | |
| One month | n (%) | 8 (11) | 11 (17) | |
| Two months | n (%) | 15 (21) | 10 (15) | |
| Three months | n (%) | 30 (43) | 12 (18) | |
| Duration of IBS: > 24 months to ≦ 48 months | N | 72 | 65 | |
| IBS symptoms | | | | 0.008** |
| Zero months | n (%) | 17 (24) | 28 (43) | |
| One month | n (%) | 7 (10) | 8 (12) | |
| Two months | n (%) | 16 (22) | 11 (17) | |
| Three months | n (%) | 32 (44) | 18 (28) | |
| Abdominal pain/discomfort | | | | 0.111 |
| Zero months | n (%) | 15 (21) | 20 (31) | |
| One month | n (%) | 10 (14) | 11 (17) | |
| Two months | n (%) | 15 (21) | 12 (18) | |
| Three months | n (%) | 32 (44) | 22 (34) | |
| Abnormal bowel habits | | | | 0.038* |
| Zero months | n (%) | 20 (28) | 28 (43) | |
| One month | n (%) | 10 (14) | 6 (9) | |
| Two months | n (%) | 10 (14) | 15 (23) | |
| Three months | n (%) | 32 (44) | 16 (25) | |
| Duration of IBS: > 48 months to ≦ 120 months | N | 133 | 116 | |
| IBS symptoms | | | | <0.001** |
| Zero months | n (%) | 38 (29) | 68 (59) | |
| one month | n (%) | 19 (14) | 13 (11) | |
| Two months | n (%) | 26 (20) | 18 (16) | |
| Three months | n (%) | 50 (38) | 17 (15) | |
| Abdominal pain/discomfort | | | | <0.001** |
| Zero months | n (%) | 34 (26) | 57 (49) | |
| One month | n (%) | 15 (11) | 13 (11) | |
| Two months | n (%) | 23 (17) | 20 (17) | |
| Three months | n (%) | 61 (46) | 26 (22) | |
| Abnormal bowel habits | | | | <0.001** |
| Zero months | n (%) | 39 (29) | 65 (39) | |
| One month | n (%) | 15 (11) | 16 (14) | |
| Two months | n (%) | 29 (22) | 17 (15) | |
| Three months | n (%) | 50 (38) | 15 (13) | |
| Duration of IBS: > 120 months | N | 102 | 121 | |
| IBS symptoms | | | | <0.001** |
| Zero months | n (%) | 35 (34) | 68 (56) | |
| One month | n (%) | 17 (17) | 15 (12) | |
| Two months | n (%) | 12 (12) | 19 (16) | |
| Three months | n (%) | 38 (37) | 19 (16) | |
| Abdominal pain/discomfort | | | | 0.005** |
| Zero months | n (%) | 32 (31) | 56 (46) | |
| One month | n (%) | 13 (13) | 19 (16) | |
| Two months | n (%) | 18 (18) | 18 (15) | |
| Three months | n (%) | 39 (38) | 28 (23) | |
| Abnormal bowel habits | | | | <0.001** |
| Zero months | n (%) | 36 (35) | 74 (61) | |
| One month | n (%) | 17 (17) | 10 (8) | |
| Two months | n (%) | 12 (12) | 19 (16) | |
| Three months | n (%) | 37 (36) | 18 (15) | |

*Significant at the 0.050 level for treatment effect;
**Significant at the 0.010 level for treatment effect.
*P-values for treatment effect are based on a non-parametric ANOVA model with an effect for treatment.
Note:
Percentages are based on the number of subjects in the ITT Population.
Note:
A subject is defined as a responder for a month if he/she reports adequate relief (‘yes’) for at least two of the four weeks during that month.
Note:
Weekly diary observations made on or after the date of randomization and up through two days after the date of last dose of study medication during the 12-week treatment period (or the date of first dose of study medication during the rerandomization period if earlier) are included in this summary.
Data source: CSR Table 10.1.2.1.11 Left, Vol. 9 Righ

Analyses by duration of IBS of the number of months a subject was a responder for the weekly diary questions on adequate relief showed statistically significant treatment group differences in favor of cilansetron 2 mg TID compared with placebo for each of the three weekly diary questions except that the treatment group difference was not significant for abdominal pain/discomfort in the IBS duration >24 months to ≦48 months category. No consistent trends by duration of IBS were noted for any of the three weekly diary questions.

The overall responder rate for the weekly diary questions on adequate relief during the three-month treatment period (observed case) is presented for the ITT population by duration of IBS in Table 2.7.3:47.

TABLE 2.7.3:47
Overall Responder Rate for the Weekly Diary Questions on Adequate
Relief During the Three-Month Treatment Period by Duration of IBS
(Observed Case)
Study S241.3.011 (ITT Population)

| Number of subjects who were responders during the | | Cilansetron 2 mg TID | Placebo | |
|---|---|---|---|---|
| three-month treatment period | Statistic | (N = 377) | (N = 369) | P-Value^{a} |
| Duration of IBS: ≦24 months | N | 70 | 66 | |
| Adequate relief of IBS symptoms | n (%) | 45 (64) | 20 (30) | <0.001** |
| Adequate relief of abdominal pain/discomfort | n (%) | 43 (61) | 24 (36) | 0.004** |
| Adequate relief of abnormal bowel habits | n (%) | 45 (64) | 20 (30) | <0.001** |
| Duration of IBS: >24 months to ≦48 months | N | 72 | 65 | |
| Adequate relief of IBS symptoms | n (%) | 44 (61) | 28 (43) | 0.035* |
| Adequate relief of abdominal pain/discomfort | n (%) | 44 (61) | 34 (52) | 0.301 |
| Adequate relief of abnormal bowel habits | n (%) | 43 (60) | 27 (42) | 0.034* |
| Duration of IBS: >48 months to ≦120 months | N | 133 | 116 | |
| Adequate relief of IBS symptoms | n (%) | 69 (52) | 30 (26) | <0.001** |
| Adequate relief of abdominal pain/discomfort | n (%) | 79 (59) | 41 (35) | <0.001** |
| Adequate relief of abnormal bowel habits | n (%) | 69 (52) | 29 (25) | <0.001** |
| Duration of IBS: >120 months | N | 102 | 121 | |
| Adequate relief of IBS symptoms | n (%) | 48 (47) | 30 (25) | <0.001** |
| Adequate relief of abdominal pain/discomfort | n (%) | 55 (54) | 37 (31) | <0.001** |
| Adequate relief of abnormal bowel habits | n (%) | 46 (45) | 29 (24) | <0.001** |

*Significant at the 0.050 level for treatment effect;
**Significant at the 0.010 level for treatment effect.
^{a}P-values for treatment effect are based on a non-parametric ANOVA model with an effect for treatment.
Note:
Percentages are based on the number of subjects in the ITT Population within the appropriate subgroup.
Note:
A responder for the three-month treatment period is defined as a subject who answers the weekly question with “yes” for at least half of the answers available with a minimum of four-week treatment duration during the 12-week treatment period. If a subject has less than a four-week treatment duration, the subject will be considered a non-responder.
Note:
Weekly diary observations made on or after the date of randomization and up through two days after the date of last dose of study medication during the 12-week treatment period (or the date of first dose of study medication during the rerandomization period if earlier) are included in this summary.
Data source: CSR Table 10.1.2.2.11 Left. Vol. 9 Righ

Analyses by duration of IBS showed that the overall responder rate during the three-month treatment period was significantly higher in the cilansetron 2 mg TID group compared with the placebo group for each of the three weekly diary questions in each of the four IBS duration categories except that the treatment group difference was not significant for abdominal pain/discomfort in the IBS duration >24 months to ≦48 months category. No consistent trends in treatment group differences were noted by IBS duration.

Study S241.3.009

The number of months a subject was a responder for the weekly diary questions on adequate relief during Months 1 to 3 (monthly LOCF Imputation) is presented for the ITT population by duration of IBS in Table 2.7.3:48.

TABLE 2.7.3:48
Number of Months a Subject Was a Responder for the Weekly Diary
Questions on Adequate Relief During Months 1 to 3 by Duration of
IBS (Monthly LOCF Imputation)
Study S241.3.009 (ITT Population)

| Number of months a subject was a | | Cilansetron 2 mg TID | Placebo | |
|---|---|---|---|---|
| responder during Months 1 to 3 | Statistic | (N = 395) | (N = 397) | P-Value* |
| Duration of IBS: ≦24 months | N | 96 | 119 | |
| IBS symptoms | | | | <0.001** |
| Zero months | n (%) | 18 (19) | 43 (36) | |
| One month | n (%) | 16 (17) | 25 (21) | |
| Two months | n (%) | 16 (17) | 27 (23) | |
| Three months | n (%) | 46 (48) | 24 (20) | |
| Abdominal pain/discomfort | | | | <0.001** |
| Zero months | n (%) | 17 (18) | 43 (36) | |
| One month | n (%) | 11 (11) | 27 (23) | |
| Two months | n (%) | 13 (14) | 24 (20) | |
| Three months | n (%) | 55 (57) | 25 (21) | |
| Abnormal bowel habits | | | | <0.001** |
| Zero months | n (%) | 18 (19) | 42 (35) | |
| One month | n (%) | 9 (9) | 24 (20) | |
| Two months | n (%) | 18 (19) | 26 (22) | |
| Three months | n (%) | 51 (53) | 27 (23) | |
| Duration of IBS: >24 months to ≦48 months | N | 96 | 87 | |
| IBS symptoms | | | | 0.012* |
| Zero months | n (%) | 31 (32) | 41 (47) | |
| One month | n (%) | 9 (9) | 11 (13) | |
| Two months | n (%) | 21 (22) | 16 (18) | |
| Three months | n (%) | 35 (36) | 19 (22) | |
| Abdominal pain/discomfort | | | | 0.011* |
| Zero months | n (%) | 26 (27) | 36 (41) | |
| One month | n (%) | 14 (15) | 15 (17) | |
| Two months | n (%) | 19 (20) | 16 (18) | |
| Three months | n (%) | 37 (39) | 20 (23) | |
| Abnormal bowel habits | | | | 0.002** |
| Zero months | n (%) | 24 (25) | 35 (40) | |
| One month | n (%) | 11 (11) | 16 (18) | |
| Two months | n (%) | 25 (26) | 20 (23) | |
| Three months | n (%) | 36 (38) | 16 (18) | |
| Duration of IBS: >48 months to ≦120 months | N | 131 | 117 | |
| IBS symptoms | | | | 0.024* |
| Zero months | n (%) | 41 (31) | 43 (37) | |
| One month | n (%) | 16 (12) | 30 (26) | |
| Two months | n (%) | 28 (21) | 16 (14) | |
| Three months | n (%) | 46 (35) | 28 (24) | |
| Abdominal pain/discomfort | | | | 0.016* |
| Zero months | a (%) | 42 (32) | 50 (43) | |
| One month | n (%) | 21 (16) | 21 (18) | |
| Two months | n (%) | 23 (18) | 23 (20) | |
| Three months | n (%) | 45 (34) | 23 (20) | |
| Abnormal bowel habits | | | | 0.003** |
| Zero months | n (%) | 35 (27) | 44 (38) | |
| One month | n (%) | 19 (15) | 27 (23) | |
| Two months | n (%) | 26 (20) | 19 (16) | |
| Three months | n (%) | 51 (39) | 27 (23) | |
| Duration of IBS: >120 months | N | 72 | 74 | |
| IBS symptoms | | | | 0.021* |
| Zero months | n (%) | 20 (28) | 32 (43) | |
| One month | n (%) | 14 (19) | 16 (22) | |
| Two months | n (%) | 15 (21) | 12 (16) | |
| Three months | n (%) | 23 (32) | 14 (19) | |
| Abdominal pain/discomfort | | | | 0.390 |
| Zero months | n (%) | 20 (28) | 30 (41) | |
| One month | n (%) | 15 (21) | 9 (12) | |
| Two months | n (%) | 15 (21) | 13 (18) | |
| Three months | n (%) | 22 (31) | 22 (30) | |
| Abnormal bowel habits | | | | 0.035* |
| Zero months | n (%) | 21 (29) | 30 (41) | |
| One month | n (%) | 10 (14) | 15 (20) | |
| Two months | n (%) | 14 (19) | 12 (16) | |
| Three months | n (%) | 27 (38) | 17 (23) | |

*Significant at the 0.050 level for treatment effect;
**Significant at the 0.010 level for treatment effect.
*P-values for treatment effect are based on a non-parametric ANOVA model with an effect for treatment.
Note:
Percentages are based on the number of subjects in the ITT Population.
Note:
A subject is defined as a responder for a month if he/she reports adequate relief (‘yes’) for at least two of the four weeks during that month.
Note:
Weekly diary observations made on or after the date of randomization and up through two days after the date of last dose of study medication are included in this summary.
Data source: CSR Table 10.1.2.1.11 Left, Vol. 9 Righ

Analyses by duration of IBS of the number of months a subject was a responder for the weekly diary questions on adequate relief showed statistically significant treatment group differences in favor of cilansetron compared with placebo for each of the three weekly diary questions except that the treatment group difference was not significant for abdominal pain/discomfort in the IBS duration >120 months category.

The overall responder rate for the weekly diary questions on adequate relief during the six-month treatment period (observed case) is presented for the ITT population by duration of IBS in Table 2.7.3:49.

TABLE 2.7.3:49
Overall Responder Rate for the Weekly Diary Questions on Adequate
Relief During the 26-Week Treatment Period by Duration of IBS
(Observed Case)
Study S241.3.009 (ITT Population)

| Number of subjects who were responders during | | Cilansetron 2 mg TID | Placebo | |
|---|---|---|---|---|
| the 26-week treatmet period | Statistic | (N = 395) | (N = 397) | P-Value^{a} |
| Duration of IBS: ≦24 months | N | 96 | 119 | |
| Adequate relief of IBS symptoms | n (%) | 66 (69) | 49 (41) | <0.001** |
| Adequate relief of abdominal pain/discomfort | n (%) | 70 (73) | 53 (45) | <0.001** |
| Adequate relief of abnormal bowel habits | n (%) | 69 (72) | 47 (39) | <0.001** |
| Duration of IBS: >24 months to ≦48 months | N | 96 | 87 | |
| Adequate relief of IBS symptoms | n (%) | 56 (58) | 39 (45) | 0.069 |
| Adequate relief of abdominal pain/discomfort | n (%) | 60 (63) | 42 (48) | 0.054 |
| Adequate relief of abnormal bowel habits | n (%) | 63 (66) | 43 (49) | 0.027* |
| Duration of IBS: >48 months to ≦120 months | N | 131 | 117 | |
| Adequate relief of IBS symptoms | n (%) | 72 (55) | 60 (51) | 0.563 |
| Adequate relief of abdominal pain/discomfort | n (%) | 68 (52) | 53 (45) | 0.300 |
| Adequate relief of abnormal bowel habits | n (%) | 79 (60) | 59 (50) | 0.119 |
| Duration of IBS: >120 months | N | 72 | 74 | |
| Adequate relief of IBS symptoms | n (%) | 40 (56) | 31 (42) | 0.100 |
| Adequate relief of abdominal pain/discomfort | n (%) | 41 (57) | 33 (45) | 0.137 |
| Adequate relief of abnormal bowel habits | n (%) | 42 (58) | 33 (45) | 0.098 |

*Significant at the 0.050 level for treatment effect;
**Significant at the 0.010 level for treatment effect.
^{a}P-values for treatment effect are based on a non-parametric analysis of Variance (ANOVA) model with an effect for treatment.
Note:
Percentages are based on the number of subjects in the ITT Population within the appropriate subgroup.
Note:
Weekly diary observations made on or after the date of randomization and up through two days after the date of last dose of study medication are included in this summary.
Data source: CSR Table 10.1.2.2.1.19 dLeft. Vol. 9 Righ

Analyses by duration of IBS showed that the overall responder rate during the 26-week treatment period was higher in the cilansetron 2 mg TID group compared with the placebo group for each of the three weekly diary questions in each of the four IBS duration categories. However, subjects with a longer duration of IBS demonstrated smaller treatment group differences compared with subjects with a shorter duration of IBS (IBS duration ≦24 months, N=215, 28% to 33% treatment group difference across the diary questions; >24 months to ≦48 months, N=183, 13% to 17% difference across the diary questions; >48 months to ≦120 months, N=248, 4% to 10% difference across the diary questions; and >120 months, N=146, 12% to 14% difference across the diary questions).

Analyses by Tobacco Use

Study S241.3.006

The number of months a subject was a responder for the weekly diary questions on adequate relief during the three-month treatment period (monthly LOCF Imputation) is presented for the ITT population by use of tobacco in Table 2.7.3.6.1.1. Analyses by tobacco use of the number of months a subject was a responder for the weekly diary questions on adequate relief showed statistically significant treatment group differences in favor of cilansetron 2 mg TID compared with placebo for the question on adequate relief of abnormal bowel habits in both subgroups of tobacco use (yes/no). The number of months a subject was a responder for the weekly diary questions on adequate relief of IBS symptoms and abdominal pain/discomfort showed treatment group differences in favor of cilansetron 2 mg TID compared with placebo in both subgroups of tobacco use (yes/no); however, the treatment group differences were not statistically significant among subjects using tobacco (p=0.061 and 0.071 for EBS symptoms and abdominal pain/discomfort, respectively).

The overall responder rate for the weekly diary questions on adequate relief during the three-month treatment period (observed case) is presented for the ITT population by use of tobacco in Table 2.7.3.6.2.1. Analyses by tobacco use showed that the overall responder rate during the three-month treatment period was higher in the cilansetron 2 mg TID group compared with the placebo group for each of the three weekly diary questions in subjects with tobacco use (N=148, 5% to 17% treatment group difference across the diary questions) and no tobacco use (N=544, 18% to 27% treatment group difference across the diary questions). The treatment differences were statistically significant for each of the three weekly diary questions on adequate relief in subjects not using tobacco and for the weekly diary question on abnormal bowel habits in subjects using tobacco.

Study S241.3.011

The number of months a subject was a responder for the weekly diary questions on adequate relief during the three-month treatment period (monthly LOCF Imputation) is presented for the ITT population by use of tobacco in CSR Table 10.1.2.1.12. Analyses by tobacco use of the number of months a subject was a responder for the weekly diary questions on adequate relief showed statistically significant treatment group differences in favor of cilansetron 2 mg TID compared with placebo for each of the three weekly diary questions in both subgroups of tobacco use (yes/no).

The overall responder rate for the weekly diary questions on adequate relief during the three-month treatment period (observed case) is presented for the ITT population by use of tobacco in CSR Table 10.1.2.2.12. Analyses by tobacco use showed that the overall responder rate during the three-month treatment period was higher in the cilansetron 2 mg TID group compared with the placebo group for each of the three weekly diary questions in subjects with tobacco use (N=152, 17% to 25% treatment group difference across the diary questions) and no tobacco use (N=593, 23% to 27% treatment group difference across the diary questions). The treatment differences were statistically significant for each of the three weekly diary questions on adequate relief in subjects both using and not using tobacco.

Study S241.3.009

The number of months a subject was a responder for the weekly diary questions on adequate relief during the three-month treatment period (monthly LOCF Imputation) is presented for the ITT population by use of tobacco in Table 2.7.3.6.1.2. Analyses by tobacco use of the number of months a subject was a responder for the weekly diary questions on adequate relief showed statistically significant treatment group differences in favor of cilansetron 2 mg TID compared with placebo for each of the three weekly diary questions in both subgroups of tobacco use (yes/no).

The overall responder rate for the weekly diary questions on adequate relief during the six-month treatment period (observed case) is presented for the 11 population by use of tobacco in Table 2.7.3.6.2.2. Analyses by tobacco use showed that the overall responder rate during the six-month treatment period was higher in the cilansetron 2 mg TID group compared with the placebo group for each of the three weekly diary questions in subjects with tobacco use (N=187, 10% to 17% treatment group difference across the diary questions) and no tobacco use (N=605, 14% to 18% treatment group difference across the diary questions). The treatment differences were statistically significant for each of the three weekly diary questions on adequate relief in subjects not using tobacco and for the weekly diary questions on IBS symptoms and abnormal bowel habits in subjects using tobacco.

Analyses by Pooled Center and by Pooled Country

Study S241.3.006

The number of months a subject was a responder for the weekly diary questions on adequate relief during the three-month treatment period (monthly LOCF imputation) is presented for the ITT population by pooled center in CSR Table 10.1.2.1.7. Centers were pooled by geographical region. The treatment differences across the pooled centers in the number of months a subject was a responder for the weekly diary questions on adequate relief showed cilansetron 2 mg TID consistently with a more favorable distribution over placebo.

The overall responder rate for the weekly diary questions on adequate relief during the three-month treatment period (observed case) is presented for the ITT population by pooled center in CSR Table 10.1.2.2.7. Centers were pooled by geographical region. The treatment differences across all pooled centers showed cilansetron 2 mg TID consistently with higher responder rates over placebo.

Study S241.3.011

The number of months a subject was a responder for the weekly diary questions on adequate relief during the three-month treatment period (monthly LOCF Imputation) was assessed. Centers were pooled by geographical region. The treatment differences across the pooled centers in the number of months a subject was a responder for the weekly diary questions on adequate relief showed cilansetron 2 mg TID consistently with a more favorable distribution over placebo with the exception of Peru. Of note, only 4% of subjects (16 subjects in both groups) were randomized in Peru.

The treatment group differences across all pooled centers showed cilansetron 2 mg TID consistently with higher responder rates over placebo for each diary question, except for Peru (abdominal pain/discomfort: 69% versus 81%; abnormal bowel habits, 75% versus 81%) and Argentina and Chile (abdominal pain/discomfort: 65% versus 67%). Of note, these two pooled centers enrolled only 4% (Peru) and 5% (Argentina and Chile) of the subject population.

Study S241.3.009

The number of months a subject was a responder for the weekly diary questions on adequate relief during Months 1 to 3 (monthly LOCF Imputation) was assessed for the treatment group. The treatment differences across the country groups in the number of months a subject was a responder for the weekly diary questions on adequate relief showed cilansetron 2 mg TID consistently with more favorable distribution over placebo, except South Africa and Romania where subjects in the cilansetron 2 mg TID group showed less favorable distribution compared to subjects in the placebo group.

The overall responder rate for the weekly diary questions on adequate relief during the six-month treatment period (observed case) was assessed for the treatment group. Countries were pooled by geographical region. The largest treatment differences in the overall responder rate in favor of cilansetron were seen in Ukraine (N=143, 36% to 39% difference across the diary questions) and the pooled country group of Australia, Canada, and New Zealand (N=1 11, 22% to 34% difference across the diary questions). The treatment differences across the other country groups showed cilansetron 2 mg TID consistently with higher responder rates over placebo, except the country groups of Romania and South Africa where the overall responder rate was, in fact, lower in the cilansetron 2 mg TID group compared with the placebo group for each diary question (Romania, N=53, −29% to −8% difference across the diary questions; South Africa, N=29, −23% treatment group difference for each diary question). Of note, these two country groups had the least number of subjects (Romania, N=53 [7%] and South Africa, N=29 [4%]; all other regions, N=74 [9%] to 143 [18%]). Placebo response was higher in Romania (79% to 83% across the diary questions) and South Africa (65% for each diary question) compared with all other regions. Analysis of treatment by pooled country interactions did not reveal any patterns across pooled countries that could provide an explanation for this phenomenon.

Deterioration of Effect Analyses

The deterioration of effect analyses focus is to evaluate the percentage of subjects who worsened from Baseline. Clinical limits were set in order to capture a clinically meaningful number of subjects who worsened from Baseline. This clinical limit was also used in identifying those who improved from Baseline which led to a much larger percentage of subjects meeting this criterion. Percentages of subjects who improved from Baseline for these analyses are discussed in order to understand the context of the percentage of subjects who worsened.

In general, the percentage of subjects who worsened from Baseline was low in both treatment groups, with incidence rates between 2% and 17%. Cilansetron 2 mg TID showed a consistently lower incidence of worsening from Baseline of 2% to 9% compared to placebo of 7% to 17% in all deterioration of effect analyses.

Deterioration of Effect Based on Daily Abdominal Pain

A summary of deterioration of effect for Study S241.3.006 based on daily abdominal pain is presented in Table 2.7.3:50 (observed value), Table 2.7.3.6.9.1 (observed value), and Table 2.7.3.6.9.2 (LOCF value). In both treatment groups, the percentage of subjects who worsened from Baseline was the same at 13% for Weeks 1-2. Over time, the percentage of subjects who worsened from Baseline decreased in incidence for cilansetron 2 mg TID to between 7% and 9% compared to placebo of 11% to 17%. Consistently, cilansetron 2 mg TID showed higher incidence of improvement from Baseline compared to placebo throughout the 12 weeks and when adjusted to those who worsened from Baseline the difference between treatment groups was even greater. Similar comparisons between cilansetron 2 mg TID and placebo were seen for the LOCF values.

A summary of deterioration of effect for Study S241.3.009 based on daily abdominal pain is presented in Table 2.7.3:50 (observed value), Table 2.7.3.6.10.1 (observed value), and Table 2.7.3.6.10.2 (LOCF value). In both treatment groups, the percentage of subjects who worsened from Baseline was largest for Weeks 1-2, with 13% in cilansetron 2 mg TID and 15% in placebo for the observed values. Over time, the percentage of subjects who worsened from Baseline decreased in incidence for both treatment groups with cilansetron 2 mg TID showing lower incidence between 4% and 8% compared to placebo with 7%.to 11%. Consistently cilansetron 2 mg TID showed higher incidence of improvement from Baseline compared to placebo. throughout the 26 weeks. Similar comparisons between cilansetron 2 mg TID and placebo were seen for the LOCF values, except that there was less difference between the treatment groups on the incidence of improved from Baseline.

TABLE 2.7.3:50
Deterioration of Effect Analyses: Daily Abdominal Pain (Observed
Value)

| Percentage of | S241.3.006 | | S241.3.009 | |
|---|---|---|---|---|
| Subjects Who Worsened | Cilansetron | | Cilansetron | |
| from Baseline | 2 mg TID | Placebo | 2 mg TID | Placebo |
| Weeks 1-2 | 13% | 13% | 13% | 15% |
| Weeks 3-4 | 8% | 11% | 8% | 10% |
| Weeks 5-6 | 9% | 11% | 8% | 9% |
| Weeks 7-8 | 7% | 11% | 7% | 9% |
| Weeks 9-10 | 8% | 17% | 4% | 10% |
| Weeks 11-12 | 8% | 13% | 7% | 8% |
| Weeks 13-26 | — | — | 6-8% | 7-11% |

Note:
“Worsened” is defined as the difference between the two-week post-Baseline and the Baseline average score ≧0.20.
Note:
Calculation of two-week average score requires a minimum of seven non-missing daily responses.
Note:
Percentages are based on the number of subjects in the ITT population with non-missing Baseline and post-Baseline two-week average scores.
Data Source: Study S241.3.006: Table 2.7.3.6.9.1 Left. Vol. 9 Righ; Study S241.3.009: Table 2.7.3.6.10.1 Left. Vol. 9 Righ

Deterioration of Effect Based on Daily Urgency

A summary of deterioration of effect for Study S241.3.006 based on daily urgency is presented in Table 2.7.3:51 (observed value), Table 2.7.3.6.11.1 (observed value), and Table 2.7.3.6.11.2 (LOCF value). At Weeks 1-2, the percentage of subjects who worsened from Baseline in cilansetron 2 mg TID was 5% compared to 12% in placebo for the observed values. Over time, this difference in the percentage of subjects who worsened from Baseline continued to favor cilansetron 2 mg TID with incidence between 5% and 8% compared to placebo of 11% to 17%. Consistently, cilansetron 2 mg TID showed higher incidence of improvement from Baseline compared to placebo throughout the 12 weeks and when adjusted to those who worsened from Baseline the difference between treatment groups was even greater.

Similar comparisons between cilansetron 2 mg TID and placebo were seen for the LOCF values. A summary of deterioration of effect for Study S241.3.009 based on daily urgency is presented in Table 2.7.3:51 (observed value), Table 2.7.3.6.12.1 (observed value), and Table 2.7.3.6.12.2 (LOCF value). At Weeks 1-2, the percentage of subjects who worsened from Baseline in cilansetron 2 mg TID was 7% compared to 11% in placebo for the observed values. Over time, this difference in the percentage of subjects who worsened from Baseline continued to favor cilansetron 2 mg TID with incidence between 4% and 8% compared to placebo of 7% to 13%. Consistently, cilansetron 2 mg TED showed higher incidence of improvement from Baseline compared to placebo throughout the 26 weeks and when adjusted to those who worsened from Baseline, the difference between treatment groups was even greater. Similar comparisons between cilansetron 2 mg TID and placebo were seen for the LOCF values.

TABLE 2.7.3:51
Deterioration of Effect Analyses: Daily Urgency (Observed Value)

| Percentage of | S241.3.006 | | S241.3.009 | |
|---|---|---|---|---|
| Subjects Who Worsened | Cilansetron | | Cilansetron | |
| from Baseline | 2 mg TID | Placebo | 2 mg TID | Placebo |
| Weeks 1-2 | 5% | 12% | 7% | 11% |
| Weeks 3-4 | 5% | 13% | 5% | 11% |
| Weeks 5-6 | 8% | 11% | 5% | 12% |
| Weeks 7-8 | 5% | 11% | 4% | 13% |
| Weeks 9-10 | 8% | 17% | 6% | 9% |
| Weeks 11-12 | 8% | 14% | 4% | 13% |
| Weeks 13-26 | — | — | 5-8% | 7-11% |

Note:
“Worsened” is defined as the difference between the two-week post-Baseline and the Baseline average score ≧0.20.
Note:
Calculation of two-week average score requires a minimum of seven non-missing daily responses.
Note:
Percentages are based on the number of subjects in the ITT population with non-missing Baseline and post-Baseline two-week average scores.
Data source: Study S241.3.006: Table 2.7.3.6.11.1 Left. Vol. 9 Righ; Study S241.3.009: Table 2.7.3.6.12.1 Left. Vol. 9 Righ

Deterioration of Effect Based on Interruption of Activities

A summary of deterioration of effect for Study S241.3.006 based on interruption of activities is presented in Table 2.7.3:52 (observed value), Table 2.7.3.6.13.1 (observed value), Table 2.7.3.6.13.2 (Baseline Score=0, observed value), Table 2.7.3.6.13.3 (Baseline Score=4, observed value), Table 2.7.3.6.13.4 (LOCF value), Table 2.7.3.6.13.5 (Baseline Score=0, LOCF value), and Table 2.7.3.6.13.6 (Baseline Score=4, LOCF value). At Month 1, the percentage of subjects who worsened from Baseline in cilansetron 2 mg TID was 8% compared to 16% in placebo for the observed values. Over time, this difference in the percentage of subjects who worsened from Baseline continued to favor cilansetron 2 mg TID with incidence between 4% and 5% compared to placebo of 14% to 17%. Consistently, cilansetron 2 mg TID showed higher incidence of improvement from Baseline compared to placebo throughout the three months and when-adjusted to those who worsened from Baseline, the difference between treatment groups was even greater. Subjects whose Baseline score was a 4 showed greater incidence of improvement from Baseline for cilansetron 2 mg TID of 80% to 86% compared to 70% to 75% for placebo. There was insufficient sample size to discuss subjects who had a zero at Baseline, i.e. could only stay the same or get worse. Similar comparisons between cilansetron 2 mg TID and placebo were seen for the LOCF values.

A summary of deterioration of effect for Study S241.3.009 based on interruption of activities is presented in Table 2.7.3:52 (observed value), Table 2.7.3.6.14.1 (observed value), Table 2.7.3.6.14.2 (Baseline Score=O, observed value), Table 2.7.3.6.14.3 (Baseline Score=4, observed value), Table 2.7.3.6.14.4 (LOCF value), Table 2.7.3.6.14.5 (Baseline Score=O, LOCF value), Table 2.7.3.6.14.6 (Baseline Score=4, LOCF value). At Month 1, the percentage of subjects who worsened from Baseline in cilansetron 2 mg TID was 5% compared to 9% in placebo for the observed values. Over time, this difference in the percentage of subjects who worsened from Baseline continued to favor cilansetron 2 mg TID with incidence between 2% and 3% compared to placebo of 7%. Consistently cilansetron 2 mg TIED showed higher incidence of improvement from Baseline compared to placebo throughout the six months and when adjusted to those who worsened from Baseline the difference between treatment groups was even greater. There was insufficient sample size to discuss subjects who had a zero at Baseline, i.e. could only stay the same or get worse, but consistently the cilansetron 2 mg TID group had 16% to 20% worsened from Baseline compared to 41% to 44% on placebo. Subjects whose Baseline score was a 4 showed similar incidence of improvement from Baseline for cilansetron 2 mg TID of 83% to 91% and placebo of 88% to 92%, but sample sizes were much less than those for the core observed value analysis. Sirnilar comparisons between cilansetron 2 mg TID and placebo were seen for the LOCF values.

TABLE 2.7.3:52
Deterioration of Effect Analyses: Interruption of Activities (Observed
Value)

| Percentage of | S241.3.006 | | S241.3.009 | |
|---|---|---|---|---|
| Subjects Who Worsened | Cilansetron | | Cilansetron | |
| from Baseline | 2 mg TID | Placebo | 2 mg TID | Placebo |
| Month 1 | 8% | 16% | 5% | 9% |
| Month 2 | 5% | 14% | — | — |
| Month 3 | 4% | 17% | 3% | 7% |
| Month 6 | — | — | 2% | 7% |

Note:
“Worsened” is defined as the post-Baseline Score > Baseline score.
Note:
Percentages are based on the number of subjects in the ITT population with a Baseline score < 4 and non-missing post-Baseline score.
Data Source: Study S241.3.006: Table 2.7.3.6.13.1 Left. Vol. 9 Righ; Study S241.3.009: Table 2.7.3.6.14.1 Left. Vol. 9 Righ

Deterioration of Effect Based on the IBS-QOL Overall Score

A summary of deterioration of effect for Study S241.3.006 based on the IBS-QOL Overall Score is presented in Table 2.7.3:53 and Table 2.7.3.6.15.1. At Month 3, the percentage of subjects who worsened from Baseline in cilansetron 2 mg TID was 5% compared to 10% in placebo. Cilansetron 2 mg TID showed higher. incidence of improvement from Baseline compared to placebo and when adjusted to those who worsened from Baseline, the difference between treatment groups was even greater. Similar comparisons between cilansetron 2 mg TID and placebo were seen for the Endpoint values.

A summary of deterioration of effect for Study S241.3.009 based on the IBS-QOL Overall Score is presented in Table 2.7.3:53 and Table 2.7.3.6.15.2. At Month 6, the percentage of subjects who worsened from Baseline in cilansetron 2 mg TID was 5% compared to 10% in placebo. Cilansetron 2 mg TID showed higher incidence of improvement from Baseline compared to placebo and when adjusted to those who worsened from Baseline, the difference between treatment groups was even greater. Similar comparisons between cilansetron 2 mg TID and placebo were seen for the Endpoint values.

TABLE 2.7.3:53
Deterioration of Effect Analyses: IBS-QOL Overall Score (Observed
Value)

| Percentage of | S241.3.006 | | S241.3.009 | |
|---|---|---|---|---|
| Subjects Who Worsened | Cilansetron | | Cilansetron | |
| from Baseline | 2 mg TID | Placebo | 2 mg TID | Placebo |
| Month 3 | 5% | 10% | — | — |
| Month 6 | — | — | 5% | 10% |
| Endpoint | 6% | 11% | 5% | 12% |

Note:
“Worsened” is defined as the difference between the post-Baseline and the Baseline overall score ≦ −7.
Note:
Endpoint is defined as the last post-Baseline overall score.
Note:
Percentages are based on the number of subjects in the ITT population with non-missing Baseline and post-Baseline overall scores.
Data Source: Study S241.3.006: Table 2.7.3.6.15.1 Left. Vol. 9 Righ; Study S241.3.009: Table 2.7.3.6.15.2 Left. Vol. 9 Righ

Analysis of Clinical Information Relevant to Dosing Recommendations

The justification for the 2 mg TID cilansetron dose originates from three trials: S241.2.112, S241.2.113, and S241.3.001. Studies S241.2.112 and S241.2.113 were placebo-controlled dose response studies in non-constipated IBS subjects satisfying the Rome criteria (1992) with minimum abdominal pain/discomfort, stool frequency and consistency determined during the run-in period. Note that there were no criteria for diarrhea-predominant IBS at the time of these studies. The relevance of these data for diarrhea-predominant IBS subjects is supported by the high proportion (approximately 80%) of subjects fulfilling the criteria for diarrhea-predominant IBS determined by applying the Rome II criteria to the Rome I responses a posteriori.

Efficacy results from Study S241.2.112 with a total of 490 subjects randomized, showed that cilansetron in doses of 1 mg and 16 mg TID, and to a lesser but clinically meaningful extent in doses of 2 mg and 8 mg TID, is efficacious in achieving adequate relief of IBS symptoms (abdominal pain/discomfort, abnormal bowel habits) in non-constipated IBS subjects. In addition, trends towards improvement in abdominal pain reduction and improvement in bowel habits (stool frequency and consistency) were also noted, mainly with the 2 mg TID cilansetron treatment. Similarly, efficacy results from Study S241.2.113, with a total of 485 subjects randomized, showed statistically significant adequate relief of IBS symptoms (abdominal pain/discomfort, abnormal bowel habits), as assessed by subject response, in the cilansetron 2 mg TID group (22% better than placebo). Dosages of 1 mg, 4 mg, and 16 mg TID resulted in responder rates of 11%, 8%, and 18% higher than found with placebo, but statistical significance was not achieved at these doses. Furthermore, the 2 mg TID cilansetron dose showed the largest improvement on the daily assessment of abdominal pain. Cilansetron increased stool firmness, especially with the 4 mg and 16 mg TID dosages, and decreased stool frequency with no apparent effect difference between the dosages.

Safety results from Study S241.2.112 and Study S241.2.113 showed that cilansetron was safe and well-tolerated in the population investigated. Constipation was also the only dose-dependent AE up to the 4 mg dose (Study S241.2.113) or 8 mg dose (Study S241.2.112), although the incidence of constipation for the 16 mg TID group was still higher compared with the placebo group and the 2 mg TID group in both studies.

Based on the Phase II dose-ranging studies, it was decided to start the initial Phase HI program with 16 mg TID and 2 mg TID. Study S241.3.001 (discussed above) was the first study of the initial Phase III program. This was a double-blind, placebo-controlled, randomized, multicenter study in non-constipated IBS subjects who were treated with cilansetron 16 mg TID or placebo for 12 weeks. However, the study was terminated prematurely by the sponsor. Blood samples were collected during periodic study visits in a subset of subjects for the evaluation of the pharmacokinetics of cilansetron and its 4-hydroxy metabolites. Due to the early termination and the small number of subjects completed, no pharmacokinetic or efficacy analyses were performed.

Persistence of Efficacy and/or Tolerance Effects

Efficacy-data over a treatment period of six months were provided by the double-blind, placebo controlled, randomized, multinational pivotal efficacy. This study provided evidence that the beneficial effect of cilansetron 2 mg TID remained stable throughout the six months of treatment. There were no tolerance effects of cilansetron 2 mg TID detected over a treatment period of six months.

What is claimed is:

1. A method of treatment of diarrhea-predominant IBS in a subject, comprising:
administering a composition comprising cilansetron or a pharmaceutically acceptable derivative thereof to achieve a plasma cilansetron concentration between about 0.1 ng/mL and about 25 ng/mL.

2. The method of claim 1, wherein the plasma cilansetron concentration is achieved between about 0.01 hours and about 18 hours following the administering of the composition.

3. The method of claim 1, wherein the composition comprises about 2 mg of cilansetron.

4. The method of claim 1, wherein the composition is administered three or more times daily.

5. The method of claim 1, wherein the composition comprises:
(i) about 1.5 mg to about 3 mg cilansetron.HCl.H₂0;
(ii) about 40 mg to about 60 mg corn starch;
(iii) about 70 mg to about 100 mg mannitol;
(iv) about 3 mg to about 7 mg povidone;
(v) about 0.05 mg to about 1 mg citric acid monohydrate;
(vi) about 1 mg to about 5 mg crospovidone;
(vii) about 0.05 mg to about 2 mg colloidal silica; and
(viii) about 1 mg to about 3 mg stearic acid.

6. A method of treatment of diarrhea-predominant IBS in a subject, comprising:
administering a sufficient amount of a composition comprising cilansetron or a pharmaceutically acceptable derivative thereof to reach a mean plasma cilansetron concentration across a statistically significant population of subjects of between about 0.1 ng/mL and about 25 ng/mL.

7. The method of claim 6, wherein the mean plasma concentration across a statistically significant population of subjects is achieved in a mean time between about 0.01 hours and about 18 hours following the administering of the composition to the subject.

8. A method of treatment of diarrhea-predominant IBS in a subject, comprising:
administering a daily dose of a composition comprising cilansetron or a pharmaceutically acceptable derivative thereof in an amount sufficient to substantially maintain a plasma cilansetron concentration of between about 0.1 ng/mL and about 25 ng/mL.

9. The method of claim 8, wherein the daily dose is sufficient to maintain the plasma cilansetron concentration, after at least 7 consecutive days of administration, for at least 12 hours after the subject has been administered the daily dose.

10. A method for treatment of nonconstipated IBS in subject, comprising:
administering a composition comprising cilansetron or a pharmaceutically acceptable derivative thereof to achieve a plasma cilansetron concentration between about 0.1 ng/mL and about 25 ng/mL.

11. The method of claim 10, wherein the plasma cilansetron concentration is achieved between about 0.01 hours and about 18 hours following the administering of the composition.

12. A method for treatment of nonconstipated IBS in subject, comprising:
administering a sufficient amount of a composition comprising cilansetron or a pharmaceutically acceptable derivative thereof to reach a mean plasma cilansetron concentration across a statistically significant population of subjects-of between about 0.1 ng/mL and about 25 ng/mL.

13. The method of claim 12, wherein the mean plasma concentration across a statistically significant population of subjects is achieved in a mean time between about 0.01 hours and about 18 hours following the administering of the composition to the subject.

14. A method for treatment of nonconstipated IBS in subject, comprising:
administering a daily dose of a composition comprising cilansetron or a pharmaceutically acceptable derivative thereof in an amount sufficient to substantially maintain a plasma cilansetron concentration of between about 0.1 ng/mL and about 25 ng/mL.

15. The method of claim 14, wherein the daily dose is sufficient to maintain the plasma cilansetron concentration, after at least 7 consecutive days of administration, for at least 12 hours after the subject has been administered the daily dose.

16. A method of improving quality of life in a subject having diarrhea-predominant IBS, comprising:
administering a composition comprising cilansetron or a pharmaceutically acceptable derivative thereof to achieve a plasma cilansetron concentration between about 0.1 ng/mL and about 25 ng/mL.

17. A method of improving quality of life in a subject having diarrhea-predominant IBS, comprising:
administering a sufficient amount of a composition comprising cilansetron or a pharmaceutically acceptable derivative thereof to reach a mean plasma cilansetron concentration across a statistically significant population of subjects of between about 0.1 ng/mL and about 25 ng/mL.

18. A method of improving quality of life in a subject having diarrhea-predominant IBS, comprising:
administering a daily dose of a composition comprising cilansetron or a pharmaceutically acceptable derivative thereof in an amount sufficient to substantially maintain a plasma cilansetron concentration of between about 0.1 ng/mL and about 25 ng/mL.

19. A method of treatment of diarrhea-predominant IBS in a subject receiving selective serotonin reuptake inhibitor (SSRI) therapy, comprising:
administering a composition comprising cilansetron or a pharmaceutically acceptable derivative thereof to achieve a plasma cilansetron concentration between about 0.1 ng/mL and about 25 ng/mL.

20. The method of claim 19, wherein the SSRI is selected from paroxetine and fluvoxamine.
